SEQUENCE LISTING (15 sequences)

SEQ ID NO: 1 - DNA, 20 bp, Artificial Sequence
  Synthetic Polynucleotide
ctgatcaacatgtacctgcc

SEQ ID NO: 2 - DNA, 22 bp, Artificial Sequence
  Synthetic Polynucleotide
ccgtcttctcctccttgtcacc

SEQ ID NO: 3 - DNA, 21 bp, Artificial Sequence
  Synthetic Polynucleotide
ctcggccacagagtactccac

SEQ ID NO: 4 - DNA, 22 bp, Artificial Sequence
  Synthetic Polynucleotide
tgaactacgacaagctgagccg

SEQ ID NO: 5 - DNA, 22 bp, Artificial Sequence
  Synthetic Polynucleotide
tccttggggaagagcaaaagtg

SEQ ID NO: 6 - DNA, 20 bp, Artificial Sequence
  Synthetic Polynucleotide
gccattaccactagacgggg

SEQ ID NO: 7 - DNA, 22 bp, Artificial Sequence
  Synthetic Polynucleotide
agatccagctgtggcagtttct

SEQ ID NO: 8 - DNA, 22 bp, Artificial Sequence
  Synthetic Polynucleotide
accagacaaggattgagggagc

SEQ ID NO: 9 - DNA, 22 bp, Artificial Sequence
  Synthetic Polynucleotide
cgtgcatctggaaagctacgtg

SEQ ID NO: 10 - DNA, 22 bp, Artificial Sequence
  Synthetic Polynucleotide
cttgaagaagtcgtggcgcttc

SEQ ID NO: 11 - DNA, 20 bp, Artificial Sequence
  Synthetic Polynucleotide
ggttcttgactaccgtaatt

SEQ ID NO: 12 - DNA, 20 bp, Artificial Sequence
  Synthetic Polynucleotide
ggggccactagggacaggat

SEQ ID NO: 13 - DNA, 70 bp, Artificial Sequence
  Synthetic Polynucleotide
atgagacagagcggcgcctcccagcccctgctgatcaacatgtacctgccaggtaccagggaccagccgg

SEQ ID NO: 14 - DNA, 69 bp, Artificial Sequence
  Synthetic Polynucleotide
atgagacagagcggcgcctcccagcccctgctgatcaacatgtaccgccaggtaccagggaccagccgg

SEQ ID NO: 15 - DNA, 71 bp, Artificial Sequence
  Synthetic Polynucleotide
atgagacagagcggcgcctcccagcccctgctgatcaacatgtaccttgccaggtaccagggaccagccgg

(19) United States
(12) Patent Application Publication
(10) Pub. No.: US 2021/0340499 A1
(43) Pub. Date: Nov. 4, 2021

(54) Production and Enrichment of Pancreatic Endocrine Progenitor Cells

(71) Applicant: THE REGENTS OF THE UNIVERSITY OF CALIFORNIA, Oakland, CA (US)

(72) Inventors: Julie Beth Sneddon, San Francisco, CA (US); Lauren E. Byrnes, San Francisco, CA (US); Daniel M. Wong, San Francisco, CA (US)

(21) Appl. No.: 17/280,076

(22) PCT Filed: Sep. 25, 2019
(86) PCT No.: PCT/US19/52958

§ 371 Date: Mar. 25, 2021

Related U.S. Application Data
(60) Provisional application No. 62/736,237, filed on Sep. 25, 2018.

(51) Int. Cl. C12N 5/071; G01N 33/50

(52) U.S. Cl. CPC C12N 5/0678; G01N 2333/575; G01N 33/507

(57) ABSTRACT

The disclosure provides methods for enriching for pancreatic endocrine progenitor cells, such as human pancreatic endocrine progenitor cells, including alpha cell progenitors, beta cell progenitors, delta cell progenitors, PP cell progenitors and epsilon cell progenitors. The disclosure provides mammalian, such as human, Fev⁺ pancreatic endocrine progenitor cells, including Fev⁺ alpha cell progenitors, Fev⁺ beta cell progenitors, Fev⁺ delta cell progenitors, Fev⁺ PP cell progenitors, and Fev⁺ epsilon cell progenitors. The disclosure further provides methods for producing or inducing such cells, including in vitro differentiation methods, and the cells so produced.

### CROSS-REFERENCE TO RELATED APPLICATION

This application claims the priority benefit under 35 U.S.C. § 119(e) of U.S. Provisional Patent Application No. 62/736,237, filed Sep. 25, 2018, the disclosure of which is incorporated herein by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “53514A_Seqlisting.txt”, which was created on Sep. 24, 2019 and is 3,474 bytes in size. The subject matter of the Sequence Listing is incorporated herein in its entirety by reference.

### FIELD

The disclosure relates generally to the fields of cell biology and organogenesis, and more particularly, to methods of generating or enriching for pancreatic endocrine progenitor cells, such as those progenitors that give rise to beta cells capable of controlled insulin production.

### BACKGROUND

Pancreatic organogenesis is a complex and dynamic process that ultimately results in the generation of multiple cell lineages that perform the functions of the mature organ: the regulation of glucose homeostasis by the endocrine compartment and the production of digestive enzymes by the exocrine compartment. In the mouse, all known epithelial lineages of the pancreas derive from a small field of epithelial precursor cells within the foregut endoderm specified by the expression of pancreatic duodenal transcription factor 1 (Pdx1) (FIG. 1a)¹. These Pdx1⁺ cells evaginate into a cap of surrounding mesenchymal cells around embryonic day 9 (E9), proliferate, and begin the process of branching morphogenesis. Further epithelial lineage diversification continues with the specification of Pdx1⁺ cells into tip and trunk domains by E12 and progresses to the restriction of tip cells to a digestive enzyme-producing acinar fate and of trunk cells to either a ductal or endocrine cell fate¹. Within the trunk domain, induction of Neurogenin 3 (Ngn3) expression defines the cells that will differentiate into one of five endocrine lineages: alpha, beta, delta, gamma, or epsilon, as well as the recently described gastrin⁺ cells^{1,2}. Despite previous work focused on the formation of the endocrine compartment, the precise timing and coordination of lineage decisions are not completely understood. Furthermore, it is unknown if there exist additional intermediate progenitor states through which endocrine cells transit along their trajectory to becoming differentiated, hormone-expressing cells.

Although the pancreatic mesenchyme is required for the proper differentiation, proliferation, and morphogenesis of the epithelial network¹, little is known about the cell identities and lineages that compose the pancreatic mesenchyme during development. Even less is known about the mechanisms by which these distinct mesenchymal cell types interact with one another and with the cells of the epithelial compartment during development and in the adult organ. Therefore, a deeper understanding of the full diversity of the mesenchymal cell types, as well as their global gene expression profiles, will serve as the basis for understanding these key cellular interactions.

The successful production of glucose-sensitive, insulin-producing beta cells was a major leap forward, but there are several key limitations that remain before hESC-derived beta cells can be used as a therapeutic intervention for diabetes. First, the number of beta cells required for transplantation into one diabetic patient is on the order of one billion (Jacobson and Tzanakakis, 2017; Lock and Tzanakakis, 2007). Although current hESC-derived beta cell differentiation protocols are capable of differentiating millions of hESCs at a time, the efficiency of generating beta cells is batch-dependent and low, at approximately 30-40% (Pagliuca et al., 2014; Rezania et al., 2014; Russ et al., 2015) and even reported purification methods are low-throughput and labor-intensive (Nair et al., 2019; Veres et al., 2019).

Improvements in the efficiency of functional beta cell production are needed for these regenerative-based therapies to be scalable and of consistent quality. These challenges in beta cell functionality and differentiation efficiency in hESC-derived beta cell differentiation protocols can be addressed by better understanding the gene expression programs that drive beta cell differentiation and maintain beta cell identity in vivo. Currently available methods may be failing to activate other transcriptional programs not yet uncovered to be required for proper beta cell differentiation and function. Additionally, although current differentiation protocols for generating hESC-derived beta cells recapitulate key developmental stages and genetic programs used to make beta cells in vivo, the programs may not fully mimic the exact developmental path taken in vivo in in vitro approaches.

Thus, existing directed differentiation protocols to generate beta cells from hESCs fail to produce sufficient numbers of functional beta cells that maintain glucose-sensing, insulin-secreting capabilities over time. Therefore, a need continues to exist in the art for pancreatic beta cells capable of controllable or regulated insulin production and methods of producing such pancreatic beta cells, as well as methods of enriching for such beta cells, including autologous pancreatic beta cells.

### SUMMARY

Disclosed herein is the discovery of an endocrine progenitor cell population, marked by differential expression of a transcription factor named Fev (or Pet1), that gives rise to endocrine cells, including insulin-producing beta cells, in mouse pancreatic development. This Fev⁺ population has also been identified in human fetal pancreata during stages at which beta cell differentiation occurs, indicating that this Fev⁺ population is relevant to not only mouse, but also human beta cell development. In addition, we have found a Fev-expressing cell population present in our in vitro platform for performing directed differentiation of human embryonic stem cells (hESCs) to insulin-producing beta cells. Also disclosed herein is a Fev-reporter hESC line useful in enriching for this Fev⁺ endocrine progenitor population during directed differentiations of hESCs to beta cells.

The findings disclosed herein have been extended to the postnatal period, when beta cells undergo massive expansion, and when we also identified FevHigh (Fev^{HI}) cells in pancreatic islets. Although the current model in the field is that new beta cells arise by duplication of pre-existing beta cells, we have demonstrated with genetic fate mapping experiments that these FevHigh islet cells give rise to insulin-producing beta cells. This raises the distinct possibility that the Fev^{HI} post-natal endocrine progenitor population may represent a novel source of beta cells after birth—during homeostasis and/or during injury or disease.

Recent studies of late embryonic, postnatal, and adult alpha and beta cells have demonstrated the power of single-cell transcriptomic profiling for unraveling endocrine lineage heterogeneity and revealing distinct transcriptional states of beta cell maturation³⁻⁵. Here, we perform droplet-based, single-cell RNA sequencing of entire murine embryonic pancreata at earlier developmental time points to describe the cellular diversity and dynamics of gene expression in both the epithelial and mesenchymal compartments. We further validate the existence of novel populations within mouse and human pancreatic tissue, as well as human embryonic stem cell (hESC)-derived endocrine progenitor cells. Finally, we predict novel lineage relationships, identify previously unappreciated intermediate progenitor cells, and validate our methodology using in vivo genetic lineage tracing.

In one aspect, the disclosure provides a method of enriching the pancreatic endocrine progenitor cell population in a cell sample comprising (a) detecting cells in the sample expressing a pancreatic endocrine progenitor cell marker; and (b) separating a pancreatic endocrine progenitor cell from at least one cell that does not express the pancreatic endocrine progenitor cell marker, thereby enriching the pancreatic endocrine progenitor cell population of the cell sample. In some embodiments, the pancreatic endocrine progenitor cell is a human cell. In some embodiments, the pancreatic endocrine progenitor cell is an alpha cell progenitor, a beta cell progenitor, a delta cell progenitor, a PP cell progenitor, or an epsilon cell progenitor. In some embodiments, the pancreatic endocrine progenitor cell marker is the E26 transformation-specific transcription factor Fev. In some embodiments, the pancreatic endocrine progenitor cell is a beta cell progenitor, such as a Fev⁺ beta cell progenitor. In some embodiments, the Fev⁺ beta cell progenitor further comprises Gng12⁺, Tssc4⁺, Ece1⁺, Tmcm108⁺, Wipi1⁺, or Papss2⁺. In some embodiments, the beta cell progenitor is Fev⁺, Gng12⁺. In some embodiments, the Fev⁺ beta cell progenitor further comprises Pax4⁺, Chga⁺, Chgb⁺, Neurod1⁺, Runx1t1⁺, or Vim⁺. In some embodiments, the Fev⁺ beta cell progenitor does not express detectable Ngn3, Ins1 or Gcg. In some embodiments, the beta cell progenitor is Fev⁺, Ngn⁻. In some embodiments, the Fev⁺, Ngn⁻ beta cell progenitor expresses a gene in the serotonin pathway, the insulin signaling pathway, sphingosine-1-phosphate signaling pathway, or Activating Transcription Factor-2. In some embodiments, the Fev⁺ beta cell progenitor further comprises Pdx1⁺ or Mafb⁺. In some embodiments, the at least one cell that does not express the pancreatic endocrine progenitor cell marker is a CD140⁺ mesenchyme cell. In some embodiments, the beta cell progenitor cell is a human cell.

Another aspect of the disclosure is a method of producing a pancreatic endocrine progenitor cell comprising culturing a stem cell under conditions that induce differentiation of the stem cell into a pancreatic endocrine progenitor cell. In some embodiments, the stem cell is an embryonic stem cell (ESC) or an inducible pluripotent stem cell (iPSC). In some embodiments, the pancreatic endocrine progenitor cell is an alpha cell progenitor, a beta cell progenitor, a delta cell progenitor, a PP cell progenitor, or an epsilon cell progenitor. In some embodiments, the pancreatic endocrine progenitor cell marker is the E26 transformation-specific transcription factor Fev, such as a Fev⁺ beta cell progenitor. In some embodiments, the pancreatic endocrine progenitor cell is a beta cell progenitor. In some embodiments, the Fev⁺ beta cell progenitor further comprises Gng12⁺, Tssc4⁺, Ece1+, Tmcm108⁺, Wipi1⁺, or Papss2⁺. In some embodiments, the beta cell progenitor is Fev⁺, Gng12⁺. In some embodiments, the Fev⁺ beta cell progenitor further comprises Pax4⁺, Chga⁺, Chgb⁺, Neurod1⁺, Runx1t1⁺, or Vim⁺. In some embodiments, the Fev⁺ beta cell progenitor does not express detectable Ngn3, Ins1 or Gcg. In some embodiments, the beta cell progenitor is Fev⁺, Ngn⁻. In some embodiments, the Fev⁺, Ngn⁻ beta cell progenitor expresses a gene in the serotonin pathway, the insulin signaling pathway, sphingosine-1-phosphate signaling pathway, or Activating Transcription Factor-2. In some embodiments, the Fev⁺ beta cell progenitor further comprises Pdx1⁺ or Mafb⁺. In some embodiments, the Fev⁺ beta cell progenitor is a human cell.

Yet another aspect of the disclosure is an isolated Fev⁺ pancreatic endocrine progenitor cell produced according to the methods disclosed herein. An exemplary Fev⁺ pancreatic endocrine progenitor cell produced according to the methods disclosed herein is an isolated Fev⁺ beta cell progenitor.

Still another aspect of the disclosure is a method of inducing formation of a hormone-producing cell comprising contacting a progenitor of a hormone-producing cell with an effective amount of Fev to produce a hormone-producing cell. In some embodiments, the hormone-producing cell is an INS+ cell. In some embodiments, the hormone-producing progenitor cell is an ES4 cell. In some embodiments, the hormone-producing cell is a beta cell. In some embodiments thereof, the hormone-producing progenitor cell is a beta-like cell, for example a beta-like cell at the end stage of the directed differentiation of hESCs to the beta cell lineage. In some embodiments, the method is performed in vitro. In some embodiments, the method further comprises removing a cell expressing at least one of PHOX2A, TLX2 or TBX2.

Another aspect of the disclosure is a method of screening for a signaling compound that induces FEV+ progenitor cell replication comprising: (a) contacting a FEV+ progenitor cell with a candidate compound; (b) culturing the FEV+ progenitor cell under conditions suitable for cell proliferation; (c) measuring the cell proliferation of the FEV+ progenitor cell in the presence or absence of the candidate compound; and (d) identifying the compound as a signaling compound for FEV+ progenitor cell proliferation if the cell proliferation in the presence of the compound is greater than the cell proliferation in the absence of the compound. In some embodiments, the FEV+ progenitor cell is a FEV-MYC progenitor cell, a FEV-GFP progenitor cell, a FEV-KO progenitor cell, or a FEV-tNFGR progenitor cell.

In still another aspect, the disclosure provides a method of screening for a signaling compound that enhances FEV+ progenitor cell differentiation into beta cells comprising: (a) contacting FEV+ progenitor cells with a candidate compound; (b) incubating the FEV+ progenitor cells under conditions suitable for cell differentiation; (c) measuring the level of differentiation of the FEV+ progenitor cells to beta cells in the presence or absence of the candidate compound; and (d) identifying the compound as a signaling compound for FEV+ progenitor cell differentiation into beta cells if the cell differentiation in the presence of the compound is greater than the cell differentiation in the absence of the compound. In some embodiments, the FEV+ progenitor cell is a FEV-MYC progenitor cell, a FEV-GFP progenitor cell, a FEV-KO progenitor cell, or a FEV-tNFGR progenitor cell.

Other features and advantages of the disclosure will be better understood by reference to the following detailed description, including the drawing and the examples.

### BRIEF DESCRIPTION OF THE DRAWINGS

FIG. 1. Single-cell sequencing identifies broad patterns of cellular heterogeneity in E14.5 murine pancreas. (a) Overview of murine pancreatic development. (b) Schematic of experimental approach. (c) t-Distributed Stochastic Neighbor Embedding (t-SNE) visualization of predicted populations from pooled E14.5 mouse pancreata (n=14). Each dot represents the transcriptome of a single cell, color-coded according to its cellular identity (epithelial, mesenchymal, or immune/vascular). Each cell compartment contains multiple sub-populations, represented by varying degrees of color shading. (d) Established marker genes identify epithelial cells (Cdh1⁺), endocrine cells (Chga⁺), mesenchymal cells (Vim⁺ and Col3a1⁺), endothelial cells (Pecam1⁺), and immune cells (Rac2⁺). (e) Heatmap depicting greater than 2-fold differentially expressed genes in each cluster compared to all other clusters. Cells are represented in columns, and genes in rows. Specific genes used to annotate clusters are indicated to the right of the heatmap.

FIG. 2. Identification of multiple novel, uncharacterized mesenchymal populations. (a) t-SNE visualization of subclustered E14.5 mesenchymal clusters (from n=14 pancreata). (b) Density plot depicting Pearson's correlation values (depicted in heatmap in FIG. 10b) within the epithelial and mesenchymal populations based on average gene expression in each cluster. (c) Dot plot of top differentially expressed markers of each mesenchymal population. Bars are color-coded by cluster identity in (a). The grey bar represents pan-mesenchymal markers. The size of each dot represents the proportion of cells within a given population that expresses the gene; the intensity of color indicates the average level of expression. (d) Pathway analysis of genes greater than 2-fold differentially expressed by cells in clusters 1, 2, 4, and 5. (e) Expression of genes marking clusters 1 (Cav1), 2 (Stmn2), 4 (Cxcl12), and 5 (Barx1) in all E14.5 mesenchymal cells. Color intensity indicates level of expression. (f-h) Multiplexed fluorescent ISH combined with Epcam IF validates clusters 2 and 5 (e) and cluster 1 (f-g) predicted by single-cell sequencing. Epcam marks pancreatic epithelium. In (f), Barx1⁺ cells (red arrows, cluster 5) are distinct from Stmn2⁺ cells (green arrows, cluster 2), as predicted by the single-cell data. In (g), Cav1⁺ cells (red arrows, cluster 1) are distinct from Stmn2⁺ cells (green arrows, cluster 2). In (h), Barx1⁺ cells that do not express Cav1 (red arrows) represent cluster 5, whereas Barx1⁺/Cav1⁺ cells (yellow arrows) represent cluster 1. Cav1⁺ cells that do not express Barx1 are also identified (green arrows), likely representing endothelial cells⁸⁰. Scale bar represents 50 um in f-h.

FIG. 3. Mesothelial cells are dynamic over developmental time and are predicted to give rise to vascular smooth muscle populations. (a) t-SNE visualization of merged mesenchymal clusters from E12.5 (n=18 pancreata), E14.5 (n=14 pancreata for batch 1; n=11 for batch 2), and E17.5 (n=8 pancreata) tissue. Mesenchymal clusters were identified at each time point, subclustered, merged together, and reanalyzed. Cells are colored by cluster or time point. Dotted circle highlights time point-segregated mesothelial clusters. (b) Dot plot of top differentially expressed genes in time point-specific mesothelial clusters (clusters 1, 11, and 17). (c) ISH for Pitx2 and Msln in E12.5 and E17.5 pancreata. Pitx2 expression was detected in E12.5 but not E17.5 mesothelium, whereas Msln was detected in E17.5 but not E12.5 mesothelium. Vimentin (Vim) IF staining depicts pancreatic mesenchyme. Dotted line indicates tissue boundary. Yellow arrows identify Pitx2⁺ mesothelial cells. Red arrows identify Msln⁺ mesothelial cells. Scale bar represents 50 um. (d) Expression levels of VSM-related genes in merged mesenchymal clusters. Color intensity indicates level of expression. (e) Pseudotime ordering of mesothelial and VSM-related merged mesenchymal clusters. Colors correspond to t-SNE in (a). All clusters are individually plotted in FIG. 10j. (f) Cluster proportions over pseudotime. Pseudotime was binned into 10 groups and the proportion of each cluster within that bin of pseudotime was calculated. (g) Model of lineage relationships among mesothelial, vascular smooth muscle, and VSM-related mesenchymal populations based on pseudotime ordering in (e).

FIG. 4. Identification of known and novel epithelial cell populations in E14.5 mouse pancreas. (a) t-SNE visualization of epithelial groups only, as defined in FIG. 1. (b) Dot plot depicting known and novel markers of epithelial populations, as well as markers specific to the Fev^{Hi} population. Size of the dot represents proportion of the population that expresses each specified marker. Color indicates level of expression. (c) Expression of Fev and Ngn3 within epithelial cells. Color indicates level of expression. (d) Gene expression comparison between the Ngn3⁺ and Fev^{Hi} population. Genes greater than 2-fold differentially expressed are highlighted in dark blue (higher in Fev^{Hi} cells) or light blue (higher in Ngn3⁺ cells). (e) Pathway analysis of genes greater than 2-fold differentially expressed in Ngn3⁺ and Fee populations (f) t-SNE visualization of the 661 cells of the endocrine lineage (Ngn3⁺, Fev^{Hi}, alpha, beta, and epsilon populations). (g) Pseudotime ordering of Ngn3⁺, Fev⁺/Pax4⁺, Fev^{Hi}, alpha, and beta cell populations place Fev⁺ cells between Ngn3⁺ and hormone populations.

FIG. 5. Fev^{Hi} cells are novel endocrine progenitors. (a) In situ hybridization (ISH) for Ngn3, Fev, and Isl1 in lineage-traced Ngn3-Cre; Rosa26^{mTmG} E14.5 pancreata where Ngn3 lineage-traced cells are mGFP⁺. Gray arrowheads identify Ngn3⁺ cells, presumably not yet Ngn3-lineage labeled due to the transient nature of Ngn3 expression and the delay of Cre-mediated recombination that permits expression of mGFP. Blue arrowheads identify Ngn3+/Fev⁺ cells that are Ngn3-lineage-traced. Yellow arrowheads identify Ngn3-lineage-traced cells that are Fev⁺ but do not express Ngn3 or Isl1. Purple arrowheads identify Fev⁺/Isl1⁺ cells that are Ngn3-lineage-traced. Magenta arrowheads identify Isl1⁺ cells that are Ngn3-lineage-traced. (b-c) Dual ISH/immunofluorescence (IF) for NGN3 and FEV mRNA and CHGA protein in human fetal pancreas at 23 weeks of gestation (n=1 pancreas). Gray arrowheads identify NGN3⁺ cells. Yellow arrowheads identify FEV⁺ cells. Purple arrowheads identify FEV⁺/ISL1⁺ cells. Magenta arrowheads identify ISL1⁺ cells. (d) Multiplexed fluorescent ISH for NGN3, FEV, and ISL1 mRNA in hESC-derived endocrine progenitor cells. Blue arrowheads identify NGN3⁺/FEV⁺ cells. Yellow arrowheads identify FEV⁺ cells. Purple arrowheads identify FEV⁺/ISL1⁺ cells. (e) Quantification of each population detected in (a) as percentage of Ngn3-lineage-traced cells (n=464 cells, 6 pancreata). Data are represented as mean+standard deviation (SD). (f) Quantification of each population detected in (d) as a percentage of total stained cells (n=418 cells). Data are represented as mean+SD. Analysis on differentiation of hESCs was performed once. (g) Proposed model for the derivation of Fev^{Hi} endocrine cells from Ngn3⁺ cells, and their differentiation into hormone⁺/Fev^{Lo} endocrine cells. Colors of arrowheads and bars in a-f correspond to cell identity in g. (a and d) Scale bar: 10 um. (b and c) Scale bar: 20 um. (h) t-SNE visualization of v2 merged endocrine time course (E12.5, E14.5, aggregated E17.5). Clusters are annotated based on correlation with v1 dataset or top differentially expressed genes. (i) Time point labels for v2 merged endocrine time course data. t-SNE is the same as FIG. 5h. (j) Cell type proportions at each time point, calculated from the clusters depicted in FIG. 5h.

FIG. 6. Differentiated, hormone⁺ endocrine cells transit through a Fev-expressing stage during pancreatic development. (a-e) Dual IF (for membrane GFP) and fluorescent ISH for all major hormones in Fev-Cre; ROSA26^{mTmG} lineage traced animals at E14.5. n=46 cells of 4 pancreata for Ins1 (100% labeled-lineage); n=103 of 4 pancreata cells for Gcg (100% lineage-labeled); n=6 cells of 4 pancreata for Sst (100% lineage-labeled); n=21 cells of 4 pancreata for Ghrl/Gcg (23.8% lineage-labeled); n=64 cells of 4 pancreata for Ppy (89.1% lineage-labeled). Scale bar represents 10 um. (f) Schematic of E14.5 Fev-Cre; ROSA26^{mTmG} FACS sorting and single-cell RNA sequencing. (g) Representative FACS plots of sorted single, live GFP+ and TdTomato⁺/GFP⁻ cells from dissociated pancreata used for single-cell sequencing. (h) t-SNE visualization of endocrine cells in Fev⁻ lineage traced E14.5 mouse pancreata (n=3). (i) Expression of major markers of endocrine cell types. Color indicates level of expression, except for the eGFP plot, which indicates presence or absence of eGFP counts.

FIG. 7. Identification of candidate regulators of beta and alpha cell fate decisions. (a) Pseudotime ordering of the endocrine cells at E14.5 depicted in FIG. 6h yields a bifurcated tree in which the two main branches terminate in cells that highly express Ins1 (beta cell branch) or Gcg (alpha cell branch). (b) Heatmap depicting the expression of genes along each branch, in pseudotime. An independent expression pattern is calculated across the entire pseudotime trajectory for each branch. Therefore, the portion of the trajectory before the branch point is displayed for each branch separately. Genes are clustered based on expression pattern across pseudotime; selected known and novel genes with differential expression along the branches are highlighted to the right. (c) Gene expression plots depicting the kinetic trends along each branch. (d-e) Multiplexed fluorescent ISH for Fev, Gng12, and Islet1 (d) or Fev, Peg10, and Islet1 (e) in lineage-traced E14.5 Ngn3-Cre; ROSA26^{mTmG} pancreas. Arrowheads identify lineage-traced Fev⁺/Islet1⁻ cells with Gng12 (d, teal-graded arrowheads) or Peg10 (e, indigo-graded arrowheads) expression. (f) Multiplexed fluorescent ISH for Fev, Gng12, and Ins1. Teal arrowheads identify lineage-traced Ins1⁺ beta cells that express Gng12. (g) Multiplexed fluorescent ISH for Fev, Peg10, and Gcg. Indigo arrowheads identify lineage-traced Gcg alpha cells that express Peg10. (h) Model for Fev^{HI} (yellow) cell differentiation into distinct alpha or beta cells. Peg10 and Gng12 expression in Fev^{Hi} cells may represent progenitors pre-fated towards the alpha and beta lineages, respectively, during endocrine lineage allocation. (d-g) Scale bars represent 10 um. Blue staining represents DAPI-labeled nuclei. Colors of arrowheads match colors of cells represented in (h).

FIG. 8. Quality control for version 1 single-cell RNA-sequencing runs. (a) Representative FACS plot of single, live cells sorted from dissociated Swiss Webster embryonic pancreata and used for single-cell sequencing. (b) Quality control statistics for all single-cell sequencing runs prepared with the Chromium Single Cell 3′ Reagent Version 1 Kit. The “valid barcodes” metric indicates the percentage of cells with barcodes that match a known barcode contained on a bead. “Mapped reads to transcriptome” refers to the percentage of reads that confidently map to a unique gene in the reference transcriptome. “Fraction Reads in Cells” is the percentage of reads that contain a cell-associated barcode. (c) Cellranger cell calls based on the number of UMIs. The dropoff indicates the threshold for the number of UMIs required for a barcode to be assigned to a cell. (d) Histogram of the number of genes per cell in all single-cell runs pre-filtering steps. (e) Histogram of the number of genes per cell in all single-cell runs post-filtering steps. E17.5 Batch 2 contained a large number of red blood cells, which expressed fewer than 200 genes, resulting in their removal during minimum gene threshold filtering (see Example 1).

FIG. 9. Single-cell RNA-sequencing batch information from E14.5 pancreata. (a) Selection of variable genes in the E14.5 v1 dataset (all cells) by Seurat's MeanVarPlot function. (b) t-SNE visualization of merged E14.5 batches, color-coded by batch. Batch 1 and 2 contribute to all clusters, reflecting a successful batch correction. (c) Pearson's correlation of E14.5 batch 1 cells with E14.5 batch 2 cells within each cluster based on average expression of variable genes. Batch 1 cells correlate most highly with batch 2 cells within the same cluster, indicating proper merging of the two batches. (d) Cell type proportions in E14.5 batch 1 and 2 with exocrine (acinar and ductal) clusters included (top panel) and excluded (bottom panel). All cell types except the exocrine compartment show high correlation between the two batches. (e) Pearson's correlation between clusters from the E14.5 batch 1 full dataset and those from the E14.5 batch 1 dataset downsampled to 50% of the reads, based on average expression of shared variable genes. (f) Maintenance of the number of median genes/cell after random downsampling of reads, indicating sufficient sequencing depth. (g) Maintenance of cluster structure after random downsampling of UMIs is reflected by the similar percentage of cells found within the same cluster with fewer UMIs.

FIG. 10. Transcriptional signatures and lineage dynamics among mesenchymal populations. (a) t-SNE visualization of E14.5 v1 biological replicates, colored by batch, demonstrating effectiveness of batch correction across mesenchymal cells. (b) Pearson's correlation of E14.5 epithelial and mesenchymal clusters based on average expression of variable genes. (c) Comparison of bimodal likelihood ratio test adjusted p-values to adjusted p-values calculated by either MAST (left panel) or Wilcoxon rank sum (right panel) tests for all greater than 2-fold differentially-expressed genes. Pearson's correlation value is shown in top left corner. (d, e) IF validation of (d) mesothelium (Wt1+) and (e) vascular smooth muscle (Acta2+) cells in E14.5 pancreata. Ecadherin marks epithelium, and Vimentin (Vim) marks mesenchyme. Scale bar: 50 um. (f) Expression of secreted factors by the mesothelium. Color intensity indicates level of expression. (g) t-SNE visualization of merged mesenchymal time course dataset. E14.5 biological replicates are colored, serving as a measure of batch correction effectiveness within the merged mesenchymal time course dataset. Grey dots represent both E12.5 and E17.5 cells. (h) Correlation of E14.5 mesenchymal populations with merged (E12.5, E14.5 and E17.5) mesenchymal clusters based on average expression of the variable genes from all datasets. Merged populations were matched with E14.5 (FIG. 2) by highest correlation and assigned the same cluster identity (cluster 1-10). Remaining merged clusters were assigned cluster identities 11-17. (i) Dot plot of differentially expressed genes from each merged mesenchymal cluster. The size of each dot represents the proportion of cells within a given population that expresses the gene; the intensity of color indicates the average level of expression. Colored bars correspond to t-SNE in FIG. 3a (j). Contribution of cells from each time point is mapped onto pseudotime plots. Expression of proliferation markers, Birc5 and Top2a, in the pseudotime trajectory. Color indicates level of expression. Contribution of cells from each time point is broken down by individual cluster and mapped onto pseudotime plots. Colors correspond to cell clusters in FIG. 3a,e.

FIG. 11. Identification of epithelial cell populations in E14.5 pancreas. (a) t-SNE visualization of E14.5 v1 epithelial batches, colored by batch. Significant overlap, and clusters that include cells from both batches, reflects successful batch correction. (b) Comparison of bimodal likelihood ratio test adjusted p-values to adjusted p-values calculated by either MAST (left panel) or Wilcoxon rank sum (right panel) tests for all greater than 2-fold differentially expressed genes. Pearson's correlation value is shown in the top left corner. (c) Expression maps of Ppy and Sst hormones within E14.5 epithelial dataset. (d) Dot plot of endocrine lineage genes across the epithelial populations. The size of each dot represents the proportion of cells within a given population that expresses the gene; the intensity of color indicates the average level of expression. (e) Heatmap depicting genes over 2-fold differentially-expressed in Ngn3⁺ and Fev⁺ populations. Differentially expressed genes were determined from the endocrine dataset depicted in FIG. 4f and only Ngn3⁺ and Fev⁺ populations are shown in the heatmap. (f) Expression of selected markers of early- and late-Fev⁺ populations in all endocrine cells. (g) Pseudotime ordering of Ngn3⁺, Fev⁺/Pax4⁺, FevHi, alpha, and beta cell populations, colored by batch. (h) Expression of Islet1 (Isl1) in E14.5 epithelial cells is largely confined to hormone+ populations. (i) Quantification of FEV expression by quantitative RT-PCR in pluripotent hESCs, mid- and late-stage endocrine progenitor cells, beta-like cells (BLCs), and adult human islets. FEV expression is normalized to GAPDH. Error bars represent standard deviation. N.D=not detected. Bars represent average of three technical replicates from one hESC differentiation.

FIG. 12. Epithelial populations over developmental time. (a) t-SNE visualization of merged version 1 epithelial clusters from E12.5 (n=18 pancreata), E14.5 (n=14 pancreata for batch 1; n=11 for batch 2), and E17.5 (n=8 pancreata). All panels depict the same t-SNE plot. In the far-left panel, cluster identity is denoted by different colors. In the three remaining panels, cells from each indicated time point are represented by black dots; all cells from the other time points are gray. (b) FACS plots depicting negative selection against CD140a from E12.5 (n=14), E14.5 (n=13), and E17.5 (n=13) pancreata. CD140a-negative cells were used for single-cell sequencing. (c) Quality control statistics for 10× Chromium version 2 single-cell RNA sequencing runs, referred to as v2 datasets. Two technical replicates of E17.5 cells were run from the same pancreata on two separate wells on the 10× Chromium machine. The two E17.5 runs were aggregated and analyzed as one dataset. (d) Individual t-SNE plots of v2 E12.5, E14.5, and E17.5 (aggregated) exocrine dataset. Clusters were annotated based on gene expression. (e) Individual t-SNE plots of v2 E12.5, E14.5, and E17.5 (aggregated) endocrine dataset. Clusters are annotated based on correlation with v1 datasets and differentially expressed genes. (f) Pearson's correlation among clusters from v1 merged endocrine time course and v2 merged endocrine time course. (g) Dot plot of top differentially expressed genes for clusters in the v2 merged endocrine dataset. The size of each dot represents the proportion of cells within a given population that expresses the gene; the intensity of color indicates the average level of expression. Clusters correspond to those depicted in t-SNE in FIG. 5h.

FIG. 13. Lineage tracing of Fev-expressing cells in E17.5 mouse pancreata in vivo. (a-d) Representative images showing immunofluorescence (IF) for hormones Ins1 (100% lineage-labeled), Gcg (100% lineage-labeled), Sst (96.7% lineage-labeled), and Ppy (100% lineage-labeled) in Fev-Cre; ROSA26mTmG lineage-traced embryos at E17.5 (total n=86 cells from 5 pancreata for Ins1; n=57 cells from 5 pancreata for Gcg; n=30 cells from 5 pancreata for Sst; n=47 cells from 5 pancreata for PP). (e) Multiplexed IF (for membrane-GFP) and fluorescent ISH for Ghrl and Gcg in Fev-Cre; ROSA26mTmG lineage-traced embryos at E17.5 (total n=23 cells of 2 pancreata). Ghrl⁺/Gcg⁻ cells (47.8% lineage-labeled) represent the epsilon population. Non-lineage-labeled epsilon cells are denoted by the arrowheads, and lineage-labeled epsilon cells are denoted by the arrows. Scale bar represents 10 urn in a-e.

FIG. 14. Lineage tracing of Fev-expressing cells in adult mouse pancreata in vivo. (a-d) Representative IF for adult hormones in 6-week Fev-Cre; ROSA26^{mTmG} lineage-traced pancreas. From two animals: n=407 cells for Ins1 (99.6% lineage-labeled); n=120 cells for Gcg (99.0% lineage-labeled); n=116 cells for Sst (97.9% lineage-labeled); n=68 cells for PP (100% lineage-labeled). Scale bar represents 10 urn in a-d.

FIG. 15. Identification of candidate genes and pathways enriched along beta and alpha cell lineages. (a) Pseudotime ordering trajectory of v1 time course dataset, including E12.5, E14.5 (batch 1 and batch 2), and E17.5 datasets. (b) Gene expression plots depicting the kinetic curves of individual genes (from FIG. 7b) across pseudotime in the alpha or beta branches. (c) Pathway analysis for clusters of genes from Monocle BEAM analysis. Gene clusters correspond to FIG. 7b. (d) SPRING plots for Fev-lineage traced-dataset, including all endocrine cells. Colors match those in FIG. 6h and FIG. 7a. Expression of selected genes predicted from the BEAM analysis.

FIG. 16. Expression of candidate lineage regulators within the endocrine lineage prior to establishment of alpha or beta cell identity. (a) Multiplex fluorescent ISH for Fev (yellow), Peg10 (cyan), and Ins1 (magenta) in lineage-traced E14.5 Ngn3-Cre; ROSA26^{mTmG} pancreas. Indigo gradient arrows highlight lineage-traced Fev⁺/Peg10⁺ cells that do not express Ins1. Teal arrows highlight Ins1⁺ beta cells that do not express Peg10. (b) Multiplex fluorescent ISH for Fev (yellow), Gng12 (cyan), and Gcg (magenta) in lineage-traced E14.5 Ngn3-Cre; ROSA26^{mTmG} pancreas. Teal gradient arrows highlight lineage-traced Fev⁺/Gng12⁺ cells that do not express Gcg. Indigo arrows highlight Gcg alpha cells that are not enriched for Gng12.

FIG. 17. Single-cell RNA-sequencing identifies diverse cellular compartments in 12wpc human fetal pancreas. (a) UMAP-based clustering of single cells organized into 22 distinct clusters from one 12wpc human fetal pancreas. Each dot represents a single cell and is colored/shaded based on its assigned cluster identity. Plot to the left represents UMAP-based clustering of cells from each of two technical replicate samples run on two different wells of the 10× Chromium single-cell sequencing chip. Each technical replicate shows even contribution of cells to each cluster within the merged UMAP. (b) Expression patterns of known marker genes of epithelial cells (CHD1+), ductal cells (SOX9+), acinar cells (CPA1+), endocrine cells (CHGA+), mesenchymal cells (COLA1A+), endothelial cells (PECAM1+), immune cells (PTPRC+), and nerve (SOX10+) cells, revealing the identities of all 22 clusters from UMAP-based clustering. Intensity of color/shading indicates level of gene expression. (c) Heatmap showing the top 50 differentially-expressed genes in each cluster compared to all other clusters. Individual cells are represented in each column, and columns of cells derived from the same cluster are grouped together. Cluster numbers are consistent with cluster numbering system as shown in (a). Genes are represented by rows.

FIG. 18. Endocrine sub-clustering identifies known and novel cell populations in 12wpc human fetal pancreas. (a) UMAP-based sub-clustering of CHGA+ clusters, as defined in FIG. 17b, organized into 10 distinct populations. Inset shows CHGA+ clusters from FIG. 17b. (b) Known markers of the endocrine lineage, including NGN3, INS, GCG, SST, and GHRL, identifies NGN3+ endocrine progenitors, INS+ beta cells, GCG+ alpha cells, SST+ delta cells, and GHRL+ epsilon cells, respectively. FEV expression is also plotted to highlight novel populations previously uncharacterized in human pancreatic development. (c) Dot plot displaying the top five differentially-expressed genes from each cluster and their expression levels across all 10 endocrine lineage clusters. Size of each dot represents the proportion of each population that expresses each specified gene. Color intensity reflects average level of gene expression. Genes highlighted in red (shading of “0” cluster in (a)) are referred to in the Examples.

FIG. 19. Identification of pre-beta and pre-alpha progenitors in 12wpc human fetal pancreas. (a) Violin plots depicting distribution of expression of NGN3, FEV, INS, and GCG, in single cells from clusters 6 (common endocrine progenitors), 8 (pre-beta progenitors), 0 (beta population #1), 2 (beta population #2), 4 (beta population #3), 9 (pre-alpha progenitors), and 1 (alpha population). Each dot represents the gene expression of a single cell, and the colored distributions (“violins”) represent the spread of gene expression within each cluster. (b) Pseudotemporal ordering using Monocle 3 of common endocrine progenitors, pre-beta progenitors, pre-alpha progenitors, beta cells, and alpha cells (defined as clusters 6, 8, 9, 0, 2, and 1). Pseudotime begins at the vertex of the trajectory with common endocrine progenitors (cluster 6). Two differentiation arcs emanate from this cluster, leading to alpha and beta linages. Pre-beta progenitors (cluster 8) are placed immediately before differentiation into beta cells along pseudotime, and pre-alpha progenitors (cluster 9) are positioned immediately before differentiation into the alpha cell lineage. Second plot represents pseudotemporal ordering using the third beta cell population (Beta 3) as an additional input. Third plot represents pseudotemporal ordering of all endocrine lineages found in our 12wpc human fetal pancreas. (c) Gene expression intensity plots depicting gene expression in individual cells placed along pseudotime. Color/shading intensity reflects level of gene expression.

FIG. 20. Transcriptomic profile comparison among pre-beta, pre-alpha, and common endocrine progenitors. (a) Pseudotemporal ordering highlighting cell populations (common endocrine progenitors, pre-beta progenitors, and pre-alpha progenitors) used for pairwise comparisons. (b-d) Pairwise comparisons among clusters 6 (common endocrine progenitors), 8 (pre-beta progenitors), and 9 (pre-alpha progenitors). Heatmaps depict the genes expressed 2-fold or greater in each comparison.

FIG. 21. Identification of candidate regulators of beta lineage allocation in human endocrine cell development. (a) Heatmap depicting gene expression of cells (represented by columns) as a function of pseudotime across beta cell differentiation. Pseudotime begins with common endocrine progenitors (cell cluster 6), followed by pre-beta progenitors (cell cluster 8), and ends with beta cell populations (cell clusters 0 and 2). Each individual row on the heatmap represents a gene, and the color intensity represents its expression along pseudotime. Genes that change significantly as a function of pseudotime are grouped in 7 main gene clusters: those that are highly expressed at the beginning of pseudotime (gene clusters 2, 3, and 4), genes that are upregulated during the pre-beta progenitor stage but taper in expression as beta cell identity is acquired (gene clusters 6 and 7), and genes that are upregulated during the pre-beta progenitor stage and remain expressed in the differentiated beta cell stage (gene clusters 1 and 5). (b-e) Pseudotime gene expression plots highlighting genes known to be downregulated as differentiation into the beta lineage occurs (b), genes known to be upregulated during beta cell differentiation (c), novel candidate regulators of beta cell lineage allocation that are imprinted (d), genes previously identified in the development and function of the nervous system (e), and those have known functions as a DNA-binding protein or in canonical signaling pathways, such as Activin A signaling (e). In (b-e), each dot represents the gene expression a single cell placed along pseudotime, and the color of the dot denotes its original cluster identity, which corresponds to the cell differentiation scheme outlined in (a). The black curve maps the average gene expression as a function of pseudotime. (f) Model for beta cell differentiation in human fetal pancreatic development.

FIG. 22. Identification of candidate regulators of alpha lineage allocation in human endocrine cell development. (a) Heatmap depicting average gene expression of cells as a function of pseudotime during alpha cell differentiation. Pseudotime begins with common endocrine progenitors (cluster 6), followed by pre-alpha progenitors (cluster 9), and ends with differentiated alpha cells (cluster 1). Each individual row on the heatmap represents a gene, and the color intensity represents its expression along pseudotime. Genes that change significantly as a function of pseudotime are grouped in 6 main gene clusters: those that are highly expressed at the beginning of pseudotime (gene clusters 2 and 3), genes that are upregulated during the pre-alpha progenitor stage but taper in expression as alpha cell identity is acquired (gene cluster 5), and genes that are upregulated during the pre-alpha progenitor stage and remain expressed in the differentiated alpha cell stage (gene clusters 1, 4, and 6). (b-e) Pseudotime gene expressions plots highlighting genes known to be expressed by the adult alpha cell lineage (b), genes known to be upregulated specifically during alpha cell differentiation (c), and novel candidate regulators of alpha cell lineage allocation that have previously been identified in the development and function of the nervous system (d) or are cell surface markers and channels (e). In (b-e), each dot represents the gene expression a single cell placed along pseudotime, and the color of the dot denotes its original cluster identity, which corresponds to the cell differentiation scheme outlined in (a). The black curve maps the average gene expression as a function of pseudotime. (f) Model for alpha cell differentiation in human fetal pancreatic development.

FIG. 23. Human fetal pancreatic populations over developmental time. (a) UMAP-based clustering of merged cell populations from 12wpc_1, 12 wpc_2, 15.5wpc, and 16wpc samples. Merging of these four datasets was accomplished using Seurat 3's Integration method. (b) UMAP clustering of each sample and technical replicate (if any) showing contribution of designated sample to overall merged UMAP clustering shown in (a). The 12wpc_1, 12wpc_2, and 16wpc samples were each run on two lanes of the 10× Chromium single-cell sequencing chip, while the 15.5wpc sample, which was enriched for EPCAM+ cells, was run on only one lane of the 10× chip. (c) Known marker genes of epithelial cells (EPCAM+), ductal cells (50×9+), acinar cells (CPA1+), endocrine cells (CHGA+), mesenchymal cells (COLA1A+), endothelial cells (PECAM1+), immune cells (PTPRC+), and nerves (SOX10+), showing the identities of all 31 clusters from UMAP-based clustering. Color indicates level of gene expression.

FIG. 24. Human fetal endocrine populations over developmental time. (a) UMAP-based clustering resulting from endocrine sub-clustering of CHGA+ clusters from merged dataset (FIG. 23) organized into 16 distinct populations. (b) Sample ID mapped onto UMAP depicts contribution of each time point (and associated technical replicate, if any) to each merged cluster. (c) Gene expression intensity plots highlighting NGN3+ progenitors, FEV+ cells, INS+ beta cells, GCG+ alpha cells, SST+ delta cells, and GHRL+ epsilon cells. (d) Dot plot displaying the top three differentially-expressed genes from each cluster and their expression levels across all 16 endocrine lineage clusters. Size of each dot represents the proportion of each population that expresses each specified gene. Color/shading intensity reflects average level of gene expression. (e) Pseudotime ordering with Monocle 3 using endocrine progenitors and the alpha and beta lineages as input. Both the sample ID and mitochondrial content are mapped onto the pseudotime ordering trajectory, highlighting significant batch effect that is not corrected due to inclusion of 15.5wpc sample. 15.5wpc sample displays significantly lower mitochondrial content, which drives batch effect.

FIG. 25. Single-cell RNA-sequencing identifies heterogeneous cellular compartments in hESC-derived endocrine progenitor populations. (a) Schematic depicting the six stages of the protocol for in vitro beta cell differentiation, highlighting End Stage 4 pancreatic progenitors and Stage 5 endocrine progenitors taken for single-cell RNA-sequencing. (b) UMAP-based clustering of ES4 (end stage 4), 55D4, and S5D7 cells. (c-e) Blended expression plots highlight cells that express either both PDX1 and NKX6-1 or NEURDO1 and CHGA. Gene expression plots of NGN3, TOP2A, CDX2, INS, and GCG highlight endocrine progenitors, replicating cells, cells that mis-differentiated into a CDX2+ intestinal lineage, and hormone-expressing populations, respectively.

FIG. 26. Single-cell RNA-sequencing identifies heterogeneous cellular compartments in hESC-derived beta-like stage cells. (a) Schematic depicting the six stages of the in vitro beta cell differentiation, highlighting Stage 6 beta-like cells taken for single-cell RNAsequencing. (b) UMAP-based clustering of S6D4 and S6D10 beta-like stage cells organized into distinct clusters. (c, d) Blended expression plots highlight cells that express either both PDX1 and NKX6-1 or NEURDO1 and CHGA. Gene expression plots of CDX2, INS, and GCG cells that represent cells mis-differentiated into a CDX2+ intestinal lineage or hormone-expressing populations, respectively.

FIG. 27. Emergence of FEV+ cells during in vitro beta cell differentiation. (a) qPCR (Taqman) data depicting FEV expression throughout the directed differentiation of hESCs towards the beta lineage. FEV expression in isolated adult human islets shown as a comparator. (b) Dual in situ hybridization and immunofluorescence on S5D3 and S6D11 hESCderived clusters. FEV transcript is represented in red, and DAPI staining is in blue. Those of skill in the art will identify the shading corresponding to DAPI staining based on the known pattern of cell staining for DAPI, and by subtraction will identify the shading corresponding to Fev staining. For S5D3 clusters, green represents PDX1. For S6D11 clusters, green represents CHGA. (c) FEV expression plots from ES4, S5D4, S5D7, S6D4, and S6D10 single-cell RNA-sequencing. (d) Heatmaps depicting the results of a Pearson's correlation analysis comparing FEV+ progenitors from 12wpc_1 and 12wpc_2 human fetal datasets with FEV+ clusters found in each sampled time point of directed differentiation. Color/shading denotes level of correlation with FEV+ progenitors from 12wpc_1 and 12wpc_2 human fetal datasets, where shades of red reflect high transcriptional correlation.

FIG. 28. Reconstruction of lineage relationships among hESC-derived endocrine cells during in vitro beta cell differentiation. (a) UMAP-based clustering of merged CHGA+ clusters from ES4, S5D4, S5D7, S6D4, and S6D10 time points. (b) Time point IDs are mapped onto UMAP clusters to illustrate the contribution of each time point to each resulting merged cluster. (c) Pseudotemporal ordering of the merged dataset using Monocle 3 depicts one main differentiation trajectory. Time point IDs mapped onto the trajectory reveals a cluster of ES4 cells at one end of the trajectory, which was designated as the beginning of the pseudotime ordering analysis. (d) Monocle trajectory with highlighted clusters labeled by their cluster identity as found in (a). (e) Gene expression intensity plots depicting INS, GCG, and SST expression in individual cells placed along pseudotime highlight the poly-hormonal and INS+ beta cell branches in the differentiation trajectory. FEV gene expression intensity plot depicts uniform FEV expression throughout the majority of pseudotime. (f) Gene expression intensity plots depicting PHOX2A, TLX2, and TBX2 expression in individual cells placed along pseudotime highlight the restriction of these genes in the hormone-negative branch that contains the hESC-derived cells that are predicted to have mis-differentiated. (g) PHOX2A, TLX2, and TBX2 expression plots from merged, endocrine sub-clustered UMAP from FIG. 24a. (h) Model of a bifurcation event of PDX1+/NKX6.1+ progenitors into a mis-differentiated FEV+ lineage and hormone-expressing endocrine lineages.

FIG. 29. Assessing the function of FEV in beta cell differentiation and maturation. (a, b) Schematic illustrating the FEV locus and the use of CRISPR/Cas9-mediated genomic editing to generate a FEV-KO hESC clonal line. A FEV-KO gRNA was designed to target exon 1. Genomic editing with this FEV-KO gRNA led to the generation of a FEV-KO hESC clonal line with a 1-bp deletion in one allele and a 1-bp insertion in the second allele, leading a homozygous mutation in the FEV locus. (c) Illustration of the directed differentiation of FEV-KO hESCs towards a beta lineage. (d) FACS analysis of wild-type and FEV-KO pluripotent cells quantifies percentage of cells with pluripotency markers OCT4 and TRA-1-60. FACS analysis of wild-type and FEV-KO cells at the completion of Stage 1 of the directed differentiation quantifies percentage of cells that have entered into the SOX17+/FOXA2+ definitive endoderm stage. FACS analysis of wild-type and FEV-KO cells at Stage 6, Day 11 (S6D11) determines the percentage of CHGA+/CPEP+ beta cells present.

FIG. 30. Strategy for identifying transcriptional targets of FEV. (a) Schematic illustrating the use of CRISPR/Cas9-mediated genomic editing to generate a FEV-MYC hESC line. A FEV-KI (knock-in) gRNA was designed to target the 3′ end of exon 3 of the FEV locus. A targeting template containing a 3×MYC sequence flanked by homology arms was also designed and commercially synthesized. Through use of genomic editing and homology-directed repair, the 3×MYC sequence was knocked-in in frame with the endogenous FEV locus, leading to the expression of a FEV-MYC fusion protein to be used for ChIP-seq. (b) Illustration of future plans for the directed differentiation of FEV-MYC hESCs towards a beta lineage. Endocrine progenitor-stage cells will be harvested and ChIP-seq will be performed using an antibody against MYC. The MYC antibody will pull down FEV transcription factor bound to DNA, and sequencing of bound DNA will identify transcriptional targets of FEV specifically during this endocrine progenitor stage prior to beta cell lineage determination.

FIG. 31. Identifying and isolating FEV-expressing cells during in vitro beta cell differentiation. (a) Schematic illustrating the use of CRISPR/Cas9-mediated genomic editing to generate two FEV reporter hESC lines: a FEV-GFP and a FEV-tNFGR line. A FEV-KI gRNA targets the 3′ end of exon 3 of the FEV locus, and a targeting template containing either a T2AGFP or T2A-tNGFR sequence flanked by homology arms was also designed and commercially synthesized. Through use of genomic editing and homology-directed repair, the T2A-GFP or T2A-tNGFR sequence was knocked-in in frame with the endogenous FEV locus, leading to bicistronic translation of the FEV transcription factor and reporter protein (GFP or tNGFR). (b) Illustration depicting future use of the FEV reporter lines, such as the FEV-GFP line, to perform small molecule library screens to identify signaling compounds that induce FEV+ progenitor replication or enhance beta cell differentiation from FEV+ progenitors. (c) Illustration showing future use of the FEV reporter lines to sort and isolate different FEV+ populations, such as a predicted mis-differentiated FEV+ cell population, throughout in vitro beta cell differentiation.

FIG. 32. Development of a platform to functionally validate candidate beta lineage regulators. (a) Illustration depicting isolation of endocrine progenitor-stage cells for geneknockdown via CRISPR/Cas9 genomic editing. Endocrine progenitor-stage clusters will be dissociated and nucleofected with Cas9 and a gRNA targeting a candidate beta lineage regulator. These edited endocrine progenitor-stage cells are then re-aggregated and differentiated towards the beta lineage to determine if knockdown of specific candidate beta lineage regulators result in reduced beta cell differentiation.

### DETAILED DESCRIPTION

Organogenesis requires the complex interactions of multiple cell lineages that coordinate their expansion, differentiation, and maturation over time. Utilizing a combination of single-cell RNA sequencing, immunofluorescence, in situ hybridization, and genetic lineage tracing, we profile the cell types within the epithelial and mesenchymal compartments of the murine pancreas across developmental time. We identify previously underappreciated cellular heterogeneity of the developing mesenchyme and reconstruct potential lineage relationships among the pancreatic mesothelium and novel mesenchymal cell types. Within the epithelium, we find a novel endocrine progenitor population, as well as an analogous population in both human fetal tissue and human embryonic stem cells differentiating towards a pancreatic beta cell fate. Further, we identify candidate transcriptional regulators along the differentiation trajectory of this population towards the alpha or beta cell lineages. This work establishes a roadmap of pancreatic development and demonstrates the broad utility of this approach for understanding lineage dynamics in developing organs.

The mesenchyme is critical for epithelial specification and proliferation throughout pancreatic development⁴⁸⁻⁵⁰, yet the individual cell types responsible for these processes remain unidentified. Our single-cell dataset has enabled the identification of multiple novel mesenchymal populations, highlighted the transcriptional dynamism of the pancreatic mesothelium, and predicted lineage relationships among the mesothelium and VSM populations. Secreted factors, such as mesothelial-derived Fgf9, may play a similar role in the pancreas as in the lung, where it regulates mesenchymal cell proliferation and vascular formation⁵¹. While previous studies identified Cxcl12 (highly expressed in our dataset in cluster 4) as a regulator of pancreatic epithelial specification, differentiation, and adult regeneration^{52,53}, these studies focused on the epithelium and did not define a role for mesenchymally-derived Cxci12. Finally, secretion of Wnt antagonists by cluster 5 may regulate processes regulated by Wnt signaling in the developing pancreas, including epithelial specification, expansion, and exocrine development⁵⁴. Future work can focus on uncovering the functions of these individual mesenchymal populations in development, physiology, and pathology of the pancreas.

With the various cell types of the mesenchyme now enumerated and their markers identified, we can begin to elucidate the maturation and lineage relationships of the pancreatic mesenchymal compartment. Our time course data have provided evidence of maturation within the mesothelial population. Genes such as Pitx2, kallikren 13 (Kik13) and 8 (Kik8), were differentially expressed in younger, E12.5, mesothelial cells. Pitx2 regulates differentiation in multiple systems^{27,57-60}, and the kallikren family are serine proteases that are involved in extracellular matrix and adhesive molecule degradation⁵⁵. Expression of these genes leads to the expectation that the E12.5 mesothelial population is primed for migration and differentiation. In contrast, the E17.5 mesothelial population expressed genes related to barrier or immune function, such as dermokine (Dmkn)^{56,57}, bone marrow stromal antigen 2 (Bst2), and retinoic acid receptor responder 2 (Rarres2)⁵⁸. These results establish stage-dependent roles for the mesothelium throughout development.

The different roles for the mesothelium across time are also evident from our pseudotime analysis, which predicts that the mesothelium serves as a progenitor of other mesenchymal cell types during development. Indeed, the mesothelium is a critical mesenchymal progenitor population in other organs, such as the heart, intestine, lung, and liver¹⁴⁻¹⁷. The data disclosed herein indicate that mesothelial progenitor activity occurs at E12.5 or earlier during pancreatic development, consistent with other organ systems^{11,14,16}. Indeed, a recent study identified that parietal mesothelial cells can function as progenitor cells prior to pancreatic specification⁵⁹. In vivo lineage tracing studies will verify the predictions from these pseudotime analyses, and the transcriptomic information obtained by this study will allow the development of tools to target individual populations within the mesenchyme and perform lineage tracing, ablation, and expression studies.

The study of the mesothelium in development is also relevant for fibrotic diseases of adult organs, as factors secreted by mesothelial cells and mesothelial-derived, disease-driving myofibroblasts modulate organ responses to injury⁶⁰⁻⁶². Fibrotic diseases of the adult pancreas are characterized by aberrant recapitulation of developmental pathways within the epithelium^{63,64}. We can now utilize our developmental dataset to probe the mesenchymal populations during adult homeostasis and disease states, and compare to the populations detected throughout development. Therefore, this dataset serves as a broad resource for the implementation of future studies in pancreatic mesenchymal biology.

Within the epithelial compartment, our identification of a novel Fev^{Hi} endocrine progenitor population provides increased resolution of endocrine differentiation. The relative timing of expression of canonical endocrine lineage genes can now be mapped onto these additional differentiation stages. Several lines of evidence identify the gene Fev as a direct target of Ngn3: Fev is the transcription factor most strongly expressed in Ngn3⁺ endocrine progenitors⁶⁵, and Ngn3 knockout embryos do not exhibit Fev expression in the developing pancreas²⁴. Known target genes of Ngn3, such as Pax4⁶⁶ and Runx1t1i⁶⁷, are expressed by the early-stage Fev⁺/Pax4⁺ population. Additionally, Pax6 was upregulated within the Fev^{Hi} population. Although Chga and Chgb are often utilized as markers of differentiated endocrine lineages, we found that Chga and Chgb are expressed in the Fev^{Hi} population prior to hormone acquisition. This result is consistent with previous work that identified Chga⁺, hormone⁻ cells in rodent pancreatic development⁶⁸. The Fev^{Hi} cell stage likely represents the cell stage during endocrine differentiation preceding specialized hormone production and may now serve as a cellular landmark for understanding endocrine lineage gene expression dynamics.

The gene Fev has been previously studied mainly in serotonergic neurons, where it is a master transcription factor required for cellular differentiation and maturation, as well as serotonin synthesis²⁸. Fev switches transcriptional targets from differentiation genes during development to maturation genes postnatally in serotonergic neurons⁶⁹. In an insulinoma cell line, Fev directly binds to the regulatory regions of serotonergic genes, such as Tph1, Tph2, Ddc, Slc18a2, and Slc6a4, as well as the Ins1 promoter itself²⁴. Future ChIP-seq studies of embryonic pancreas will globally identify direct targets of Fev and Fev-regulated transcriptional networks in developing endocrine cells.

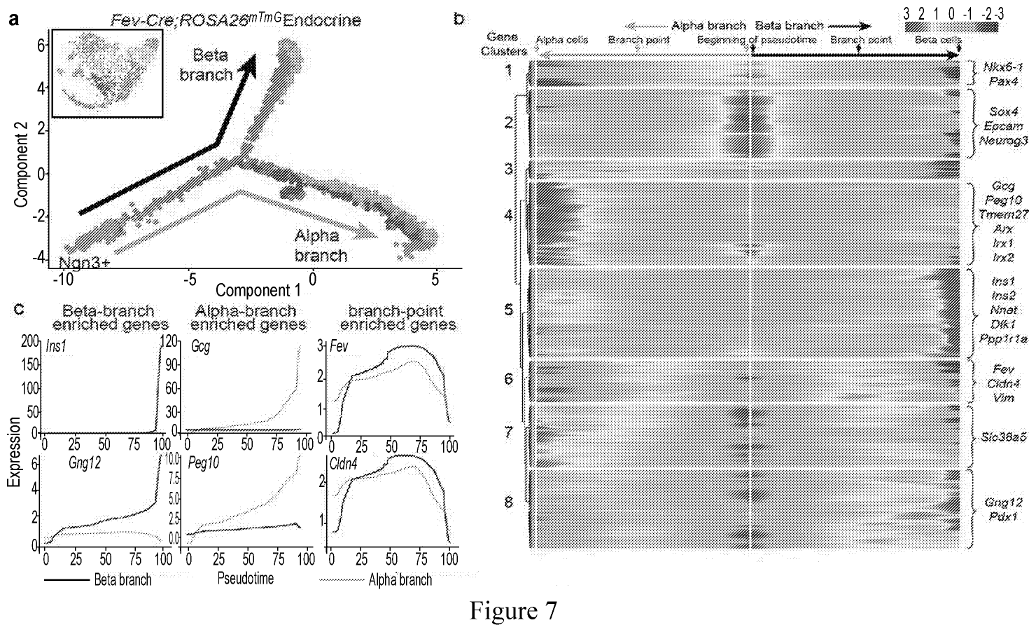

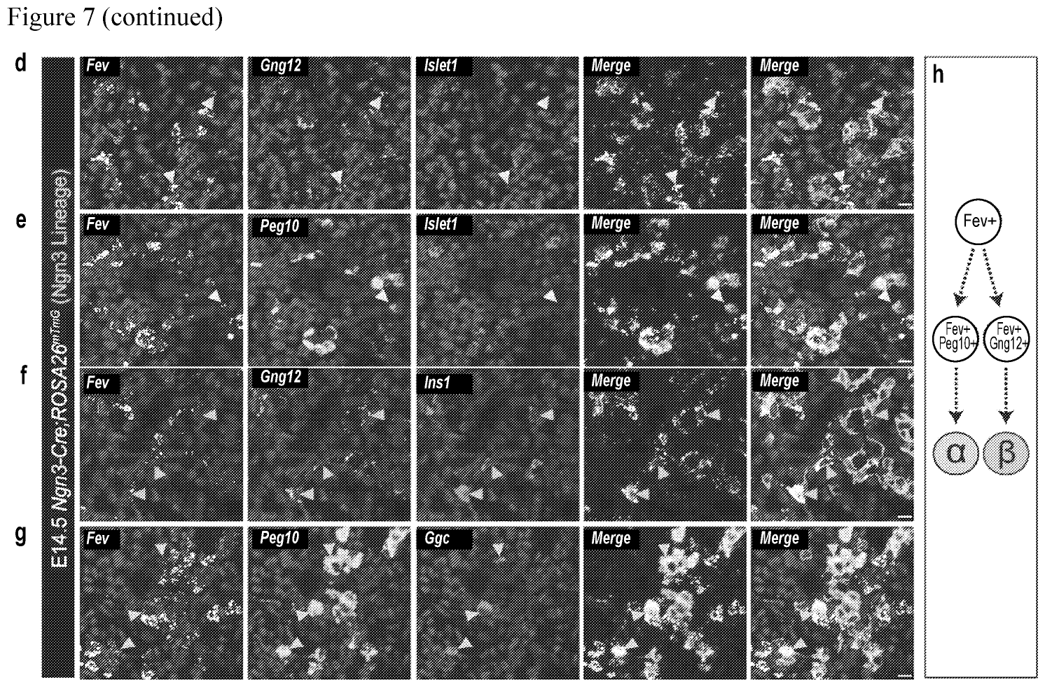

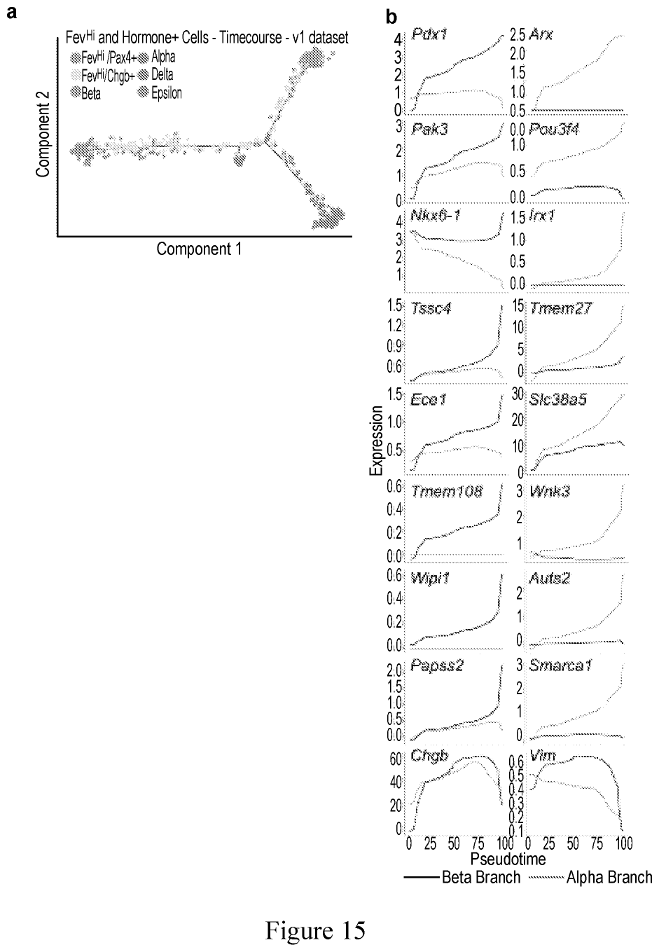

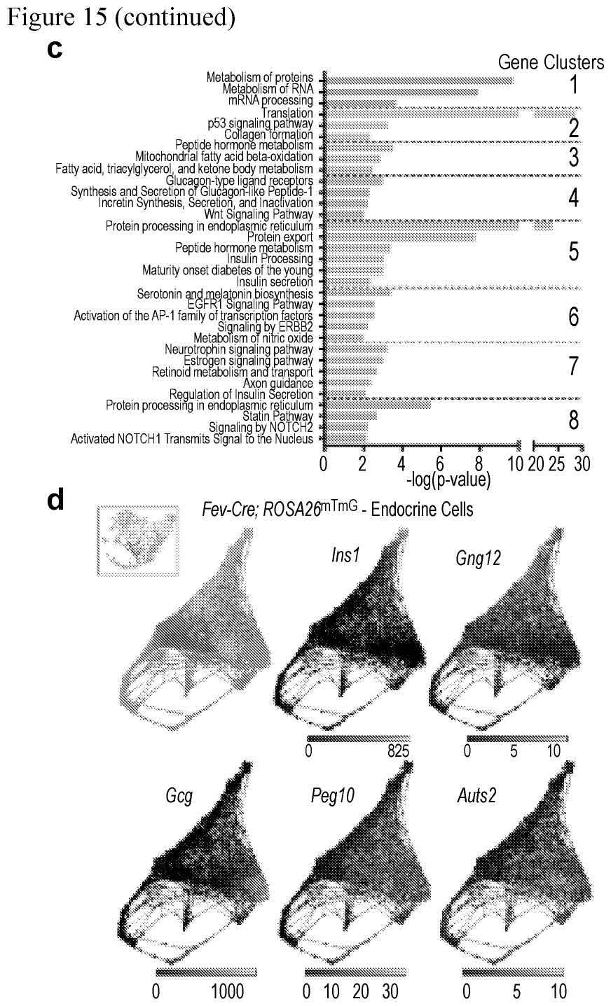

Using genetic lineage tracing in vivo, we have demonstrated that all five endocrine lineages of the developing pancreas transit through a Fev-expressing stage, and that Fev⁻ lineage cells contribute not only to embryonic, but also to adult pancreatic endocrine cells. The fraction of epsilon cells that are not derived from a Fev⁻ lineage may represent the subset of ghrelin⁺ cells previously reported to give rise to cells of the ductal and exocrine lineages 30. Given that all adult gamma cells are Fev⁻ lineage labeled, the small subset of gamma cells that are not lineage traced during pancreatic development may represent those that do not persist in the adult pancreas. Further highlighting the relevance of Fev^{Hi} progenitors during pancreatic development, our pseudotime analysis revealed that Fev-expressing cells may be pre-specified towards an alpha or beta cell fate (FIG. 7 and FIG. 15). As expected, we found expression of Ins1 and Gcg at the termini of the beta and alpha branches, and upregulation of Pdx1 and Arx, which are known regulators of endocrine cell fate decisions, earlier in pseudotime. In addition, our pseudotime analysis identified novel genes that are enriched along the alpha or beta branch and expressed prior to upregulation of hormones. These genes warrant further study as potential novel regulators of the acquisition of alpha or beta cell identity.

For the eventual application of this knowledge to human therapeutics, it is important to validate that the predicted relationships hold true in the context of human pancreatic development. Our staining of human fetal pancreas identified the analogous FEV^{Hi} population, consistent with our findings in murine pancreata. Directed differentiation of hESCs towards endocrine cell fates will provide a platform for modeling and manipulating the predicted lineage regulators found in this study. Indeed, we have identified a FEV⁺ population within hESC-derived endocrine progenitor cells. Deeper knowledge of these lineage decisions may substantially improve directed differentiation efforts to efficiently generate functional beta cells for cellular replacement therapy for patients with diabetes. This study highlights the power of combining single-cell transcriptomic information with in vivo lineage tracing to reconstruct developmental trajectories within cellular compartments. Identification of novel populations and their lineage relationships will promote discovery of the mechanisms that drive lineage decisions and commitment.

The following examples are presented by way of illustration and are not intended to limit the scope of the subject matter disclosed herein.

### EXAMPLES

### Example 1

Materials and Methods

Mice

All mouse procedures were approved by the University of California, San Francisco (UCSF) Institutional Animal Care and Use Committee (IACUC). Mice were housed in a 12-hour light-dark cycle in a controlled temperature climate. Noon of the day of vaginal plug was considered embryonic day 0.5.

Timed-pregnant Swiss Webster mice were obtained from Charles River Laboratories. Ngn3-Cre⁷⁰, Fev-Cre⁷¹, ROSA26mTmG 31 mice have been previously described and were maintained in a C57BL/6J background.

Human Tissue Procurement and Isolation

Human fetal pancreata were harvested from post-mortem fetuses at 23 weeks of gestation with permission from the ethical committee of the University of California, San Francisco (UCSF). Tissue was fixed in 4% paraformaldehyde overnight at 4° C. After three washes in 1×PBS, tissue was either cryopreserved in 30% sucrose solution at 4° C. overnight and embedded in OCT, or placed in 40% ethanol then 70% ethanol before paraffin embedding. 8 um sections were cut on the cryostat or microtome. In situ hybridization and immunofluorescence were then performed as described below.

Adult human islets were isolated from cadaveric donor tissue by the UCSF Islet Production Core with permission from the UCSF ethical committee. Consented cadaver donor pancreata were provided by the nationally recognized organization UNOS via local organ procurement agencies. The identifiers were maintained at the source only, and the investigators received de-identified specimens.

Informed consent was obtained for all human (fetal and adult) tissue collection, and protocols were approved by the Human Research Protection Program Committee on Human Research of the University of California, San Francisco (UCSF).

Embryonic Stem Cell Culture and Differentiation to the Endocrine Lineage

The human embryonic stem cell (hESC) line HUES8 was obtained from Harvard University and used for the generation of hESC-derived β-like cells (BLCs). Pluripotent HUES8 cells were maintained as spherical clusters in suspension in mTeSR-1 (StemCell Technologies) in 500 mL spinner flasks (Corning, VWR) on a magnetic stir plate (Dura-Mag) within a 37° C. incubator with 5% CO2, 100% humidity, and a rotation rate of 70 rpm. Cells were screened for mycoplasma contamination using the MycoProbe Mycoplasma Detection Kit (R&D Systems), according to the manufacturer's instructions.

hESC-derived endocrine progenitor cells were generated as previously described³². In brief, HUES8 cells were seeded into a spinner flask at a concentration of 8×10⁵ cells/mL in mTeSR-1 media with 101.iM Rock inhibitor Y27632 to allow formation of spherical clusters. Differentiation was initiated 72 hours later. Differentiation was achieved in a step-wise fashion using the following growth factors and/or small molecules: definitive endoderm cells (Stage 1) (Activin A 100 ng/mL, R&D Systems; CHIR99021 141.ig/mL, Stemgent); gut tube endoderm cells (Stage 2) (KGF 50 ng/mL, Peprotech); early pancreatic progenitors (Stage 3) (LDN193189 200 nM, Fisher Scientific; KGF 50 ng/mL, Peprotech; Sant-1 0.251.iM, Sigma; Retinoic Acid 21.iM, Sigma; PdbU 500 nM, EMD Biosciences); later pancreatic progenitors (Stage 4) (KGF 50 ng/mL, Peprotech; Sant-1 0.251.iM, Sigma; Retinoic Acid 0.11.iM, Sigma); endocrine progenitors (Stage 5) (Sant-1 0.251.iM, Sigma; Retinoic Acid 0.11.iM, Sigma; XXI 11.iM, EMD Millipore; Alk5i 101.iM, Axxora; T3 11.iM, EMD Biosciences; Betacellulin 20 ng/mL, Fisher Scientific), BLCs (Stage 6) (Alk5i; T3). Successful differentiation was assessed at the definitive endoderm, pancreatic progenitor 1, pancreatic progenitor 2, and endocrine progenitor stages via immunofluorescence or FACS for stage-specific marker genes.

To measure the expression of FEV at various stages of human endocrine differentiation, aliquots of clusters were removed from the flask and analyzed at several time points: after 5 days in Stage 5 (“mid-stage endocrine progenitors”), after 7 days in Stage 5 (“late-stage endocrine progenitors”), and after 5 days at the BLC stage. As a comparator, pluripotent, undifferentiated hESCs in mTeSR-1, as well as human adult islets, were also analyzed for FEV expression.

Immunofluorescence

Embryonic mouse pancreata were dissected in cold 1×PBS and fixed in zinc-buffered formalin (Anatech LTD) at room temperature (RT) for 30-90 minutes or overnight at 4° C. After three washes in 1×PBS, tissue was processed for either cryopreservation or paraffin embedding. Cryopreserved pancreata were placed in 30% sucrose solution at 4° C. overnight before embedding in OCT. Paraffin-embedded pancreata were placed in 40% ethanol and 70% ethanol before paraffin tissue processing. 8 um sections were cut on the cryostat or microtome. For immunofluorescence on paraffin sections, slides were baked at 55° C. for 30 minutes, deparaffinized in xylene, and rehydrated in decreasing concentrations of ethanol. Heat-mediated antigen retrieval was performed using Antigen Retrieval Citra Solution (Biogenex Laboratories). Tissue sections were blocked in 5% normal donkey serum (NDS; Rockland Immunochemicals) and Mouse-on-Mouse IgG blocking reagent (Vector Laboratories) when appropriate in 0.2% Triton X-100 in PBS (PBT) for 1 hour and then stained overnight at 4° C. using the following primary antibodies: Acta2 (1:200, Abcam ab21027), Cav1 (1:200, Abcam ab2910), Chromogranin A (1:100, Abcam ab15160), E-cadherin (1:200, BD Transduction Lab 610182), Glucagon (1:100, Abcam ab82270), Insulin (1:50, DAKO A0564), Vimentin (1:200, Abcam ab92547), and Wt1 (1:100, Abcam ab89901). All antibodies have been validated by the manufacturer. The next day, sections were washed three times in 0.1% Tween 20 in 1×PBS and then incubated with species-specific Alexa Fluor 488-, 594-, or 647-conjugated secondary antibodies (1:500, Jackson ImmunoResearch) and DAPI in 5% NDS in 0.2% PBT for 1 hour at RT. Sections were washed three times in 0.1% Tween 20 in 1×PBS, rinsed in 1×PBS, and then mounted in Fluoromount-G mounting medium (Southern Biotech). Slides were stored at 4° C.

For immunofluorescence on cryosections, slides were removed from −80° C. storage and allowed to reach RT. Sections were rinsed in 1×PBS three times and permeabilized in 0.5% PBT for 10 minutes at RT. Tissue sections were blocked in 5% NDS and, if needed, Mouse-on-Mouse IgG blocking reagent in 0.1% PBT for 1 hour and then stained overnight at 4° C. using the following primary antibodies: Epcam (1:200, BD Transduction Lab 552370), Glucagon (1:2000, Millipore 4031-01F), Insulin (1:250, DAKO A0564), Somatostatin (1:500, Santa Cruz Biotechnology sc-7819, Ghrelin (1:1500, Santa Cruz Biotechnology sc-10368), Pancreatic Polypeptide (PPY; 1:250, Abcam ab77192), and Vimentin (1:200, Abcam ab92547). All antibodies have been validated by manufacturer. Sections were washed the next day three times in 1×PBS and then incubated with species-specific Alexa Fluor 488-, 555-, 594-, or 647-conjugated secondary antibodies and DAPI in 5% NDS in 0.1% PBT for 1 hour at RT. Sections were washed three times in 1×PBS and mounted in Fluoromount-G mounting medium. Slides were stored at 4° C.

Images were captured on a Zeiss Apotome Widefield microscope with optical sectioning capabilities or Leica confocal laser scanning SP8 microscope. Maximum intensity z-projections were then prepared using ImageJ, where brightness, contrast, and pseudo-coloring adjustments were applied equally across all images in a given series.

In Situ Hybridization

In situ hybridization was performed on 8 um sections as previously describee using RNAscope technology (Advanced Cell Diagnostics)⁷³ according to the manufacturer's instructions. In situ probes against mouse Ngn3 (422409-C2), Fev (413241-C3), Isl1 (451931), Ins1 (414661-C4), Gcg (400601), Sst (404631-C3), Ghrl (415301-C2), Ppy (482701), Peg10 (512921-C4), Gng12 (462521-C2), Nnat (432631-C2), Barx1 (414681), Pitx2 (412841-C2), Stmn2 (498391-C3), Msln (443241) and human NGN3 (505791-C4), FEV (471421-C3), and ISL1 (478591-C2) were used in combination with the RNAscope Multiplex Fluorescent Reagent Kit v2 for target detection. Following signal amplification of the target probes, sections were washed in 1×PBS three times and blocked in 5% NDS in 0.1% PBT for 1 hour at RT. Tissue sections were then stained with primary and secondary antibodies as described above in the “immunofluorescence” section.

For in situ hybridization of hESC-derived clusters, cells were fixed with 4% PFA for 15 minutes at RT, washed with PBS, and cryoprotected in 30% sucrose overnight. The next day, clusters were embedded in a small sphere of 1.5% low-melting temperature agarose; these were again cryoprotected in 30% sucrose overnight. The following day, the agarose spheres were soaked in OCT and frozen in a dry ice bath. In situ hybridization was then performed on 8 um sections using human NGN3, FEV, and ISL1 RNAscope probes.

Quantification of Cell Proportions

Quantification of pancreata was performed by manual counting using ImageJ software. Cell populations present at less than 1% in Ngn3-lineage-traced E14.5 replicates were deemed artifact and excluded from further analysis.

Quantitative RT-PCR

hESCs from various stages of directed differentiation were collected and RNA extracted with the RNeasy Mini Kit (Qiagen). Reverse transcription was performed with the Clontech RT-PCR kit. RT-PCR was run on a 7900HT Fast Real-Time PCR instrument (Applied Biosystems) with Taqman probes for FEV (assay ID: Hs00232733_m1) and GAPDH (assay ID: Hs02758991_g1) in triplicate. Data were normalized to GAPDH. Error bars represent standard deviation.

Dissociation and FACS of Embryonic Pancreas

Embryonic mouse pancreata were dissected and placed in 1×PBS on ice, then dissociated into single cells using TrypLE Express dissociation reagent (Thermo Fisher) at 37° C. with pipet trituration at 5-minute intervals during incubation. For v1 datasets, E12.5 pancreata were dissociated for 10 minutes, E14.5 pancreata for 15 minutes, and E17.5 pancreata for 30 minutes. For batch 1, we pooled 14 E14.5 pancreata from one litter. For batch 2, which was collected on a different day, we pooled tissue from each time point separately: 18 E12.5 pancreata from two litters, 11 E14.5 pancreata from one litter, and 8 E17.5 pancreata from one litter. Dissociations were neutralized with FACS buffer (10% FBS+2 mM EDTA in phenol-red free HBSS). Dissociated cells were passed through a 30 um cell strainer and stained with Sytox live/dead stain (Thermo Fisher). Stained cells were washed twice in FACS buffer and then sorted using a BD FACS Aria II. After size selection to remove doublets, all live cells were collected.

For version 2 10× datasets, we pooled tissue from each time point separately, each performed on a different day: 14 E12.5 pancreata from one litter, 13 E14.5 pancreata from one litter, and 13 E17.5 pancreata from one litter. For the E14.5 Fev-Cre; ROSA26^{mTmG} 10× sample, we pooled 3 pancreata from one litter. Dissociations were performed as described above. Cells undergoing a CD140a negative selection were stained with CD140a-APC (1:50; eBiosciences, cat. 17-1401-81; validated by manufacturer). Stained cells were washed twice in FACS buffer and then sorted using a BD FACS Aria II. After size selection to remove doublets, all live CD140a⁻ cells were collected. For the E14.5 Fev-Cre; mTmG pancreata, live GFP⁺ cells and GFP⁻/TdTomato⁺ cells were collected. All 4,000 GFP⁺ (Fev-lineage-traced) cells were loaded onto the 10× Genomics platform, supplemented with an additional 21,000 TdTomato⁺/GFP⁻ (non-lineage-traced).

Single-Cell Capture and Sequencing

To capture individual cells, we utilized the Chromium Single Cell 3′ Reagent Version 1 Kit (10× Genomics)⁷⁴. For batch 1, 12,800 cells from E14.5 pancreata were loaded into one well of the 10× chip, while for batch 2, 18,000 cells per time point were each loaded into their own respective wells to produce Gel Bead-in-Emulsions (GEMs). GEMs underwent reverse transcription to barcode RNA before cleanup and cDNA amplification. Libraries were prepared with the Chromium Single Cell 3′ Reagent Version 1 Kit. Each sample was sequenced on 2 (Batch 1) or 1 (Batch 2) lanes of the HiSeq2500 (Illumina) in Rapid Run Mode with paired-end sequencing parameters: Read1, 98 cycles; Index1, 14 cycles; Index2, 8 cycles; and Read2, 10 cycles.

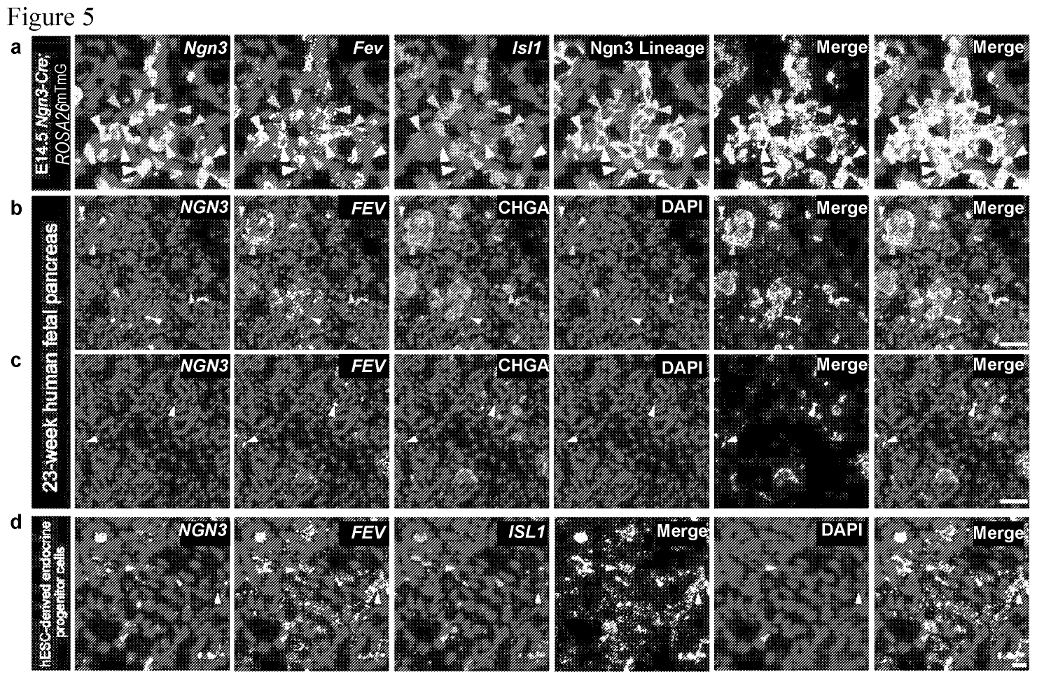

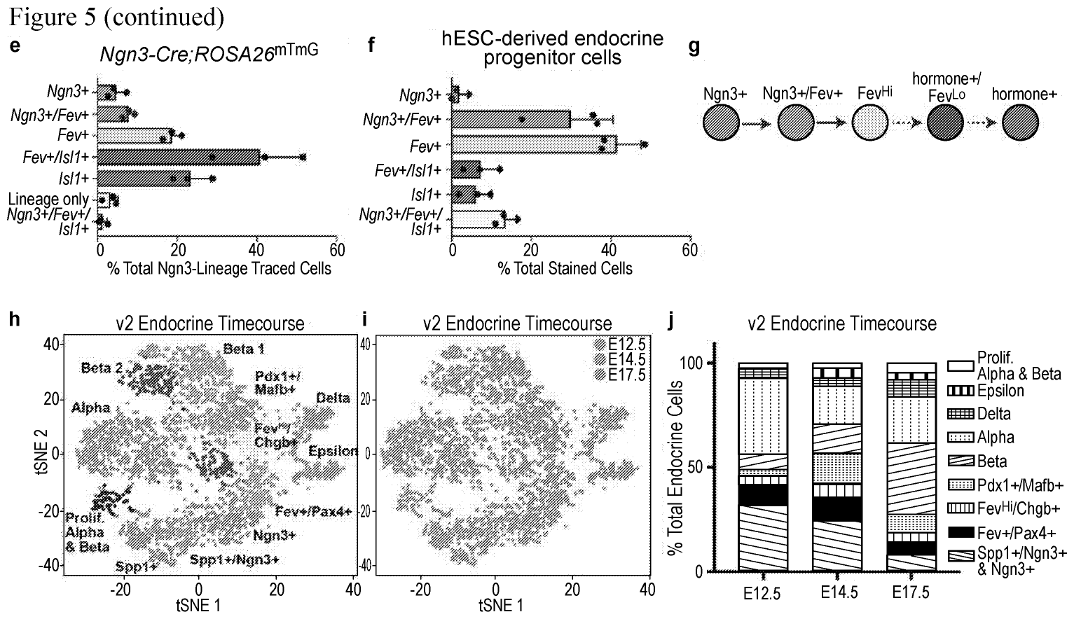

The CD140a-depleted E12.5, E14.5, and E17.5 datasets and Fev-Cre; ROSA26mTmG dataset in FIGS. 5 and 7 were generated with Chromium Single Cell 3′ Reagent Version 2 kits (10× Genomics). 27,000 cells were loaded onto their respective wells and underwent the same processing as the Version 1 kits, according to manufacturer instructions for Version 2 kits. Libraries were sequenced on the NovaSeq (Illumina) with the same sequencing parameters as above.

Single-Cell Analysis

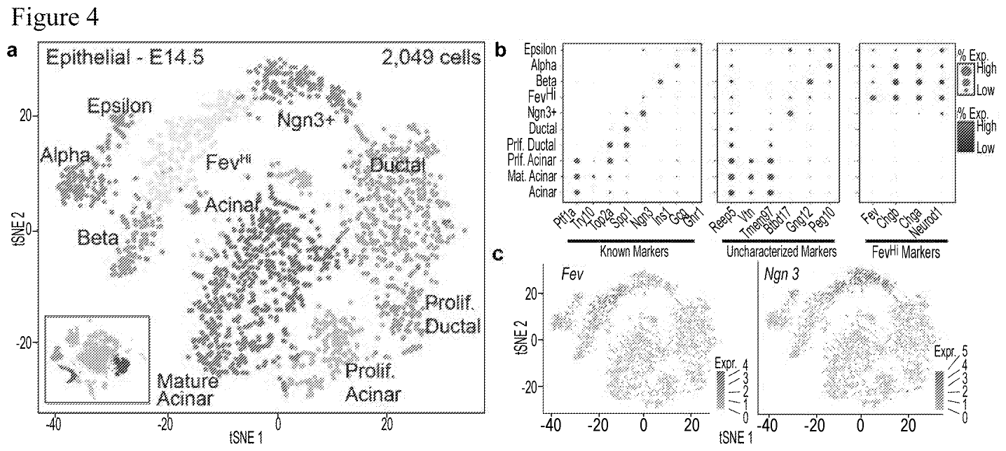

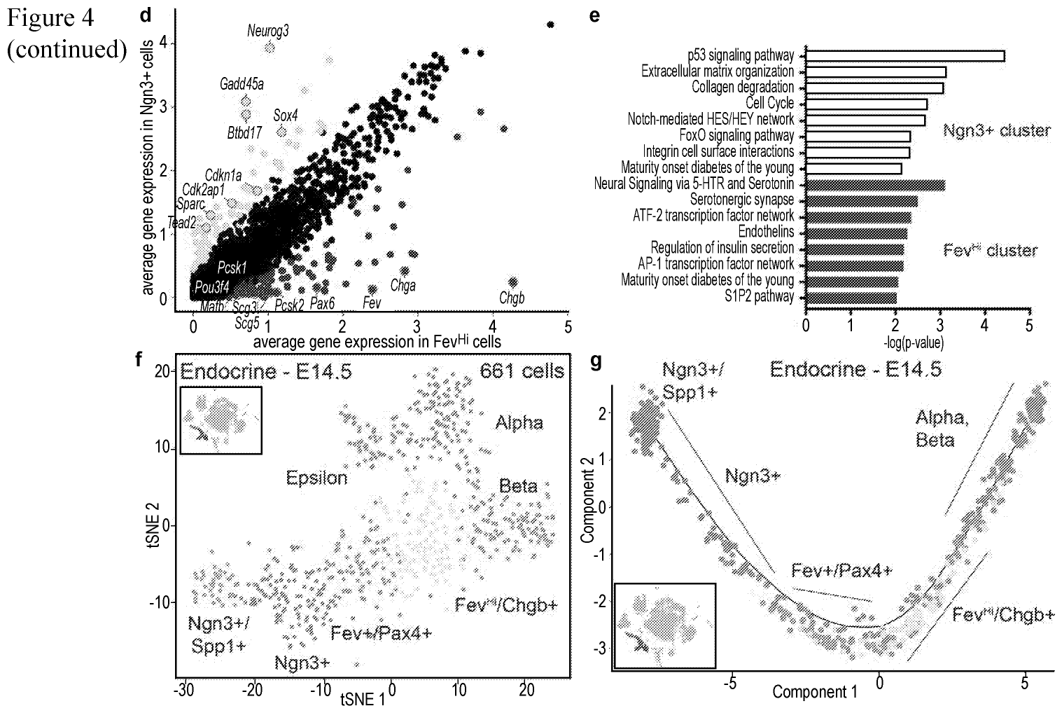

For the v1 datasets, we utilized CellRanger v1.1.0 software for v1 datasets and v2.1.0 for v2 datasets with default settings for de-multiplexing, aligning reads to the mouse genome (10× Genomics pre-build mm10 reference genome) with STAR′ and counting unique molecular identifiers (UMIs) to build transcriptomic profiles of individual cells. For the v1 datasets, gene barcode matrices were analyzed with the R package Seurat v1.4, using the online tutorial as a guide^{7,76}. We first performed a filtering step, retaining only the cells that expressed a minimum of 200 genes and only the genes that were expressed in at least 3 cells. A large number of cells did not meet this threshold in the E17.5 time point and were determined to be red blood cells by the high expression of hemoglobin genes. Variable genes were determined by mean-variance relationship to identify highly expressed and variable genes with the Seurat function MeanVarPlot with default settings. UMI counts were log-normalized, and linear regression was performed with RegressOut to account for differences in the number of UMIs between cells. Principal component analysis (PCA) was then utilized to determine sources of variability in the dataset with PCAfast. Significant PCs were determined based on the Scree plot and utilized for Seurat's graph-based clustering algorithm (function FindClusters) with default parameters, except for the resolution parameter. To vary cluster numbers, the resolution parameter in FindClusters was adjusted from 0.6-3.0, and resulting clusters analyzed as follows. Clusters were visualized with t-distributed stochastic neighbor embedding (t-SNE) with Seurat's RunTSNE function with default settings⁷⁷. Differentially expressed genes were determined with the FindAllMarkers function, which uses a bimodal likelihood ratio test⁸. We confirmed differential gene expression analysis with the Wilcox rank sum test and MAST⁹ utilizing Seurat v2's FindMarkers function with default settings. These tests calculate adjusted p-values for multiple comparisons. To determine the final number of clusters, clusters were required to have at least 9 significantly (p<0.05) differentially expressed genes with a 2-fold difference in expression in comparison to all other clusters. Clusters were manually curated for differential gene expression, and those that did not meet this threshold were manually merged with the nearest cluster based on the phylogenetic tree from Seurat's BuildClusterTree. In some cases, clusters met the 9-gene threshold but appeared to have very similar differentially expressed genes to another cluster. This is likely a result of the comparison of individual clusters against all other clusters in determining differentially expressed genes. In these cases, a pairwise comparison between the two clusters was performed and the same 9-gene threshold applied. An exception to the 9-gene threshold was made to annotate the proliferating population in early stages of the cell cycle within the E14.5 mesenchymal analysis (FIG. 4, cluster 8). Additionally, cluster 10 in the E14.5 mesenchymal dataset did not meet the 9-gene threshold. Rather, clusters 1-9 had distinct transcriptomic signatures (with at least 9 differentially expressed genes) that distinguished them from cluster 10. Lists of at least 2-fold differentially expressed genes for individual analyses are provided in Table 1, Appendix. For v2 datasets, Seurat v2.2 and v2.3 was utilizing to perform the analysis. Cells with less than 200 genes and genes expressed in fewer than 3 cells were removed, as above. UMI counts were normalized with NormalizeData using default settings. Variable genes were determined with FindVariableGenes, using the following cut-offs consistent with the online tutorial (x.low.cutoff=0.0125, x.high.cutoff=3, y.cutoff=0.5). Data was scaled and UMI counts regressed out with the ScaleData function. Principal component analysis was performed with RunPCA, and significant PCs determined based on the Scree plot. t-SNE analysis and clustering was performed as described above for the v1 datasets. For the E12.5 exocrine dataset, the ductal population did not meet the 9-gene threshold. All other populations within this dataset could be distinguished from the ductal population by at least 9-differentially expressed genes, therefore we still annotated this cluster. Some of the clusters depicted for the Fev-Cre; ROSA26^{mTmG} dataset do not meet the 9-gene threshold. We chose to visualize these clusters in order to better illustrate their placement along the pseudotime trajectory.

Custom Genome Build

The custom genome for alignment of reads to eGFP and TdTomato sequences from the mTmG mouse line was created according to instructions provided by 10× Genomics reference support (https://support.10×genomics.com/single-cell-gene-expression/software/pipelines/latest/advanced/references). eGFP and TdTomato sequences were concatenated to the mm10-2.1.0 reference genome (FASTA file) provided by 10× Genomics. eGFP and TdTomato annotations were then concatenated to the mm10 annotations (GTF file) provided by 10× Genomics. The Cellranger mkref command was then utilized with the genome and annotations with eGFP and TdTomato, as described in the above link.

Pathway Analysis

Pathway analysis and calculation of associated p-values were performed using the ConsensusPathDB over-representation analysis for pathway-based sets category (http://cpdb.molgen.mpg.de)⁷⁸.

Aggregating E17.5 v2 Datasets

E17.5 technical replicates from the v2 dataset were aggregated with Cellranger v2.1, utilizing the aggr function with default settings. The aggregated dataset was used for analysis and merging with the E12.5 and E14.5 v2 datasets.

Sub-Clustering and Merging Datasets

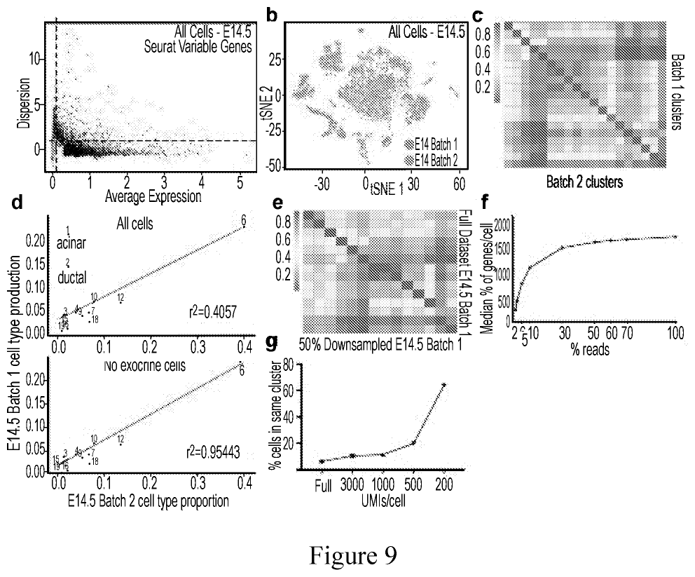

Sub-clustering was performed by isolating clusters of interest with the Seurat function SubsetData and reanalyzing as described above. Cells were classified as epithelial based on the expression of E-cadherin (Cdh1) and other known epithelial population markers. Cells that were Cdh1⁻; Vim⁺, and collagen3a1 (Col3a1)⁺ were classified as mesenchymal. Multiple batches were merged with the MergeSeurat function. The merged dataset was reanalyzed as above, with batch included as a latent variable in the RegressOut function. The v1 E14.5 batch 1 and batch 2 clusters were robust to the sampling differences between batches as evidenced by the contribution of cells from both batches to each cluster (FIG. 9b). We find high correlation of cell type proportion between batches in all populations except the exocrine compartment (acinar and ductal) (FIG. 9c), possibly due to technical challenges of pancreatic dissociation. Within each cluster, batch 1 cells correlated most highly with those of batch 2 contained in the same cluster, indicating proper cluster calling with the merged datasets (FIG. 9d).

For v2 datasets (E12.5, E14.5 and E17.5), multiple canonical correlation analysis (multiCCA) from Seurat v2.3 was utilized to merge the epithelial datasets³⁶. The top 1,000 most highly variable genes that were variable in at least 2 datasets were used for the alignment, as recommended in the Seurat tutorial. The shared correlation strength of each CC was measured with Seurat's MetageneBicorPlot, and those before the drop-off were used for alignment, analogous to the Scree plot in choosing significant PCs. We then aligned the datasets with AlignSubspace and ran an integrated t-SNE and clustering analysis, as outlined in the Seurat tutorial. Clusters were required to have 9 significantly differentially expressed genes as described above. Clusters with similar differentially expressed genes were verified with pairwise comparisons to the most related clusters (based on BuildClusterTree) and merged if they did not meet the pairwise 9-gene threshold. The Beta 2 cluster in the v2 endocrine merged time course data met the 9-gene threshold for 2 out of the 3 differential expression tests (Bimodal likelihood ratio and Wilcox rank sum tests), but had only 8 differentially expressed genes for the MAST test. Doublets were identified based on co-expression of two mutually exclusive genes, such as both mesenchymal and epithelial genes, and removed from further analysis. In the v2 datasets, rare cells (4 cells in E12.5 and 13 cells in E14.5 endocrine datasets) with high levels of hemoglobin gene expression were removed from the analysis.

Downsampling Analysis

To determine if the sequencing depth was sufficient for calling clusters, downsampling analysis was performed for the v1 E14.5 batch 1 dataset. Reads were randomly downsampled from the 10× Cellranger bam file output to a specified percentage, then grouped based on UMI to generate a count profile for each cell. The number of genes with greater than 0 counts was then calculated. UMI downsampling was performed with the SampleUMI function. A new Seurat object was created with the downsampled matrix and reanalyzed as above.

The number of UMIs/cell was downsampled from an average of 4,600 UMIs/cell in the full dataset to 200 UMIs/cell, and the median number of genes/cell and clustering robustness was then calculated. Clustering robustness was determined as the percentage of cells within the same cluster, with clusters required to maintain at least 9 genes with a 2-fold change in expression in comparison to all other clusters. Within this dataset, robust clustering was maintained all the way down to 500 UMIs/cell, when the percentage of cells in the same cluster began to climb, indicating collapsing of individual clusters. Both of these downsampling analyses indicate that sufficient sequencing depth was reached.

Pseudotemporal Ordering

We utilized Monocle 2.6.4⁷⁹ to order cells in “pseudotime” based on their transcriptomic similarity. For v1 time course datasets, batch-corrected values and variable genes from Seurat analysis were used as input, utilizing the gaussianff expressionFamily, and clusters were projected onto the minimum spanning tree after ordering.

For the Fev-lineage-traced dataset, UMI counts and variable genes from the Seurat analysis were used as input, utilizing the negBinom expressionFamily. To find genes differentially expressed across the branch point in the trajectory, we used monocle's internal BEAM analysis and selected genes with an FDR cutoff of 0.001. Gene expression patterns were plotted with plot_genes_branched_heatmap and plot_multiple_branches_pseudotime.

Code Availability

Scripts are available at https://github.com/sneddonucsf/2018-Developmental-single-cell-RNA-sequencing.

Data Availability

The accession number for the raw data files of the single-cell RNA sequencing analyses disclosed herein is GEO: GSE101099. The sequence data is incorporated herein by reference.

### Example 2

Cellular Heterogeneity in the Murine Pancreas

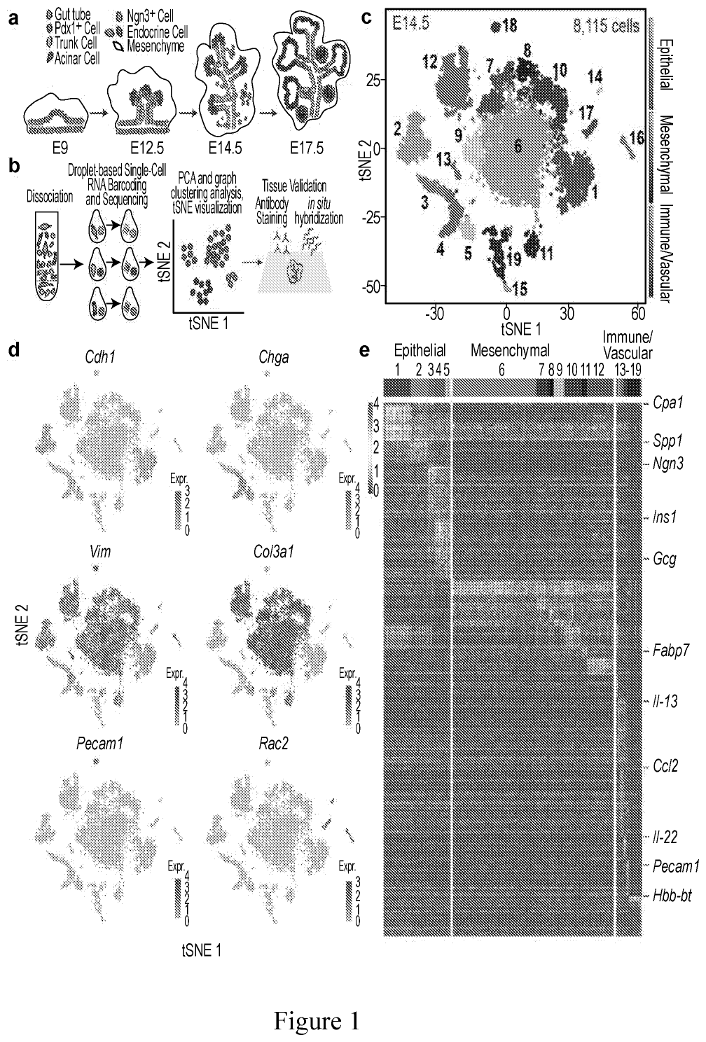

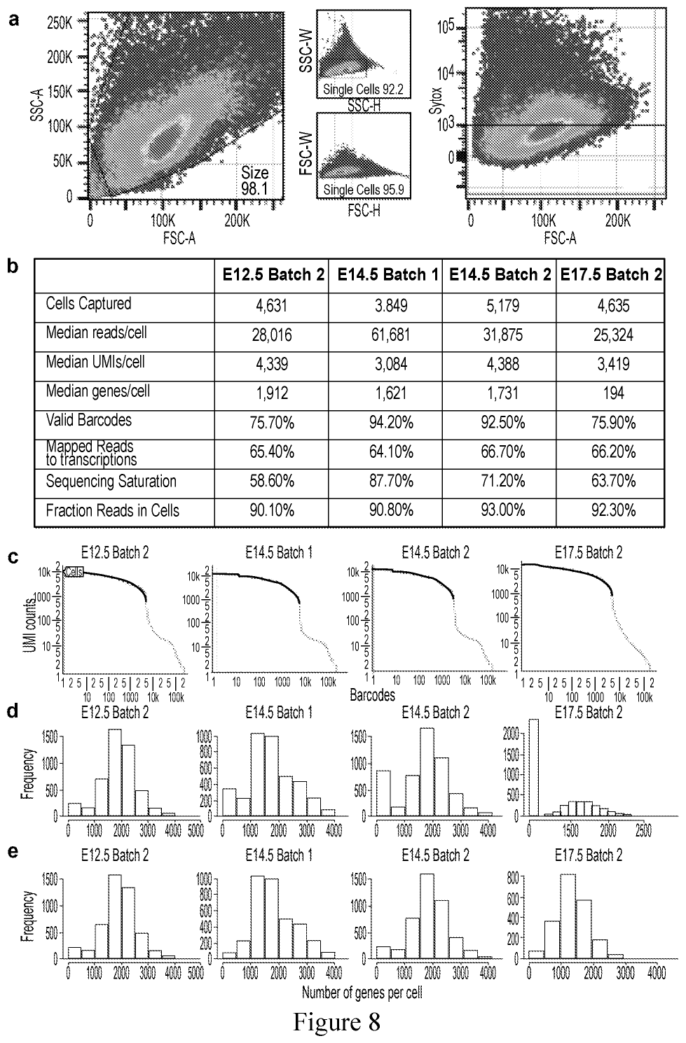

We first set out to characterize the major sources of cellular heterogeneity in the developing pancreas, in the most unbiased fashion possible. Two batches of mouse pancreata at E14.5, a particularly active time of expansion, morphogenesis, and diversification⁶ (FIG. 1a), were dissected from individual litters, dissociated into single-cell suspensions, sorted for live cells, and sequenced using the 10× Chromium Single-Cell platform with version 1 (v1) kits (FIG. 1b and FIG. 8a-e). We performed filtering, normalization, variable gene identification, linear regression for batch, and Principal Component Analysis (PCA) with the R package, Seurat (FIG. 8d,e and 9a,b). Graph-based clustering⁷ of batch-adjusted, merged data identified 19 distinct cell populations, classified as epithelial, mesenchymal, immune, or vascular populations based on the expression of known markers (FIG. 1c,d and Table 1). We were able to identify expected populations, including endocrine (alpha and beta), exocrine (acinar and ductal), and endothelial cells (FIG. 1e). We further found that the cell type proportion for endocrine, mesenchymal, immune, and vascular populations were similar between E14.5 batches (FIG. 9b-d). Downsampling analysis confirmed that sufficient sequencing depth had been reached for calling clusters (FIG. 9e-f). These results reveal the power of single-cell RNA sequencing to identify a broad range of cell types during development.

### Example 3

Identification of Novel Mesenchymal Populations

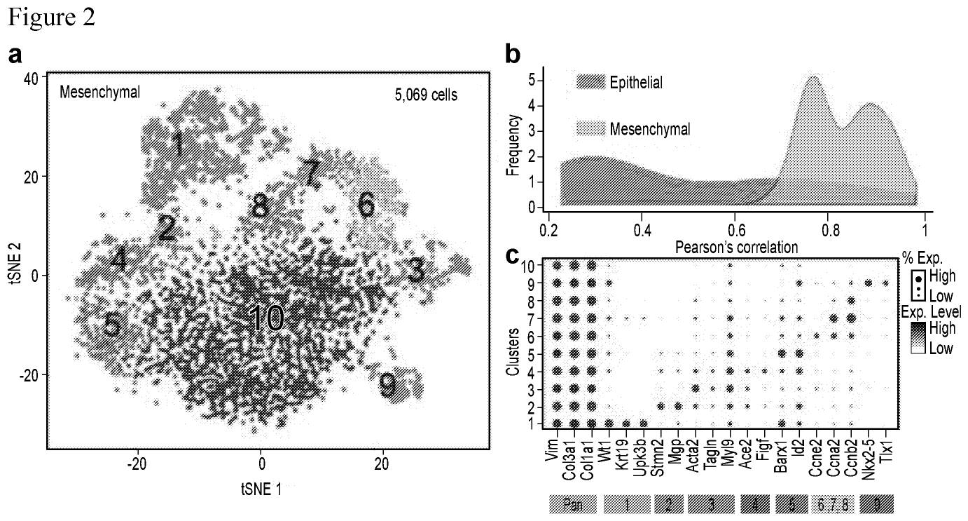

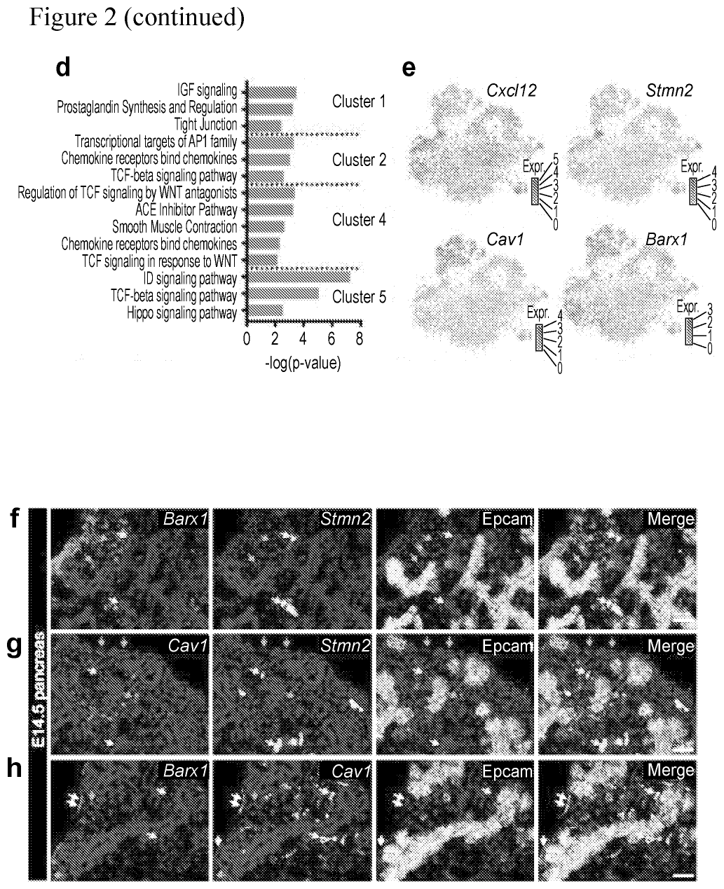

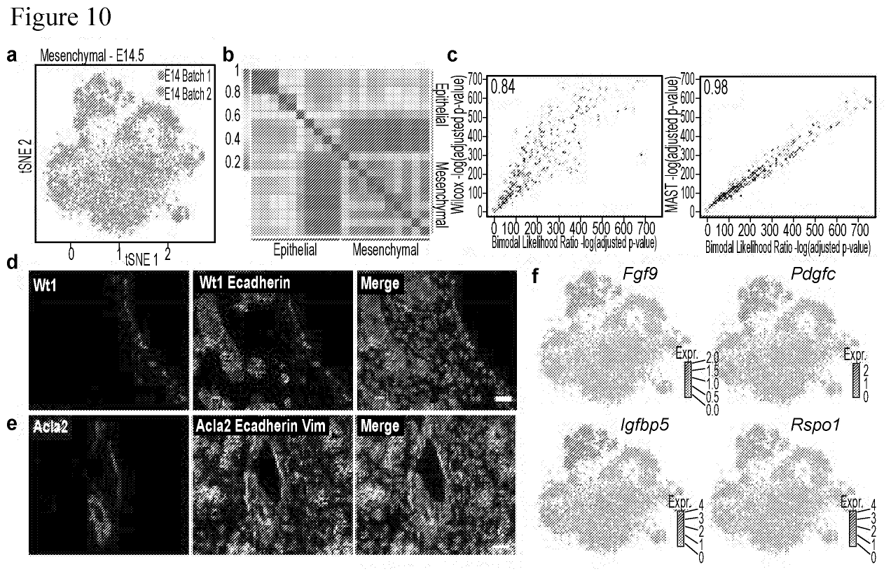

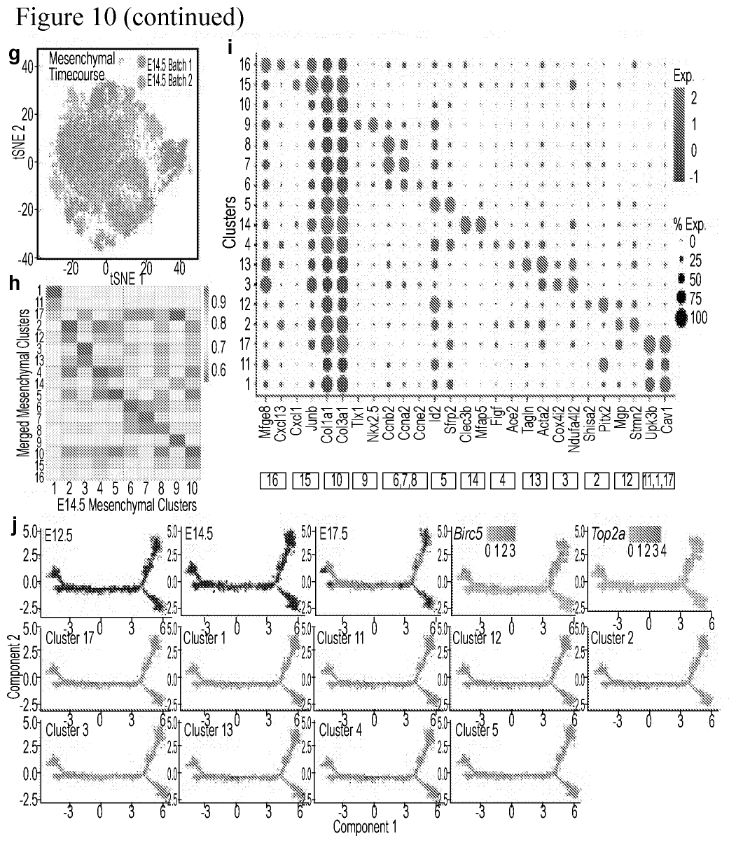

While previous studies have identified numerous markers of the various pancreatic epithelial populations⁶, comparatively little is known about heterogeneity among pancreatic mesenchymal cells or how they change over developmental time. We therefore turned our attention to the mesenchymal compartment by sub-clustering only mesenchymal cells (5,069 cells) and re-performing the clustering analysis (FIG. 2a and FIG. 10a). Despite being less divergent from one another than were cells in the epithelial compartment (FIG. 2b and FIG. 10b), mesenchymal cells could still be sub-divided into 10 transcriptionally distinct mesenchymal clusters (FIG. 2a,c and Table 1). We verified the differential gene expression analysis with three separate tests: bimodal likelihood ratio test⁸, Wilcox rank sum, and MAST⁹ (FIG. 10c and Table 1). We annotated two clusters based on the expression of known marker genes: cluster 1 is pancreatic mesothelial cells (Wt1, Krt19, and Upk3b)^{10,11} and cluster 3 represents vascular smooth muscle (VSM) cells (Acta2, Tagln, and Myl9) (FIG. 2c and Table 1)¹². Indeed, in E14.5 in vivo pancreas, Wt1 expression was restricted to the tissue edge, as expected for mesothelial cells, while Acta2 expression was localized to cells surrounding vessels, as expected for VSM cells (FIG. 10d,e). Cells in the mesothelial cluster were also characterized by the expression of secreted factors Fgf9, Pdgfc, Rspo1, and Igfbp5 (FIG. 10f) and by genes in pathways related to prostaglandin hormone signaling and tight junctions (FIG. 2d and Table 2, Appendix).

The remaining mesenchymal clusters included proliferating mesenchymal cells (clusters 6, 7, and 8), a large cluster (cluster 10) that expressed pan-mesenchymal markers, and four clusters (clusters 2, 4, 5, and 9) each expressing a distinct signature that segregated them from cluster 10 (FIG. 2a,c and Table 1). Cluster 2 was defined by differential expression of Stathmin 2 (Stmn2), a gene expressed during the differentiation of numerous cell types¹³⁻¹⁷. We also found two populations, clusters 4 and 5, that differentially expressed multiple secreted factors. Cluster 4 expressed angiotensin I converting enzyme 2 (Ace2), the chemokines Cxcl12 and Cxcl13, and vascular endothelial growth factor d (Vegfd), while cluster 5 expressed high levels of Wnt antagonists, secreted Frizzled-related protein 1 and 2 (Sfrp1 and Sfrp2) (FIG. 2c-e and Table 1). Cluster 5 also expressed the transcription factor Barx1 and members of the Id DNA-binding protein family (FIG. 2c-e and Table 1). Cluster 9 expressed Nk2 homeobox 5 (Nkx2-5) and T cell leukemia homeobox 1 (Tlx1), transcription factors previously reported to contribute to splenic development during a window in which the embryonic pancreas and spleen share a mesenchymal compartment (FIG. 2c)¹³. Pathway analysis identifies multiple signaling pathways that may be functionally relevant in these populations (FIG. 2d and Table 2). We validated a subset of these distinct clusters using dual in situ hybridization/immunofluorescence (ISH/IF) on E14.5 pancreas for differentially expressed markers of clusters 1 (Cav1 and Barx1), 2 (Stmn2), and 5 (Barx1) (FIG. 2e-h). These gene expression profiles demonstrate a previously underappreciated level of heterogeneity in the mesenchymal compartment of the developing pancreas.

### Example 4

Mesothelial Cells Undergo Transcriptional Changes Across Developmental Time

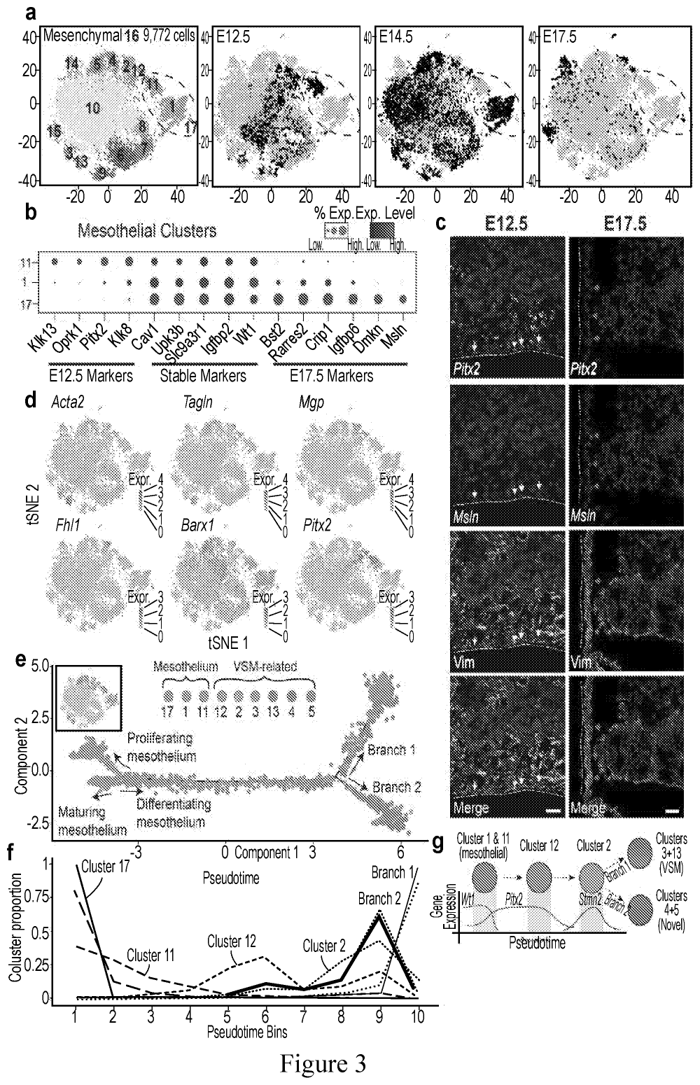

During organogenesis, the dynamics of each lineage are defined by the expansion, differentiation, and maturation of its constituent cells. To begin addressing how these processes change across chronological time within the developing pancreas, we performed single-cell sequencing of pancreas at two additional time points, E12.5 and E17.5 (FIG. 3a). We identified mesenchymal cells from E12.5, E14.5, and E17.5 time points, merged them into one dataset, and re-performed the clustering analysis. We identified the clusters detected in our E14.5 analysis (clusters 1-10) along with seven new clusters (clusters 11-17) (FIG. 3a, FIG. 10g-i, and Table 1). The addition of E12.5 and E17.5 cells revealed further sub-division of the mesothelium (clusters 1, 11, and 17) into time point-specific clusters, each with unique transcriptomic signatures (FIG. 3a,b). We validated the expression of time point-specific markers, Pitx2 and mesothelin (Msln), in E12.5 and E17.5 mesothelium in vivo (FIG. 3c). As predicted, Pitx2 expression was detected in mesothelium at E12.5 but not at E17.5, while Msln was found in mesothelium at E17.5 but not at E12.5 (FIG. 3c). These data provide evidence of transcriptional maturation over developmental time within the mesothelial compartment.

While the mesothelium is a well-established mesenchymal progenitor cell population for VSM and fibroblasts in multiple other organs, both the role of the mesothelium and the origin of the mesenchymal cell types within the pancreas remain uncharacterized¹⁴⁻¹⁷. We utilized our single-cell mesenchymal dataset to determine whether the pancreatic mesothelium may function as a mesenchymal progenitor cell population during development. We found six populations (clusters 2, 3, 4, 5, 12, and 13) that expressed VSM cell genes, such as Acta2 and Tagin, or genes known to regulate VSM development, such as Mgp¹⁸, Fhl1^{19,20}, Barx1²¹, and Pitx2²² (FIG. 3d). Based on these VSM-related gene expression profiles, we expected that these populations could represent VSM progenitors derived from the pancreatic mesothelium.

To test the lineage relationships among these populations, we ordered cells in pseudotime based on their transcriptional similarity²³. This analysis placed mesothelial cells on one side of the pseudotime trajectory (FIG. 3e). Mesothelial branches corresponded to either a maturation process, based on placement of E17.5 cells at the branch terminus, or proliferating mesothelium, based on expression of proliferation genes (FIG. 3e and FIG. 10j). VSM-related populations were placed on the other side of the trajectory (FIG. 3e and FIG. 10j). We calculated the proportion of each population over pseudotime to assess the distribution of clusters within the trajectory (FIG. 3f). We found a transition from the E12.5 mesothelial population (cluster 11) to cluster 12, both of which share expression of the gene Pitx2 (FIG. 3e-g). Cluster 12 then transitioned into the Stmn2-expressing cluster 2, which split into a branch composed of VSM populations, clusters 3 and 13 (Branch 1), and a branch composed of clusters 4 and 5 (Branch 2) (FIG. 3e-g). Thus, this analysis predicted clusters 2 and 12 as potential mesothelial-derived mesenchymal progenitor populations that can contribute to the VSM lineages (FIG. 3g). Therefore, our analysis has identified and validated multiple novel mesenchymal subtypes, as well as predicted lineage relationships, within the mesenchymal compartment of the developing pancreas.

### Example 5

Identification of a Novel Endocrine Progenitor Population

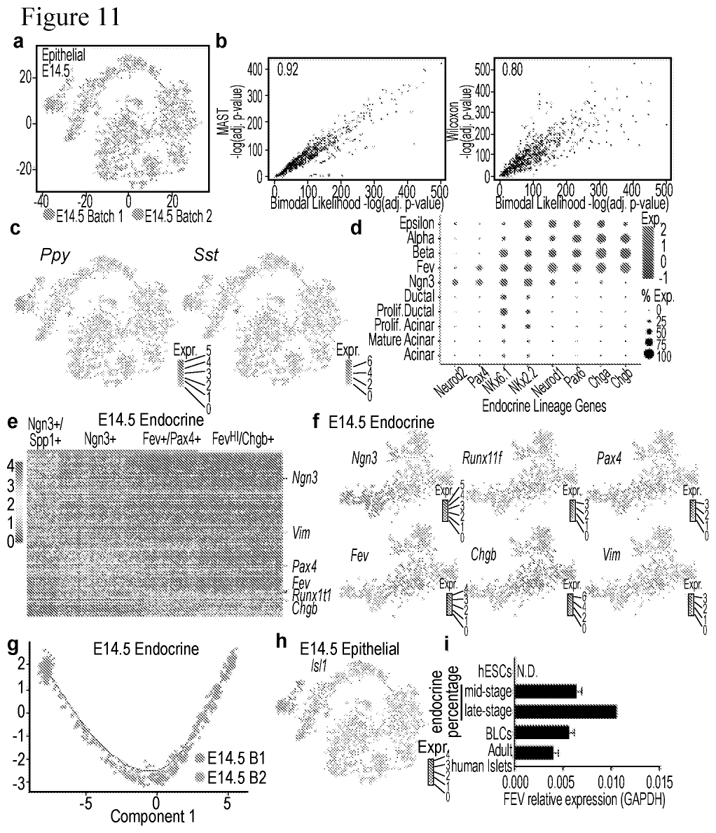

After assessing the heterogeneity within the mesenchymal compartment, we next focused on the epithelial cells. We first sub-clustered the 2,049 cells from our E14.5 dataset that comprised just the epithelial populations (FIG. 4a and FIG. 11a). We identified 10 clusters, including acinar, ductal, beta, alpha, and Ngn3⁺ progenitor populations, as revealed by differential expression of known markers (FIG. 4a,b, FIG. 11b, and Table 1). Our analysis also highlighted previously uncharacterized markers of acinar, Ngn3⁺, beta, and alpha cell populations, such as Reep5, Btbd17, Gng12, and Peg10, respectively (FIG. 4b and Table 1). We also found Sst⁻ and Ppy⁻ expressing cells, but they did not cluster into their own populations (FIG. 11c).

After the ductal, acinar, Ngn3⁺, and hormone⁺ populations had been accounted for, there still remained one population that eluded classification based on known marker genes. This novel population could be distinguished from all other epithelial populations by high-level expression of the E26 transformation-specific (ETS) transcription factor Fev, previously shown to be expressed within the developing pancreas but not described as a marker of a distinct population of epithelial cells²⁴ (FIG. 4a,b and Table 1). This Fev⁺ population expressed genes that mark cells in the endocrine lineage, such as Paired box 4 (Pax4), chromogranins A/B (Chga/b) and Neurod1¹ (FIG. 11d), but not mature endocrine markers, such as insulin1 (Ins1) or glucagon (Gcg), or the transitory early endocrine lineage marker, Ngn3 (FIG. 4b,c and Table 1). Pairwise comparison between this Fev⁺ cluster and the Ngn3⁺ cluster identified 99 genes more highly expressed in Fev⁺ and 87 more highly expressed in Ngn3⁺ cells, suggesting that the Fev⁺ and Ngn3⁺ clusters are distinct populations (FIG. 4d). This Fev⁺, Ngn3⁻, hormone⁻ cluster will henceforth be referred to as the Fev^{Hi} population. Pathway analysis of the Ngn3⁺ and Fev^{Hi} populations revealed enrichment of cell cycle and Notch signaling pathways in Ngn3⁺ cells (FIG. 4e and Table 3, Appendix), likely reflecting the exit of Ngn3⁺ progenitors from the cell cycle²⁵ and the role of Ngn3 in Notch signaling²⁶. The Fev^{Hi} cluster was distinguished by the expression of genes in pathways related to serotonin and insulin signaling, Activating Transcriptional Factor 2 (ATF-2) signaling, and sphingosine-1-phosphate signaling, which have been reported to regulate endocrine differentiation²⁷. This relationship to serotonin is consistent with prior work establishing Fev as a critical transcription factor in serotonergic neurons^{24,28}.

Further sub-clustering of all cells within the endocrine lineage (661 cells) revealed additional sub-groups of Fev-expressing cells. The first was marked by high expression of Pax4 and Runx1 Translocation Partner 1 (Runx1t1) and lower levels of Ngn3. The second was marked by Chgb and Vimentin (Vim) (FIG. 4f, FIG. 11e,f, and Table 1). Therefore, our data have predicted the existence of multiple novel intermediate states, marked by Fev, within the endocrine lineage. The Fev gene was also expressed at lower levels in a subset of the hormone-producing alpha, beta, and epsilon cell populations, which will collectively be referred to as hormone⁺/Fev^{Lo} populations (FIG. 4b).

Given that the novel Fev⁺ populations expressed endocrine lineage genes, we utilized pseudotime ordering²³ to test the expectation that both Fev⁺ populations were lineage-related to the Ngn3+ progenitors that give rise to the endocrine compartment of the pancreas²⁹. This de novo reconstruction of the developmental trajectory placed both the Fev⁺/Pax4⁺ and Fev^{Hi}/Chgb⁺ cells between Ngn3⁺ endocrine progenitors and alpha and beta cells (FIG. 4g and FIG. 11g), indicating that these Fev^{Hi} cells comprise a new progenitor stage of differentiation following Ngn3 expression and before hormone acquisition. The Fev⁺/Pax4⁺ population was placed closer in pseudotime to the Ngn3⁺ population and was followed by the Fev^{Hi}/Chgb⁺ population (FIG. 4g), indicating that the former represents an earlier progenitor cell state. Unlike alpha and beta cells, epsilon cells were found throughout the trajectory populated by the Fev⁺/Pax4⁺ and Fev^{Hi}/Chgb⁺ populations (FIG. 4g, magenta dots), possibly reflecting their function as multipotent progenitor cells for alpha and gamma lineages during development³⁰.

To validate these predicted lineage relationships, we performed an in vivo lineage trace of Ngn3⁺ cells. In E14.5 Ngn3-Cre; ROSA26^{mTmG} mouse pancreata, where lineage-traced cells are membrane-GFP⁺³¹, approximately 20% of all Ngn3-lineage-traced cells were identified as the predicted Fev^{HI} population by the presence of Fev and the absence of both Ngn3 and the pan⁻ differentiated endocrine cell marker Islet1 (Isl1) (FIG. 5a,e, yellow arrows and bar, and FIG. 11h). We also detected the hormone⁺/Fev^{Lo} population predicted by our single-cell data (FIG. 5a, purple arrows), as well as cells that co-expressed Fev and Ngn3 (blue arrows), consistent with a model in which Fev^{Hi} cells represent an intermediate progenitor state following Ngn3⁺ cells but prior to differentiated endocrine cells (FIG. 5g).

We next tested if the Fev^{Hi} population was also present in developing human pancreatic tissue. In human fetal pancreas at 23 weeks of gestation, we observed cells that only expressed NGN3 (FIG. 5b, gray arrows), cells that only expressed CHGA (magenta arrows), a marker of all hormone-expressing endocrine cells, and those that co-expressed FEV and CHGA (purple arrows). We also detected cells that expressed FEV but not NGN3 or CHGA (FIG. 5c, yellow arrows), representing a FEV⁺/hormone⁻ population. The existence of these cellular states in human development indicates that the lineage relationships identified herein can be generalized beyond murine pancreatic organogenesis to that of human, as well.

We then probed hESCs undergoing directed differentiation towards the pancreatic beta cell lineage in vitro³². FEV transcript was detected in endocrine progenitor-stage cells and beta-like cells (BLCs) at levels comparable to adult human islets, but it was not detected in undifferentiated hESCs (FIG. 11i). Further, we observed FEV⁺ (NGN3⁻/ISL1⁻) (yellow arrows), FEV⁺/ISL1⁺ (NGN3⁻) (purple arrows), and NGN3⁺/FEV⁺ (ISL1⁻) (blue arrows) populations in differentiating hESC-derived cells mid-way through the endocrine progenitor stage (FIG. 5d,f). While endocrine differentiation progresses as a wave throughout development³³ in vivo, it is more synchronized in the hESC differentiation platform in vitro^{32,34,35}. At a time point directly preceding beta cell differentiation, we found that nearly 70% of hESC-derived cells were either NGN3⁺/FEV⁺ or FEV⁺ (FIG. 5f, blue and yellow bars). These data place the FEV⁺ population at a time point consistent with an endocrine progenitor population during human beta cell differentiation in vitro.

### Example 6

Endocrine Dynamics Over Developmental Time

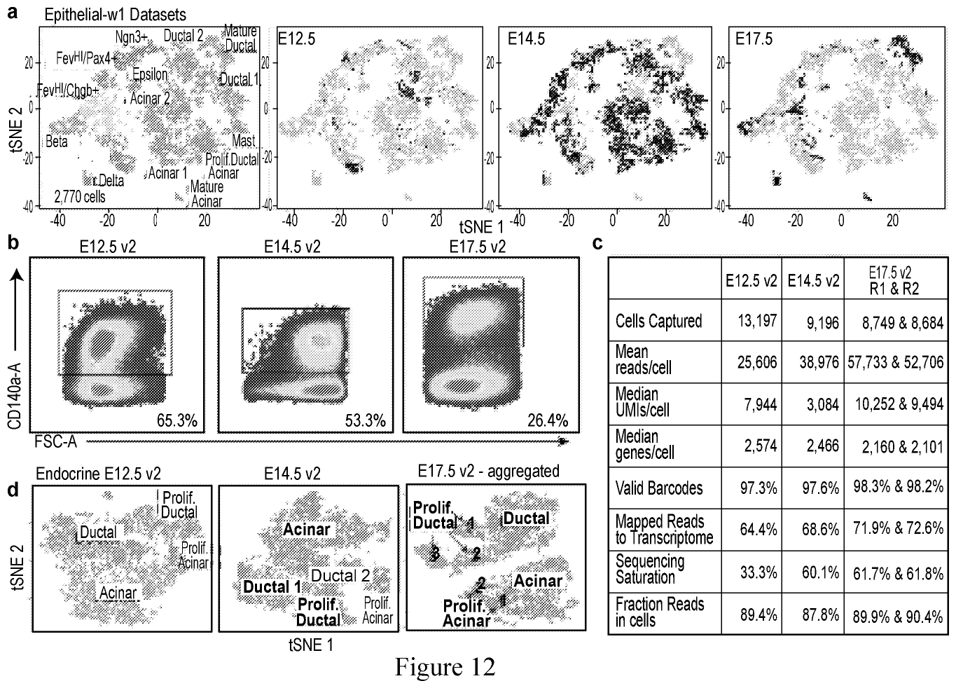

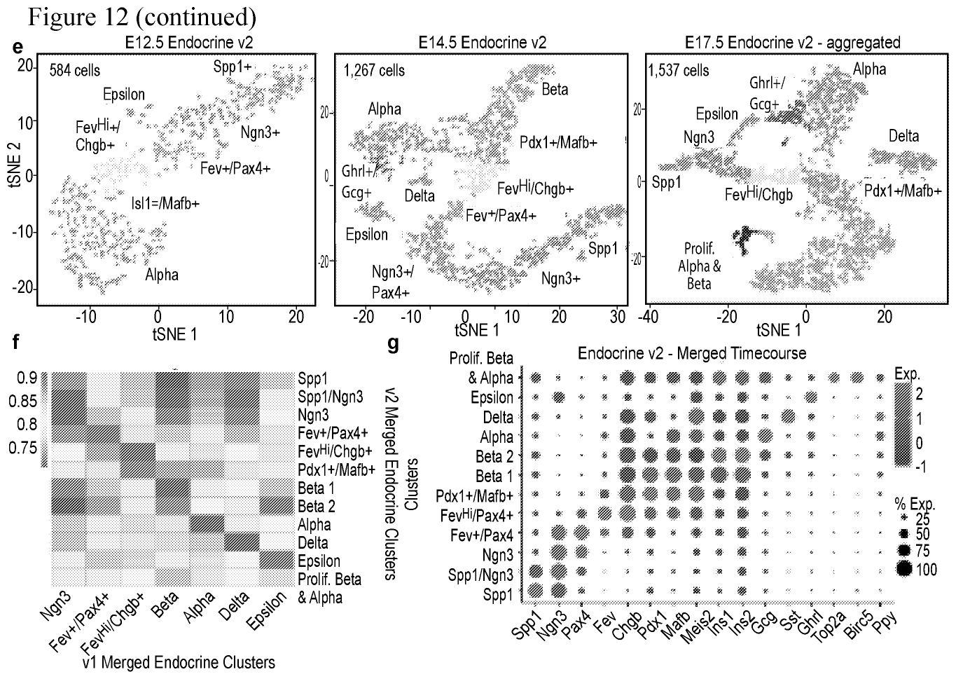

Although we had captured comparatively fewer epithelial cells at E12.5 and E17.5 than at E14.5, we could still identify the Fev^{Hi} cells at both time points (FIG. 12a). To capture more epithelial cells and account for those that were missing from E12.5 and E17.5 version 1 (v1) runs, we re-performed an entirely new (version 2) set of single-cell RNA sequencing experiments at E12.5, E14.5, and E17.5 after depletion of CD140a⁺ mesenchymal cells in order to enrich for epithelial cells (FIG. 12b,c). Given the high numbers of red blood cells at E17.5, we ran two wells of E17.5 cells (replicates 1 and 2) to increase our capture of epithelial cells and then aggregated the datasets. We first analyzed the exocrine compartment and identified acinar, ductal, and proliferating populations of both at all time points (FIG. 12d and Table 4, Appendix). We then focused on the endocrine compartment, where we captured 584, 1,267, and 1,837 endocrine cells at E12.5, E14.5, and E17.5, respectively. We found similar gene expression topologies as in our v1 dataset but gained additional resolution with increased cell numbers and transcriptomic coverage (FIG. 12e and Table 4).

To analyze how endocrine populations change overtime, we merged all three v2 time points into one dataset using canonical correlation analysis³⁶. We correlated the v2 dataset to the v1 dataset and could identify all populations present in the v1 dataset (FIG. 12g). We also find additional populations, including a cluster characterized by decreased expression of Fev and increased expression of Pdx1 and Mafb, genes with known roles in endocrine lineage decisions (FIG. 5h). This Pdx1⁺/Mafb⁺ population correlates most strongly with the Fev^{Hi}/Chgb⁺ population, as well as both the alpha and beta cell populations in the v1 dataset (FIG. 12g). We also found a second beta cell population characterized by increasing expression of Ins1 and Ins2, and lower expression of Pdx1, representing perhaps a more mature group of beta cells. Indeed, this second beta cell group is almost entirely comprised of cells from the E17.5 time point. To examine how these populations shift over developmental time, we calculated the proportion of these populations at each time point (FIG. 5j). We found shifts in cell proportions that match those reported in literature, such as a high proportion of alpha cells early in development at E12.5 and increasing proportions of beta and delta cells at later timepoints³³. The Ngn3⁺ population decreased overtime, while the Fev⁺/Pax4⁺, Fev^{Hi}/Chgb⁺, and Pdx1⁺/Maf^{b}+ populations peaked at E14.5, consistent with previous studies that reported peak Ngn3 expression at approximately E14.5 and its subsequent downregulation as differentiation into endocrine lineage ensues³⁷. At E17.5, we also found an increasing proportion of proliferating endocrine cells, presumably those responsible for the expansion of endocrine cell mass in later embryonic development³⁸. These results from the larger v2 dataset confirm our initial findings from the v1 dataset and add additional resolution to the endocrine populations during pancreatic development.

### Example 7

Lineage Decisions within the Endocrine Compartment

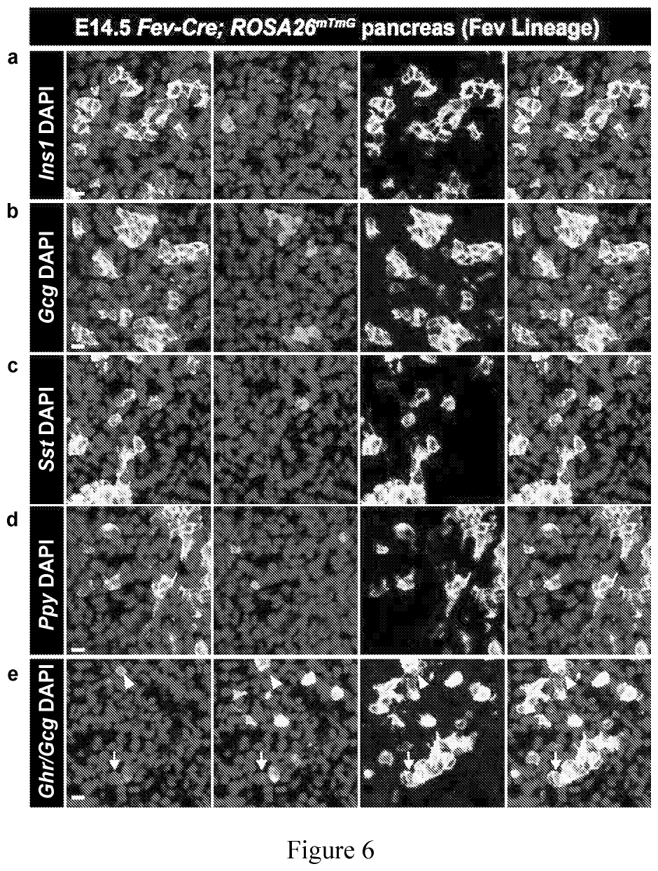

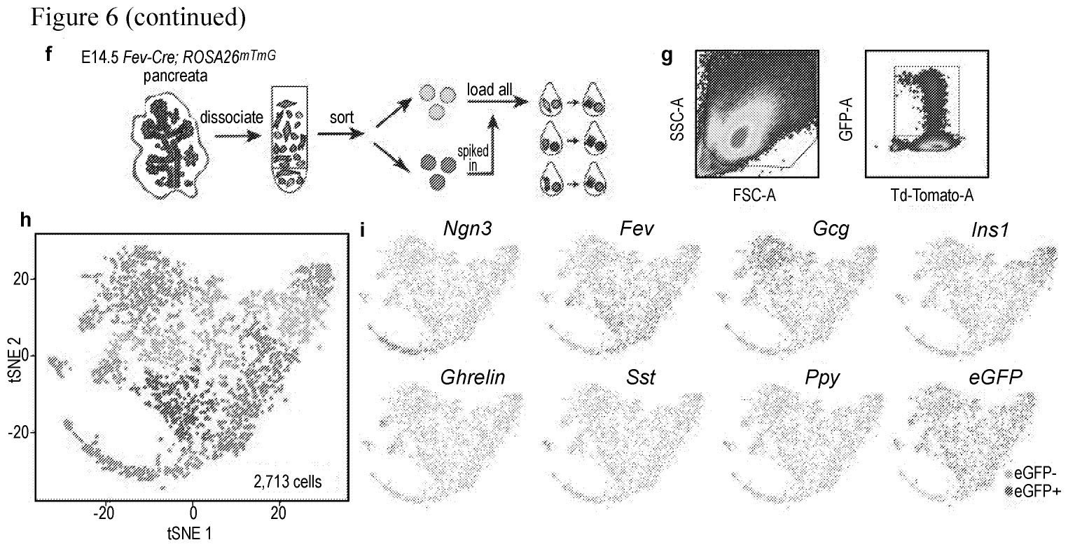

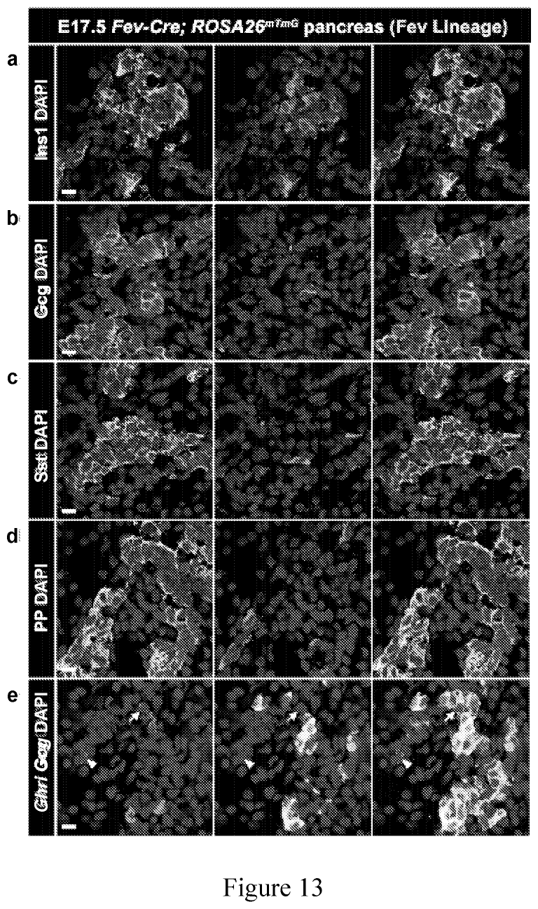

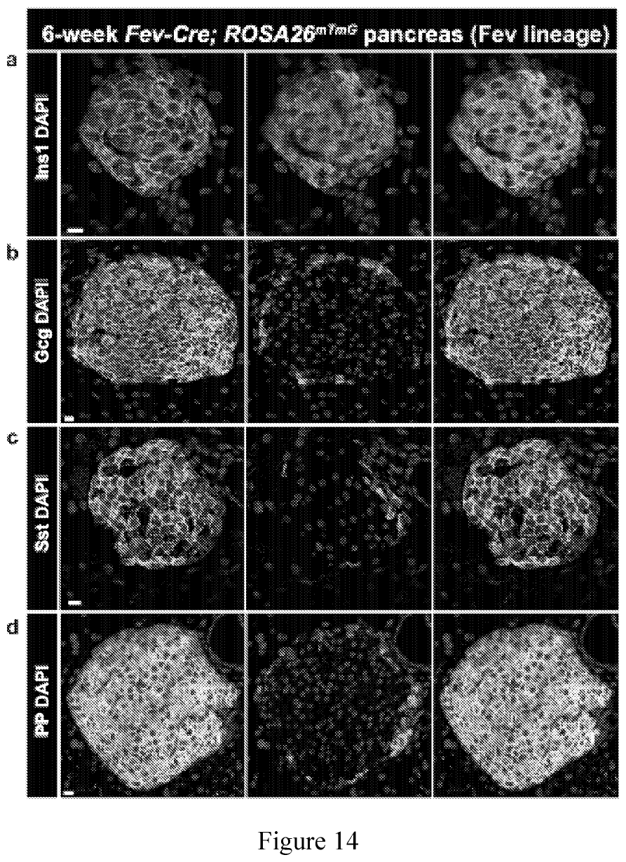

As the in vivo lineage tracing data had revealed that the Fev^{Hi} population is derived from the Ngn3⁺ population, we expected that the Fev^{Hi} population could then function as a progenitor for the endocrine populations of the developing pancreas. We utilized a Fev-Cre; ROSA26^{mTmG} lineage tracing strategy to label Fev-expressing cells and their progeny. We found 100% of alpha, 100% of beta, 100% of delta, 89.1% of gamma, and 23.8% of epsilon cells were Fev-lineage-traced in E14.5 pancreas (FIG. 6a-e). These proportions of Fev lineage-labeling held true later in development (E17.5) and in adulthood (6 weeks) (FIG. 13 and FIG. 14). Epsilon cells are rare in the adult pancreas³⁰ and still exhibited only partial Fev-lineage tracing in E17.5 pancreas (47.8% traced) (FIG. 13e). The Fev-lineage-negative epsilon population identified in this study may correspond to the proportion of epsilon cells during pancreatic development that have been proposed to be Ngn3-independent, multi-potent progenitors³⁰.

With evidence in vivo that the majority of endocrine cells pass through a Fev-expressing stage, we next combined this lineage tracing approach with single-cell RNA sequencing to identify transcriptional regulators of endocrine differentiation. We used Fev-Cre; ROSA26mTmG pancreata to enrich for Fev-expressing cells and their progeny (membrane-GFP⁺) at E14.5 with FACS sorting (FIG. 6f,g). All expected endocrine populations were identified in the resulting single-cell dataset (FIG. 6h,i). In addition, we found that eGFP reads mapped to all endocrine populations except the Ngn3⁺ population (FIG. 6i), further confirming that Fev expression turns on after Ngn3.

We next set out to predict the lineage relationships among the endocrine cells and identify transcriptional regulators of differentiation. Pseudotime ordering identified a trajectory that began with Ngn3⁺ cells, transitioned into Fev⁺ cells, and then split into two main branches (FIG. 7a; see similar branching pattern in analysis of our first v1 dataset, FIG. 15a). The termini of the branches were populated by differentiated beta and alpha cells, indicating that the branches represent a transition from a progenitor to fully differentiated hormone⁺ cell (FIG. 7a).

We next used an analysis tool in the Monocle software called branched expression analysis modeling (BEAM) to identify the genes that distinguish the paths along the two branches to either alpha or beta cells. We found gene clusters that were upregulated along different segments of the pseudotime trajectory (FIG. 7b) and performed pathway analysis to identify pathways enriched at each stage of pseudotime (FIG. 15c and Table 5, Appendix). Genes upregulated at the beginning of pseudotime in gene cluster 2 included early markers of endocrine differentiation, such as Sox4 and Ngn3 (FIG. 7b). Fev was in gene cluster 6 and increased in both branches before ultimately decreasing in expression at the branch termini (FIG. 7b,c). Gene cluster 6 also included other genes expressed within the Fev^{Hi} population, such as Cldn4, Vim, and Chgb (FIG. 7b,c and FIG. 15b). We found branch-specific clusters that included known markers of beta (Ins1) and alpha (Gcg) cells and known differentiation regulators of alpha (Arx, Pou3f4, Irx1, Sic38a5, and Tmem27) and beta cells (Pdx1, Pak3, and Nkx6-1) (FIG. 15b). These clusters also contained genes that were enriched in either the alpha or beta branch but were expressed before acquisition of hormone expression (FIG. 15b). Within the alpha cell branch, Peg10, Smarca1 Auts2, and Wnk3 increased in expression before upregulation of Gcg occurred (FIG. 15b). Peg10 and Auts2 have roles in differentiation^{39,40} and migration⁴¹ processes, but a role in endocrine differentiation or cell fate decisions has not been described. Smarca1 encodes a component of the chromatin remodeling complex and has been identified as an adult human alpha cell marker⁴². As a regulator of chromatin states, this gene may be involved in the epigenetic regulation of alpha cell differentiation during development. Within the beta cell branch, Gng12, Tssc4, Ece1, Tmem108, Wipi1 and Papss2 increased in expression before upregulation of Ins1 commenced (FIG. 15b). A role in endocrine lineage decisions have not been described for these beta branch-specific genes, although several have been associated with differentiation or maturation processes in other cell types⁴³⁻⁴⁵. We found a similar endocrine differentiation trajectory by an orthogonal method that uses force-directed layouts to visualize gene topologies and infer lineage relationships within single-cell data^{46 47} (FIG. 15d). We expect the genes identified by the analysis above to represent novel regulators of the differentiation of an endocrine progenitor to a fully differentiated hormone-expressing cell.

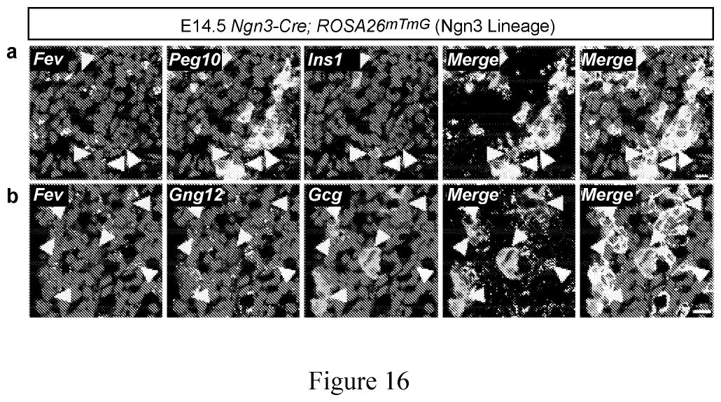

To validate the predictions of our pseudotime analysis, we performed in situ hybridization for markers that defined each branch of the trajectory. First, we confirmed the expression of Peg10 and Gng12 within the Fev^{Hi} population (FIG. 7d,e, indigo- and teal-graded arrows), validating the expression of these genes in a stage before hormone acquisition. We also validated the enrichment of Peg10 and Gng12 in alpha and beta cells, respectively (FIG. 7f,g, solid indigo and teal arrows). First, 95.8% of beta cells expressed Gng12 (n=46 cells, 6 pancreata), while 30.5% expressed Peg10 (n=71 cells, 7 pancreata) (FIG. 7f and FIG. 16a). Additionally, 100% of alpha cells expressed Peg10 (n=31 cells, 6 pancreata), while only 5.4% expressed Gng12 (n=32 cells, 4 pancreata) (FIG. 7g and FIG. 16b). The predicted lineage relationships from the pseudotime ordering, combined with the validation in vivo, led us to expect that the Fev⁺/Peg10⁺ cells are fated towards an alpha cell identity and Fev⁺/Gng12⁺ cells towards a beta cell identity (FIG. 7h). These results indicate that lineage allocation of endocrine progenitors towards alpha or beta cell fates occurs after the onset of Fev expression.

### REFERENCES

1. Shih, H. P., Wang, A. & Sander, M. Pancreas Organogenesis: From Lineage Determination to Morphogenesis. Annu. Rev. Cell Dev. Biol. 29, 81-105 (2013).
2. Suissa, Y. et al. Gastrin: A Distinct Fate of Neurogenin3 Positive Progenitor Cells in the Embryonic Pancreas. PLoS ONE 8, e70397 (2013).
3. Qiu, W.-L. et al. Deciphering Pancreatic Islet 13 Cell and a Cell Maturation Pathways and Characteristic Features at the Single-Cell Level. Cell metabolism 25, 1194-1205.e4 (2017).
4. Zeng, C. et al. Pseudotemporal Ordering of Single Cells Reveals Metabolic Control of Postnatal b Cell Proliferation. Cell metabolism 25, 1160-1175.e11 (2017).
5. Dorrell, C. et al. Human islets contain four distinct subtypes of 13 cells. Nat Comms 7, 11756 (2016).
6. Pan, F. C. & Wright, C. Pancreas organogenesis: From bud to plexus to gland. Developmental Dynamics 240, 530-565 (2011).
7. Satija, R., Farrell, J. A., Gennert, D., Schier, A. F. & Regev, A. Spatial reconstruction of single-cell gene expression data. Nature Biotechnology 33, 495-502 (2015).
8. McDavid, A. et al. Data exploration, quality control and testing in single-cell qPCR-based gene expression experiments. Bioinformatics 29, 461-467 (2012).
9. Finak, G. et al. MAST: a flexible statistical framework for assessing transcriptional changes and characterizing heterogeneity in single-cell RNA sequencing data. Genome Biol. 16, 278 (2015).
10. Kanamori-Katayama, M. et al. LRRN4 and UPK3B are markers of primary mesothelial cells. PLoS ONE 6, e25391 (2011).
11. Winters, N. & Bader, D. Development of the Serosal Mesothelium. JDB 1, 64-81 (2013).
12. Majesky, M. W., Dong, X. R., Regan, J. N., Hoglund, V. J. & Schneider, M. Vascular Smooth Muscle Progenitor Cells. Circulation Research 108, 365-377 (2011).
13. Hecksher-Sørensen, J. et al. The splanchnic mesodermal plate directs spleen and pancreatic laterality, and is regulated by Bapx1/Nkx3.2. Development 131, 4665-4675 (2004).
14. Bin Zhou et al. Epicardial progenitors contribute to the cardiomyocyte lineage in the developing heart. Nature 454, 109-113 (2008).
15. Asahina, K., Zhou, B., Pu, W. T. & Tsukamoto, H. Septum transversum-derived mesothelium gives rise to hepatic stellate cells and perivascular mesenchymal cells in developing mouse liver. Hepatology 53, 983-995 (2011).
16. Que, J. et al. Mesothelium contributes to vascular smooth muscle and mesenchyme during lung development. Proceedings of the National Academy of Sciences of the United States of America 105, 16626-16630 (2008).
17. Wilm, B., Ipenberg, A., Hastie, N. D., Burch, J. B. E. & Bader, D. M. The serosal mesothelium is a major source of smooth muscle cells of the gut vasculature. Development 132, 5317-5328 (2005).
18. Speer, M. Y. et al. Smooth Muscle Cells Give Rise to Osteochondrogenic Precursors and Chondrocytes in Calcifying Arteries. Circulation Research 104, 733-741 (2009).
19. Wang, L.-L. et al. Up-regulated FHL1 Expression Maybe Involved in the Prognosis of Hirschsprung's Disease. Int. J. Med. Sci. 11, 262-267
20. Kwapiszewska, G. et al. Fhl-1, a New Key Protein in Pulmonary Hypertension. Circulation 118, 1183-1194 (2008).
21. Jayewickreme, C. D. & Shivdasani, R. A. Control of stomach smooth muscle development and intestinal rotation by transcription factor BARX1. Developmental biology 405, 21-32 (2015).
22. Shang, Y., Yoshida, T., Amendt, B. A., Martin, J. F. & Owens, G. K. Pitx2 is functionally important in the early stages of vascular smooth muscle cell differentiation. The Journal of Cell Biology 181, 461-473 (2008).
23. Qiu, X. et al. Reversed graph embedding resolves complex single-cell developmental trajectories. bioRxiv 110668 (2017). doi:10.1101/110668
24. Ohta, Y. et al. Convergence of the Insulin and Serotonin Programs in the Pancreatic b-Cell. Diabetes 60, 3208-3216 (2011).
25. Miyatsuka, T., Kosaka, Y., Kim, H. & German, M. S. Neurogenin3 inhibits proliferation in endocrine progenitors by inducing Cdknla. PNAS 108, 185-190 (2011).
26. Shih, H. P. et al. A Notch-dependent molecular circuitry initiates pancreatic endocrine and ductal cell differentiation. Development 139, 2488-2499 (2012).
27. Han, S.-I., Yasuda, K. & Kataoka, K. ATF2 interacts with beta-cell-enriched transcription factors, MafA, Pdx1, and beta2, and activates insulin gene transcription. J. Biol. Chem. 286, 10449-10456 (2011).
28. Spencer, W. C. & Deneris, E. S. Regulatory Mechanisms Controlling Maturation of Serotonin Neuron Identity and Function. Front. Cell. Neurosci. 11, 302 (2017).
29. Gu, G., Dubauskaite, J. & Melton, D. A. Direct evidence for the pancreatic lineage: NGN3⁺ cells are islet progenitors and are distinct from duct progenitors. Development 129, 2447-2457 (2002).
30. Arnes, L., Hill, J. T., Gross, S., Magnuson, M. A. & Sussel, L. Ghrelin Expression in the Mouse Pancreas Defines a Unique Multipotent Progenitor Population. PLoS ONE 7, e52026 (2012)
31. Muzumdar, M. D., Tasic, B., Miyamichi, K., Li, L. & Luo, L. A global double-fluorescent Cre reporter mouse. Genesis 45, 593-605 (2007).
32. Pagliuca, F. W. et al. Generation of Functional Human Pancreatic 13 Cells In Vitro. Cell 159, 428-439 (2014).
33. Johansson, K. A. et al. Temporal Control of Neurogenin3 Activity in Pancreas Progenitors Reveals Competence Windows for the Generation of Different Endocrine Cell Types. Developmental Cell 12, 457-465 (2007).
34. Russ, H. A. et al. Controlled induction of human pancreatic progenitors produces functional beta-like cells in vitro. The EMBO Journal 34, 1759-1772 (2015).
35. Rezania, A. et al. Reversal of diabetes with insulin-producing cells derived in vitro from human pluripotent stem cells. Nature Biotechnology 32, 1121-1133 (2014).
36. Butler, A., Hoffman, P., Smibert, P., Papalexi, E. & Satija, R. Integrating single-cell transcriptomic data across different conditions, technologies, and species. Nature Biotechnology 36, 411-420 (2018).
37. Villasenor, A., Chong, D. C. & Cleaver, O. Biphasic Ngn3 expression in the developing pancreas. Developmental Dynamics 237, 3270-3279 (2008).
38. Bonner-Weir, S., Aguayo-Mazzucato, C. & Weir, G. C. Dynamic development of the pancreas from birth to adulthood. Upsala Journal of Medical Sciences 121, 155-158 (2016).
39. Hishida, T., Naito, K., Osada, S., Nishizuka, M. & Imagawa, M. peg10, an imprinted gene, plays a crucial role in adipocyte differentiation. FEBS Letters 581, 4272-4278 (2007).
40. Dekel, B. et al. Multiple Imprinted and Sternness Genes Provide a Link between Normal and Tumor Progenitor Cells of the Developing Human Kidney. Cancer Res 66, 6040-6049 (2006).
41. Hori, K. et al. Cytoskeletal Regulation by AUTS2 in Neuronal Migration and Neuritogenesis. Cell Reports 9, 2166-2179 (2014).
42. Muraro, M. J. et al. A Single-Cell Transcriptome Atlas of the Human Pancreas. Cell Systems 3, 385-394.e3 (2016).
43. Jiao, H.-F. et al. Transmembrane protein 108 is required for glutamatergic transmission in dentate gyrus. PNAS 114, 1177-1182 (2017).
44. Ho, H., Kapadia, R., Al-Tahan, S., Ahmad, S. & Ganesan, A. K. WIPI1 coordinates melanogenic gene transcription and melanosome formation via TORC1 inhibition. J. Biol. Chem. 286, 12509-12523 (2011).
45. Wang, W., Li, F., Wang, K., Bin Cheng & Guo, X. PAPSS2 Promotes Alkaline Phosphates Activity and Mineralization of Osteoblastic MC3T3-E1 Cells by Crosstalk and Smads Signal Pathways. PLoS ONE 7, e43475 (2012).
46. Weinreb, C., Wolock, S. & Klein, A. M. SPRING: a kinetic interface for visualizing high dimensional single-cell expression data. Bioinformatics 34, 1246-1248 (2018).
47. Tusi, B. K. et al. Population snapshots predict early haematopoietic and erythroid hierarchies. Nature Publishing Group 555, 54-60 (2018).
48. Golosow, N. & Grobstein, C. Epitheliomesenchymal interaction in pancreatic morphogenesis. Developmental biology 4, 242-255 (1962).
49. Landsman, L. et al. Pancreatic Mesenchyme Regulates Epithelial Organogenesis throughout Development. PLOS Biology 9, e1001143 (2011).
50. Bhushan, A. et al. Fgf10 is essential for maintaining the proliferative capacity of epithelial progenitor cells during early pancreatic organogenesis. Development 128, 5109-5117 (2001).
51. Yin, Y., Wang, F. & Ornitz, D. M. Mesothelial- and epithelial-derived FGF9 have distinct functions in the regulation of lung development. Development 138, 3169-3177 (2011).
52. Katsumoto, K. & Kume, S. The Role of CXCL12-CXCR4 Signaling Pathway in Pancreatic Development. Theranostics 3, 11-17
53. Kayali, A. G. et al. The stromal cell-derived factor-1a/CXCR4 ligand-receptor axis is critical for progenitor survival and migration in the pancreas. The Journal of Cell Biology 163, 859-869 (2003).
54. Murtaugh, L. C. The what, where, when and how of Wnt/β-catenin signaling in pancreas development. Organogenesis 4, 81-86 (2008).
55. Kapadia, C., Ghosh, M. C., Grass, L. & Diamandis, E. P. Human kallikrein 13 involvement in extracellular matrix degradation. Biochemical and Biophysical Research Communications 323, 1084-1090 (2004).
56. Huang, C. et al. Dermokine contributes to epithelial-mesenchymal transition through increased activation of signal transducer and activator of transcription 3 in pancreatic cancer. Cancer Science 108, 2130-2141 (2017).
57. Hasegawa, M. et al. Dermokine inhibits ELR+CXC chemokine expression and delays early skin wound healing. Journal of Dermatological Science 70, 34-41 (2013).
58. Ernst, M. C. & Sinal, C. J. Chemerin: at the crossroads of inflammation and obesity. Trends in Endocrinology & Metabolism 21, 660-667 (2010).
59. Angelo, J. R. & Tremblay, K. D. Identification and fate mapping of the pancreatic mesenchyme. Developmental biology (2018). doi:10.1016/j.ydbio.2018.01.003
60. Bin Zhou et al. Adult mouse epicardium modulates myocardial injury by secreting paracrine factors. J Clin Invest 121, 1894-1904 (2011).
61. Li, Y., Wang, J. & Asahina, K. Mesothelial cells give rise to hepatic stellate cells and myofibroblasts via mesothelial-mesenchymal transition in liver injury. Proceedings of the National Academy of Sciences of the United States of America 110, 2324-2329 (2013).
62. Zolak, J. S. et al. Pleural Mesothelial Cell Differentiation and Invasion in Fibrogenic Lung Injury. The American Journal of Pathology 182, 1239-1247 (2013).
63. Jensen, J. N. et al. Recapitulation of elements of embryonic development in adult mouse pancreatic regeneration. Gastroenterology 128, 728-741 (2005).
64. Rhim, A. D. & Stanger, B. Z. Molecular Biology of Pancreatic Ductal Adenocarcinoma Progression: Aberrant Activation of Developmental Pathways. Progress in Molecular Biology and Translational Science 97, 41-78 (2010).
65. Miyatsuka, T., Li, Z. & German, M. S. Chronology of Islet Differentiation Revealed By Temporal Cell Labeling. Diabetes 58, 1863-1868 (2009).
66. Collombat, P. et al. Opposing actions of Arx and Pax4 in endocrine pancreas development. Genes & Development 17, 2591-2603 (2003).
67. Benitez, C. M. et al. An Integrated Cell Purification and Genomics Strategy Reveals Multiple Regulators of Pancreas Development. PLOS Genetics 10, e1004645 (2014).
68. Butler, A. E. et al. 13-Cell Deficit in Obese Type 2 Diabetes, a Minor Role of 13-Cell Dedifferentiation and Degranulation. J Clin Endocrinol Metab 101, 523-532 (2016).
69. Wyler, S. C. et al. Pet-1 Switches Transcriptional Targets Postnatally to Regulate Maturation of Serotonin Neuron Excitability. J. Neurosci. 36, 1758-1774 (2016).
70. Schonhoff, S. E., Giel-Moloney, M. & Leiter, A. B. Neurogenin 3-expressing progenitor cells in the gastrointestinal tract differentiate into both endocrine and non-endocrine cell types. Developmental biology 270, 443-454 (2004).
71. Scott, M. M. et al. A genetic approach to access serotonin neurons for in vivo and in vitro studies. PNAS 102, 16472-16477 (2005).
72. Grabinski, T. M., Kneynsberg, A., Manfredsson, F. P. & Kanaan, N. M. A Method for Combining RNAscope In Situ Hybridization with Immunohistochemistry in Thick Free-Floating Brain Sections and Primary Neuronal Cultures. PLoS ONE 10, e0120120 (2015).
73. Wang, F. et al. A Novel in Situ RNA Analysis Platform for Formalin-Fixed, Paraffin-Embedded Tissues. The Journal of Molecular Diagnostics 14, 22-29 (2012).
74. Zheng, G. X. Y. et al. Massively parallel digital transcriptional profiling of single cells. bioRxiv 065912 (2016). doi:10.1101/065912
75. Dobin, A. et al. STAR: ultrafast universal RNA-seq aligner. Bioinformatics 29, 15-21 (2012).
76. R core team. R: A language and environment for statistical computing. R Foundation for Statistical Computing (2016).
77. Maaten, L. V. D. & Hinton, G. Visualizing Data using t-SNE. Journal of Machine Learning Research 9, 2579-2605 (2008).
78. Kamburov, A., Wierling, C., Lehrach, H. & Herwig, R. ConsensusPathDB—a database for integrating human functional interaction networks. Nucleic Acids Research 37, D623-D628 (2008).
79. Trapnell, C. et al. The dynamics and regulators of cell fate decisions are revealed by pseudotemporal ordering of single cells. Nature Biotechnology 32, 381-386 (2014).
80. Frank, P. G. Caveolin, Caveolae, and Endothelial Cell Function. Arteriosclerosis, Thrombosis, and Vascular Biology 23, 1161-1168 (2003).

### Example 8

Materials and Methods

The materials and methods for experiments involving human cells and tissues, as disclosed in Examples 9-18 and elsewhere in the application, are presented in this Example.

Human Cells and Tissues

Expanding on the work described in preceding Examples identifying a novel pancreatic endocrine population in mice marked by the expression of the gene Fev, we have successfully verified the existence of an analogous FEV-expressing endocrine progenitor population in human cells as well. In particular, we have identified the transcriptional profile of this FEV-expressing pancreatic endocrine progenitor population in human cells. Using genomic engineering techniques (utilizing CRISPR-Cas9 editing), a number of relevant human embryonic stem cell (hESC) lines were created, including FEV-Myc, which is an hESC line in which the FEV gene has been tagged with a protein (Myc), facilitating application of ChIP-Seq technology to identify the regions of the genome to which FEV binds in pancreatic progenitor cells. A second developed cell line is FEV-GFP, which is an hESC line in which FEV expression is reported by the presence of a green fluorescent protein. The FEV-GFP line is important for isolating FEV-expressing cells from the heterogeneous culture of hESC-derived cells as they are being directed in their differentiation towards a pancreatic beta cell fate, for instance. A third cell line developed is the FEV-KO line, which is an hESC line in which the FEV gene has been deleted (knocked out). We have now performed experiments with the FEV-KO line and shown that when the gene FEV is ablated, hESC cells suffer a significant reduction in the number of pancreatic beta cells that can be made. This provides evidence that the gene itself is functionally important in this pancreatic endocrine population.

More particularly, the data disclosed herein reveals an unknown endocrine progenitor stage that is defined by high expression of Fev, a transcription factor. The data shows that all hormone-expressing endocrine lineages of the murine pancreas transit through a Fev-expressing cell stage. The data disclosed herein further establishes that similar FEV-expressing endocrine progenitor cell populations are found in human pancreatic development.

Given that FEV+ progenitors constitute a major stage in human endocrine cell differentiation, novel tools have been developed to study both the function of FEV and FEV-expressing cells in in vitro beta cell differentiation. This work forms a foundation on which improvements to in vitro beta cell differentiation can be made to more closely reflect proper endocrine cell development in vivo, thereby increasing beta cell yield at the end of this process and generating beta cells that are functional in vitro and in vivo.

The experimental data disclosed herein establish that the novel endocrine progenitor stage defined by differential expression of the transcription factor named Fev are Fev+ endocrine progenitors derived from Ngn3+ progenitors. The Fev+ endocrine progenitors give rise to hormone-expressing lineages of the murine pancreas.

This map of in vitro beta cell differentiation uncovers a novel lineage that results from mis-differentiation of FEV-expressing progenitors and opens avenues through which current in vitro beta cell differentiation methods can be improved for greater differentiation efficiency. Given our findings of Fev/FEV+ progenitors in murine development, human fetal development, and the in vitro derivation of beta cells, valuable hESC lines were engineered to study the function of the FEV gene and FEV+ endocrine progenitors during the directed differentiation of beta cells. It is expected tat FEV+ endocrine progenitor cells can also be directed to differentiate into other hormone-expressing endocrine lineages. This work resulted in a new differentiation model for human endocrine cell development and paves the way for improved in vitro pancreatic progenitor cell derivation methods that better reflect in vivo human endocrine cell development and can lead to a variety of endocrine cell types, including beta cells, alpha cells, delta cells and the like.

Human Tissue Procurement, Isolation, and Processing

Human fetal pancreata were harvested from post-mortem fetuses with approval from the ethical committee of UCSF. Tissue was obtained through two sources: the University of Washington Birth Defects Research Laboratory (12wpc_1 and 15.5wpc samples) and Advanced Bioscience Resources, Inc (12wpc_2 and 16wpc samples). Tissue was harvested at respective clinics and shipped overnight on ice in either 1×PBS (samples from University of Washington Birth Defects Research Laboratory) or RPMI media (samples from Advanced Bioscience Resources, Inc). Following delivery, tissue was washed once with 1×PBS, minced with a sterile scalpel, and dissociated in Liberase™ and 0.1 mg/mL DNase in 1×HBSS for 30-55 minutes in a 37° C. Thermomixer programmed to shake at 1000 rpm. Dissociation was quenched with 5 mM EDTA and 10% FBS in 1×HBSS. Cell suspension was filtered through a 30 μm cell strainer. Red blood cells (RBCs) were removed from the cell suspension using immunomagnetic negative selection with STEMCELL Technologies' EasySep RBC Depletion Reagent (cat. no. 18170). Following RBC depletion, cells were counted and loaded onto the 10× Chromium Platform for single-cell RNA-sequencing.

Adult human islets were isolated from cadaveric donor tissue by the UCSF Islet Production Core with approval from the UCSF ethical committee. Consented cadaver donor pancreata were provided by the nationally recognized organization UNOS via local organ procurement agencies. The identifiers were maintained at the source only, and the investigators received de-identified specimens.

Informed consent was obtained for all human (fetal and adult) tissue collection, and protocols were approved by the Human Research protection Program Committee on Human Research of UCSF.

Embryonic Stem Cell Culture and Differentiation

The hESC line HUES8 was obtained from Harvard University and used for the generation of hESC-derived beta-like cells (BLCs). Pluripotent HUES8 cells were maintained as spherical clusters in suspension in mTeSR-1 (StemCell Technologies) in 500 mL spinner flasks (Corning, VWR) on a magnetic stir plate (Dura-Mag) within a 37° C. incubator at 5% CO2, 100% humidity, and a rotation rate of 70 rpm. Cells were screened for mycoplasma contamination using the MycoProbe Mycoplasma Detection Kit (R&D Systems), according to the manufacturer's instructions.

BLCs were generated as previously described (Pagliuca et al., 2014), with additional modifications (Millman et al., 2016). In brief, HUES8 cells were seeded into a spinner flask at a concentration of 8×10⁵ cells/mL in mTeSR1 media with 10 μM Rock inhibitor Y-27632 (STEMCELL Technologies) to allow formation of spherical clusters. Differentiation was initiated 72 hours later. Differentiation was achieved in a step-wise fashion using the following growth factors and/or small molecules: definitive endoderm (Stage 1) (1 day of 100 ng/mL Activin A (R&D Systems) and 14 μg/mL of CHIR99021 (Stemgent); 2 days of 100 ng/mL Activin A); gut tube endoderm (Stage 2) (3 days of 50 ng/mL KGF (Peprotech)); early pancreatic progenitors (Stage 3) (1 day of 200 nM LDN193189 (Fisher Scientific), 50 ng/mL KGF, 0.25 μM SANT-1 (Sigma), 2 μM Retinoic Acid (Sigma), 500 nM PdbU (EMD Biosciences), and 10 μM Rock inhibitor Y-27632 (STEMCELL Technologies); 1 day of 50 ng/mL KGF, 0.25 μM SANT-1, 2 μM Retinoic Acid, 500 nM PdbU); later pancreatic progenitors (Stage 4) (5 days of 50 ng/mL KGF, 0.25 μM SANT-1, 0.1 μM Retinoic Acid, and 10 μM Rock inhibitor Y-27632); endocrine progenitors (Stage 5) (4 days of 0.25 μM SANT-1, 0.1 μM Retinoic Acid, 1 μM XXI (EMD Millipore), 10 μM Alk5i (Axxora), 1 μM T3 (EMD Biosciences), 20 ng/mL Betacellulin (Fisher Scientific); 3 days of 25 nM Retinoic Acid, 1 μM XXI, 10 μM Alk5i, 1 μM T3, 20 ng/mL Betacellulin); BLCs (Stage 6) (6-11 days of 10 μM Alk5i; 1 μM T3). Successful differentiation was assessed at the completion of Stages 1, 3, 4, 5, and 6 via immunofluorescence or FACS for stage-specific marker genes. hESC-derived cells used for single-cell RNA-sequencing were taken at ES4 (End of Stage 4), S5D4 (Stage 5, Day 4), S5D7, S6D4, and S6D10. Cells for single-cell RNA-sequencing were dissociated with Accumax for 15-25 minutes in a 37° C. water bath. The dissociated cell suspension was neutralized with stage-specific media and filtered through a 37 μm filter. Cells were counted and then loaded onto the 10× Chromium Platform for single-cell RNA-sequencing.

In Situ Hybridization and Immunofluorescence of hESC-Derived Clusters

hESC-derived cell clusters were fixed in 4% PFA in 1×PBS for 15 minutes at room temperature (RT). Fixed clusters were washed with 1×PBS and cryoprotected overnight at 4° C. in 30% sucrose. Clusters were then embedded in OCT, and 8 μm sections were cut.

In situ hybridization was performed on 8 μm sections using RNAscope technology (Advanced Cell Diagnostics) according to the manufacturer's instructions. An in situ probe against human FEV (cat. no. 471421-C3) was used in combination with the RNAscope Multiplex Fluorescent Reagent Kit v2 for target detection. Following signal amplification of the target probes, sections were washed in 1×PBS three times and blocked in 5% normal donkey serum (NDS, Rockland Immunochemicals) in 0.1% Triton X-100 in PBS for 1 hour at RT. Tissue sections were then stained with a primary antibody against PDX1 (1:100, R&D Systems). The next day, sections were washed three times in 0.1% Tween 20 in 1×PBS and then incubated with species-specific Alexa Fluor 488-secondary antibodies (1:500, Jackson ImmunoResearch) and DAPI in 5% NDS in 0.2% PBT for 1 hour at RT. Sections were washed three times in 0.1% Tween 20 in 1×PBS, rinsed in 1×PBS, and then mounted in ProLong Gold Mounting Medium. Slides were stored at 4° C.

Images were captured on a Leica confocal laser scanning SP8 microscope. Maximum intensity z-projections were then prepared using ImageJ, where brightness, contrast, and pseudo-coloring adjustments were applied equally across all images in a given series.

Quantitative RT-PCR

hESC-derived cells at various stages of directed differentiation were collected in Trizol, and RNA was extracted with the Direct-zol RNA Miniprep kit (Zymo Research). Adult human islets were also processed this same manner for RNA extraction. Reverse transcription was performed with the Superscript IV First-Strand Synthesis System (Thermo Fisher Scientific, cat. no. 18091050) using Oligo d(T) primers and random hexamers. RT-PCR was run on an ABI Real-Time PCR System (Applied Biosystems, 384-well format) with Taqman probes for FEV (assay ID: Hs00232733_m1) and GAPDH (assay ID: Hs02758991_g1) in triplicate. Data were normalized to GAPDH.

Single-Cell Capture and Sequencing

To capture individual cells, we utilized the Chromium Single Cell 3′ Reagent Version 3 Kit (10× Genomics) (Zheng et al., 2017). Only the 15.5wpc sample was processed with the Chromium Single Cell 3′ Reagent Version 2 Kit. For all samples, 25,000 cells were loaded onto one or two wells of the 10× chip to produce Gel Bead-in-Emulsions (GEMs). GEMs underwent reverse transcription to barcode RNA before cleanup and cDNA amplification. Libraries were prepared with the Chromium Single Cell 3′ Reagent Kit. Each sample was sequenced on the NovaSeq (Illumina) in Rapid Run Mode with paired-end sequencing parameters: Read1, 98 cycles; Index1, 14 cycles; Index2, 8 cycles; and Read2, 10 cycles.

Single-Cell Analysis

CellRanger v3.0.2 software was used for all single-cell RNA-sequencing datasets with default settings for de-multiplexing, aligning reads to the human genome (10× Genomics pre-built hg38 reference genome) with STAR (Dobin et al., 2012) and counting unique molecular identifiers (UMIs) to build transcriptomic profiles of individual cells. Gene-barcode matrices were analyzed with the R package Seurat v3.0.1 (Stuart et al., 2019). We first performed a filtering step, retaining only the cells that expressed a minimum and maximum number of genes and did not exceed a specified percentage of reads that map to the mitochondrial genome. The following quality control metrics for each dataset are outlined in Table 6.

TABLE 6
Quality control metrics for human single-cell sequencing analyses.

| | Minimum | Maximum | Maximum |
|---|---|---|---|
| | Number | Number | Percentage Of |
| Sample Name | Of Genes | Of Genes | Mitochondrial Gene |
| 12wpc_1 | 200 | 4,000 | 15 |
| 12wpc_2 | 200 | 5,000 | 15 |
| 15.5wpc | 200 | 4,000 | 7.5 |
| 16wpc | 200 | 6,000 | 15 |
| ES4 | 200 | 6,000 | 10 |
| S5D4 | 200 | 6,000 | 12.5 |
| S5D7 | 200 | 6,000 | 15 |
| S6D4 | 200 | 6,000 | 15 |
| S6D10 | 200 | 6,000 | 15 |

Sample name is listed along with the minimum and maximum number of genes and maximum percentage of mitochondrial genes used for quality control thresholds.

Data were then normalized with the Seurat3 function NormalizeData with default settings. This employs a global-scaling normalization that normalizes gene expression measurements for each cell by the total expression. Genes that exhibit high cell-to-cell variation were then identified using FindVariableFeatures. The highly variable genes from this analysis were then used in downstream analysis to highlight biological signal from background noise in single-cell datasets. Data then underwent linear transformation (“scaling”), which was required prior to dimensional reduction with PCA, and this scaling was done with ScaleData. PCA (Principal Component Analysis) was performed on the scaled data with RunPCA. Significant PCs (principal components) were determined with ElbowPlot, which plots principal components based on the percentage of variance exhibited by each one. These significant PCs were utilized in Seurat3's graph-based clustering algorithms, FindNeighbors and FindClusters. The resolution parameter of FindClusters was adjusted to vary the number of clusters found by the algorithm. Clusters were visualized by UMAP with Seurat3's RunUMAP and DimPlot functions. Differentially expressed genes were determined with the FindAllMarkers function. Seurat3's VInPlot, DotPlot, and FeaturePlot functions were used to visualize of expression of genes of interest across cells and clusters.

Sub-Clustering and Merging Datasets

Sub-clustering was performed by isolating clusters of interest with the Seurat3 function Subset and reanalyzing as outlined above (finding variable genes, scaling data, and identification of significant PCs). Cells were classified as endocrine based on the expression of Chromogranin A (CHGA).

Merging of all human fetal datasets was accomplished with Seurat3's Integration workflow. This integration workflow in Seurat3 identifies “anchors” across disparate single-cell datasets in order to construct harmonized references for better merging of the data and minimization of batch effect (Stuart et al., 2019). In the integration workflow, all datasets were merged into a single Seurat object and processed to the step encompassing identification of variable genes (FindVariableFeatures). Integration anchors were then identified using the FindIntegrationAnchors and used to integrate all human fetal datasets through the IntegrateData function. Following integration, data were scaled (ScaleData), significant PCs were identified (RunPCA), and UMAP-based clustering was performed (RunUMAP, FindNeighbors, FindClusters). Gene expression of specific genes were visualized by using read levels from the “RNA” slot of the integrated Seurat object (accessed by inputting “rna_gene” into gene parameter).

Pseudotemporal Ordering

For the pseudotemporal ordering analysis of the 12wpc_1 sample, we utilized Monocle v2.99.3 (named Monocle 3 alpha). Variable genes from the Seurat3 analysis of the 12wpc_1 samples (resolution 0.8) were used as input into Monocle, utilizing the VGAM::negbinomial.size expressionFamily, and clusters were projected onto the minimum spanning tree after ordering. The beginning of pseudotime was assigned using the function orderCells based on NGN3 expression.

To conduct alpha and beta branch analysis, clusters along each branch were isolated and loaded into Monocle separately. Genes that changed significantly as a function of pseudotime were identified with Monocle's differentialGenetest function, and those that displayed a q-value less than 0.001 were selected for downstream analysis. These genes were then plotted as a heatmap (using plot_pseudotime_heatmap) that clustered genes based on similarities in expression patterns along pseudotime. The expression of individual genes was plotted using Monocle's plot_genes_in_pseudotime function.

For the pseudotemporal ordering analysis of our merged human fetal and hESC-derived cell datasets, we utilized Monocle3 v0.1.0 (named Monocle 3 beta) was used. This version of Monocle3 was used because of its internal batch correction capabilities. For the merged human fetal pseudotemporal ordering analysis, variable genes from the Seurat3 integration analysis were used as input into Monocle. Clusters were projected onto the minimum spanning tree after ordering.

For the merged hESC-derived analysis, variable genes from CHGA+ sub-clustering were used as input into Monocle. To batch correct based on sample type, the residual_model_formula_str was set to “^{˜}orig.ident” during the pre-process_cds step. To conduct branch-specific analyses, the choose_cells function was used to manually select the branches of interest in Monocle's graphical user interface. Once branches were selected, genes that changed significantly along pseudotime were identified using the graph_test function. Genes of interest were plotted along the Monocle trajectory using the plot_cells function.

Genetic Engineering of the FEV-KO hESC Line

The HUES8 hESC line was used to generate the FEV-KO line. For the FEV-KO hESC line, the FEV-KO gRNA (5′-CTGATCAACATGTACCTGCC-3′; SEQ ID NO:1) was designed on Benchling software and ordered from Dharmacon in a lyophilized format. The gRNA was suspended in nuclease-free 10 mM Tris-HCl Buffer (pH 7.4) ordered from Dharmacon (cat. no. B-006000-100) and stored as aliquots at −80° C. HUES8 hESCs were grown on Matrigel-coated tissue culture plates, and on the morning of nucleofection, media was changed to mTeSR1+10 μM Rock inhibitor Y-27632 for 2 hours prior to nucleofection. Following this incubation step, hESCs were lifted from Matrigel plates and dissociated into a single-cell suspension using TrypLE Express. Cells were incubated in TrypLE Express dissociation reagent for 6 minutes at RT. mTeSR1+10 μM Rock inhibitor Y-27632 was used to neutralize the dissociation, and cell suspension was filtered through a 37 μm filter.

To carry out the nucleofection, we mixed 2.75 μL of tracrRNA (160 uM) and 2.75 uL of the FEV-KO gRNA (160 μM) (to make the “RNA-complex”) in a PCR strip tube and incubated for 30 minutes in the 37° C. cell culture incubator. After 30 minutes, 5.5 μL of purified Cas9-NLS protein (QB3 UC Berkeley MacroLab) was added to the RNA complex, gently mixed to make the RNP (ribonucleoprotein), and incubated at 37° C. for exactly 15 minutes. After exactly 15 minutes, previously dissociated cells were resuspended in Lonza's P3 buffer from the P3 Primary Cell 4D-Nucleofector X Kit S (V4XP-3032). 10 μl of cell suspension containing 400K cells were pipetted into one well of the Lonza nucleofection strip, and 10 μl of the RNP was added. The nucleofection strip was then inserted into the Lonza 4D-Nucelofector (Lonza, AAF-1002B) and nucleofected with the CA137 setting compatible with the P3 buffer. Nucleofected cells were then transferred to a 15 mL conical with 3 mL of mTeSR1 containing 10 μM Rock inhibitor Y-27632 and pen/strep (penicillin/streptomycin). Cell viability was determined via Moxiflow, and cells were plated in one well of a 6-well plate coated with Matrigel. Cells were grown for 2-3 passages in mTeSR1 containing 10 μM Rock inhibitor Y-27632 and pen/strep to allow for recovery from nucleofection.

To determine genomic editing efficiency of the FEV-KO nucleofection experiment, genomic DNA from nucleofected cells was harvested in QuickExtract DNA Extraction (Lucigen, QE09050) and then used for PCR amplification. The following forward and reverse primers targeting the FEV-KO editing site were used to produce a 491-bp amplicon: 5′-CCGTCTTCTCCTCCTTGTCACC-3′ (SEQ ID NO:2) and 5′-CTCGGCCACAGAGTACTCCAC-3′ (SEQ ID NO:3). This amplicon is GC-rich, requiring use of a PCR polymerase capable of handling GC-rich amplicons (PrimeSTAR GXL Premix, Clontech). This DNA amplicon and a wild-type DNA amplicon were sent to Quintarabio for Sanger sequencing. The chromatographs of each sequencing run were used for TIDE (Tracking of Indels by Decomposition) analysis, which estimates the frequency of insertions and deletions (indels) in a pool of cells that has undergone genomic editing (Brinkman et al., 2014). Cutting efficiency of hESCs nucleofected with FEV-KO gRNA was then determined.

To derive a clonal FEV-KO line from this heterogeneous pool of hESCs that have no mutation in the FEV locus, a mutation(s) in one FEV allele, or mutations on both FEV alleles, these cells were clonally plated on Matrigel-coated plates. Approximately 1,500 cells were dispersed onto a 10 cm Matrigel-coated plate and allowed to grow for 9-10 days in mTeSR1. For the first 4-5 days of culture, cells were cultured in mTeSR1 containing 10 μM Rock inhibitor Y-27632. Clonal colonies were then hand-picked under a colony-picking microscope under sterile conditions. These hand-picked colonies were each transferred into one well of a 96-well plate, allowed to grow for 2-3 days, and then successively passaged into large-plate formats (96-well to 24-well to 6-well to 10 cm dish). Clonality was first determined through TIDE analysis, as outlined above, and confirmed with TOPO cloning of the FEV-KO PCR amplicon.

Genetic Engineering of the FEV-KI hESC Lines

The HUES8 hESC line was used to generate the FEV-MYC, FEV-GFP, and FEV-tNGFR lines. The MYC, GFP, and tNGFR inserts were all commercially synthesized as gene blocks from Integrated DNA Technologies. 5′ and 3′ FEV locus homology arms that were 400 bp in length were then added to each of the MYC, GFP, and tNGFR gene blocks using In-Fusion HD Cloning (Clontech, 638920). These homology arms flanked the cut site targeted by the FEV-KI gRNA. The result of In-Fusion HD cloning was a pUC19 plasmid containing a MYC, GFP, or tNGFR insert flanked by 5′ and 3′ FEV homology arms. These plasmids were transformed into Stellar Competent Cell (Clontech, 636766), and PCR amplification off of these isolated plasmids generated a PCR amplicon for use as our targeting template to knock in MYC, GFP, and tNGFR into the FEV locus. The following forward and reverse primers were used in PCR to generate each targeting template from each plasmid: 5′-TGAACTACGACAAGCTGAGCCG-3′ (SEQ ID NO:4) and 5′-TCCTTGGGGAAGAGCAAAAGTG-3′ (SEQ ID NO:5).

For knock-in of MYC, GFP, and tNGFR into the FEV locus, a FEV-KI gRNA (GCCATTACCACTAGACGGGG; SEQ ID NO:6) was designed using Benchling software and targeted the end of exon 3 of the FEV locus. This FEV-KI gRNA cut immediately preceding the FEV stop codon found at the end of exon 3 and would facilitate the knock-in of each insert in-frame with the FEV locus. On the morning of nucleofection, HUES8 hESCs were fed with mTeSR1+10 μM Rock inhibitor Y-27632 for 2 hours. Following this incubation step, hESCs were lifted from Matrigel-coated plates and dissociated into a single-cell suspension using TrypLE Express. Cells were incubated in TrypLE Express dissociation reagent for 6 minutes at RT. mTeSR1+10 μM Rock inhibitor Y-27632 was used to neutralize the dissociation, and cell suspension was filtered through a 37 μm filter.

To carry out the nucleofection, 1.25 μL of tracrRNA (160 uM), 1.25 μl of FEV-KI gRNA (160 μM), and 1 μg of either the MYC, GFP, or tNGFR targeting templates were mixed in a PCR strip tube and incubated for 30 minutes in a 37° C. cell culture incubator. After 30 minutes, 2.5 μL of purified Cas9-NLS protein (QB3 UC Berkeley MacroLab) was added, gently mixed to make the RNP complex, and incubated at 37° C. for exactly 15 minutes. Dissociated cells were pelleted at 1000 rpm for 3 minutes and resuspended in Lonza P3 buffer (Lonza, V4XP-3032). 10 μL of cell suspension containing 400,000 cells were then pipetted into a Lonza cuvette, and 10 μL of the RNP complex+targeting template was added. The cuvette was then inserted into the Lonza 4D-Nucelofector (Lonza, AAF-1002B) and nucleofected with the CA137 setting compatible with the P3 buffer. Nucleofected cells were then transferred to a 15 mL conical tube with 3 mL of mTESR containing 10 μM Rock inhibitor Y-27632 and pen/strep. Cell viability was determined via Moxiflow, and cells were plated in one well of a 6-well plate coated in Matrigel. Cells were grown for 2 passages to allow for recovery from nucleofection.

To determine if the MYC, GFP, and tNGFR inserts were successfully knocked-in, genomic DNA from nucleofected cells was harvested in QuickExtract DNA Extraction (Lucigen, QE09050) and used for PCR amplification. The following forward and reverse primers were used: MYC: 5′-AGATCCAGCTGTGGCAGTTTCT-3′ (SEQ ID NO:7) and 5′-ACCAGACAAGGATTGAGGGAGC-3′ (SEQ ID NO:8) GFP: 5′-CGTGCATCTGGAAAGCTACGTG-3′ (SEQ ID NO:9) and 5′-CTTGAAGAAGTCGTGGCGCTTC-3′ (SEQ ID NO:10) tNGFR: 5′-TGAACTACGACAAGCTGAGCCG-3′ (SEQ ID NO:4) and 5′-TCCTTGGGGAAGAGCAAAAGTG-3′ (SEQ ID NO:5). Presence of a knock-in band that was larger than the FEV wild-type band was indicative that a subset of nucleofected cells carried the insert.

To derive clonal FEV-KI lines from a heterogeneous pool of hESCs that either had the knock-in insert or not, cells were clonally plated on Matrigel-coated plates. Approximately 1,500 cells were dispersed onto a 10 cm Matrigel plate and allowed to grow for 9-10 days in mTeSR1. For the first 4-5 days of culture, cells were cultured in mTeSR1 containing 10 μM Rock inhibitor Y-27632. These hand-picked colonies were each transferred into one well of a 96-well plate, allowed to grow for 2-3 days, and then successively passaged into large-plate formats (96-well to 24-well to 6-well to 10 cm dish). Genomic DNA was isolated from each clonal line and the insert was confirmed through PCR using same primers as indicated above. Sanger sequencing of the genomic FEV locus confirmed that the MYC, GFP, and tNGFR had no mutations and were in-frame with the endogenous FEV locus.

Generation of Gene KOs During Directed Differentiation of hESCs

Approximately 100-150×10⁶ End Stage 4 (ES4) cells from the directed differentiation protocol were dissociated in Accumax for 15-25 minutes in a 37° C. water bath. The dissociated cell suspension was passed through a 37 μm filter. Cell count and viability were determined with a Moxiflow cell counter. Cells were pelleted at 1000 rpm for 3 minutes and kept in ES4 media until nucleofection.

For nucleofection, the large format of Lonza's nucleofection kits (P3 Primary Cell 4D-Nucleofector X Kit L, V4XP-3024) was used, which accommodates nucleofection of up to 20×10⁶ cells per nucleofection vessel. Four conditions were typically included in these experiments: non-nucleofected control, scramble control, hAAVS1 control, and KO of gene of interest. The non-nucleofected control group contained ES4 cells that did not go through nucleofection. The scramble control group contained ES4 cells that were nucleofected with a scramble gRNA (GGTTCTTGACTACCGTAATT; SEQ ID NO:11) that is not predicted to cut anywhere in the human genome. The hAAVS1 control included ES4 cells that were nucleofected with a gRNA targeting a safe harbor locus in the human genome AAVS1 (GGGGCCACTAGGGACAGGAT; SEQ ID NO:12. The KO of gene of interest group contained ES4 cells that were nucleofected with a gRNA targeting the gene of interest we wished to knock out. All gRNAs were ordered from Dharmacon.

For each nucleofection set of 10-20×10⁶ ES4 cells, 9.5 μL of tracrRNA (160 μM), 9.5 μL of the FEV-KI gRNA (160 μM) were mixed in a PCR tube and incubated for 30 minutes in a 37° C. cell culture incubator. After 30 minutes, 19 μL of purified Cas9-NLS protein (QB3 UC Berkeley MacroLab) was added, gently mixed to make the RNP complex, and incubated at 37° C. for exactly 15 minutes. Dissociated ES4 cells were pelleted at 1000 rpm for 3 minutes, and each set of 10-20×10⁶ ES4 cells were resuspended in 64 μL of Lonza P3 buffer (from V4XP-3024). Each set of cells were then pipetted into a large Lonza nucleofection vessel, and 36 μl of the RNP were added.

Nucleofection vessel was then inserted into the Lonza 4D-Nucleofector (Lonza, AAF-1002B) and nucleofected with the CA137 setting compatible with the P3 buffer. Nucleofected cells were then transferred to a 15 mL conical tube with 10 mL of S5D1 media. Cell viability was determined via Moxiflow.

Following nucleofection, cells were immediately re-aggregated into clusters using AggreWell 400 plates (STEMCELL Technologies, 34415). Wells in the AggreWell 400 plates were washed with an Anti-Adherence Rinsing Solutions (STEMCELL Technologies, 07010) and centrifuged in a swinging bucket rotor at 1300×g for 5 minutes. Rinsing solution was removed, and S5D1 media was used to rinse wells. S5D1 media was aspirated, and 1.2×10⁶ cells were then pipetted into each well of an AggreWell 400 plate. Plates were spun at 100×g for 3 minutes to facilitate re-aggregation of cells in each microwell and then were observed under microscope to verify even distribution of cells among microwells. Plates were placed in the 37° C. cell culture incubator, and spheroids formed by 48 hours (by S5D3). On S5D3, clusters were removed from the AggreWell plates and cultured in either miniature spinner flasks called Biotts (BWV-503A) set at a 70 rpm rotation speed or in 6-well ultra low-attachment plates (5 mL of media with approximately 5×10⁶ cells per well) placed on an orbital shaker set to 100 rpm. Directed differentiation of these nucleofected clusters was continued either in Biotts or in a 6-well ultra low-attachment plate.

FACS of hESC-Derived Cells

BLC clusters were dissociated in Accumax for 15-25 minutes in a 37° C. water bath. The dissociated cell suspension was passed through a 37 μm filter. Cells were pelleted at 1000 rpm for 3 minutes and fixed in 4% PFA for 12 minutes at RT. Cells were washed in 1×PBS, pelleted again, and resuspended in 1×PBS. Fixed cells were stored at 4° C. prior to staining for FACS.

For FACS staining, cells were permeabilized using 1× Permeabilization Buffer (Invitrogen, 00-8333-56) for 5 minutes at RT. Cells were then incubated in primary antibody diluted in Blocking reagent (0.2% Triton X-100, 5% NDS, 1% Bovine Serum Albumin (BSA) in 1×PBS) overnight at 4° C. Primary antibodies used were anti-Chromogranin A (1:500, Abcam ab15160) and anti-C-Peptide (1:200, EMD Millipore 05-1109). The next day, cells were washed in 1× Permeabilization Buffer for 5 minutes at RT and incubated in species-specific Alexa Fluor 488- and 555-conjugated secondary antibodies (1:500, Jackson ImmunoResearch) for 30 minutes at RT. Cells were then washed in 1× Permeabilization Buffer, pelleted, resuspended in 1×PBS, and analyzed with BD Fortessa Analyzer.

### Example 9

Diversity of Cell Types in the Developing Human Pancreas

Improving the ability to generate terminally differentiated cell types in all animals, including humans, will be beneficial in providing higher quality healthcare, at lower cost, and also in improving the quality of life for many. Gaining a better understanding of the cell stages required for human endocrine cell development as well as the transcriptional circuitry driving lineage allocation into distinct endocrine linages will refine our ability to generate these hormone-expressing cell types from human embryonic stem cells.

The discovery of a novel endocrine progenitor defined by high Fev expression in mouse, as disclosed herein, prompted the question of whether additional endocrine progenitor stages exist in human endocrine cell development beyond the NGN3+ endocrine progenitor stage. Defining these stages in humans can be leveraged to more properly mimic human endocrine cell development in vitro during directed differentiation protocols that harness the power of hESCs.

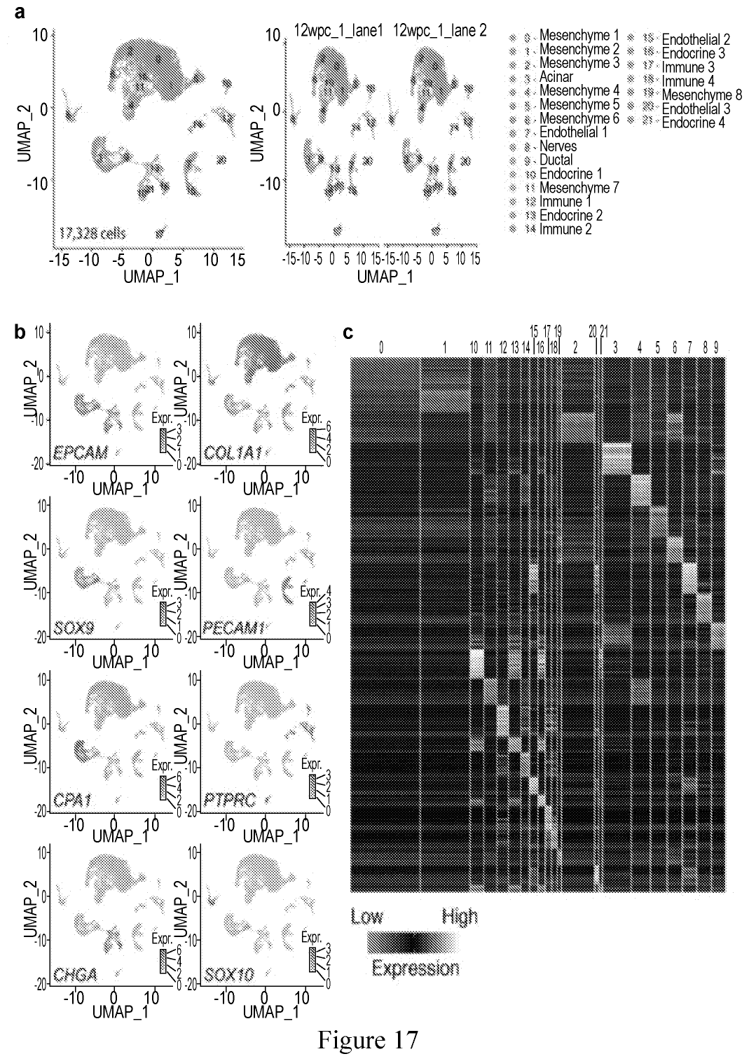

The different cellular compartments and their transcriptional profiles from human fetal pancreas were characterized. The focus was on a 12wpc time point, which represents a period of peak NGN3 expression and active cell differentiation in the developing human pancreas (Nair and Hebrok, 2015). Tissue from this 12wpc time point was dissociated into a single-cell suspension, and RBCs were removed via immunomagnetic separation. The resulting single-cell suspension was loaded onto two wells of the 10× Chromium Single-Cell Platform and prepared for sequencing using version 3 (V3) chemistry. Following sequencing and de-multiplexing of single-cell data, UMAP-based clustering of merged well replicates revealed 22 cell clusters organized into acinar, ductal, endocrine, mesenchymal, endothelial, immune, and nerve populations based on the expression of known marker genes, such as CPA1 (acinar), SOX9 (ductal), CHGA (endocrine), COL1A1 (mesenchymal), PECAM1 (endothelial), PTPRC (immune), and SOX10 (nerves) (FIG. 17a-c). The contribution of each cell cluster from each replicate well showed no batch effect based on use of different lanes on the Chromium 10× platform (FIG. 17a). Similar to the work using single-cell RNA-sequencing to interrogate the diversity of cell populations in mouse pancreatic development, transcriptomic profiling at single-cell resolution facilitated the preliminary construction of a cellular roadmap of the cell populations present in the developing human pancreas.

### Example 10

Identification of Novel Cell Stages During Human Endocrine Cell Development

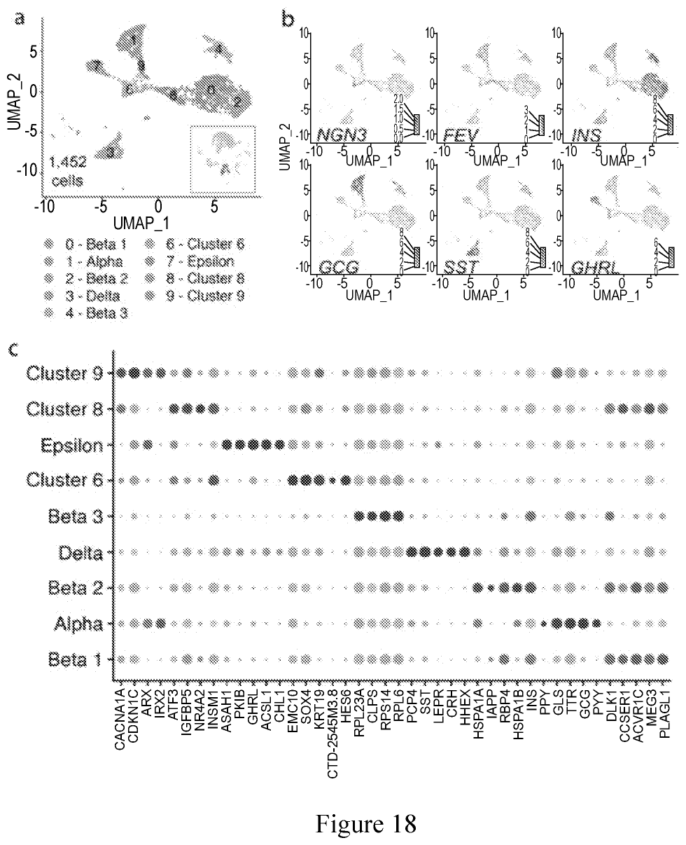

Given our previous identification of novel progenitor stages in mouse pancreatic development, we next focused on the endocrine compartment of the developing human pancreas in order to determine if additional endocrine stages exist beyond those characterized by NGN3+ progenitors and differentiated hormone+ endocrine cells. Sub-clustering of the CHGA+ cell clusters resulted in increased resolution of the endocrine lineage populations present in 12wpc human fetal pancreas, revealing 11 distinct endocrine lineage populations (FIG. 18a). We identified clusters defined by GCG, INS, SST, and GHRL, corresponding to alpha, beta, delta, and epsilon cells, respectively (FIG. 18b). By sequencing, INS transcript appeared to be also present in the alpha cell cluster (FIG. 18b). However, it is likely that differentiated alpha cells do not translate the INS transcript into protein. While only one cell cluster was identified for each of the alpha, delta, and epsilon populations, three distinct fetal INS+ beta cell populations (clusters 0, 2, and 4), defined by distinct differentially expressed genes, were observed (FIG. 3.2a-c). Many of these differentially expressed genes, such as DLK1, MEG3, and RBP4, drive beta cell heterogeneity in the adult human pancreas, indicating that sources of beta cell heterogeneity arise as early as in fetal pancreatic development (Lawlor et al., 2017; Segerstolpe et al., 2016). This heterogeneity in beta cells observed in development could serve as a major underpinning of the heterogeneity in the regulation of functional maturation and levels of ER stress seen in both mouse and human beta cells (Baron et al., 2016; Muraro et al., 2016; Qiu et al., 2017a; Zeng et al., 2017).

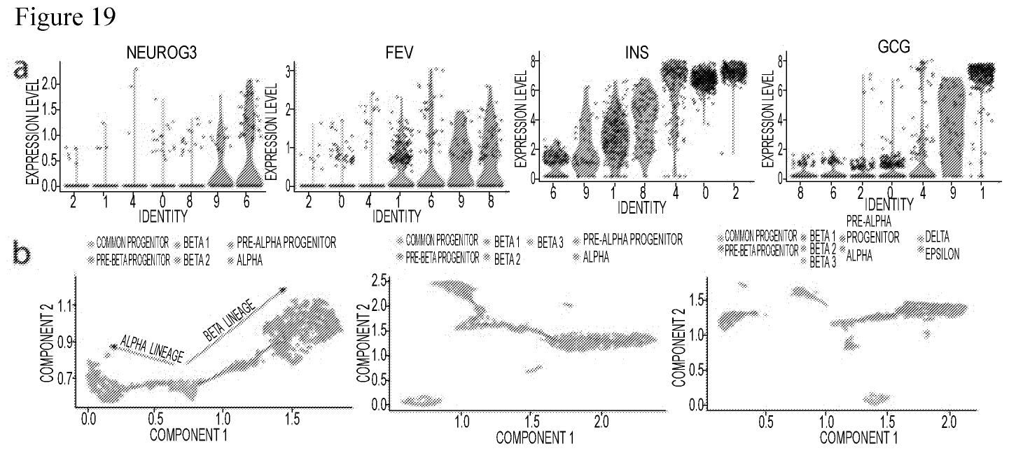

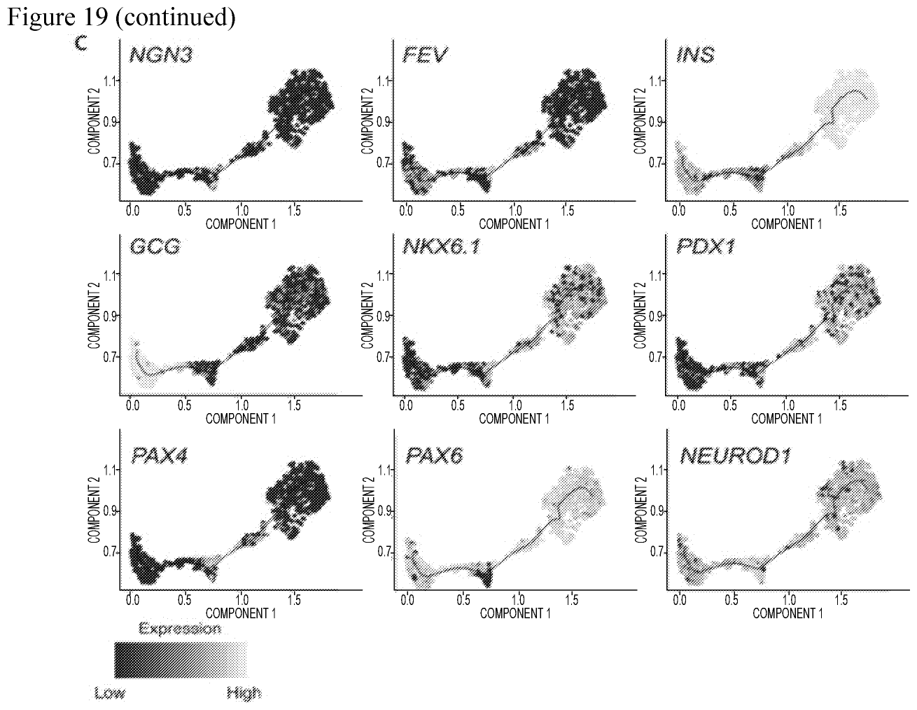

Three additional cell clusters (clusters 6, 8, and 9) that were devoid of any hormone expression (FIGS. 18a,b and 19a) were also found. Cluster 6 displayed expression of NGN3 (FIGS. 18b, 19a), and clusters 8 and 9 exhibited FEV expression (FIGS. 18b, 19a). Given the work establishing that Fev marks an endocrine progenitor population that is derived from Ngn3+ progenitors in mouse pancreatic development (Byrnes et al., 2018), the question became whether these clusters gave rise to hormone-expressing endocrine lineages. In silico reconstruction of lineage relationships was accomplished using pseudotemporal ordering with only cluster 6, cluster 8, cluster 9, beta 1, beta 2, and the alpha cell cluster as input (FIG. 19b, left panel). This analysis revealed that clusters 6, 8, and 9 were precursors to the alpha and beta lineages (FIG. 19b, c). Cluster 6 represented a common endocrine progenitor population that gave rise to both alpha and beta lineages (FIG. 19a, b). Clusters 8 and 9, on the other hand, were precursor populations that gave rise to differentiated beta and alpha cells, respectively (FIG. 19a-c). Thus, cluster 8 was identified as a pre-beta progenitor population, and cluster 9 was identified as a pre-alpha progenitor population. FEV was a top 1.5-fold differentially expressed gene in both clusters 8 and 9 (FIG. 19a), indicating that FEV-expressing progenitors give rise to both alpha and beta lineages in human endocrine cell development. FEV expression persisted into the alpha lineage but was not expressed by differentiated beta cells (FIG. 19a, c), indicating that FEV must turn off in pre-beta progenitors prior to the acquisition of beta cell identity, while this requirement is not true for pre-alpha progenitors that differentiate into the alpha lineage. This was in contrast to mouse pancreatic development in which Fev is expressed in a subset of differentiated alpha and beta cells (Byrnes et al., 2018). In examining other critical transcription factors for endocrine development, upregulation of PDX1, NKX6.1, and PAX4 expression was observed in the pre-beta progenitor stage (FIG. 19c). PAX6 and NEUROD1, on the other hand, were expressed in both the alpha and beta lineages, starting at the common progenitor stage (cluster 6) (FIG. 19c). The pseudotemporal ordering analysis thus provides additional insight into the cell stages required to generate alpha and beta cells in human pancreatic development.

The inclusion of other hormone-expressing endocrine lineages in pseudotemporal ordering did not result in a continuous differentiation trajectory (FIG. 19b, middle and right panels). Inclusion of the third beta cell population (beta 3) resulted in a trajectory that excluded this population from the main trajectory (FIG. 19b, middle panel), meaning that the pseudotemporal ordering analysis considered this beta 3 cluster as not lineage-related to any other population. Similarly, inclusion of the GHRL+ epsilon and SST+ delta clusters resulted in a disjointed trajectory that did not place these other hormone-expressing cell clusters in any lineage relationships with the progenitor populations identified herein (FIG. 19b, right panel). Sequencing of additional time points will increase the number of endocrine lineage cells captured and thus may provide increased resolution to differentiation process of these other hormone-expressing lineages.

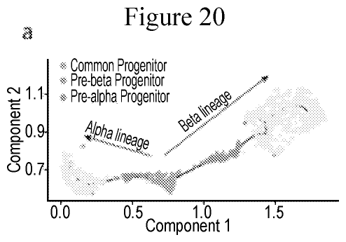

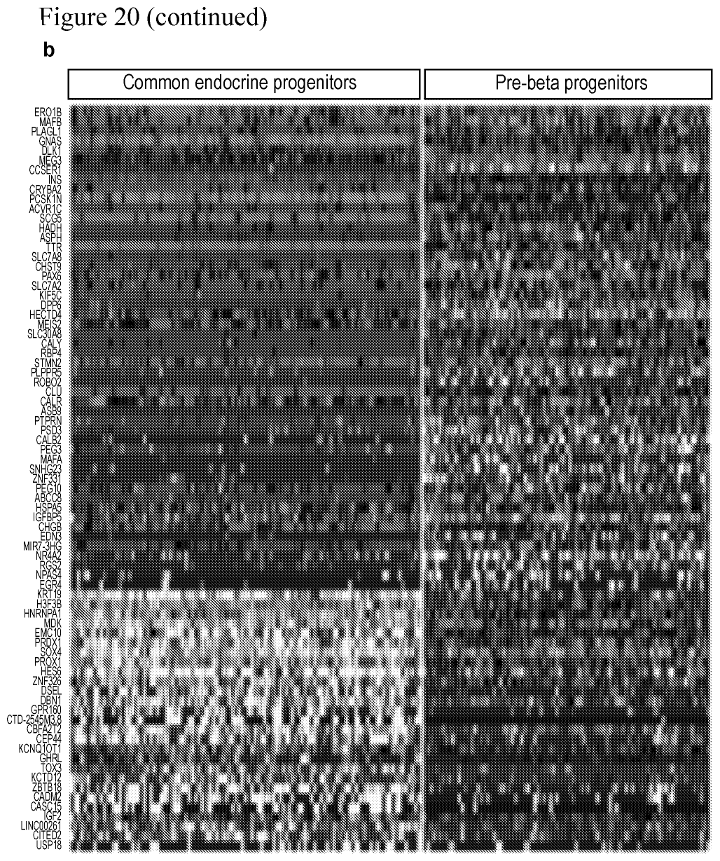

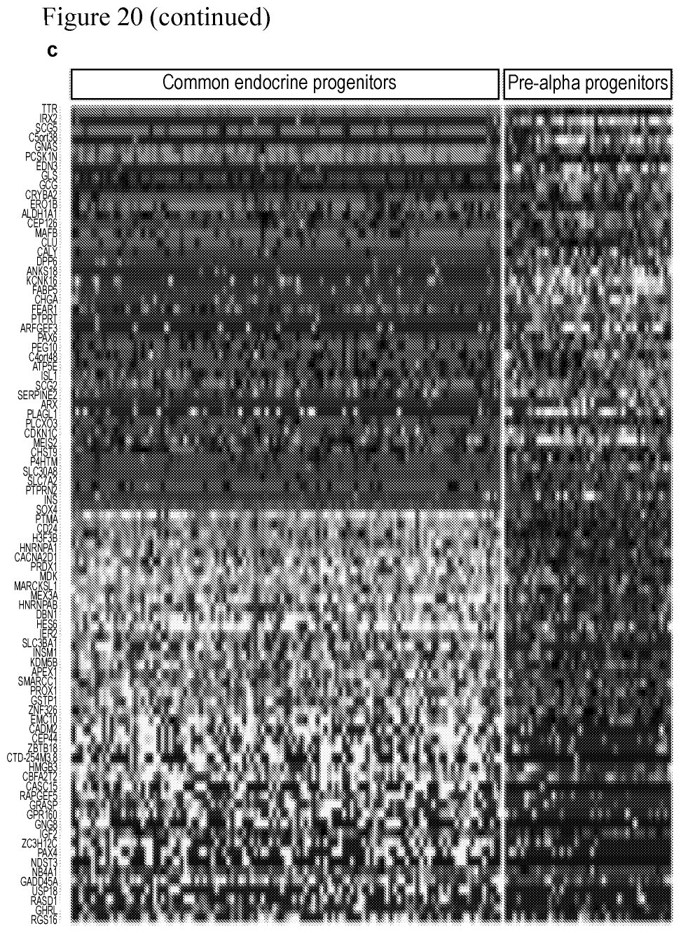

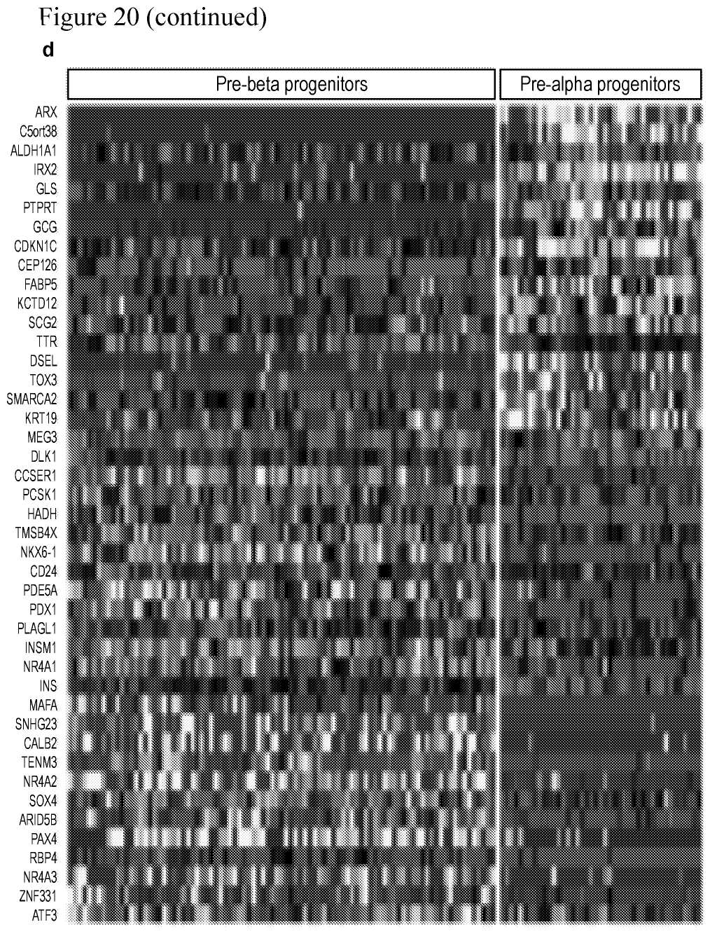

Pairwise comparisons and examination of the top differentially expressed genes of clusters 6, 8, and 9 revealed that these populations represent novel cell stages of human endocrine cell differentiation at a resolution that we have not been able to appreciate with previous techniques. NGN3 expression was concentrated within the common endocrine progenitor population (cluster 6), although NGN3 was not among the top 5 differentially expressed genes (FIG. 18b). Instead, this common endocrine progenitor cluster was defined by expression of genes such as EMC10, SOX4, and HES6 (FIGS. 18c, 20a-c). EMC10 is an ER membrane protein with reported roles as an angiogenic factor that promotes tissue repair after myocardial infarction (Reboll et al., 2017). SOX4 is a member of the SOX family of transcription factors and is a reported target of Ngn3 in mouse pancreatic development (Xu et al., 2015). ES6 suppresses HES1, which suppresses the onset of NGN3 expression that initiates endocrine cell development in the pancreas (Masjkur et al., 2016). Other notable genes whose canonical functions involve transcription and DNA binding and that were more than 1.5-fold differentially expressed in common endocrine progenitors (cluster 6) include NEUROD1, NKX2-2, PAX4, RFX3, SMARCC1, CITED2, HMGB3, KDM5B, ZBTB18, TGIF2, ARID4A, CBFA2T2, and PROX1 (FIG. 20). According to the pseudotime reconstruction of endocrine lineage relationships, these common endocrine progenitors in cluster 6 gave rise to both pre-beta and pre-alpha progenitors (clusters 8 and 9) (FIG. 19b). Although both the pre-beta and pre-alpha progenitor populations expressed hormones (FIG. 19a), their expression of either INS or GCG was markedly lower compared to the expression levels of both hormones found in differentiated beta or alpha cells (FIG. 19a), indicating that these clusters were not fully differentiated into their respective endocrine lineages. The top differentially expressed genes in pre-beta progenitors in cluster 8 were MEG3, NR4A2, and IGFBP5 (FIG. 18c). Notable factors involved in transcription that were more than 1.5-fold differentially expressed in pre-beta progenitors include SOX4, NKX6-1, PDX1, PAX6, PAX4, EGR3, ARID5B, RYBP, SIM1, MNX1, TSHZ1, ATF3, FOXA2, NR4A3, NR4A1, NR4A2, MAFB, EGR4, NPAS4, ID4, and ETS2 (FIG. 20a, b, and d). In contrast, the top differentially expressed genes in pre-alpha progenitors in cluster 9 were IRX2 and ARX, which both regulate alpha cell lineage allocation (Petri et al., 2006; Wilcox et al., 2013), as well as CDKN1C, which is a cyclin-dependent kinase inhibitor (FIG. 3.2c). Notable factors involved in transcription that were more than 1.5-fold differentially expressed in pre-alpha progenitors include NEUROD1, PAX6, ISL1, PSIP1, ST18, SIM1, MLXIPL, TOX3, PBX1, ESRRG, and ID4 (FIGS. 20a, c and d). Pairwise comparisons among these three endocrine progenitor clusters reveal that each population is transcriptionally distinct and arises at defined stages along endocrine cell differentiation.

### Example 11

Candidate Lineage Regulators of the Beta Cell Lineage

The onset of NGN3 expression marks the beginning of endocrine cell development as cells differentiate towards a hormone+ endocrine lineage. However, the transcriptional programs that guide these endocrine progenitors toward a distinct hormone-expressing endocrine lineage are not well defined in human endocrine cell differentiation. With the lack of lineage tracing tools available for in vivo human studies, single-cell RNA-sequencing data was used to make inferences about the transcriptional machinery that regulates endocrine lineage allocation. Given that we observed distinct stages of cellular differentiation leading to both alpha and beta lineages (FIG. 19b), we next investigated potential transcriptional regulators involved in mediating the acquisition of either alpha or beta cell identity.

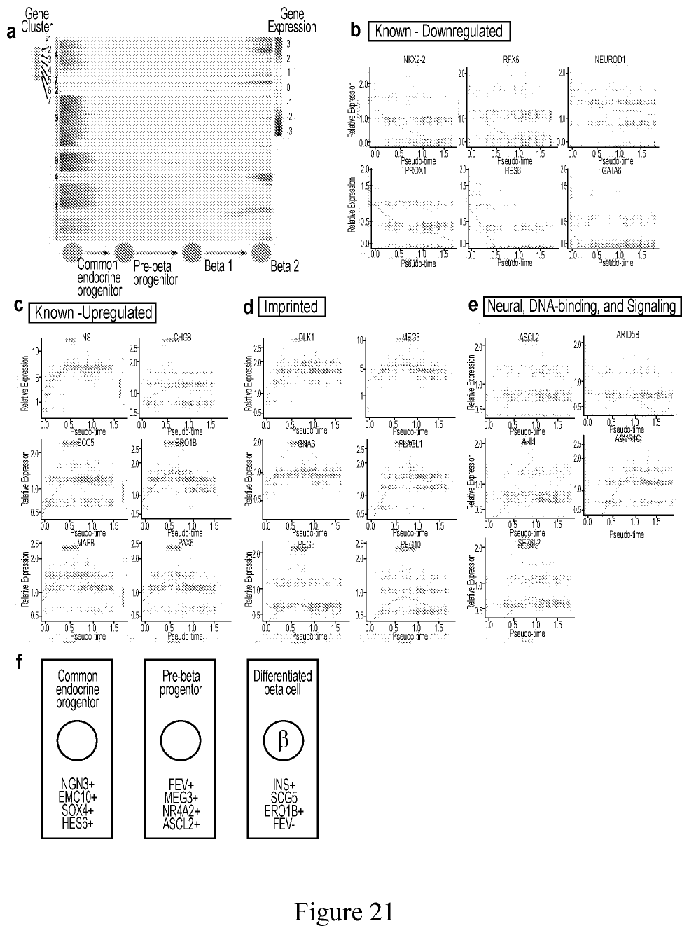

Pseudotemporal ordering was used to identify genes that were differentially expressed across a single-cell trajectory. This analysis was first applied to the beta lineage branch, which exhibited differentiation starting with cluster 6 cells (common endocrine progenitors) to cluster 8 cells (pre-beta progenitors) and finally to beta cells (FIG. 21a). Lineage branch analysis resulted in seven major gene clusters that displayed three main patterns of gene expression: genes that were highly expressed in the common progenitor stage but tapered in expression as differentiation proceeded (gene clusters 2-4), genes that turned on specifically in the pre-beta progenitor stage and were either subsequently downregulated or remained expressed (gene clusters 1, 6, and 7), and genes that turned on specifically in differentiated beta cells (gene cluster 5) (FIG. 21a). NGN3 was found in gene cluster 3, along with other genes known as endocrine progenitor markers, such as NKX2-2, RFX6, NEUROD1, PROX1, HES6, and GATA6 (FIG. 21a). These known endocrine progenitor markers were highly expressed in the common progenitor cluster (cluster 6) but were downregulated during the pre-beta progenitor stage (FIG. 21b). On the other hand, genes within gene cluster 7, which were expressed specifically in differentiated beta cells, included WNT4, which regulates beta cell proliferation, and RGS2, which regulates beta cell mass (Dong et al., 2017; Heller et al., 2011). INS, which is a definitive marker of beta cells, began to become upregulated in the pre-beta progenitor stage and reached peak expression in the differentiated beta cell stage (FIG. 21c).

In view of the data, it was expected that genes within gene clusters 1, 6, and 7, which were upregulated during the pre-beta progenitor stage, would serve as regulators of beta cell lineage allocation. FEV was found in gene cluster 7. Genes that displayed high upregulation during the pre-beta progenitor stage also included genes known to be involved in beta cell differentiation and function, such as CHGB, SCG5, ERO1B, MAFB, and PAX6 (FIG. 21c). The upregulation of known genes involved in insulin production, insulin processing, and the beta cell differentiation program confirmed that the pseudotemporal ordering analysis was robust. Genes involved in cytoskeletal remodeling and cell migration, including ROBO2, VIL1, CALY, TAGLN2, KIF5C, KIF12, and PHACTR2, were also upregulated beginning in the pre-beta progenitor stage. Without wishing to be bound by theory, these cytoskeletal remodeling and cell migration genes could be reflective of islet cell migration and formation that occurs concurrently with beta cell differentiation (Sharon et al., 2019a).

We also identified candidate regulators not previously known to be involved in beta cell lineage allocation. These genes were organized into three broad categories: those that were imprinted, those involved in neural development, and others involved in transcription and canonical signaling pathways. Imprinted genes that were upregulated during beta cell differentiation included DLK1, MEG3, GNAS, PLAGL1, PEG3, and PEG10 (FIG. 21d). The role of imprinting in endocrine cell development has not been previously explored, although loss or dysregulation of imprinted genes has been implicated in impaired pancreatic endocrine cell function. Epigenetic dysregulation of DLK1-MEG3 microRNAs has been observed in human T2D islets (Kameswaran et al., 2014). In the GNAS locus, improper mono-allelic expression of GNAS from the maternal allele results in a host of growth and metabolic disorders, including obesity (Weinstein et al., 2010). Bi-allelic expression of PLAGL1 causes 60% of all cases of transient neonatal diabetes mellitus (TNDM) (Hoffmann and Spengler, 2012; Kamiya et al., 2000). PEG3 encodes a zinc finger transcription factor that inhibits beta cell proliferation in mice (Sojoodi et al., 2016), and PEG10 was recently identified by our group to be a lineage marker of the murine alpha lineage (Byrnes et al., 2018).

Upregulated genes known to be involved in neural development included ASCL2, AHI1, and SEZ6L2 (FIG. 21e). ASCL2 (also called MASH2) is also an imprinted gene and comes from a family of bHLH transcription factors that regulates neuronal progenitor differentiation and peripheral nerve regeneration (Ge et al., 2006; Guillemot et al., 1993; Küry et al., 2002). AHI1 regulates cortical development in humans (Doering et al., 2008). Mutations in SEZ6L2 have been implicated in seizure-related phenotypes and, more recently, the gene has been identified as a marker for developing islet cells during embryogenesis (Bedoyan et al., 2010; Hald et al., 2011). Finally, ARID5B and ACVR1C were genes significantly expressed as differentiation into the beta lineage occurred (FIG. 21e). ARID5B participates as a transcriptional coactivator that is required for adipogenesis (Okuno et al., 2013). Signaling through ACVR1C, an Activin A receptor, has been reported to inhibit insulin secretion from beta cells (Bertolino et al., 2008), suggesting that signaling downstream of ACVR1C possibly blocks premature insulin secretion in pre-beta progenitors or early-forming beta cells during human endocrine cell development. The identification of these genes upregulated during the pre-beta progenitor stage begins to elucidate transcriptional circuitry involved in human beta cell differentiation that can be validated in in vitro differentiation models (FIG. 21f).

### Example 12

Candidate Lineage Regulators of the Alpha Cell Lineage

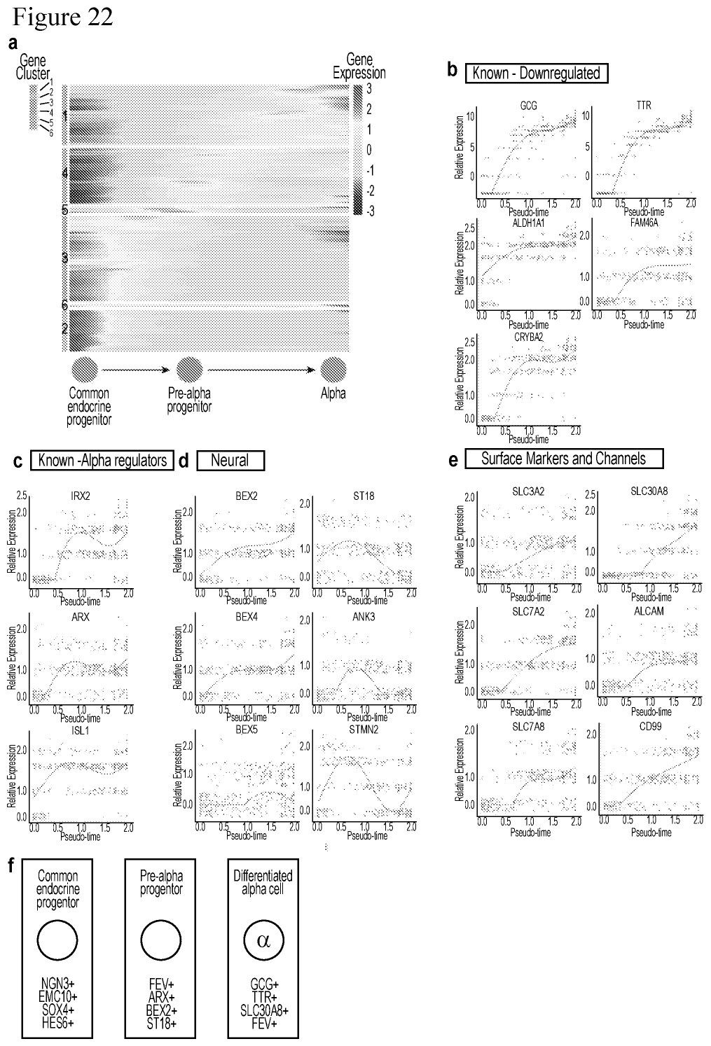

A similar analysis was applied to identify genes that were differentially expressed during differentiation into alpha cells. Based on pseudotemporal ordering, differentiation of the alpha lineage from endocrine progenitors began with cluster 6 (common progenitors) that differentiated into cluster 9 cells (pre-alpha progenitors), which then became differentiated alpha cells found in cluster 9 (FIG. 19b). Alpha lineage branch analysis resulted in six major gene clusters that, similar to the beta lineage branch analysis (FIG. 21a), displayed three main patterns of gene expression: genes that were highly expressed in the common progenitor stage but tapered in expression as differentiation proceeds (gene clusters 2 and 3), genes that turned on specifically in the pre-alpha progenitor stage and were either subsequently downregulated or remained expressed (gene clusters 1, 4, and 5), and genes that turned on specifically in differentiated alpha cells (gene cluster 6) (FIG. 22a). Genes known to be expressed by differentiated alpha cells, including GCG, TTR, ALDH1A1, FAM46A, and CRYBA2 (Dorajoo et al., 2017; Muraro et al., 2016; Su et al., 2012), displayed upregulated expression along pseudotime (FIG. 22b). Genes known to regulate alpha lineage allocation, such as IRX2, ARX, and ISL1, also were upregulated but specifically beginning in the pre-alpha progenitor cluster (FIG. 22c).

Given that gene clusters 4 and 5 contained genes that were upregulated specifically after NGN3 downregulation and the acquisition of alpha cell identity, these genes were expected to be regulators of alpha lineage allocation. Similar to the analysis of the beta cell lineage, MAFB, PAX6, ERO1B, AHI1, PEG10, SCG5, and ACVR1C were found to be upregulated along alpha cell differentiation, indicating that these markers are common genes upregulated during endocrine cell differentiation as a whole. We observed upregulation of various neural transcription factors and genes during alpha cell differentiation. BEX2, BEX4, and BEX5 were all upregulated during alpha cell fate allocation and are members of the brain-expressed X-linked transcription factor family that are highly expressed in the brain (Alvarez et al., 2005) (FIG. 3.6d). ST18 was another neuronal lineage transcription factor that promotes cholinergic motor neuron differentiation and that was upregulated during alpha cell development (Teratani-Ota et al., 2016) (FIG. 3.6d). Other genes upregulated during this process and implicated in neural function included ANK3, which is required in neurons for proper synapse structure and function (Smith et al., 2014), and STMN2, which is required for normal axonal outgrowth and regeneration in the nervous system (Klim et al., 2019) (FIG. 22d). Furthermore, we identified a number of cell surface markers that were first expressed during the pre-alpha progenitor stage and can be used to guide cell purification. These included SLC3A2, SLC7A2, SLC7A8, SLC30A8, ALCAM, and CD99 (FIG. 22e). SLC30A8 has already been shown to be required in adult alpha cells for hypoglycemia-induced glucagon secretion (Solomou et al., 2015). The analysis of the alpha differentiation trajectory (FIG. 22f), paired with that of the human beta lineage, highlights the power of pseudotemporal ordering in defining the dynamic transcriptional programs in place as endocrine progenitors become specified towards distinct hormone-expressing lineages.

### Example 13

Cellular and Transcriptional Dynamics of the Developing Endocrine Compartment

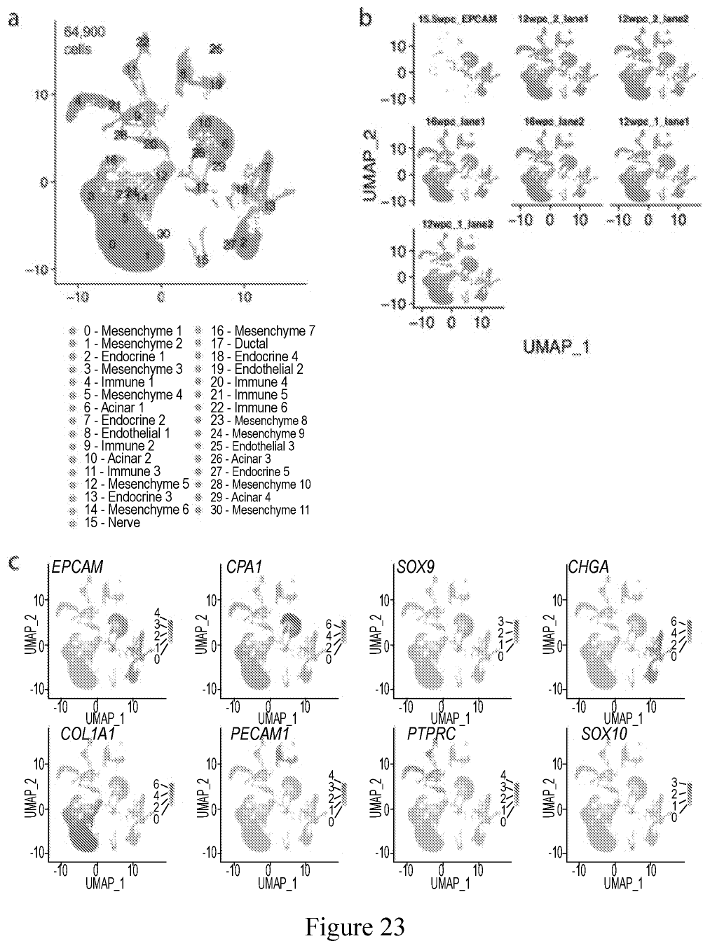

To understand endocrine cell development across actual developmental time, single-cell RNA-sequencing was performed on tissues at three additional time points to add to the analysis on 12wpc human fetal pancreas (referred to as 12wpc_1): a second biological replicate of 12wpc (referred to as 12wpc_2), 15.5wpc, and 16wpc. All datasets were depleted of red blood cells through immunomagnetic separation and were generated using the 10× Genomics version 3 (V3) sequencing chemistry, except for the 15.5wpc sample, which was enriched for EPCAM+ cells through FACS and processed using version 2 (V2) sequencing chemistry. All datasets were merged with Seurat 3's new integration method for merging and batch correction, resulting in 31 distinct clusters (FIG. 23a) that were classified as CPA1+ acinar cells, SOX9+ ductal cells, CHGA+ endocrine cells, COL1A1+ mesenchymal cells, PECAM1+ endothelial cells, PTPCR+ immune cells, and SOX10+ nerve cells (FIG. 23c). The two 12wpc samples and single 16wpc sample were represented in all clusters, highlighting the robustness of Seurat 3's new Integration method for batch correction by biological sample (FIG. 23b). As expected, given that the 15.5wpc sample was enriched for EPCAM+ cells, this time point only contributed to the EPCAM+ populations in the merged dataset (FIG. 23b, c). To focus on the endocrine compartment, CHGA+ endocrine clusters from the merged dataset were sub-clustered (FIG. 23c).

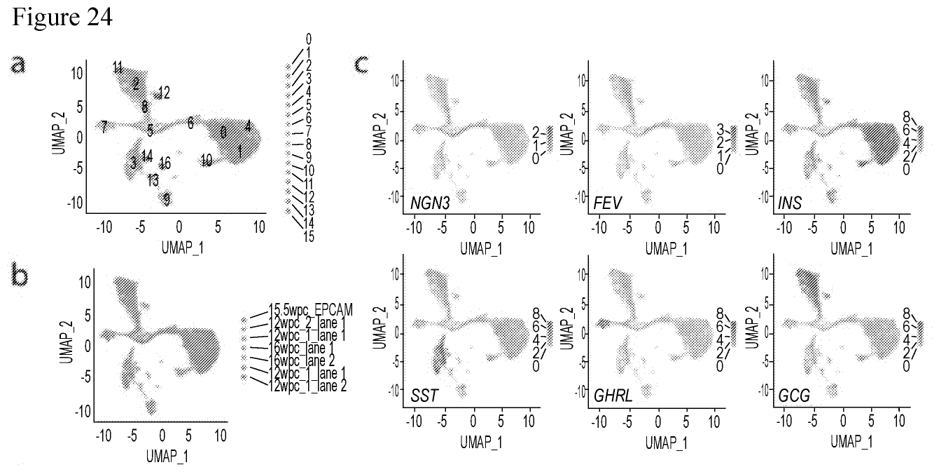

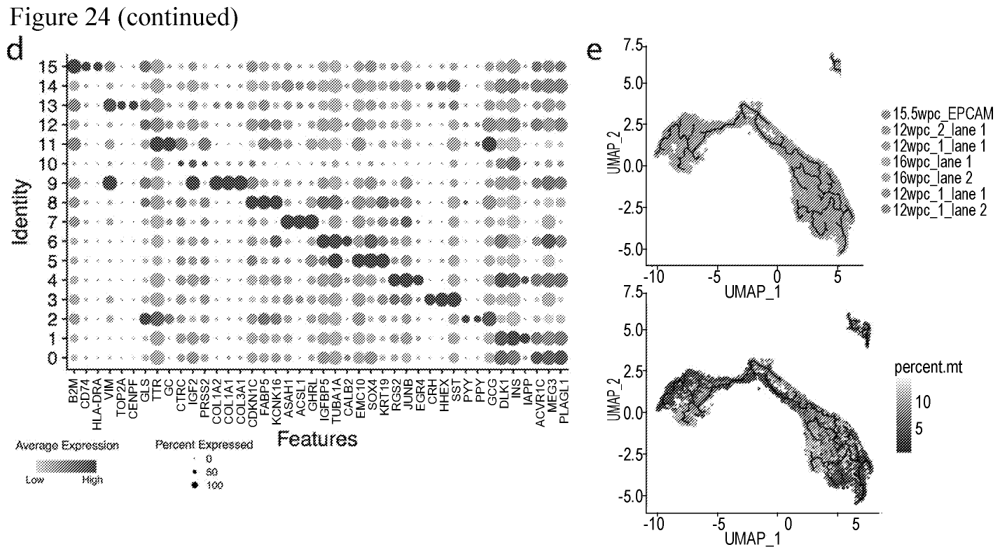

Sub-clustering of the endocrine lineage resulted in 15 distinct populations (FIG. 24a), including NGN3+ endocrine progenitors, INS+ beta cells, GCG+ alpha cells, SST+ delta cells, and GHRL+ epsilon cells (FIG. 24c). We also observed a FEV+ cluster that was not defined by any hormone expression (FIG. 24c), which is in line with the FEV+ endocrine progenitor cells identified in the 12wpc pancreas (FIG. 18b, 19a). This FEV+ cluster had representation from all four fetal time points (FIG. 24b, c), indicating that FEV+ progenitors appear as early as 12wpc and persist at least as late as 16wpc in human pancreatic development. Plotting the top three differentially expressed genes from each cluster highlighted the gene expression profile differences of each endocrine cluster (FIG. 24d).

We next sought to reconstruct lineage relationships across multiple time points through pseudotemporal ordering. Batch effect, unfortunately, is a major issue that still confounds single-cell RNA-sequencing analysis, despite multiple groups developing algorithms to address this problem (Butler et al., 2018; Haghverdi et al., 2018; Stuart et al., 2019). In our merged dataset, batch effect became problematic as our 15.5wpc sample was processed with V2 10× Genomics version chemistry as opposed to V3 chemistry, which was utilized for processing the 12wpc and 16wpc samples. V3 chemistry increased the sensitivity of gene capture, and this was particularly evident by the percentage of mitochondrial genes captured in V3 datasets, in which more mitochondrial genes were represented (FIG. 24e). This discrepancy in mitochondrial gene capture led to a batch effect in pseudotemporal ordering that was not resolved through regressing on mitochondrial content (FIG. 24e). Endocrine cells from the 15.5wpc sample clustered more closely to one another than integrating with cells from other time points, and this was driven by differences in mitochondrial content of the datasets (FIG. 24e). In order to reconstruct lineage relationships across multiple time points, additional human fetal datasets using V3 chemistry are used.

### Example 14

Understanding the Emergence of Distinct Cellular Compartments During In Vitro Beta Cell Differentiation at Single-Cell Resolution

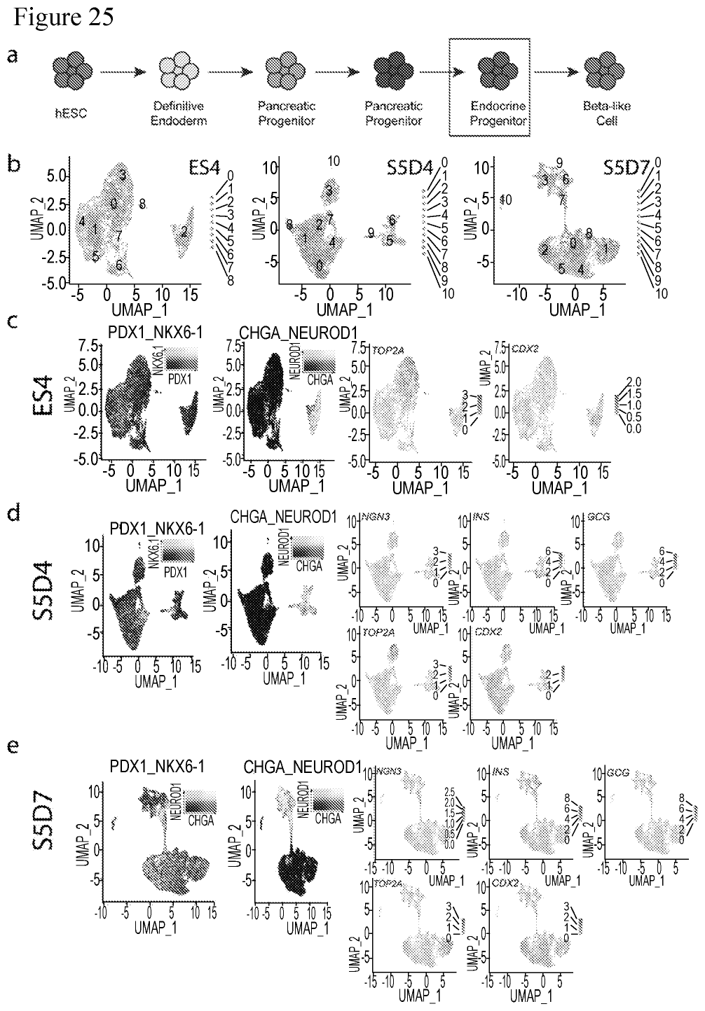

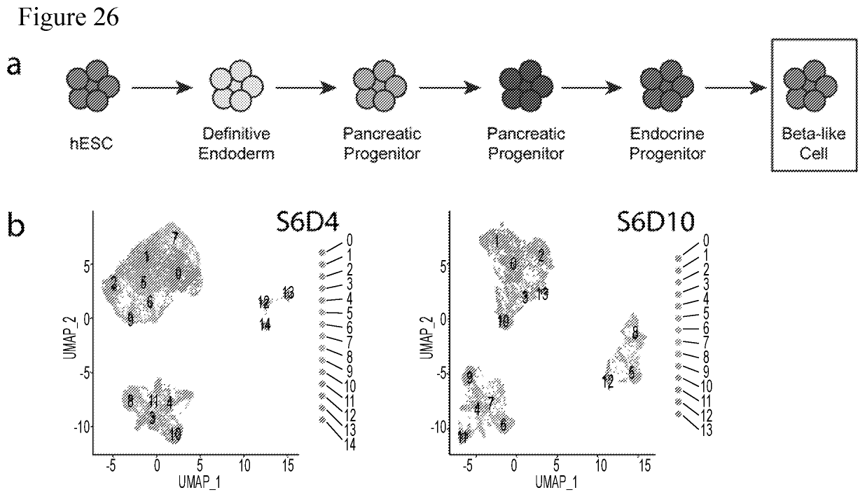

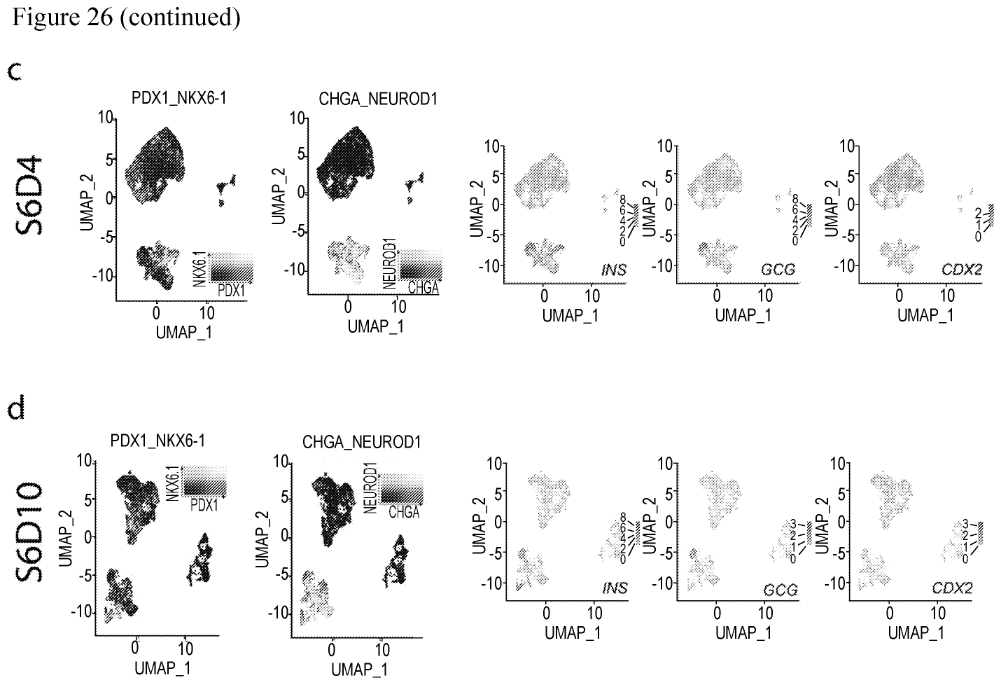

Directed differentiation of hESCs to a beta cell lineage represents a powerful approach for not only generating beta cells for diabetes but also understanding human beta cell differentiation. Given the significant heterogeneity in cells generated by directed differentiation of hESCs towards the beta lineage, single-cell RNA-sequencing was leveraged to classify distinct cellular populations that arose across five main stages of in vitro beta cell differentiation: stages containing early-, middle-, and late-stage endocrine progenitors (ES4, S5D4, and S5D7) and two stages within the beta lineage stage (S6D4 and S6D10). UMAP-based clustering of all five time points revealed the presence of PDX1+ clusters, reflecting induction towards the pancreatic lineage during the directed differentiation towards the beta lineage (FIGS. 25, 26). In ES4, proliferating PDX1+ pancreatic progenitors, PDX1+/NKX6.1+ pancreatic progenitors, early-induced endocrine cells marked by CHGA and NEUROD1, and CDX2+ clusters were identified as likely representing intestinal lineages that arose from improper differentiation (FIG. 25c). Of mid- to late-stage endocrine progenitors in Stage 5, only a small percentage expressed the endocrine progenitor marker NGN3 (FIG. 25d, e), which was expected given the transient nature of NGN3 expression. Instead, we observed PDX1+/NKX6.1+ progenitors, replicating PDX1+/NKX6.1+ progenitors, the persistence of CDX2+ cluster, and CHGA+/NEUROD1+ endocrine clusters that began to express hormones, such as INS and GCG (FIG. 25d, e). During Stage 6, which is defined as the beta cell stage, we again observed CDX2+ clusters and CHGA+/NEUROD1+ endocrine clusters that contain INS- and GCG-producing cells (FIG. 26a-d).

### Example 15

hESC-Derived FEV+ Cells are Transcriptionally Similar to In Vivo FEV+ Progenitors

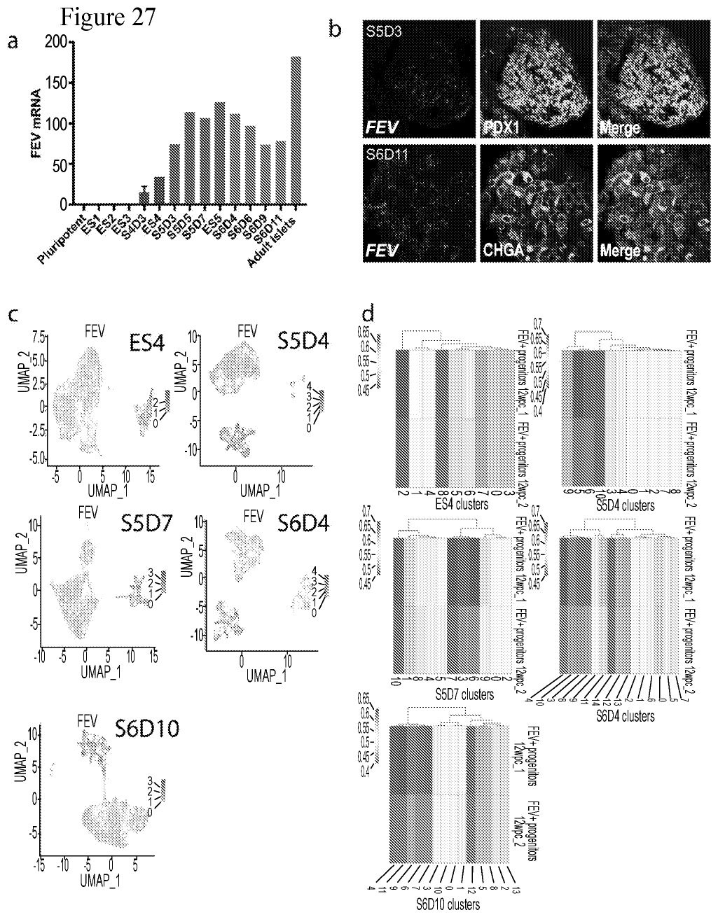

Given that we had identified an endocrine progenitor stage defined by FEV expression in both mouse (Byrnes et al., 2018) and human fetal beta cell development (FIG. 18b), we examined whether the derivation of hESC-derived beta cells also involved transit through a FEV-expressing cell stage. From qPCR, it was determined that FEV began to be expressed starting in Stage 4 pancreatic progenitors and was robustly expressed in Stage 5 endocrine progenitors (FIG. 27a). FEV expression persisted in cells at the Stage 6 beta cell stage (FIG. 27a), which was in contrast to differentiated beta cells in human pancreatic development that did not express FEV (FIG. 18b, 19a). This was consistent with in situ hybridization for FEV, in which FEV transcript was observed in S5D3 and S6D11 clusters (FIG. 27b). Consistent therewith, in the transcriptomic profiling of cells from the end of Stage 4 to Stage 6, FEV+ cells were present at each sampled time point (FIG. 27c). To determine how transcriptionally similar the FEV+ cells found in beta cell differentiation in vitro were to FEV+ progenitors found in human endocrine cell development in vivo, a Pearson's correlation analysis was performed. Correlation analysis of FEV+ progenitors from the 12wpc_1 and 12wpc_2 datasets compared to the hESC-derived FEV+ cells revealed higher transcriptional correlation of all FEV+ clusters than compared to all FEV− clusters (FIG. 27d), indicating that cells undergoing in vitro differentiation to a beta cell fate transit through a FEV-expressing stage similar to that found in vivo.

### Example 16

Mapping In Vitro Beta Cell Differentiation at Single-Cell Resolution

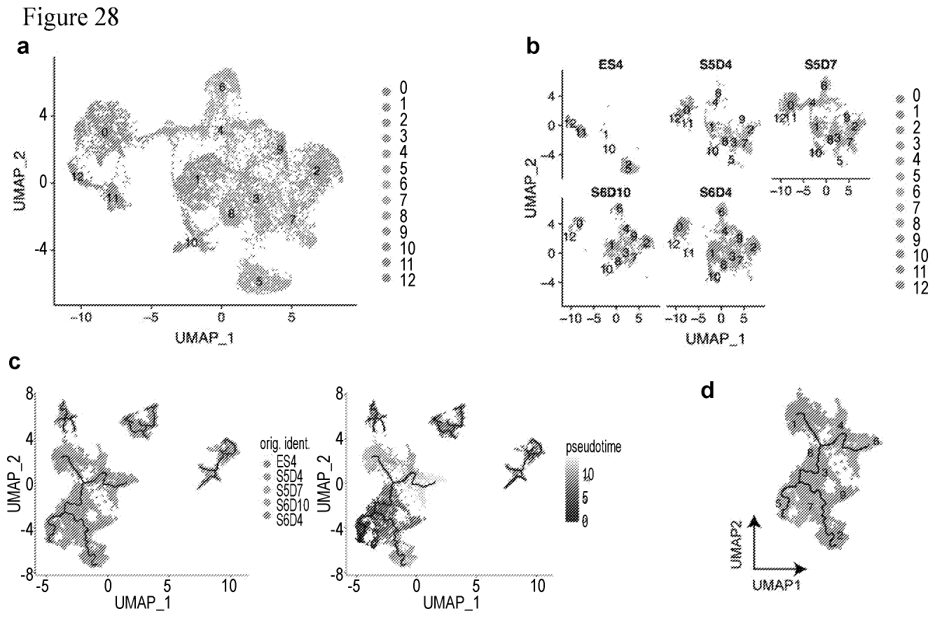

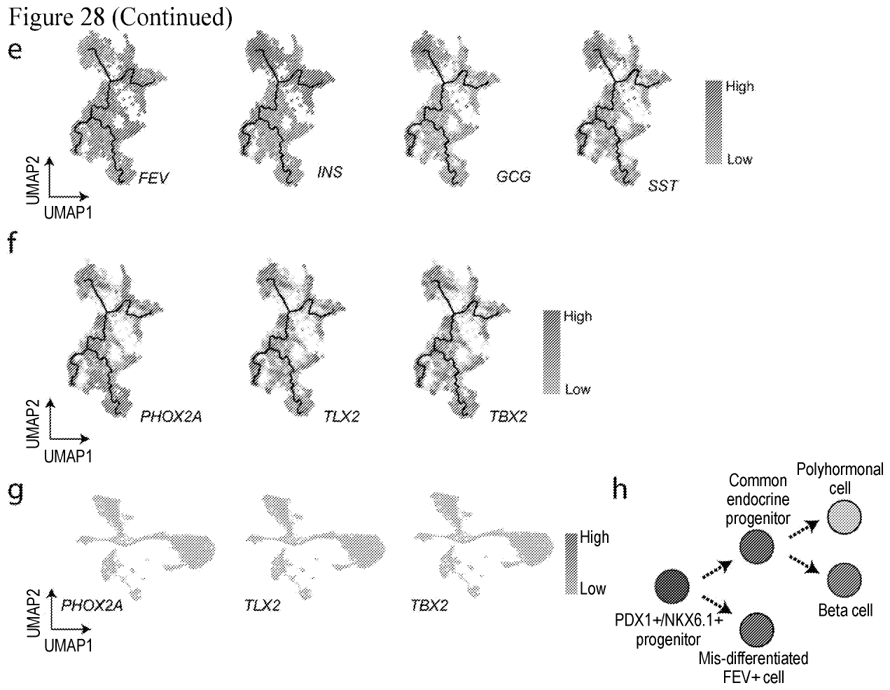

The lineage relationships among hESC-derived endocrine cells during in vitro beta cell differentiation were also reconstructed. First, all CHGA+ endocrine clusters from each sampled time point were merged using Seurat 3 (FIG. 28a, b). Given that batch correction via Seurat 3's integration method is currently not compatible with pseudotemporal ordering by Monocle 3, we utilized Monocle 3's internal batch correction method to merge hESC-derived endocrine clusters from different time points and subsequently performed pseudotemporal ordering. The result was one main trajectory, which we used in subsequent analyses, that began with ES4 cells, our first sampled time point and the designated start of our pseudotemporal ordering (FIG. 28c, d). As pseudotime progressed, three main endpoints of the differentiation trajectory were observed (FIG. 28c, d). As expected, a beta cell lineage that expressed INS constituted one endpoint and was primarily composed of S6D4 and 056D10 cells (FIG. 28e). A second endpoint was composed of poly-hormonal cells that expressed INS, GCG, and SST (FIG. 28e). These polyhormonal cells also were derived from the S6D4 and S6D10 timepoints (FIG. 28c). A third endpoint in the differentiation trajectory surprisingly resulted from a bifurcation event early in pseudotime before the acquisition of hormone identity (FIG. 28f). The cells at this third endpoint did not express INS, GCG, or SST (FIG. 28e). Instead, this population appeared to be mis-differentiated and expressed transcription factors such as PHOX2A, TLX2, and TBX2 (FIG. 28f), all of which regulate differentiation and function of cells in the nervous system. These transcription factors were not expressed in a large fraction of the endocrine compartment of human fetal pancreata (FIG. 28g). PHOX2A is required for proper differentiation of neurons in the autonomic nervous system, as it is crucial for the development of neural crest-derived cells (Borghini et al., 2006; Hirsch et al., 1998; Lo et al., 1999; Pattyn et al., 1997; Tiveron et al., 1996). TLX2 is a transcriptional target of the PHOX2 family of transcription factors and is also required for proper development of the neural crest lineage and thus, also the enteric nervous system (Borghini et al., 2006). TBX2 promotes anterior neural specification by suppressing FGF signaling (Cho et al., 2017). FEV was still expressed in this mis-differentiated lineage (FIG. 28e), indicating that there may have been a subset of FEV+ endocrine progenitors during in vitro beta cell differentiation that improperly differentiated into a neural lineage (FIG. 28h). The expression of these transcription factors in this blocked cell type within our in vitro beta cell differentiation indicates that these cells have mis-differentiated into a neural identity.

### Example 17

FEV Appears to be Required for Proper Human Beta Cell Differentiation and Function

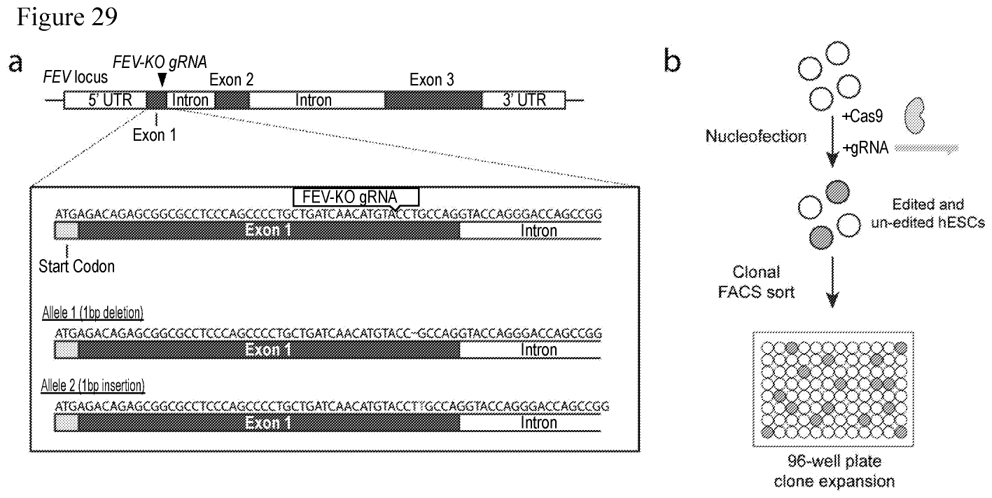

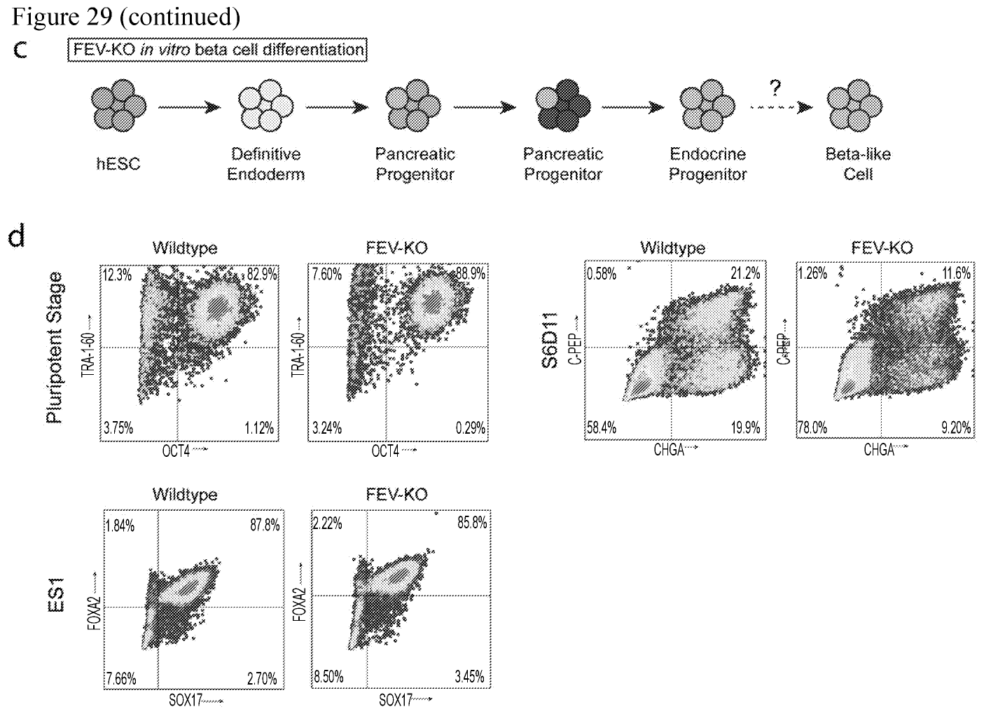

In addition to identifying FEV as a marker for endocrine progenitor stages in human endocrine cell development, we wanted to determine if FEV had any functional role in beta cell differentiation. In Fev knockout (KO) mice, glucose clearance from the blood following a glucose challenge was significantly slowed, and the insulin content of beta cells was decreased (Ohta et al., 2011). Given that this study utilized a whole-body Fev KO, the defects in glucose homeostasis and the reduction in insulin content could have been a result of a requirement for FEV in non-pancreas cells or for FEV function in multiple stages in the lifetime of a beta cell. To test the requirement of FEV in human beta cell differentiation and function, the in vitro beta cell differentiation platform was used to first generate a FEV-KO hESC line through CRISPR (clustered regularly interspaced short palindromic repeats)/Cas9-mediated genomic editing (FIG. 29a). The human FEV locus contains three exons, and the guide RNA (gRNA) was designed to target the end of exon 1 (FIG. 29a). Wild-type hESCs were nucleofected with Cas9 and a FEV-KO gRNA, cultured for 2 passages following nucleofection to allow for recovery, and then clonally plated (FIG. 29b). One clone was identified that exhibited a 1-bp insertion in one FEV allele and a 1-bp insertion in the second allele, both located at the end of exon 1 (FIG. 29a). Both genomic edits resulted in frameshift mutations that changed the entire amino acid sequence following the amino acids encoded by exon 1. The DNA-binding domain normally found in exon 3 was predicted to no longer be properly translated in the new amino acid sequence following these frameshift mutations. This FEV-KO clonal hESC line, along with a WT (wild-type) control clonal line, was expanded and subsequently adapted to suspension-based culture for the in vitro beta cell differentiation platform. Directed differentiations of both FEV-KO and WT control hESCs were then performed (FIG. 29c). Ablation of FEV did not affect pluripotency of hESCs, as measured by OCT4 and TRA-1-60 staining (FIG. 29d). Ablation of FEV also did not inhibit differentiation into Stage 2 definitive endoderm as marked by SOX17 and FOXA2 (FIG. 29d), which is concordant with the lack of FEV expression before Stage 2. By the Stage 6 beta-like cell stage, however, FEV deficiency did result in a reduction of CHGA+/CPEP+ cells (21.2% in WT differentiation versus 11.6% in FEV-KO differentiation) (FIG. 29d), indicating that FEV is needed for proper differentiation into the hormone-expressing beta lineage.

### Example 18

Generation of New Tools and Platforms for Understanding Human Beta Cell Development: Identification of FEV Transcriptional Targets, Isolation of FEV-Expressing Cells During In Vitro Beta Cell Differentiation, and Validation of Novel Candidate Regulators of Beta Cell Lineage Allocation and Function

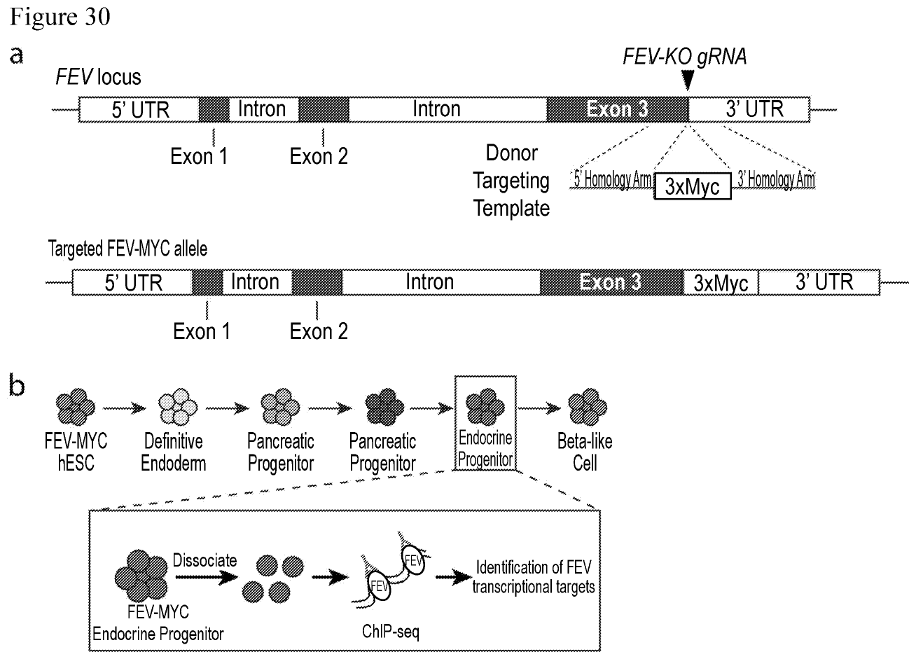

The discovery that the transcription factor FEV is expressed in hESC-derived endocrine progenitors and beta-like cells prompted us to generate tools through which we could interrogate the function of the FEV gene. Through CRISPR/Cas9-mediated genomic editing, a FEV-MYC hESC line was constructed in which a MYC epitope tag was fused to the endogenous FEV transcription factor at the C-terminus (FIG. 30a). This tagging would allow ChIP-seq to be performed to identify transcriptional targets of FEV (FIG. 30b). To engineer this line, a FEV-KI (knock-in) gRNA that targeted the end of exon 3 of the FEV locus was engineered (FIG. 30a). A commercially-synthesized DNA targeting template containing three sequential MYC epitopes separated by small genomic spacers (termed 3×MYC) was obtained (FIG. 30a). This 3×MYC sequence was flanked by homology arms found around the cut site in the endogenous FEV locus (FIG. 30a). The 3×MYC sequence with the flanking homology arms was cloned into a pUC19 vector and transformed into competent cells in order to obtain sufficient DNA quantity for PCR amplification of the targeting template. This targeting template along with the FEV-KI gRNA and Cas9 protein were then nucleofected into wild-type hESCs, cultured for 2 passages following nucleofection to allow for recovery, and then clonally plated. The FEV locus of isolated clones was screened for successful knock-in of the 3×MYC by PCR amplification of the knock-in region and Sanger sequencing the resulting PCR amplicon. This screening strategy identified a FEV-MYC hESC clonal line with one FEV allele that showed successful knock-in of the 3×MYC tag in frame with the FEV locus.

Given that FEV is a transcription factor that is required for proper endocrine differentiation in an in vitro beta cell differentiation platform, the generation of this FEV-MYC hESC line is expected to be valuable in interrogating the mechanism through which FEV regulates proper human beta cell differentiation. ChIP-seq on FEV+ endocrine progenitors at Stage 5 of our in vitro differentiation can identify transcriptional targets of FEV (FIG. 30b), which will serve as candidate effectors of proper endocrine cell differentiation. Additionally, FEV is expressed in Stage 6 cells when cells of the beta lineage begin to form. In Stage 6, non-beta, FEV-expressing cells were found that were blocked in their differentiation potential (FIG. 30b). ChIP-seq on these blocked cells is expected to identify transcriptional targets of FEV that mediate improper beta cell differentiation. Fev-KO mouse studies demonstrated that Fev binds to the insulin promoter to promote Ins transcription (Ohta et al., 2011). ChIP-seq on sorted INS+ beta cells from Stage 6 will also identify FEV targets that regulate beta cell function and can confirm if INS is also a target of FEV in human beta cells.

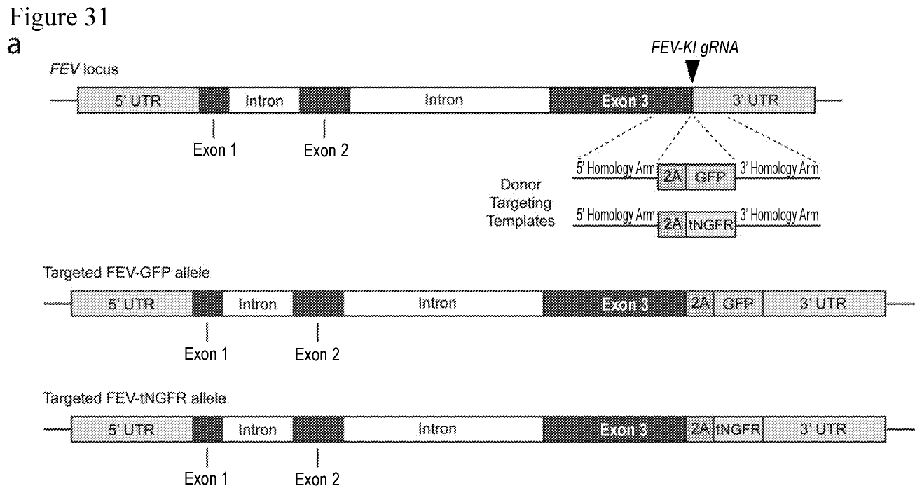

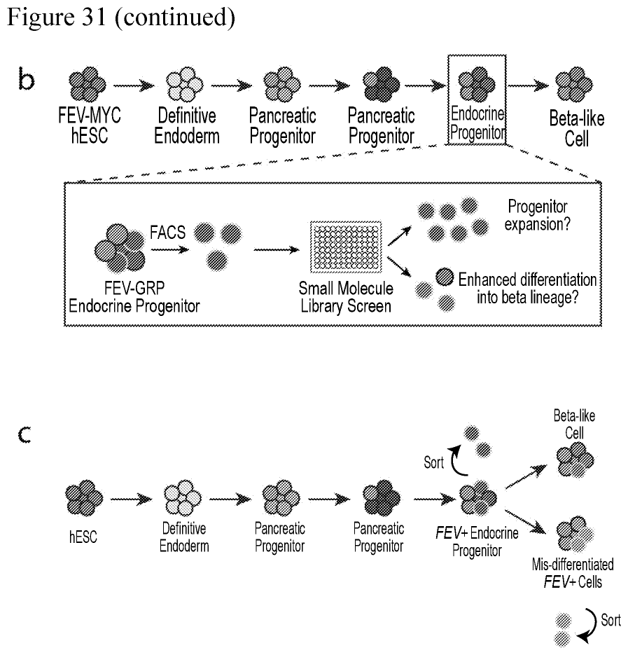

As disclosed herein, tools to isolate and characterize the FEV-expressing cell population during human beta cell differentiation have been developed. Two FEV reporter hESC lines have been constructed: a FEV-GFP line and a FEV-tNGFR (truncated Nerve Growth Factor Receptor) line (FIG. 31a). The FEV-GFP line will allow for fluorescent-based isolation of FEV-expressing cells during in vitro beta cell differentiation. For applications that require quicker isolations of much larger quantities of FEV-expressing cells than fluorescent-based sorting can provide, the FEV-tNGFR line can be utilized. The tNGFR is a surface marker in which the cytoplasmic intracellular signaling domain of the NGFR is removed and thus can be leveraged for magnetic bead-based isolation methods (Dever et al., 2016). This tNGFR enrichment strategy has already been implemented in human clinical studies for the isolation of large quantities of tNGFR-tagged cells (Bonini et al., 2003; Oliveira et al., 2015). Both the GFP and the tNGFR sequences used for these KI lines were preceded by a T2A element that would enable bicistronic translation of FEV and the reporter protein. Similar to our strategy with the FEV-MYC hESC line generation, we obtained commercially-synthesized DNA targeting templates containing either the 2A-GFP or 2A-tNGFR sequences flanked by homology arms around the cut site. Following cloning and subsequent PCR amplification of the FEV-GFP or FEV-tNGFR targeting templates, they were nucleofected into wild-type hESCs along with the same FEV-KI gRNA used for the FEV-MYC line generation and Cas9 protein. The nucleofected hESCs were cultured for 2 passages to allow for recovery and then clonally plated. The FEV loci of isolated clones were screened for successful knock-in of the 2A-GFP or 2A-tNGFR sequences by PCR amplification of the knock-in region and Sanger sequencing of the resulting PCR amplicon. This screening strategy identified both a FEV-GFP and a FEV-tNGFR hESC clonal line with one FEV allele that showed successful knock-in of the reporter in-frame with the FEV locus.

Purification of FEV-expressing cells at defined stages of the in vitro beta cell differentiation process will enable us to understand the differences among FEV-expressing populations at each differentiation stage. Specifically, purifying FEV+ endocrine progenitors at stage 5 of our differentiation program will permit small molecule screens to identify compounds that can either induce progenitor expansion prior to beta cell lineage commitment or enhance differentiation toward the beta lineage (FIG. 31b). Similarly, given that FEV-expressing cells were found that mis-differentiated in Stage 6, we can also harness the utility of these reporter lines to isolate these mis-differentiated cells and screen for compounds that can correct their differentiation into the beta lineage or block their emergence altogether (FIG. 31c). Further, given that beta cells in human pancreatic development transit through a FEV-expressing precursor stage, we can test whether enriching for FEV+ endocrine progenitor-stage cells during Stage 5 will yield higher efficiencies of beta cell differentiation at Stage 6. Given that large quantities of cells would be needed, utilizing the FEV-tNGFR line would provide for isolation of enough FEV+ endocrine progenitors in Stage 5 to allow re-aggregation into clusters for differentiation into Stage 6. Thus, the ability to isolate FEV-expressing cells throughout in vitro beta cell differentiation will refine our understanding of the cellular heterogeneity that emerges during this directed differentiation process.

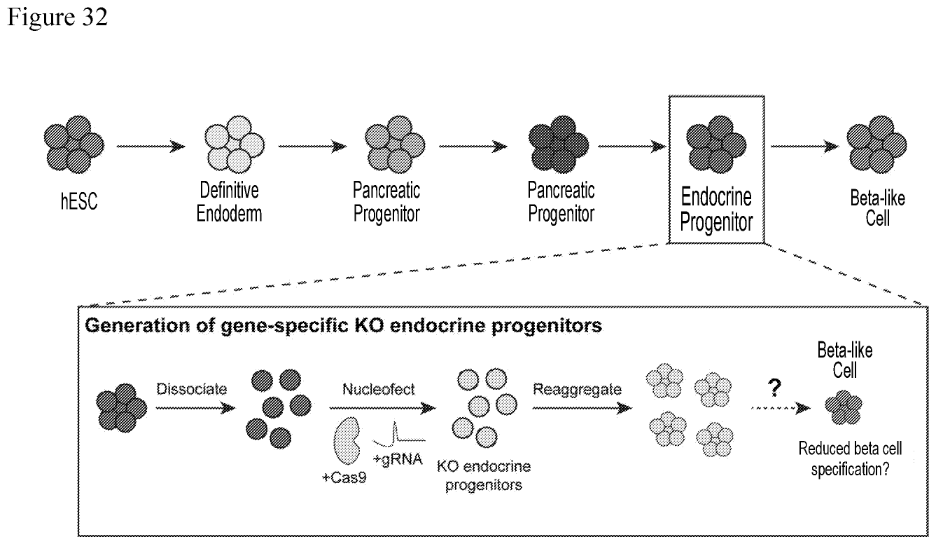

The in vivo and in vitro single-cell RNA-sequencing analyses have resulted in the identification of candidate beta cell lineage regulators. In order to functionally validate these candidate regulators, we developed a flexible platform on which we can test whether these genes regulate beta cell lineage allocation (FIG. 32a). In this platform, different gRNAs are designed that will introduce a frameshift mutation within the genomic locus of each candidate regulator for functional testing. Given that these genes are candidate regulators of endocrine lineage allocation, we utilize the endocrine progenitor-stage (Stage 5) clusters from the in vitro beta cell differentiation and dissociate them into a single-cell suspension. Endocrine progenitor-stage cells are then nucleofected with Cas9 and a specific gRNA against the candidate regulator of interest. Because editing is not 100% efficient, nucleofection of these gRNAs will lead to a knockdown, not a full knock-out, of the candidate regulator of interest. Following nucleofection, endocrine progenitor-stage cells are reaggregated into clusters for directed differentiation towards the beta lineage. This platform, leveraging both in vitro beta cell differentiation and temporally controlled CRISPR/Cas9-mediated genomic editing, provides a versatile solution to functionally validate the candidate regulators identified through in silico methods.

The preceding Examples establish a platform and methodologies useful in promoting and optimizing protocols for directing progenitor cells into a differentiation pathway leading to mature, functional alpha and beta cells. The present disclosure contains several discrete advances useful in achieving these goals.

Redefining the NGN3+ Endocrine Progenitor Population in Human Pancreatic Development

In human endocrine cell development, NGN3 has long been thought to mark the endocrine progenitor population, given the function of Ngn3 in mouse pancreatic development. Indeed, NGN3 is required for endocrine cell differentiation in human endocrine cell development, as inactivating mutations of NGN3 lead to neonatal diabetes (Pinney et al., 2011; Wang et al., 2006). Beta cell mass is suspected to be reduced, not absent, in human cases of inactivating NGN3 mutations given that C-peptide is detected in the blood, albeit at low levels (Pinney et al., 2011). This is in contrast to mouse development, in which NGN3 ablation halts beta cell generation altogether (Gradwohl et al., 2000). Through the studies of human endocrine cell development disclosed herein, NGN3 did not appear to be the most robust marker of the endocrine progenitor population common to hormone-expressing lineages, such as the alpha and beta lineages, in our 12wpc_1 human fetal pancreas dataset. Other markers that appeared to more faithfully label this common endocrine progenitor population included EMC10, SOX4, HES6, and KRT19. CTD-2545M3.8 also emerged from our differential gene expression analysis as a marker specific to this common endocrine progenitor population, but awaits functional characterization.

In murine endocrine development, Ngn3+ endocrine progenitors give rise to all five hormone-expressing lineages of the pancreas (Gradwohl et al., 2000; Heller et al., 2005). However, in the human fetal pancreas, the NGN3-expressing common endocrine progenitor population appeared to only give rise to alpha and beta lineages. In our pseudotemporal ordering analysis, there was no trajectory that connected NGN3-expressing progenitors to the SST-expressing delta lineage or the GHRL-expressing epsilon lineages. A distinct PPY-expressing gamma population was not observed, as all PPY-expressing cells also expressed GCG and thus were annotated as alpha cells. If the difference in differentiation potential between human and mouse cells reflects true lineage relationships of NGN3-expressing endocrine progenitors in the developing human pancreas, this would depart from the dogma established by findings of the lineage potential of Ngn3+ progenitors in murine pancreatic development.

Identification of Novel Pre-Alpha and Pre-Beta Cell Stages in Human Pancreatic Development

Mapping endocrine cell development at a higher resolution using single-cell RNA-sequencing can be leveraged for developing new methods to generate endocrine cell types more efficiently from stem cell sources. Disclosed herein is the identification of pre-alpha and pre-beta progenitor stages that provide increased resolution regarding the steps required to differentiate into alpha or beta lineages in human pancreatic development. The work disclosed herein on human fetal pancreatic development offers novel endocrine progenitor stages onto which we can compare and contrast the biological relevance of the murine progenitor stages to those of human. The advent of single-cell RNA-sequencing has led to the discovery of several endocrine progenitor stages in mouse pancreatic development. One example is the work disclosed herein, which reveals an intermediate endocrine progenitor population defined by high Fev expression. Fev expression has also been identified in endocrine progenitor populations reported by several other single-cell RNA-sequencing studies of murine pancreatic development (Krentz et al., 2018; Scavuzzo et al., 2018), confirming the reproducibility of our finding. This Fev+ endocrine progenitor is derived from a Ngn3⁺ population, and differentiated endocrine lineages in the murine pancreas transit through a Fev− expressing cell stage (Byrnes et al., 2018).

Within the Fev+ progenitor population, cells that appeared to be pre-specified towards an alpha or beta cell fate were found. This is analogous to human pancreatic development in which we not only identified endocrine progenitors that expressed FEV but also observed that these FEV-expressing progenitors appeared to be already lineage-specified towards an alpha or beta cell fate. The in silico reconstruction of endocrine lineage relationships indicated that endocrine cell fate decisions in progenitors occurs at the Fev/FEV-expressing cell stage in both mouse and human.

Beyond this Fev-expressing endocrine progenitor stage, there are additional endocrine progenitor stages that have been identified in murine development. In particular, four distinct endocrine progenitor stages (termed EP1-4) have been proposed in mouse endocrine cell development (Yu et al., 2019). Expression of Ngn3, the canonical pro-endocrine lineage marker in pancreatic development, increased in EP1, peaked in EP2, decreased in EP3, and was not observed in EP4. Expression of Fev was found in EP3 and EP4 stages only (Yu et al., 2019), which is concordant with Fev being downstream of Ngn3 (Byrnes et al., 2018; Miyatsuka et al., 2014). Interestingly, many of the differentially expressed genes in each EP stage were also identified as top differentially expressed genes in either our human endocrine progenitor clusters or during pseudotemporal ordering. Specifically, Krt19 and Gadd45a, two genes that defined a human common endocrine progenitor stage in our dataset of 12wpc_1 human fetal pancreas, were found to be differentially expressed in EP2. Several candidate beta lineage regulators in human fetal development were also found in EP1 (Arid5b), EP3 (Ahi1), and EP4 (Rbp4, Peg10, Acvr1c, Sez6l2) (Yu et al., 2019). Similarly, several candidate alpha lineage regulators in human fetal development were found in EP3 and EP4 (Arx, Irx2, Fam46a, Slc30a8, Slc7a2, Slc7a8, Cryba2, St18, and Alcam) (Yu et al., 2019). Thus, these EP stages found in murine endocrine development appear to also have relevance to endocrine progenitor stages found in human fetal pancreatic development.

Transcriptional Mechanisms Underlying Fate Decisions are Shared Across Tissues

The single-cell RNA-sequencing analysis of human endocrine lineage allocation identified many candidate regulators previously identified and studied in the nervous system. Despite their derivation from different germ layers, both the pancreatic endocrine and neural lineages employ many of the same transcription factors that regulate their own development, including Ngn3, NeuroD1, Nkx2.2, Nkx6.1, Pax family of transcriptional regulators, and Fev (Blake and Ziman, 2014; Churchill et al., 2017; Gradwohl et al., 2000; Hendricks et al., 1999; Mastracci et al., 2013; Napolitano et al., 2015; Ohta et al., 2011; Pataskar et al., 2016; Prakash et al., 2009; Qi et al., 2001; Schaffer et al., 2010; Simon-Areces et al., 2010; St-Onge et al., 1997). These transcriptional similarities have an evolutionary basis, as the main source of insulin in invertebrates is in neurons (Wong et al., 2014). Thus, from an evolutionary perspective, it is not surprising that additional genes previously identified to be required for proper nervous system development and function are also implicated in pancreatic endocrine development and, more specifically, lineage allocation.

The development of enteroendocrine cells (EEs) in the intestine also shares striking similarity to pancreatic endocrine cell development. Proper differentiation of EEs in the intestine during development requires transcription factors also critical for pancreatic endocrine cell differentiation, including Ngn3 (Jenny et al., 2002; López-Díaz et al., 2007; Schonhoff et al., 2004), Nkx2.2 (Gross et al., 2016), Isl1 (Terry et al., 2014), NeuroD1 (Naya et al., 1997), Pax4 (Beucher et al., 2012a). As in pancreatic endocrine cell development, the EE lineage comprises multiple hormone-expressing cell types that are derived from a common progenitor cell defined by Ngn3 (Jenny et al., 2002). Recent work applying single-cell RNA-sequencing to murine EE development uncovered novel markers and lineage-specific regulators of the multiple EE lineages (Gehart et al., 2019), and many of these genes overlapped with the markers and candidate transcriptional regulators that we identified in mouse and human endocrine cell development and lineage allocation. Ngn3+ EE progenitors differentially express Sox4, Tox3, and Gadd45a (Gehart et al., 2019), all of which were also defining markers of our common endocrine progenitor in human endocrine cell development. Known hormone-specific lineage regulators in pancreatic endocrine cell development, such as Arx, Pax6, and Isl1, were also identified as EE-specific lineage regulators (Gehart et al., 2019). Interestingly, a number of novel candidate lineage regulators that we identified in mouse and human endocrine lineage allocation were also found to be lineage-specific regulators of the different EE lineages (Gehart et al., 2019). These include Nr4a2, Smarca1, Peg3, In, S100a1, and Klf4 (Gehart et al., 2019).

Timing of Endocrine Lineage Fate Decisions

The timing of endocrine lineage fate commitment in pancreatic development is not fully understood. The Ngn3+ endocrine progenitor stage has long been regarded as the master stage prior to endocrine cell differentiation, but the single-cell RNA-sequencing studies of pancreatic development disclosed herein have identified additional progenitor stages that arise between initial Ngn3 expression and acquisition of differentiated cell identity. This increased resolution of endocrine cell differentiation has provided us with new cell stages that we can interrogate for determining when endocrine lineage decisions are made. From both the mouse and human studies of endocrine cell development disclosed herein, Fev/FEV-expressing endocrine progenitors were already specified towards an alpha or beta cell fate. This heterogeneity in Fev/FEV-expressing progenitors suggests that endocrine lineage specification occurs at or before this Fev/FEV-expressing progenitor stage. The single-cell RNA-sequencing combined with pseudotemporal ordering identified endocrine progenitor populations that appeared to be fated towards one specific endocrine lineage.

The timing of endocrine fate decisions can also be regulated by extrinsic signals derived from the surrounding microenvironment. In murine development, the developmental time at which Ngn3+ progenitors form corresponds to their ultimate hormone lineage selection (Johansson et al., 2007). The competence window for alpha differentiation occurs earliest in murine pancreatic development, resulting in alpha cells being the first emerging endocrine lineage, followed by beta and gamma cells, and then lastly followed by delta cells (Johansson et al., 2007). In contrast, in human pancreatic development, the beta lineage is the earliest endocrine cell type to be detected (at 6wpc), followed by alpha cells (at 8-9wpc), delta cells (10wpc), and gamma cells (at 17wpc) (Jeon et al., 2009; Piper et al., 2004). Without wishing to be bound by theory, the differences in timing of emergence of endocrine lineages between mouse and human could be a direct result of the changing microenvironment during development that can be providing dynamic cues that promote one endocrine lineage over the other. From murine studies, we know that several compartments of the microenvironment influence pancreatic development, including vasculature, nerves, and mesenchyme (Borden et al., 2013; Golosow and Grobstein, 1962; Landsman et al., 2011; Magenheim et al., 2011; Reinert et al., 2013). However, the cellular composition of each microenvironment compartment can widely differ between that of mouse and human. From the single-cell profiling of human fetal pancreas provided herein, we identified several populations of endothelial cells whose transcriptional expression profiles changed throughout the course of development. These changes may influence the competency of endocrine progenitors to differentiate into distinct hormone lineages, either through secreted signaling molecules or direct interactions. Our single-cell profiling in both mouse and human pancreatic development also reflects different mesenchymal and nerve populations whose dynamics may regulate endocrine differentiation at distinct periods in development.

FEV in Human Endocrine Cell Differentiation and Function

Disclosed herein is an investigation of the role of FEV in human endocrine cell differentiation and function, which highlights potential differences between Fev/FEV in mouse versus human. In both mouse and human endocrine cell development, Fev/FEV was expressed in an intermediate progenitor stage that followed initial NGN3 expression and preceded hormone acquisition (Byrnes et al., 2018). FEV was also expressed in endocrine progenitor stage cells during in vitro beta cell differentiation from hESCs, which was concordant with FEV expression in endocrine progenitors in human fetal pancreatic development. Notably, although Fev-KO mice do not exhibit obvious differentiation defects in the islet lineages during development, we did observe a reduction in the differentiation into CHGA+/CPEP+ beta cells in the in vitro beta cell differentiation model. This indicates that FEV is required for human beta cell differentiation and is dispensable for mouse beta cell differentiation. Notable differences were also observed between Fev/FEV in mouse and human differentiated endocrine cells. While Fev expression persists in the alpha and beta lineages during mouse pancreatic development, FEV expression was downregulated in beta cells and only maintained in the alpha lineage in human pancreatic development. Single-cell RNA-sequencing of adult human islets has indicated that FEV is expressed in alpha cells and not beta cells (Segerstolpe et al., 2016). This is in contrast to the in vitro beta cell differentiation system, in which beta cells maintained FEV expression following differentiation of the FEV+ endocrine progenitor stage. In mouse beta cells, FEV binds to the insulin promoter to regulate Insulin transcription and, thus, insulin production. Given that FEV turns off in differentiated human beta cells in vivo, it is possible that FEV is either not needed for beta cell function or FEV inhibits beta cell function. In the in vitro beta cell differentiation system, we observed a subset of INS+ beta cells that did not express FEV, whereas another subset of INS+ beta cells did express FEV. The INS+/FEV− hESC-derived beta cells may correspond to bona fide beta cells found in vivo, and the INS+/FEV+ hESC-derived beta cells may either be mis-differentiated or on their way towards a FEV− state.

Identification of FEV transcriptional targets provides a clearer picture of its function. In the human beta cell lineage, loss of FEV coincided with a reduction in beta cell differentiation. Given that FEV was expressed in pre-beta progenitors in vivo and hESC-derived endocrine progenitor stage cells, FEV is expected to serve as a key transcriptional regulator for differentiation from a progenitor to a beta cell. Using the FEV-MYC hESC line during in vitro beta cell differentiation and performing ChIP-seq on FEV+ endocrine progenitor stage cells identified transcriptional targets expected to mediate the transition from a pre-beta progenitor to a differentiated beta cell. A transcriptional map of FEV transcription factor activity enables modification of current in vitro beta cell differentiation protocols to one that promotes the expression of key FEV-regulated transcriptional circuits that promote beta cell differentiation from endocrine progenitors. Identification of transcriptional targets in hESC-derived FEV+ beta cells also illuminated the function of FEV in differentiated beta cells.

Suppressing the Formation of hESC-Derived Blocked Endocrine Progenitors

Tremendous effort has been devoted to determining the molecular cues that will make derivation of the beta lineage from hESCs more efficient. Disclosed herein is a hESC-derived FEV-expressing population that appeared to be mis-differentiated during in vitro beta cell differentiation. The top differentially-expressed gene of this blocked, FEV-expressing cell population was the transcription factor PHOX2A. Interestingly, PHOX2A was also previously reported to mark a non-endocrine population that emerged in in vitro beta cell differentiation but was not described as mis-directed in differentiation potential (Veres et al., 2019). PHOX2A is a pro-neural homeodomain transcription factor and a key regulator of neural progenitor differentiation into noradrenergic neurons of the central nervous system (CNS) and the peripheral nervous system (PNS) (Lo et al., 1998; Morin et al., 1997). Noradrenergic neurons are characterized by synthesis and storage of catecholamines, including norepinephrine, which serve as neurotransmitters (Hayashida and Eisenach, 2018). In this differentiation process, BMP2 and cyclic AMP (cAMP) signaling synergistically induce noradrenergic neuron differentiation through Phox2a transcription and Phox2a activation (Benjanirut et al., 2006; Chen et al., 2005; Paris et al., 2006). Given the expression of PHOX2A specifically in cells occupying the mis-differentiated trajectory in the pseudotemporal ordering analysis disclosed herein, it is expected that the PDX1+/NKX6.1+ pancreatic progenitors in the in vitro beta cell differentiation process have mis-differentiated towards this PHOX2A+ noradrenergic neural lineage.

Inhibition of BMP2 and cAMP signaling represents possible avenues through which in vitro beta cell differentiation can avoid entering this mis-directed differentiation path that resembled the noradrenergic neural lineage.

Reported Enterochromaffin Cells in In Vitro Beta Cell Differentiation

Currently, in vitro beta cell differentiation does not result in 100% purity of beta cells, and there are other cell types that arise during the directed differentiation of hESCs to the beta lineage. Recently, a population deemed enterochromaffin cells (ECs) has been described as arising in in vitro beta cell differentiation (Veres et al., 2019). ECs reside along the epithelial lining of the intestine and are the most abundant cell type among the enteroendocrine cells found in the intestine (Lund et al., 2018). The main functions of ECs are to regulate intestinal motility required for digestion and modulate the activity of the enteric nervous system through the production and secretion of the neurotransmitter serotonin. Although ECs make up less than 1% of the total intestinal epithelium, they produce more than 90% of the body's serotonin (Gershon, 2013; Mawe and Hoffman, 2013). Unlike neurons, ECs utilize tryptophan hydroxylase 1 (TPH1) and not TPH2 to synthesize serotonin, and instead of employing small neurosecretory vesicles, ECs store serotonin in large dense core vesicles (LDCVs) with the help of CHGA and CHGAB (Cote et al., 2003; Machado et al., 2010; Walther and Bader, 2003). Thus, ECs resemble lineages of both the nervous system and hormone-secreting pancreatic islets.

ECs are defined by the expression of markers that also are expressed by both serotonergic neurons and pancreatic endocrine cells. These markers include Fev, Lmx1a, Lmx1b, and Tph1 (Ding et al., 2003; Kiyasova and Gaspar, 2011; Liu et al., 2010; Maurer et al., 2004; Ohta et al., 2011; Wyler et al., 2016; Zhang et al., 2017). Proper differentiation of ECs in the intestine during development also requires transcription factors critical for pancreatic endocrine cell differentiation, including Ngn3 (Jenny et al., 2002; López-Díaz et al., 2007; Schonhoff et al., 2004), Nkx2.2 (Gross et al., 2016), Isl1 (Terry et al., 2014), NeuroD1 (Naya et al., 1997), Pax4 (Beucher et al., 2012a). Interestingly, Fev is also expressed by ECs but is not required for EC differentiation in mice (Wang et al., 2010b). Given the striking similarity in gene expression profiles of ECs and EC differentiation to those of pancreatic endocrine cells, it is not surprising to observe ECs generated in in vitro beta cell differentiation. The mis-differentiation of hESC-derived endocrine progenitors towards similar lineages, such as that of the EC, is not surprising, given that in vitro beta cell differentiation is not 100% efficient. It is likely that the ECs observed during in vitro beta cell differentiation represent another mis-differentiation process, similar to that observed with mis-differentiated PHOX2A+ cells. Given that these hESC-derived ECs also express FEV, these ECs could be mistaken for the FEV+ endocrine progenitors identified in human pancreatic development. However, this is not the case, given that a separate population of FEV+ hESC-derived endocrine progenitors is found that give rise to the beta lineage in the in vitro differentiation process disclosed herein. It is likely that these FEV+ ECs are derived from the same hESC-derived FEV+ endocrine progenitors that gave rise to beta cells.

### REFERENCES (EXAMPLES 8-18)

1. Tokarz, V. L., MacDonald, P. E. & Klip, A. The Journal of Cell Biology 217, 2273-2289 (2018).
2. Yoon, J.-W. & Jun, H.-S. Am J Ther 12, 580-591 (2005).
3. Prentki, M. & Nolan, C. J. J. Clin. Invest. 116, 1802-1812 (2006).
4. Gilchrist, J. A., Best, C. H. & Banting, F. G. Can Med Assoc J 13, 565-572 (1923).
5. Trikkalinou, A., Papazafiropoulou, A. K. & Melidonis, A. World J Diabetes 8, 120-129 (2017).
6. Zheng, Y., Ley, S. H. & Hu, F. B. Nat Rev Endocrinol 14, 88-98 (2018).
7. Huang, E. S., Brown, S. E. S., Ewigman, B. G., Foley, E. C. & Meltzer, D. O. Diabetes Care 30, 2478-2483 (2007).
8. Sneddon, J. B. et al. Cell Stem Cell 22, 810-823 (2018).
9. Giorgakis, E. et al. World J Transplant 8, 237-251 (2018).
10. Kelly, W. D., Lillehei, R. C., Merkel, F. K., Idezuki, Y. & Goetz, F. C. Surgery 61, 827-837 (1967).
11. Shapiro, A. M. et al. N. Engl. J. Med. 343, 230-238 (2000).
12. Shapiro, A. M. J., Pokrywczynska, M. & Ricordi, C. Nat Rev Endocrinol 13, 268-277 (2016).
13. Pagliuca, F. W. et al. Cell 159, 428-439 (2014).
14. Rezania, A. et al. Nat. Biotechnol. 32, 1121-1133 (2014).
15. Russ, H. A. et al. EMBO J. 34, 1759-1772 (2015).
16. Veres, A. et al. Nature 569, 368-373 (2019).
17. Da Silva Xavier, G. J Clin Med 7, 54 (2018).
18. Suissa, Y. et al. PLoS ONE 8, e70397 (2013).
19. Pan, F. C. & Wright, C. Dev. Dyn. 240, 530-565 (2011).
20. Gu, G., Brown, J. R. & Melton, D. A. Mechanisms of Development 120, 35-43 (2003).
21. Pictet, R. L., Clark, W. R., Williams, R. H. & Rutter, W. J. Dev. Biol. 29, 436-467 (1972).
22. Herrera, P. L. Development 127, 2317-2322 (2000).
23. Kesavan, G. et al. Cell 139, 791-801 (2009).
24. Villasenor, A., Chong, D. C., Henkemeyer, M. & Cleaver, O. Development 137, 4295-4305 (2010).
25. Zhou, Q. et al. Dev. Cell 13, 103-114 (2007).
26. Seymour, P. A. et al. Proc. Natl. Acad. Sci. U.S.A. 104, 1865-1870 (2007).
27. Solar, M. et al. Dev. Cell 17, 849-860 (2009).
28. Schaffer, A. E., Freude, K. K., Nelson, S. B. & Sander, M. Dev. Cell 18, 1022-1029 (2010).
29. Apelqvist, A. et al. Nature 400, 877-881 (1999).
30. Shih, H. P. et al. Development 139, 2488-2499 (2012).
31. Gu, G., Dubauskaite, J. & Melton, D. A. Development 129, 2447-2457 (2002).
32. Heller, R. S. et al. Dev. Biol. 286, 217-224 (2005).
33. Wang, S. et al. Dev. Biol. 339, 26-37 (2010).
34. Beucher, A. et al. Dev. Biol. 361, 277-285 (2012).
35. Haumaitre, C. et al. PNAS 102, 1490-1495 (2005).
36. Zhang, H. et al. Mechanisms of Development 126, 958-973 (2009).
37. Villasenor, A., Chong, D. C. & Cleaver, O. Dev. Dyn. 237, 3270-3279 (2008).
38. Herrera, P. L., Orci, L. & Vassalli, J. D. Mol. Cell. Endocrinol. 140, 45-50 (1998).
39. Desgraz, R. & Herrera, P. L. Development 136, 3567-3574 (2009).
40. Miyatsuka, T., Kosaka, Y., Kim, H. & German, M. S. Proc. Natl. Acad. Sci. U.S.A. 108, 185-190 (2011).
41. Johansson, K. A. et al. Dev. Cell 12, 457-465 (2007).
42. Gouzi, M., Kim, Y. H., Katsumoto, K., Johansson, K. & Grapin-Botton, A. Dev. Dyn. 240, 589-604 (2011).
43. Puri, S. & Hebrok, M. Dev. Biol. 306, 82-93 (2007).
44. Sharon, N. et al. Cell 176, 790-804.e13 (2019).
45. Huang, H. P. et al. Mol. Cell. Biol. 20, 3292-3307 (2000).
46. Naya, F. J. et al. Genes Dev. 11, 2323-2334 (1997).
47. Mellitzer, G. et al. EMBO J. 25, 1344-1352 (2006).
48. Gierl, M. S., Karoulias, N., Wende, H., Strehle, M. & Birchmeier, C. Genes Dev. 20, 2465-2478 (2006).
49. Soyer, J. et al. Development 137, 203-212 (2010).
50. Smith, S. B. et al. Nature 463, 775-780 (2010).
51. Du, A. et al. Diabetes 58, 2059-2069 (2009).
52. Collombat, P. et al. Genes Dev. 17, 2591-2603 (2003).
53. Sosa-Pineda, B., Chowdhury, K., Torres, M., Oliver, G. & Gruss, P. Nature 386, 399-402 (1997).
54. Collombat, P. et al. Cell 138, 449-462 (2009).
55. Collombat, P. et al. J. Clin. Invest. 117, 961-970 (2007).
56. St-Onge, L., Sosa-Pineda, B., Chowdhury, K., Mansouri, A. & Gruss, P. Nature 387, 406-409 (1997).
57. Sander, M. et al. Development 127, 5533-5540 (2000).
58. Schaffer, A. E. et al. PLoS Genet. 9, e1003274 (2013).
59. Prado, C. L., Pugh-Bernard, A. E., Elghazi, L., Sosa-Pineda, B. & Sussel, L. Proc. Natl. Acad. Sci. U.S.A. 101, 2924-2929 (2004).
60. Artner, I. et al. Proc. Natl. Acad. Sci. U.S.A. 104, 3853-3858 (2007).
61. Zhao, L. et al. Journal of Biological Chemistry 280, 11887-11894 (2005).
62. Hang, Y. & Stein, R. Trends Endocrinol. Metab. 22, 364-373 (2011).
63. Nishimura, W. et al. Dev. Biol. 293, 526-539 (2006).
64. Matsuoka, T.-A. et al. PNAS 101, 2930-2933 (2004).
65. Artner, I. et al. Diabetes 59, 2530-2539 (2010).
66. Nishimura, W. et al. Dev. Biol. 314, 443-456 (2008).
67. Wang, H., Brun, T., Kataoka, K., Sharma, A. J. & Wollheim, C. B. Diabetologia 50, 348-358 (2007).
68. O'Rahilly, R. & Müller, F. Cells Tissues Organs (Print) 192, 73-84 (2010).
69. Jennings, R. E., Berry, A. A., Strutt, J. P., Gerrard, D. T. & Hanley, N. A. Development 142, 3126-3137 (2015).
70. Jennings, R. E. et al. Diabetes 62, 3514-3522 (2013).
71. Sarkar, S. A. et al. Diabetologia 51, 285-297 (2008).
72. Salisbury, R. J. et al. Islets 6, e954436 (2014).
73. Piper, K. et al. Journal of Endocrinology 181, 11-23 (2004).
74. Lyttle, B. M. et al. Diabetologia 51, 1169-1180 (2008).
75. Piper Hanley, K. et al. J. Endocrinol. 207, 151-161 (2010).
76. Kim, A. et al. Islets 1, 129-136 (2009).
77. Hebrok, M., Kim, S. K. & Melton, D. A. Genes Dev. 12, 1705-1713 (1998).
78. Slack, J. M. Development 121, 1569-1580 (1995).
79. Lammert, E., Cleaver, 0. & Melton, D. Science 294, 564-567 (2001).
80. Yoshitomi, H. & Zaret, K. S. Development 131, 807-817 (2004).
81. Pierreux, C. E. et al. Dev. Biol. 347, 216-227 (2010).
82. Sand, F. W. et al. Dev. Biol. 352, 267-277 (2011).
83. Magenheim, J. et al. Development 138, 4743-4752 (2011).
84. Villasenor, A. & Cleaver, O. Semin. Cell Dev. Biol. 23, 685-692 (2012).
85. Borden, P., Houtz, J., Leach, S. D. & Kuruvilla, R. Cell Rep 4, 287-301 (2013).
86. GOLOSOW, N. & GROBSTEIN, C. Dev. Biol. 4, 242-255 (1962).
87. Landsman, L. et al. PLoS Biol. 9, e1001143 (2011).
88. Bhushan, A. et al. Development 128, 5109-5117 (2001).
89. Larsen, B. M., Hrycaj, S. M., Newman, M., Li, Y. & Wellik, D. M. Development 142, 3859-3868 (2015).
90. Ahnfelt-Rønne, J., Ravassard, P., Pardanaud-Glavieux, C., Scharfmann, R. & Serup, P. Diabetes 59, 1948-1956 (2010).
91. Hay, E. D. Dev. Dyn. 233, 706-720 (2005).
92. D'Amour, K. A. et al. Nat. Biotechnol. 23, 1534-1541 (2005).
93. Stainier, D. Y. R. Genes Dev. 16, 893-907 (2002).
94. D'Amour, K. A. et al. Nat. Biotechnol. 24, 1392-1401 (2006).
95. Lau, J., Kawahira, H. & Hebrok, M. Cell. Mol. Life Sci. 63, 642-652 (2006).
96. Nair, G. G. et al. Nature Cell Biology 21, 263-274 (2019).
97. Jacobson, E. F. & Tzanakakis, E. S. J Biol Eng 11, 21 (2017).
98. Lock, L. T. & Tzanakakis, E. S. Tissue Eng. 13, 1399-1412 (2007).
99. Blum, B. et al. Nat. Biotechnol. 30, 261-264 (2012).
100. Dorrell, C. et al. Nat Commun 7, 11756 (2016).
101. Johnston, N. R. et al. Cell Metab. 24, 389-401 (2016).
102. Basta, G. et al. Diabetes Care 34, 2406-2409 (2011).
103. Millman, J. R. et al. Nat Commun 7, 11463 (2016).
104. Meivar-Levy, I. et al. Stem Cell Res Ther 10, 53-10 (2019).
105. Hayden, M. R. et al. J Cardiometab Syndr 3, 234-243 (2008).
106. Lammert, E., Cleaver, O. & Melton, D. Mechanisms of Development 120, 59-64 (2003).
107. Reinert, R. B. et al. Development 141, 1480-1491 (2014).
108. Bruin, J. E. et al. Stem Cell Reports 5, 1081-1096 (2015).
109. Sneddon, J. B., Borowiak, M. & Melton, D. A. Nature 491, 765-768 (2012).
110. Penko, D. et al. Islets 3, 73-79 (2011).
111. Shih, H. P., Wang, A. & Sander, M. Annu. Rev. Cell Dev. Biol. 29, 81-105 (2013).
112. Qiu, W.-L. et al. Cell Metab. 25, 1194-1205.e4 (2017).
113. Zeng, C. et al. Cell Metab. 25, 1160-1175.e11 (2017).
114. Satija, R., Farrell, J. A., Gennert, D., Schier, A. F. & Regev, A. Nat. Biotechnol. 33, 495-502 (2015).
115. McDavid, A. et al. Bioinformatics 29, 461-467 (2012).
116. Finak, G. et al. Genome Biol. 16, 278 (2015).
117. Kanamori-Katayama, M. et al. PLoS ONE 6, e25391 (2011).
118. Winters, N. & Bader, D. JDB 1, 64-81 (2013).
119. Majesky, M. W., Dong, X. R., Regan, J. N., Hoglund, V. J. & Schneider, M. Circ. Res.
108, 365-377 (2011).
120. Chiellini, C. et al. Biochemical and Biophysical Research Communications 374, 64-68 (2008).
121. Grenningloh, G., Soehrman, S., Bondallaz, P., Ruchti, E. & Cadas, H. Journal of Neurobiology 58, 60-69 (2003).
122. Hecksher-Sørensen, J. et al. Development 131, 4665-4675 (2004).
123. Wilm, B., Ipenberg, A., Hastie, N. D., Burch, J. B. E. & Bader, D. M. Development 132, 5317-5328 (2005).
124. Speer, M. Y. et al. Circ. Res. 104, 733-741 (2009).
125. Wang, L.-L. et al. Int. J. Med. Sci. 11, 262-267
126. Kwapiszewska, G. et al. Circulation 118, 1183-1194 (2008).
127. Jayewickreme, C. D. & Shivdasani, R. A. Dev. Biol. 405, 21-32 (2015).
128. Shang, Y., Yoshida, T., Amendt, B. A., Martin, J. F. & Owens, G. K. The Journal of Cell Biology 181, 461-473 (2008).
129. Qiu, X. et al. Nature Methods 2017 14:10 14, 979-982 (2017).
130. Ohta, Y. et al. Diabetes 60, 3208-3216 (2011).
131. Han, S.-I., Yasuda, K. & Kataoka, K. Journal of Biological Chemistry 286, 10449-10456 (2011).
132. Serafimidis, I. et al. PLoS Biol. 15, e2000949 (2017).
133. Spencer, W. C. & Deneris, E. S. Front Cell Neurosci 11, 215 (2017).
134. Arnes, L., Hill, J. T., Gross, S., Magnuson, M. A. & Sussel, L. PLoS ONE 7, e52026 (2012).
135. Muzumdar, M. D., Tasic, B., Miyamichi, K., Li, L. & Luo, L. Genesis 45, 593-605 (2007).
136. Butler, A., Hoffman, P., Smibert, P., Papalexi, E. & Satija, R. Nat. Biotechnol. 36, 411-420 (2018).
137. Bonner-Weir, S., Aguayo-Mazzucato, C. & Weir, G. C. Ups. J. Med. Sci. 121, 155-158 (2016).
138. Stanescu, D. E., Yu, R., Won, K.-J. & Stoffers, D. A. Physiol. Genomics 49, 105-114 (2017).
139. Petri, A. et al. Journal of Molecular Endocrinology 37, 301-316 (2006).
140. Hishida, T., Naito, K., Osada, S., Nishizuka, M. & Imagawa, M. FEBS Lett. 581, 4272-4278 (2007).
141. Dekel, B. et al. Cancer Res 66, 6040-6049 (2006).
142. Hori, K. et al. CellReports 9, 2166-2179 (2014).
143. Muraro, M. J. et al. Cell Syst 3, 385-394.e3 (2016).
144. Weinreb, C., Wolock, S. & Klein, A. M. Bioinformatics 34, 1246-1248 (2018).
145. Tusi, B. K. et al. Nature Publishing Group 555, 54-60 (2018).
146. Ahlgren, U., Pfaff, S. L., Jessell, T. M., Edlund, T. & Edlund, H. Nature 124, 4243-4252 (1997).
147. Yin, Y., Wang, F. & Ornitz, D. M. Development 138, 3169-3177 (2011).
148. Murtaugh, L. C. Organogenesis 4, 81-86 (2008).
149. Hernandez-Torres, F., Rodriguez-Outeiriño, L., Franco, D. & Aranega, A. E. Front. Cell Dev. Biol. 5, 211 (2017).
150. Cao, H. et al. Development 140, 3348-3359 (2013).
151. Kapadia, C., Ghosh, M. C., Grass, L. & Diamandis, E. P. Biochemical and Biophysical Research Communications 323, 1084-1090 (2004).
152. Huang, C. et al. Cancer Science 108, 2130-2141 (2017).
153. Hasegawa, M. et al. Journal of Dermatological Science 70, 34-41 (2013).
154. Ernst, M. C. & Sinal, C. J. Trends in Endocrinology & Metabolism 21, 660-667 (2010).
155. Bin Zhou et al. J. Clin. Invest. 121, 1894-1904 (2011).
156. Que, J. et al. Proc. Natl. Acad. Sci. U.S.A. 105, 16626-16630 (2008).
157. Angelo, J. R. & Tremblay, K. D. Dev. Biol. 435, 15-25 (2018).
158. Jensen, J. N. et al. Gastroenterology 128, 728-741 (2005).
159. Rhim, A. D. & Stanger, B. Z. Progress in Molecular Biology and Translational Science 97, 41-78 (2010).
160. Miyatsuka, T., Li, Z. & German, M. S. Diabetes 58, 1863-1868 (2009).
161. Benitez, C. M. et al. PLoS Genet. 10, e1004645 (2014).
162. Butler, A. E. et al. The Journal of Clinical Endocrinology & Metabolism 101, 523-532 (2016).
163. Wyler, S. C. et al. J. Neurosci. 36, 1758-1774 (2016).
164. Schonhoff, S. E., Giel-Moloney, M. & Leiter, A. B. Dev. Biol. 270, 443-454 (2004).
165. Scott, M. M. et al. Proc. Natl. Acad. Sci. U.S.A. 102, 16472-16477 (2005).
166. Wang, F. et al. J Mol Diagn 14, 22-29 (2012).
167. Zheng, G. X. Y. et al. Nat Commun 8, 14049 (2017).
168. Dobin, A. et al. Bioinformatics 29, 15-21 (2012).
169. Maaten, L. V. D. & Hinton, G. Journal of Machine Learning Research 9, 2579-2605 (2008).
170. Kamburov, A., Wierling, C., Lehrach, H. & Herwig, R. Nucleic Acids Res. 37, D623-D628 (2008).
171. Frank, P. G. Arteriosclerosis, Thrombosis, and Vascular Biology 23, 1161-1168 (2003).
172. Stoffers, D. A., Zinkin, N. T., Stanojevic, V., Clarke, W. L. & Habener, J. F. Nat. Genet. 15, 106-110 (1997).
173. Pinney, S. E. et al. The Journal of Clinical Endocrinology & Metabolism 96, 1960-1965 (2011).
174. Wang, J. et al. N. Engl. J. Med. 355, 270-280 (2006).
175. Gehart, H. et al. Cell 176, 1158-1173.e16 (2019).
176. Rubio-Cabezas, O. et al. Diabetes 59, 2326-2331 (2010).
177. Flanagan, S. E. et al. Cell Metab. 19, 146-154 (2014).
178. Solomon, B. D. et al. Am. J. Med. Genet. A 149A, 2543-2546 (2009).
179. Bonnefond, A. et al. Diabetes Metab. 39, 276-280 (2013).
180. Gradwohl, G., Dierich, A., LeMeur, M. & Guillemot, F. Proc. Natl. Acad. Sci. U.S.A. 97, 1607-1611 (2000).
181. Sussel, L. et al. Development 125, 2213-2221 (1998).
182. Nair, G. & Hebrok, M. Curr. Opin. Genet. Dev. 32, 171-180 (2015).
183. Lawlor, N. et al. Genome Res. 27, 208-222 (2017).
184. Segerstolpe, A. et al. Cell Metab. 24, 593-607 (2016).
185. Baron, M. et al. Cell Syst 3, 346-360.e4 (2016).
186. Byrnes, L. E. et al. Nat Commun 9, 3922 (2018).
187. Reboll, M. R. et al. Circulation 136, 1809-1823 (2017).
188. Xu, E. E. et al. Diabetologia 58, 1013-1023 (2015).
189. Masjkur, J. et al. Diabetes 65, 314-330 (2016).
190. Wilcox, C. L., Terry, N. A., Walp, E. R., Lee, R. A. & May, C. L. PLoS ONE 8, e66214 (2013).
191. Heller, C. et al. Am. J. Physiol. Endocrinol. Metab. 301, E864-72 (2011).
192. Dong, H. et al. Cell Death Dis 8, e2821-e2821 (2017).
193. Kameswaran, V. et al. Cell Metab. 19, 135-145 (2014).
194. Weinstein, L. S., Xie, T., Qasem, A., Wang, J. & Chen, M. Int J Obes (Lond) 34, 6-17 (2010).
195. Hoffmann, A. & Spengler, D. Mol. Cell. Biol. 32, 2549-2560 (2012).
196. Kamiya, M. et al. Hum. Mol. Genet. 9, 453-460 (2000).
197. Sojoodi, M. et al. Diabetologia 59, 1474-1479 (2016).
198. Küry, P., Greiner-Petter, R., Cornely, C., Jürgens, T. & Müller, H. W. J. Neurosci. 22, 7586-7595 (2002).
199. Guillemot, F. et al. Cell 75, 463-476 (1993).
200. Ge, W. et al. PNAS 103, 1319-1324 (2006).
201. Doering, J. E. et al. J. Comp. Neurol. 511, 238-256 (2008).
202. Hald, J. et al. Diabetologia 55, 154-165 (2011).
203. Bedoyan, J. K. et al. Am. J. Med. Genet. A 152A, 1567-1574 (2010).
204. Okuno, Y. et al. Diabetes 62, 1426-1434 (2013).
205. Bertolino, P. et al. Proc. Natl. Acad. Sci. U.S.A. 105, 7246-7251 (2008).
206. Su, Y. et al. FEBS Lett. 586, 4215-4222 (2012).
207. Dorajoo, R. et al. Sci Rep 7, 5024 (2017).
208. Alvarez, E., Zhou, W., Witta, S. E. & Freed, C. R. Gene 357, 18-28 (2005).
209. Teratani-Ota, Y. et al. In Vitro Cell. Dev. Biol. Anim. 52, 961-973 (2016).
210. Smith, K. R. et al. Neuron 84, 399-415 (2014).
211. Klim, J. R. et al. Nat. Neurosci. 22, 167-179 (2019).
212. Solomou, A. et al. J. Biol. Chem. 290, 21432-21442 (2015).
213. Stuart, T. et al. Cell 177, 1888-1902.e21 (2019).
214. Haghverdi, L., Lun, A. T. L., Morgan, M. D. & Marioni, J. C. Nat. Biotechnol. 36, 421-427 (2018).
215. Hirsch, M. R., Tiveron, M. C., Guillemot, F., Brunet, J. F. & Goridis, C. Development 125, 599-608 (1998).
216. Tiveron, M. C., Hirsch, M. R. & Brunet, J. F. J. Neurosci. 16, 7649-7660 (1996).
217. Pattyn, A., Morin, X., Cremer, H., Goridis, C. & Brunet, J. F. Development 124, 4065-4075 (1997).
218. Borghini, S. et al. Biochem. J. 395, 355-361 (2006).
219. Lo, L., Morin, X., Brunet, J. F. & Anderson, D. J. Neuron 22, 693-705 (1999).
220. Cho, G.-S., Park, D.-S., Choi, S.-C. & Han, J.-K. Dev. Biol. 421, 183-193 (2017).
221. Dever, D. P. et al. Nature 539, 384-389 (2016).
222. Oliveira, G. et al. Sci Transl Med 7, 317ra198-317ra198 (2015).
223. Bonini, C. et al. Nature Medicine 9, 367-369 (2003).
224. Krentz, N. A. J. et al. Stem Cell Reports 11, 1551-1564 (2018).
225. Scavuzzo, M. A. et al. Nat Commun 9, 3356 (2018).
226. Yu, X.-X. et al. EMBO J. 38, e100164 (2019).
227. Miyatsuka, T. et al. Diabetes 63, 3388-3393 (2014).
228. Mastracci, T. L., Anderson, K. R., Papizan, J. B. & Sussel, L. PLoS Genet. 9, e1003278 (2013).
229. Churchill, A. J. et al. eLife 6, R106 (2017).
230. Napolitano, T. et al. Semin. Cell Dev. Biol. 44, 107-114 (2015).
231. Pataskar, A. et al. EMBO J. 35, 24-45 (2016).
232. Simon-Areces, J., Membrive, G., Garcia-Fernandez, C., Garcia-Segura, L. M. & Arevalo, M.-A. J. Comp. Neurol. 518, 1814-1824 (2010).
233. Qi, Y. et al. Development 128, 2723-2733 (2001).
234. Prakash, N. et al. Development 136, 2545-2555 (2009).
235. Blake, J. A. & Ziman, M. R. Development 141, 737-751 (2014).
236. Hendricks, T., Francis, N., Fyodorov, D. & Deneris, E. S. J. Neurosci. 19, 10348-10356 (1999).
237. Wong, D. M., Shen, Z., Owyang, K. E. & Martinez-Agosto, J. A. PLoS ONE 9, e115297 (2014).
238. Jenny, M. et al. EMBO J. 21, 6338-6347 (2002).
239. Lopez-Diaz, L. et al. Dev. Biol. 309, 298-305 (2007).
240. Gross, S. et al. Development 143, 2616-2628 (2016).
241. Terry, N. A., Walp, E. R., Lee, R. A., Kaestner, K. H. & May, C. L. Am. J. Physiol.
Gastrointest. Liver Physiol. 307, G979-91 (2014).
242. Beucher, A. et al. PLoS ONE 7, e36449 (2012).
243. Jeon, J., Correa-Medina, M., Ricordi, C., Edlund, H. & Diez, J. A. J. Histochem. Cytochem. 57, 811-824 (2009).
244. Reinert, R. B. et al. Diabetes 62, 4154-4164 (2013).
245. Haugas, M., Tikker, L., Achim, K., Salminen, M. & Partanen, J. Development 143, 4495-4508 (2016).
246. Morin, X. et al. Neuron 18, 411-423 (1997).
247. Lo, L., Tiveron, M. C. & Anderson, D. J. Development 125, 609-620 (1998).
248. Hayashida, K.-I. & Eisenach, J. C. Adv. Exp. Med. Biol. 1099, 93-100 (2018).
249. Benjanirut, C. et al. Journal of Biological Chemistry 281, 2969-2981 (2006).
250. Chen, S., Ji, M., Paris, M., Hullinger, R. L. & Andrisani, O. M. Journal of Biological Chemistry 280, 41025-41036 (2005).
251. Paris, M., Wang, W.-H., Shin, M.-H., Franklin, D. S. & Andrisani, O. M. Mol. Cell. Biol. 26, 8826-8839 (2006).
252. Shin, M.-H. et al. Mol. Cell. Biol. 29, 4878-4890 (2009).
253. Lund, M. L. et al. Mol Metab 11, 70-83 (2018).
254. Gershon, M. D. Curr Opin Endocrinol Diabetes Obes 20, 14-21 (2013).
255. Mawe, G. M. & Hoffman, J. M. Nat Rev Gastroenterol Hepatol 10, 473-486 (2013).
256. Walther, D. J. & Bader, M. Biochem. Pharmacol. 66, 1673-1680 (2003).
257. Cote, F. et al. PNAS 100, 13525-13530 (2003).
258. Machado, J. D. et al. Cell. Mol. Neurobiol. 30, 1181-1187 (2010).
259. Zhang, Y. et al. FASEB J 31, 5342-5355 (2017).
260. Ding, Y.-Q. et al. Nat. Neurosci. 6, 933-938 (2003).
261. Maurer, P. et al. Neurosci. Lett. 357, 215-218 (2004).
262. Liu, C. et al. Nat. Neurosci. 13, 1190-1198 (2010).
263. Kiyasova, V. & Gaspar, P. Eur. J. Neurosci. 34, 1553-1562 (2011).
264. Wang, Y.-C. et al. Endocr. Relat. Cancer 17, 283-291 (2010).
265. Brinkman, E. K., Chen, T., Amendola, M. & van Steensel, B. Nucleic Acids Res. 42, e168-e168 (2014).
266. Sharon, N. et al. Cell Rep 27, 2281-2291.e5 (2019).
267. Rezania, A. et al. Diabetes 60, 239-247 (2011).

All publications and patents mentioned in the application are herein incorporated by reference in their entireties or in relevant part, as would be apparent from context. Various modifications and variations of the disclosed subject matter will be apparent to those skilled in the art without departing from the scope and spirit of the disclosure. Although the disclosure has been described in connection with specific embodiments, it should be understood that the invention as claimed should not be unduly limited to such specific embodiments. Various modifications of the described modes for making or using the disclosed subject matter that are obvious to those skilled in the relevant field(s) are intended to be within the scope of the following claims.

### APPENDIX

TABLE 1

| | SEURAT | BIMODAL | | | |
|---|---|---|---|---|---|
| | FOLD | LIKELIHOOD | | | |
| | CHANGE | RATIO | WILCOXON | MAST | |
| | CLUSTER | ADJUSTED | ADJUSTED | ADJUSTED | |
| GENE NAME | X VS. ALL | P-VALUE | P-VALUE | P-VALUE | CLUSTER ID |
| SPP1 | 26.54 | 1.09E−36 | 1.78E−45 | 6.53E−36 | NGN3+/SPP1+ |
| SERPINA6 | 6.54 | 1.15E−25 | 2.99E−22 | 8.33E−21 | NGN3+/SPP1+ |
| TMEM171 | 6.23 | 7.57E−22 | 1.63E−41 | 9.20E−22 | NGN3+/SPP1+ |
| MT2 | 6.11 | 4.20E−15 | 5.47E−24 | 1.20E−13 | NGN3+/SPP1+ |
| ID1 | 5.54 | 1.57E−16 | 9.57E−21 | 7.54E−16 | NGN3+/SPP1+ |
| MT1 | 4.99 | 8.49E−16 | 3.55E−14 | 1.33E−15 | NGN3+/SPP1+ |
| H19 | 4.99 | 1.72E−25 | 1.78E−23 | 2.65E−26 | NGN3+/SPP1+ |
| CLU | 4.72 | 3.22E−20 | 2.57E−16 | 1.49E−19 | NGN3+/SPP1+ |
| PHLDA1 | 4.72 | 8.58E−21 | 7.12E−17 | 2.31E−19 | NGN3+/SPP1+ |
| ALDH1B1 | 4.59 | 6.73E−22 | 4.61E−35 | 3.02E−22 | NGN3+/SPP1+ |
| CLPS | 4.44 | 4.00E−08 | 7.60E−07 | 1.85E−06 | NGN3+/SPP1+ |
| RASGRP3 | 4.38 | 6.80E−13 | 6.61E−24 | 3.84E−10 | NGN3+/SPP1+ |
| TINAGL1 | 4.32 | 3.44E−14 | 1.23E−28 | 1.32E−12 | NGN3+/SPP1+ |
| DBI | 4.26 | 2.46E−25 | 7.53E−22 | 4.94E−24 | NGN3+/SPP1+ |
| WFDC2 | 4.20 | 4.57E−18 | 1.66E−23 | 1.46E−17 | NGN3+/SPP1+ |
| CXCL12 | 3.97 | 9.42E−15 | 5.63E−18 | 3.27E−12 | NGN3+/SPP1+ |
| SPARC | 3.97 | 3.48E−15 | 1.60E−19 | 1.03E−14 | NGN3+/SPP1+ |
| CPA1 | 3.89 | 4.65E−10 | 1 | 1 | NGN3+/SPP1+ |
| LDHA | 3.86 | 4.65E−15 | 6.42E−13 | 1.42E−11 | NGN3+/SPP1+ |
| CPA2 | 3.84 | 1.95E−12 | 1 | 1 | NGN3+/SPP1+ |
| CD24A | 3.78 | 1.21E−14 | 1.58E−14 | 1.23E−14 | NGN3+/SPP1+ |
| MGST1 | 3.78 | 2.81E−17 | 4.47E−29 | 8.60E−18 | NGN3+/SPP1+ |
| IFITM3 | 3.73 | 4.05E−16 | 1.32E−25 | 6.10E−15 | NGN3+/SPP1+ |
| CAT | 3.53 | 2.07E−12 | 2.37E−17 | 1.97E−11 | NGN3+/SPP1+ |
| GADD45A | 3.48 | 2.34E−11 | 2.66E−06 | 1.18E−07 | NGN3+/SPP1+ |
| SOX4 | 3.46 | 8.28E−15 | 2.35E−13 | 7.27E−15 | NGN3+/SPP1+ |
| RBP1 | 3.29 | 3.13E−11 | 2.79E−13 | 3.15E−10 | NGN3+/SPP1+ |
| HES1 | 3.07 | 6.17E−09 | 2.57E−17 | 8.66E−10 | NGN3+/SPP1+ |
| NEUROG3 | 3.07 | 1.32E−12 | 1.00E−10 | 4.65E−09 | NGN3+/SPP1+ |
| AMOTL2 | 3.05 | 3.10E−12 | 1.44E−20 | 4.12E−12 | NGN3+/SPP1+ |
| SERPINH1 | 3.01 | 5.59E−16 | 1.91E−13 | 5.27E−16 | NGN3+/SPP1+ |
| WSB1 | 2.93 | 4.18E−11 | 3.21E−11 | 1.54E−08 | NGN3+/SPP1+ |
| PTN | 2.89 | 1.05E−10 | 0.009197842 | 7.31E−13 | NGN3+/SPP1+ |
| ACOT1 | 2.89 | 3.67E−10 | 7.98E−09 | 3.49E−09 | NGN3+/SPP1+ |
| MIA | 2.89 | 1.34E−08 | 2.08E−05 | 4.02E−06 | NGN3+/SPP1+ |
| SPHK1 | 2.85 | 1.71E−09 | 8.93E−22 | 1.90E−09 | NGN3+/SPP1+ |
| GSTA3 | 2.77 | 4.93E−14 | 1.16E−37 | 8.08E−16 | NGN3+/SPP1+ |
| EIF1A | 2.73 | 5.61E−06 | 1 | 1 | NGN3+/SPP1+ |
| MPZL1 | 2.73 | 1.18E−11 | 8.31E−05 | 2.47E−10 | NGN3+/SPP1+ |
| GSTM1 | 2.69 | 0.000117653 | 0.099474698 | 0.059317455 | NGN3+/SPP1+ |
| SMCO4 | 2.68 | 1.75E−08 | 1.52E−11 | 3.19E−09 | NGN3+/SPP1+ |
| IFITM2 | 2.68 | 1.45E−12 | 6.51E−12 | 4.15E−12 | NGN3+/SPP1+ |
| ADK | 2.64 | 5.41E−06 | 4.61E−06 | 0.000209261 | NGN3+/SPP1+ |
| PEBP1 | 2.64 | 7.73E−22 | 2.86E−13 | 6.08E−22 | NGN3+/SPP1+ |
| GM10260 | 2.62 | 3.53E−10 | 2.77E−07 | 3.79E−12 | NGN3+/SPP1+ |
| GM8773 | 2.60 | 0.002145093 | 6.04E−06 | 0.155022311 | NGN3+/SPP1+ |
| ASS1 | 2.57 | 1.58E−05 | 1.33E−07 | 2.04E−06 | NGN3+/SPP1+ |
| IDH2 | 2.57 | 9.00E−14 | 1.41E−11 | 2.15E−14 | NGN3+/SPP1+ |
| TEAD2 | 2.57 | 1.00E−06 | 3.17E−11 | 5.23E−07 | NGN3+/SPP1+ |
| QSOX1 | 2.57 | 3.50E−07 | 1.16E−07 | 7.49E−06 | NGN3+/SPP1+ |
| ANXA2 | 2.51 | 2.09E−06 | 2.32E−12 | 1.44E−07 | NGN3+/SPP1+ |
| CBFA2T3 | 2.51 | 0.000158643 | 1.23E−06 | 0.000331285 | NGN3+/SPP1+ |
| RPL35 | 2.50 | 8.22E−09 | 1.40E−06 | 8.65E−11 | NGN3+/SPP1+ |
| COL18A1 | 2.46 | 2.09E−06 | 5.05E−15 | 1.85E−06 | NGN3+/SPP1+ |
| MEST | 2.45 | 0.002155698 | 0.001897746 | 0.000190086 | NGN3+/SPP1+ |
| ENO1 | 2.45 | 1.60E−09 | 9.36E−05 | 4.39E−09 | NGN3+/SPP1+ |
| SPINK4 | 2.45 | 0.003319279 | 2.11E−07 | 0.001074251 | NGN3+/SPP1+ |
| IGF2 | 2.39 | 9.01E−05 | 4.02E−06 | 9.88E−05 | NGN3+/SPP1+ |
| GALK1 | 2.36 | 8.62E−06 | 7.37E−18 | 2.06E−06 | NGN3+/SPP1+ |
| WFDC15B | 2.35 | 2.76E−05 | 7.02E−12 | 7.72E−06 | NGN3+/SPP1+ |
| BCL2 | 2.35 | 3.08E−08 | 7.33E−19 | 1.09E−07 | NGN3+/SPP1+ |
| ADCK5 | 2.35 | 0.005234971 | 0.002236532 | 0.148023088 | NGN3+/SPP1+ |
| COL9A3 | 2.33 | 0.000353329 | 1.62E−06 | 0.001544085 | NGN3+/SPP1+ |
| LITAF | 2.31 | 0.00116332 | 1.47E−06 | 0.001570313 | NGN3+/SPP1+ |
| KRT18 | 2.30 | 7.16E−07 | 7.27E−05 | 1.74E−06 | NGN3+/SPP1+ |
| DDB1 | 2.30 | 0.001138057 | 1 | 1 | NGN3+/SPP1+ |
| EPCAM | 2.28 | 2.18E−08 | 2.49E−05 | 2.46E−08 | NGN3+/SPP1+ |
| ACAA2 | 2.28 | 4.85E−05 | 3.23E−10 | 9.73E−07 | NGN3+/SPP1+ |
| GRIN3A | 2.25 | 4.49E−13 | 8.16E−42 | 7.41E−14 | NGN3+/SPP1+ |
| AKT1 | 2.25 | 0.000168468 | 1 | 1 | NGN3+/SPP1+ |
| PRDX4 | 2.25 | 6.13E−06 | 0.326316303 | 1.08E−05 | NGN3+/SPP1+ |
| APOE | 2.23 | 0.00680732 | 0.000167881 | 0.00542927 | NGN3+/SPP1+ |
| PPP1R7 | 2.22 | 7.72E−05 | 1 | 1 | NGN3+/SPP1+ |
| GPC3 | 2.20 | 0.014643133 | 6.60E−06 | 0.001450543 | NGN3+/SPP1+ |
| GADD45G | 2.19 | 0.01234775 | 0.001320417 | 0.024717193 | NGN3+/SPP1+ |
| FUCA1 | 2.19 | 0.004378699 | 0.185649299 | 0.019969619 | NGN3+/SPP1+ |
| LINGO1 | 2.16 | 0.015855507 | 0.01126812 | 0.455678237 | NGN3+/SPP1+ |
| CASP6 | 2.14 | 0.006806863 | 0.001305367 | 0.017983122 | NGN3+/SPP1+ |
| DLL1 | 2.14 | 0.000498752 | 1.71E−09 | 0.000492282 | NGN3+/SPP1+ |
| CD82 | 2.13 | 0.006741105 | 0.004020626 | 0.083878393 | NGN3+/SPP1+ |
| RPL36A | 2.13 | 4.72E−10 | 1.02E−08 | 4.09E−10 | NGN3+/SPP1+ |
| HEBP1 | 2.11 | 0.000514995 | 0.040451013 | 0.232891767 | NGN3+/SPP1+ |
| NPTX2 | 2.11 | 0.600691538 | 0.075334124 | 1 | NGN3+/SPP1+ |
| PRPF4B | 2.11 | 0.00016325 | 1 | 1 | NGN3+/SPP1+ |
| LAMB1 | 2.11 | 0.006103244 | 0.002218251 | 0.053334127 | NGN3+/SPP1+ |
| TUBA4A | 2.10 | 0.000732535 | 1 | 1 | NGN3+/SPP1+ |
| HMGB3 | 2.10 | 0.020006079 | 0.000938831 | 0.001565509 | NGN3+/SPP1+ |
| UBA52 | 2.08 | 0.00272053 | 0.000103442 | 0.000396055 | NGN3+/SPP1+ |
| DONSON | 2.08 | 0.000265499 | 0.031969214 | 0.101836399 | NGN3+/SPP1+ |
| VIM | 2.07 | 1.59E−05 | 5.39E−08 | 4.24E−05 | NGN3+/SPP1+ |
| RCN2 | 2.07 | 3.16E−05 | 1.33E−05 | 3.31E−05 | NGN3+/SPP1+ |
| CRP | 2.07 | 2.97E−06 | 2.42E−16 | 0.000127285 | NGN3+/SPP1+ |
| SNRPG | 2.06 | 1.20E−05 | 0.000221458 | 3.12E−05 | NGN3+/SPP1+ |
| BAMBI | 2.06 | 1 | 1 | 1 | NGN3+/SPP1+ |
| PRSS23 | 2.04 | 0.000525454 | 1.59E−10 | 0.000338145 | NGN3+/SPP1+ |
| APOC1 | 2.04 | 0.608170266 | 0.000723679 | 0.456146098 | NGN3+/SPP1+ |
| FOXA3 | 2.03 | 1.46E−05 | 1.64E−07 | 1.28E−05 | NGN3+/SPP1+ |
| CLDN12 | 2.03 | 0.009399822 | 5.42E−08 | 0.010828941 | NGN3+/SPP1+ |
| ATOX1 | 2.01 | 0.0008086155 | 0.00427101 | 0.000245496 | NGN3+/SPP1+ |
| NEUROG3 | 6.45 | 6.29E−82 | 1.91E−59 | 1.65E−64 | NGN3+ |
| BTBD17 | 6.32 | 2.20E−70 | 7.08E−56 | 6.87E−66 | NGN3+ |
| TGM7 | 5.98 | 3.66E−18 | 9.32E−27 | 7.34E−17 | NGN3+ |
| GADD45A | 5.90 | 4.18E−66 | 2.15E−53 | 6.65E−62 | NGN3+ |
| TMSB4X | 4.59 | 1.10E−62 | 1.66E−43 | 2.27E−60 | NGN3+ |
| MDK | 4.32 | 2.60E−74 | 1.86E−46 | 8.43E−61 | NGN3+ |
| NEUROD2 | 3.68 | 2.53E−33 | 7.24E−44 | 1.40E−32 | NGN3+ |
| CCK | 3.51 | 2.16E−19 | 5.81E−15 | 2.55E−18 | NGN3+ |
| SOX4 | 3.43 | 5.44E−32 | 5.19E−31 | 1.49E−30 | NGN3+ |
| MFNG | 3.39 | 2.35E−37 | 1.60E−37 | 7.26E−36 | NGN3+ |
| IGFBPL1 | 3.32 | 1.70E−34 | 3.27E−32 | 3.12E−33 | NGN3+ |
| HES6 | 3.29 | 1.19E−42 | 5.82E−34 | 3.86E−41 | NGN3+ |
| SMARCD2 | 3.27 | 3.03E−32 | 6.15E−30 | 3.84E−31 | NGN3+ |
| SNRK | 3.23 | 8.40E−18 | 1.27E−18 | 2.12E−15 | NGN3+ |
| CLDN6 | 3.20 | 8.49E−37 | 6.07E−31 | 2.43E−35 | NGN3+ |
| KIRREL2 | 3.18 | 2.18E−24 | 4.06E−31 | 5.50E−22 | NGN3+ |
| GRASP | 3.14 | 3.07E−23 | 4.03E−25 | 2.41E−21 | NGN3+ |
| IFITM2 | 2.93 | 2.36E−31 | 1.62E−28 | 5.49E−30 | NGN3+ |
| TECR | 2.87 | 2.52E−44 | 1.19E−28 | 1.14E−42 | NGN3+ |
| PPP1R14A | 2.87 | 3.65E−24 | 1.30E−34 | 3.58E−23 | NGN3+ |
| TTLL6 | 2.83 | 4.24E−08 | 1.28E−09 | 3.83E−10 | NGN3+ |
| DDIT4 | 2.81 | 1.78E−21 | 2.45E−29 | 7.47E−22 | NGN3+ |
| SELM | 2.81 | 6.73E−31 | 8.07E−26 | 2.96E−29 | NGN3+ |
| CDC14B | 2.77 | 2.05E−18 | 4.00E−19 | 1.60E−13 | NGN3+ |
| PLK3 | 2.75 | 4.72E−21 | 4.71E−30 | 6.35E−21 | NGN3+ |
| PPIB | 2.73 | 3.81E−41 | 9.77E−34 | 2.30E−39 | NGN3+ |
| AMOTL2 | 2.71 | 9.84E−18 | 9.10E−25 | 8.12E−18 | NGN3+ |
| GPX2 | 2.68 | 2.69E−17 | 1.19E−19 | 1.24E−17 | NGN3+ |
| CDK2AP1 | 2.64 | 2.37E−25 | 2.20E−25 | 4.84E−25 | NGN3+ |
| TEAD2 | 2.62 | 4.79E−19 | 1.56E−25 | 3.52E−20 | NGN3+ |
| HN1 | 2.58 | 2.52E−31 | 1.14E−26 | 2.36E−30 | NGN3+ |
| EPB42 | 2.53 | 1.08E−17 | 2.84E−27 | 3.58E−17 | NGN3+ |
| PAX4 | 2.53 | 1.76E−10 | 7.44E−12 | 1.47E−10 | NGN3+ |
| FOXA3 | 2.53 | 1.28E−15 | 1.56E−16 | 2.11E−14 | NGN3+ |
| TUBB3 | 2.51 | 9.85E−19 | 2.41E−16 | 6.41E−18 | NGN3+ |
| CER1 | 2.50 | 1.01E−12 | 2.48E−18 | 1.33E−12 | NGN3+ |
| MFAP4 | 2.43 | 9.52E−08 | 1.80E−09 | 7.47E−08 | NGN3+ |
| MTCH1 | 2.41 | 1.07E−30 | 5.90E−26 | 2.61E−29 | NGN3+ |
| CDK4 | 2.39 | 1.90E−32 | 2.94E−27 | 1.01E−30 | NGN3+ |
| COTL1 | 2.38 | 3.94E−21 | 4.22E−15 | 1.46E−20 | NGN3+ |
| SPARC | 2.36 | 1.55E−08 | 3.56E−10 | 1.21E−08 | NGN3+ |
| TMEM184A | 2.33 | 1.10E−07 | 9.81E−08 | 2.75E−06 | NGN3+ |
| 2010107G23RIK | 2.31 | 3.94E−13 | 1.31E−10 | 1.26E−13 | NGN3+ |
| LY6E | 2.28 | 1.20E−18 | 8.26E−18 | 2.10E−19 | NGN3+ |
| TPM4 | 2.28 | 8.49E−18 | 7.46E−20 | 1.59E−18 | NGN3+ |
| KRTAP17-1 | 2.27 | 7.54E−08 | 4.54E−12 | 9.55E−09 | NGN3+ |
| MARCKSL1 | 2.27 | 7.04E−29 | 1.06E−24 | 2.68E−25 | NGN3+ |
| GM42637 | 2.27 | 9.05E−12 | 3.44E−18 | 5.48E−12 | NGN3+ |
| OLFM1 | 2.27 | 7.65E−10 | 9.90E−11 | 7.52E−10 | NGN3+ |
| UPK3BL | 2.23 | 2.37E−11 | 4.75E−18 | 3.41E−12 | NGN3+ |
| KRT19 | 2.23 | 6.82E−06 | 9.60E−07 | 2.70E−07 | NGN3+ |
| CLPS | 2.22 | 1.18E−05 | 2.68E−07 | 3.06E−06 | NGN3+ |
| TMEM171 | 2.20 | 4.80E−08 | 3.57E−12 | 9.46E−08 | NGN3+ |
| RCOR2 | 2.20 | 1.97E−08 | 3.04E−08 | 1.95E−07 | NGN3+ |
| GFRA3 | 2.20 | 1.01E−08 | 2.95E−05 | 1.28E−07 | NGN3+ |
| SERPINH1 | 2.17 | 2.10E−15 | 7.85E−16 | 9.10E−15 | NGN3+ |
| SULT2B1 | 2.14 | 1.24E−16 | 6.27E−25 | 4.94E−17 | NGN3+ |
| LPAR6 | 2.13 | 3.03E−11 | 5.54E−14 | 6.75E−11 | NGN3+ |
| RRM1 | 2.13 | 5.05E−05 | 4.21E−06 | 0.000216461 | NGN3+ |
| TTC28 | 2.11 | 2.64E−11 | 3.03E−15 | 1.95E−12 | NGN3+ |
| CASP6 | 2.11 | 6.19E−14 | 1.35E−16 | 1.17E−13 | NGN3+ |
| SULF2 | 2.10 | 2.31E−10 | 1.94E−14 | 3.18E−10 | NGN3+ |
| ADAM10 | 2.08 | 3.83E−05 | 0.019849605 | 0.590924318 | NGN3+ |
| FYTTD1 | 2.07 | 3.42E−13 | 7.13E−15 | 8.61E−13 | NGN3+ |
| CBFA2T3 | 2.06 | 3.67E−08 | 4.57E−12 | 8.84E−09 | NGN3+ |
| TPST2 | 2.06 | 6.48E−10 | 6.12E−09 | 8.10E−09 | NGN3+ |
| GDPD1 | 2.03 | 2.25E−10 | 4.01E−13 | 2.54E−10 | NGN3+ |
| INSM1 | 2.01 | 5.22E−08 | 2.98E−09 | 5.22E−07 | NGN3+ |
| FEV | 4.53 | 4.34E−25 | 1.33E−22 | 8.77E−24 | FEV+/PAX4+ |
| RUNX1T1 | 3.20 | 1.97E−22 | 3.62E−21 | 1.23E−18 | FEV+/PAX4+ |
| CACNA2D1 | 3.12 | 3.05E−20 | 4.55E−19 | 1.54E−18 | FEV+/PAX4+ |
| 1110012L19RIK | 3.10 | 1.24E−17 | 1.28E−12 | 9.10E−16 | FEV+/PAX4+ |
| CLDN4 | 2.68 | 3.55E−17 | 1.66E−13 | 7.63E−14 | FEV+/PAX4+ |
| GSPT1 | 2.66 | 1.77E−12 | 7.47E−11 | 5.83E−12 | FEV+/PAX4+ |
| PAX4 | 2.51 | 2.46E−16 | 4.79E−20 | 4.04E−16 | FEV+/PAX4+ |
| KRT7 | 2.46 | 1.14E−20 | 7.08E−18 | 2.53E−18 | FEV+/PAX4+ |
| BC023829 | 2.39 | 2.32E−12 | 5.98E−13 | 1.14E−11 | FEV+/PAX4+ |
| TOX3 | 2.27 | 1.89E−15 | 1.77E−14 | 7.26E−14 | FEV+/PAX4+ |
| POU3F4 | 2.23 | 3.87E−05 | 5.78E−06 | 1.86E−05 | FEV+/PAX4+ |
| CHGB | 2.22 | 0.000955337 | 0.000784048 | 0.000366104 | FEV+/PAX4+ |
| KRT8 | 2.20 | 9.09E−19 | 3.17E−17 | 1.46E−16 | FEV+/PAX4+ |
| ELAVL4 | 2.16 | 0.000162372 | 1.31E−05 | 0.000849094 | FEV+/PAX4+ |
| JUN | 2.14 | 3.56E−09 | 7.77E−10 | 3.56E−08 | FEV+/PAX4+ |
| BTG2 | 2.07 | 0.000198515 | 0.037190083 | 0.005256782 | FEV+/PAX4+ |
| RASD1 | 2.06 | 0.01243511 | 0.012293278 | 0.027450346 | FEV+/PAX4+ |
| LHX1OS | 2.04 | 1.41E−05 | 2.83E−09 | 3.04E−05 | FEV+/PAX4+ |
| VWA5B2 | 2.04 | 3.00E−07 | 1.10E−05 | 8.83E−05 | FEV+/PAX4+ |
| LHX1 | 2.00 | 0.00231933 | 9.95E−06 | 0.005706814 | FEV+/PAX4+ |
| CHGB | 3.71 | 1.58E−40 | 1.11E−32 | 4.93E−36 | FEV+/CHGB+ |
| VIM | 3.68 | 2.24E−23 | 1.47E−23 | 2.45E−21 | FEV+/CHGB+ |
| KLF2 | 2.95 | 5.48E−15 | 8.42E−16 | 5.39E−14 | FEV+/CHGB+ |
| CRYBA2 | 2.87 | 6.88E−21 | 1.20E−19 | 1.52E−20 | FEV+/CHGB+ |
| HBB-BT | 2.83 | 0.876379076 | 1 | 1 | FEV+/CHGB+ |
| CLDN4 | 2.64 | 2.15E−22 | 3.29E−22 | 3.64E−24 | FEV+/CHGB+ |
| GCH1 | 2.57 | 7.92E−22 | 5.05E−22 | 6.19E−23 | FEV+/CHGB+ |
| USP18 | 2.36 | 3.56E−13 | 6.47E−17 | 5.20E−14 | FEV+/CHGB+ |
| FEV | 2.36 | 6.29E−30 | 1.31E−27 | 2.12E−29 | FEV+/CHGB+ |
| HMGN3 | 2.20 | 3.54E−26 | 8.10E−23 | 2.36E−25 | FEV+/CHGB+ |
| CHGA | 2.20 | 2.64E−14 | 3.32E−14 | 7.41E−15 | FEV+/CHGB+ |
| FOS | 2.16 | 4.24E−17 | 1.96E−17 | 2.43E−17 | FEV+/CHGB+ |
| RAP1B | 2.07 | 1.62E−20 | 3.08E−19 | 7.70E−20 | FEV+/CHGB+ |
| JUNB | 2.07 | 1.16E−07 | 2.97E−07 | 3.59E−07 | FEV+/CHGB+ |
| INS2 | 230.72 | 1.01E−88 | 2.70E−57 | 9.05E−68 | BETA |
| INS1 | 59.71 | 1.47E−54 | 9.45E−65 | 5.91E−48 | BETA |
| NNAT | 20.53 | 7.60E−82 | 5.16E−55 | 4.19E−66 | BETA |
| NPY | 9.13 | 4.98E−25 | 4.90E−37 | 2.19E−19 | BETA |
| PPP1R1A | 8.00 | 3.76E−65 | 2.97E−66 | 5.77E−56 | BETA |
| IAPP | 7.21 | 2.60E−47 | 5.86E−44 | 1.14E−38 | BETA |
| SDF2L1 | 5.06 | 1.76E−42 | 1.40E−34 | 1.74E−33 | BETA |
| CRELD2 | 3.86 | 5.37E−41 | 5.57E−23 | 7.14E−30 | BETA |
| GNG12 | 3.78 | 4.68E−39 | 2.55E−36 | 3.37E−37 | BETA |
| MANF | 3.66 | 2.87E−45 | 1.30E−32 | 9.48E−39 | BETA |
| ATP2A2 | 3.51 | 3.51E−43 | 4.35E−35 | 3.34E−42 | BETA |
| HADH | 2.93 | 1.80E−25 | 1.20E−25 | 2.71E−22 | BETA |
| SLC2A2 | 2.91 | 1.31E−32 | 1.03E−47 | 3.59E−26 | BETA |
| OCIAD2 | 2.89 | 2.94E−24 | 1.71E−29 | 1.48E−19 | BETA |
| G6PC2 | 2.71 | 1.24E−21 | 1.33E−30 | 5.75E−16 | BETA |
| PDIA6 | 2.68 | 1.37E−28 | 1.35E−23 | 2.44E−26 | BETA |
| DLK1 | 2.57 | 1.70E−26 | 2.41E−24 | 2.03E−25 | BETA |
| CALR | 2.55 | 7.67E−26 | 1.76E−20 | 3.53E−23 | BETA |
| SYTL4 | 2.53 | 9.16E−27 | 1.59E−38 | 2.50E−21 | BETA |
| DNAJB11 | 2.38 | 2.18E−23 | 1.45E−18 | 9.83E−25 | BETA |
| SCG2 | 2.36 | 2.40E−16 | 5.89E−23 | 3.21E−12 | BETA |
| PCSK2 | 2.35 | 3.77E−26 | 2.26E−23 | 9.95E−21 | BETA |
| PDX1 | 2.30 | 3.96E−24 | 1.37E−23 | 2.34E−22 | BETA |
| SURF4 | 2.27 | 3.03E−17 | 3.42E−14 | 2.34E−14 | BETA |
| ERO1LB | 2.22 | 1.08E−19 | 1.22E−26 | 4.61E−16 | BETA |
| OSTC | 2.20 | 9.86E−25 | 9.95E−22 | 1.44E−23 | BETA |
| HSPA5 | 2.19 | 1.95E−25 | 1.60E−23 | 7.72E−26 | BETA |
| SERP1 | 2.19 | 2.43E−19 | 6.69E−18 | 4.42E−19 | BETA |
| CDK2AP2 | 2.16 | 3.05E−17 | 1.08E−12 | 4.30E−16 | BETA |
| SEC61B | 2.13 | 4.18E−30 | 2.69E−25 | 2.95E−29 | BETA |
| MAFB | 2.08 | 1.23E−12 | 5.03E−15 | 5.78E−09 | BETA |
| PAPSS2 | 2.07 | 1.53E−15 | 4.36E−21 | 6.96E−11 | BETA |
| 1700086L19RIK | 2.07 | 7.90E−10 | 1.74E−09 | 4.17E−08 | BETA |
| DBPHT2 | 2.06 | 1.23E−08 | 7.93E−08 | 1.04E−05 | BETA |
| HSP90B1 | 2.04 | 5.21E−14 | 2.14E−12 | 4.68E−13 | BETA |
| TUBB4B | 2.01 | 6.50E−10 | 3.00E−09 | 1.50E−05 | BETA |
| TUBB2A | 2.01 | 2.10E−10 | 3.72E−12 | 2.47E−07 | BETA |
| FAM151A | 2.01 | 1.33E−11 | 9.18E−17 | 1.16E−08 | BETA |
| GCG | 111.43 | 8.65E−68 | 4.25E−55 | 5.03E−67 | ALPHA |
| GAST | 14.93 | 3.06E−33 | 4.83E−41 | 2.03E−32 | ALPHA |
| TMEM27 | 7.94 | 2.79E−72 | 7.46E−64 | 6.37E−70 | ALPHA |
| PEG10 | 6.11 | 1.92E−51 | 3.06E−56 | 1.59E−51 | ALPHA |
| PPY | 5.86 | 1.47E−10 | 7.03E−12 | 3.75E−10 | ALPHA |
| PYY | 5.82 | 1.21E−53 | 7.76E−43 | 3.57E−47 | ALPHA |
| TTR | 4.92 | 1.97E−46 | 4.05E−37 | 4.70E−44 | ALPHA |
| ZCCHC18 | 3.81 | 2.22E−35 | 5.46E−33 | 1.23E−33 | ALPHA |
| SLC38A5 | 3.29 | 2.96E−36 | 4.50E−32 | 6.56E−33 | ALPHA |
| IRX1 | 3.20 | 1.12E−34 | 1.50E−47 | 2.42E−34 | ALPHA |
| IRX2 | 3.14 | 2.83E−30 | 1.81E−40 | 2.46E−30 | ALPHA |
| TMSB15B2 | 2.93 | 4.66E−14 | 2.17E−17 | 6.47E−13 | ALPHA |
| GPX3 | 2.73 | 6.78E−25 | 1.52E−23 | 1.30E−24 | ALPHA |
| WNK3 | 2.57 | 6.25E−21 | 2.33E−28 | 3.52E−20 | ALPHA |
| PCSK1N | 2.53 | 4.37E−26 | 1.78E−24 | 4.29E−25 | ALPHA |
| PAM | 2.48 | 5.05E−23 | 9.54E−23 | 3.11E−23 | ALPHA |
| RNF130 | 2.43 | 6.04E−23 | 5.79E−17 | 2.42E−22 | ALPHA |
| PON3 | 2.39 | 6.38E−21 | 4.64E−28 | 2.15E−19 | ALPHA |
| RESP18 | 2.36 | 2.35E−12 | 5.12E−17 | 6.05E−12 | ALPHA |
| SMARCA1 | 2.36 | 3.13E−13 | 7.36E−17 | 2.87E−13 | ALPHA |
| CTXN2 | 2.30 | 1.51E−17 | 9.92E−27 | 8.62E−18 | ALPHA |
| MEIS2 | 2.30 | 1.24E−16 | 5.33E−16 | 1.71E−15 | ALPHA |
| SCT | 2.25 | 4.17E−10 | 8.32E−11 | 1.61E−09 | ALPHA |
| USH1C | 2.25 | 2.64E−15 | 3.68E−19 | 4.76E−14 | ALPHA |
| RBP4 | 2.23 | 2.36E−20 | 1.49E−18 | 2.27E−18 | ALPHA |
| CTSZ | 2.23 | 8.43E−15 | 4.41E−11 | 2.64E−14 | ALPHA |
| TMED3 | 2.19 | 8.02E−18 | 1.57E−19 | 5.52E−17 | ALPHA |
| SCG5 | 2.16 | 5.79E−12 | 1.09E−13 | 1.98E−11 | ALPHA |
| CPE | 2.16 | 3.11E−27 | 8.43E−24 | 8.65E−24 | ALPHA |
| ARX | 2.13 | 2.39E−11 | 3.80E−14 | 7.98E−12 | ALPHA |
| SLC16A10 | 2.11 | 3.41E−15 | 1.76E−21 | 5.17E−15 | ALPHA |
| PCSK2 | 2.10 | 1.39E−14 | 1.06E−14 | 7.09E−14 | ALPHA |
| GHRL | 32.45 | 1.68E−19 | 2.63E−36 | 6.82E−21 | EPSILON |
| CDKN1A | 9.65 | 3.01E−31 | 3.54E−22 | 2.14E−30 | EPSILON |
| MBOAT4 | 6.68 | 3.87E−40 | 6.50E−67 | 2.71E−39 | EPSILON |
| HHEX | 4.82 | 6.38E−07 | 1.70E−09 | 1.32E−06 | EPSILON |
| RBP4 | 4.47 | 1.89E−06 | 0.000434406 | 2.56E−07 | EPSILON |
| ISL1 | 4.38 | 4.18E−23 | 5.98E−20 | 1.02E−23 | EPSILON |
| MAGED2 | 3.94 | 2.30E−23 | 0.164097154 | 3.80E−12 | EPSILON |
| ARG1 | 3.73 | 6.94E−15 | 5.19E−23 | 6.66E−15 | EPSILON |
| PYY | 2.99 | 0.09508771 | 0.002081274 | 0.007516006 | EPSILON |
| ANPEP | 2.77 | 0.043169744 | 3.60E−06 | 0.009815685 | EPSILON |
| RGS17 | 2.77 | 1.09E−10 | 3.73E−11 | 3.49E−10 | EPSILON |
| FHL2 | 2.41 | 0.000104472 | 0.000523591 | 4.64E−05 | EPSILON |
| NEFM | 2.39 | 0.00200021 | 1.65E−09 | 0.00035343 | EPSILON |
| B630019K06RIK | 2.33 | 0.00017761 | 0.00661927 | 0.001346362 | EPSILON |
| CTSL | 2.31 | 0.000695583 | 0.123653648 | 0.030180958 | EPSILON |
| TSPAN12 | 2.27 | 0.001318175 | 1.11E−06 | 0.004086006 | EPSILON |
| PEG3 | 2.22 | 7.46E−07 | 0.000194082 | 2.84E−06 | EPSILON |
| SYNE1 | 2.22 | 2.95E−08 | 9.15E−14 | 8.18E−07 | EPSILON |
| FFAR4 | 2.20 | 5.53E−05 | 2.92E−06 | 2.09E−05 | EPSILON |
| CD200 | 2.13 | 0.006569163 | 3.25E−05 | 0.001500125 | EPSILON |
| GHR | 2.11 | 0.000557467 | 1.99E−05 | 0.000138432 | EPSILON |
| CD24A | 2.08 | 0.010616908 | 0.001186314 | 0.006341855 | EPSILON |
| GJD2 | 2.07 | 0.005998637 | 0.003444585 | 0.16872243 | EPSILON |
| NAP1L5 | 2.04 | 0.583506634 | 0.031631734 | 0.895645277 | EPSILON |
| DHRS7 | 2.04 | 0.002440467 | 0.088577295 | 0.024914348 | EPSILON |
| ATF3 | 2.00 | 1 | 0.070900166 | 1 | EPSILON |

TABLE 2

| | | | | | | | MEMBERS_{—} | | |
|---|---|---|---|---|---|---|---|---|---|
| | | - LOG | | | | MEMBERS_{—} | INPUT_{—} | | EFFEC- |
| | | (P- | | | | INPUT_{—} | OVERLAP_{—} | | TIVE_{—} |
| P-VALUE | Q-VALUE | VALUE) | PATHWAY | SOURCE | EXTERNAL_ID | OVERLAP | GENEIDS | SIZE | SIZE |
| 0.00015116 | 0.00708594 | 3.82055691 | GLUTATHIONE | HUMANCY C | PWY-4081 | GPX3; | 2878; 2882 | 9 | 9 |
| | | | REDOX | | | GPX7 | | | |
| | | | REACTIONS I | | | | | | |
| 0.00023034 | 0.00708594 | 3.6376214 | BMP2-WNT4-FOXO1 | WIKIPATH- | WP3876 | DCN; | 1634; 6422 | 11 | 11 |
| | | | PATHWAY IN | WAYS | | SFRP1 | | | |
| | | | HUMAN PRIMARY | | | | | | |
| | | | ENDOMETRIAL | | | | | | |
| | | | STROMAL CELL | | | | | | |
| | | | DIFFERENTIATION | | | | | | |
| 0.00027606 | 0.00708594 | 3.5590024 | REACTIVE | HUMANCY C | DETOX1- | GPX3; | 2878; 2882 | 12 | 12 |
| | | | OXYGEN SPECIES | | PWY-1 | GPX7 | | | |
| | | | DEGRADATION | | | | | | |
| 0.00032583 | 0.00708594 | 3.48701392 | SENESCENCE- | WIKIPATH- | WP3391 | IGFBP3; | 3490; 3486 | 13 | 13 |
| | | | ASSOCIATED | WAYS | | IGFBP7 | | | |
| | | | SECRETORY | | | | | | |
| | | | PHENOTYPE (SASP) | | | | | | |
| 0.00043748 | 0.00708594 | 3.35904343 | NEGATIVE | REACTOME | R-HSA- | SFRP2; | 6422; 6423 | 15 | 15 |
| | | | REGULATION OF | | | SFRP1 | | | |
| | | | TCF-DEPENDENT | | | | | | |
| | | | SIGNALING BY | | | | | | |
| | | | WNT LIGAND | | | | | | |
| | | | ANTAGONISTS | | | | | | |
| 0.00048313 | 0.00708594 | 3.31593424 | THYROID HORMONE | KEGG | PATH: | GPX3; | 2878; 2882; | 74 | 74 |
| | | | SYNTHESIS - | | HSA04918 | GPX7; | 481 | | |
| | | | HOMO SAPIENS | | | ATP1B1 | | | |
| | | | (HUMAN) | | | | | | |
| 0.00056517 | 0.00710505 | 3.24781779 | ACE INHIBIT | WIKIPATH- | WP554 | ACE2; | 59272; 186 | 17 | 17 |
| | | | OR PATHWAY | WAYS | | AGTR2 | | | |
| 0.00078652 | 0.00753487 | 3.1042886 | ACE INHIBIT OR | PHARMGKB | PA2023 | ACE2; | 59272; 186 | 20 | 20 |
| | | | PATHWAY, PHARMA- | | | AGTR2 | | | |
| | | | CODYNAMICS | | | | | | |
| 0.00078652 | 0.00753487 | 3.1042886 | AGENTS ACTING ON | PHARMGKB | PA165110622 | ACE2; | 59272; 186 | 20 | 20 |
| | | | THE RENINANGIO- | | | AGTR2 | | | |
| | | | TENSIN SYSTEM | | | | | | |
| | | | PATHWAY, PHARMA- | | | | | | |
| | | | CODYNAMICS | | | | | | |
| 0.00085624 | 0.00753487 | 3.06740679 | PROTEIN DIGENTION | KEGG | PATH: | ACE2; | 7373; 59272; | 90 | 90 |
| | | | AND ABSORPTION - | | HSA04974 | COL14A1; | 481 | | |
| | | | HOMO SAPIENS | | | ATP1B1 | | | |
| | | | (HUMAN) | | | | | | |
| 0.00104326 | 0.0083461 | 2.98160665 | RENINANGIO- | KEGG | PATH: | ACE2; | 59272; 186 | 23 | 23 |
| | | | TENSIN SYSTEM - | | HSA04614 | AGTR2 | | | |
| | | | HOMO SAPIENS | | | | | | |
| | | | (HUMAN) | | | | | | |
| 0.00149005 | 0.01092701 | 2.82680027 | POST-TRANSLA- | REACTOME | R-HSA- | IGFBP3; | 11098; 3486; | 110 | 109 |
| | | | TIONAL PROTEIN | | 8957275 | IGFBP7; | 3490 | | |
| | | | PHOSPHORYLATION | | | PRSS23 | | | |
| 0.00177759 | 0.01111899 | 2.75016744 | #NAME? | BIOCARTA | BARREST | AGTR2; | 186; 6387 | 30 | 30 |
| | | | | | INPATHWAY | CXCL12 | | | |
| 0.00189773 | 0.01111899 | 2.72176486 | ACTIVATION OF | BIOCARTA | GSPATHWAY | AGTR2; | 186; 6387 | 31 | 31 |
| | | | CAMP-DEPENDENT | | | CXCL12 | | | |
| | | | PROTEIN KINASE | | | | | | |
| | | | PKA | | | | | | |
| 0.00202164 | 0.01111899 | 2.6942972 | ROLE OF - | BIOCARTA | BARRMAPK | AGTR2; | 186; 6387 | 32 | 32 |
| | | | ARRESTINS IN THE | | PATHWAY | CXCL12 | | | |
| | | | ACTIVATION AND | | | | | | |
| | | | TARGETING OF | | | | | | |
| | | | MAP KINASES | | | | | | |
| 0.00202164 | 0.01111899 | 2.6942972 | WNT-NCORE | SIGNALINK | NONE | SFRP2; | 6422; 6423 | 32 | 32 |
| 0.00225618 | 0.01124011 | 2.64662575 | REGULATION OF | REACTOME | R-HSA- | SFRP1 | 11098; 3486; | 127 | 126 |
| | | | INSULIN-LIKE | | 381426 | IGFBP3; | 3490 | | |
| | | | GROWTH FACTOR | | | IGFBP7; | | | |
| | | | (IGF) TRANSPORT | | | PRSS23 | | | |
| | | | AND UPTAKE BY | | | | | | |
| | | | INSULIN-LIKE | | | | | | |
| | | | GROWTH FACTOR | | | | | | |
| | | | FACTOR BINDING | | | | | | |
| | | | PROTEINS (IGFBPS) | | | | | | |
| 0.00241577 | 0.01124011 | 2.61694474 | SMOOTH MUSCLE | REACTOME | R-HSA- | ACTA2; | 59; 10398 | 35 | 35 |
| | | | CONTRACTION | | 445355 | MYL9 | | | |
| 0.00255457 | 0.01124011 | 2.59268198 | DETOXIFICATION | REACTOME | R-HSA- | GPX3; | 2878; 2882 | 36 | 36 |
| | | | OF REACTIVE | | 3299685 | GPX7 | | | |
| | | | OXYGEN SPECIES | | | | | | |
| 0.00255457 | 0.01124011 | 2.59268198 | ROLES OF ARRESTIN | BIOCARTA | BARREST | AGTR2; | 6387; 186 | 36 | 36 |
| | | | DEPENDENT | | INSRCPATH- | CXCL12 | | | |
| | | | RECRUITMENT | | WAY | | | | |
| | | | OF SRC KINASES | | | | | | |
| | | | GPCR SIGNALING | | | | | | |
| 0.00284323 | 0.01191447 | 2.54618867 | STRIATED MUSCLE | WIKIPATH- | WP383 | ACTA2; | 10398; 59 | 38 | 38 |
| | | | CONTRACTION | WAYS | | MYL9 | | | |
| 0.00305341 | 0.01221363 | 2.51521514 | EXTRACELULAR | REACTOME | R-HSA- | DCN; LOX; | 1634; 4060; | 293 | 293 |
| | | | MATRIX | | 1474244 | COL14A1; | 7373; 4015 | | |
| | | | ORGANIZATION | | | LUM | | | |
| 0.00346431 | 0.01325476 | 2.46038305 | CHREBP | BIOCARTA | CHREBP- | AGTR2; | 186; 6387 | 42 | 42 |
| | | | REGULATION BY | | PATHWAY | CXCL12 | | | |
| | | | CARBOHYDRATES | | | | | | |
| | | | AND CAMP | | | | | | |
| 0.00396797 | 0.01454921 | 2.40143204 | ACTIVATION OF | BIOCARTA | CSKPATH- | AGTR2; | 186, 6387 | 45 | 45 |
| | | | CSK BY CAMP- | | WAY | CXCL12 | | | |
| | | | DEPENDENT | | | | | | |
| | | | PROTEIN KINASE | | | | | | |
| | | | INHIBITS SIGNALING | | | | | | |
| | | | THROUGH THE | | | | | | |
| | | | T CELL RECEPTOR | | | | | | |
| 0.00432154 | 0.0152118 | 2.36436192 | ION CHANNELS | BIOCARTA | RACCPATH- | AGTR2; | 186; 6387 | 47 | 47 |
| | | | AND THEIR | | WAY | CLCL12 | | | |
| | | | FUNCTIONAL ROLE | | | | | | |
| | | | IN VASCULAR | | | | | | |
| | | | ENDOTHELIUM | | | | | | |
| 0.0048783 | 0.01651116 | 2.31173164 | CHEMOKINE | REACTOME | R-HSA- | CXCL13; | 6387; 10563 | 50 | 50 |
| | | | RECEPTORS BIND | | | CXCL12 | | | |
| | | | CHEMOKINES | | | | | | |
| 0.00526691 | 0.01716623 | 2.27844398 | ONE CARBON | WIKIPATH- | WP3940 | GPX3; | 2882; 2878 | 52 | 52 |
| | | | METABOLISM AND | WAYS | | GPX7 | | | |
| | | | RELATED PATHWAYS | | | | | | |
| 0.00566934 | 0.01781793 | 2.24646741 | GLUTATHIONE | KEGG | PATH: | GPX3; | 2878; 2882 | 54 | 54 |
| | | | METABOLISM - | | HSA00480 | GPX7 | | | |
| | | | HOMO SAPIENS | | | | | | |
| | | | (HUMAN) | | | | | | |
| 0.00629864 | 0.01911312 | 2.200753 | ECM | REACTOME | R-HSA- | DCN; LUM | 4060; 1634 | 57 | 57 |
| | | | PROTEOGLYCANS | | 3000178 | | | | |
| 0.00692947 | 0.02032643 | 2.15930023 | TCF DEPENDENT | REACTOME | R-HSA- | RSPO3; | 6422; 84870; | 190 | 188 |
| | | | SIGNALING IN | | 201681 | SFRP2; | 6423 | | |
| | | | RESPONSE TO WNT | | | SFRP1 | | | |
| 0.00741496 | 0.02043259 | 2.12989121 | ARACHIDONIC | KEGG | PATH: | GPX3; | 2878; 2882 | 62 | 62 |
| | | | ACID METABOLISM - | | HSA00590 | GPX7 | | | |
| | | | HOMO SAPIENS | | | | | | |
| | | | (HUMAN) | | | | | | |
| 0.00755484 | 0.02043259 | 2.12177496 | PEPTIDE | REACTOME | R-HSA- | AGTR2; | 10563; 6387; | 194 | 194 |
| | | | LIGAND-BINDING | | 375276 | CXCL13; | 186 | | |
| | | | RECEPTORS | | | CXCL12 | | | |
| 0.00766222 | 0.02043259 | 2.11564526 | MUSCLE | REACTOME | R-HSA- | ACTA2; | 59; 10398; | 195 | 195 |
| | | | CONTRACTION | | 397014 | ATP1B1; | 481 | | |
| | | | | | | MYL9 | | | |
| 0.00899427 | 0.02327928 | 2.04603416 | G ALPHA (I) | REACTOME | R-HSA- | AGTR2; | 186; 10563; | 399 | 398 |
| | | | SIGNALLING EVENTS | | 418594 | CXCL13; | 5947; 6387 | | |
| | | | | | | CXCL12; | | | |
| | | | | | | RBP1 | | | |
| 0.00989439 | 0.02487732 | 2.00461101 | LINOLEATE | EHMN | LENOLEATE | GPX3; | 2878; 2882 | 74 | 72 |
| | | | METABOLISM | | METABOLISM | GPX7 | | | |

TABLE 3

| | | | | | | MEM- | MEMBERS_{—} | | |
|---|---|---|---|---|---|---|---|---|---|
| | | | | | | BERS_{—} | INPUT_{—} | | EFFEC- |
| | | - LOG | | | EXTER- | INPUT_{—} | OVERLAP_{—} | | TIVE_{—} |
| P-VALUE | Q-VALUE | (P-VALUE) | PATHWAY | SOURCE | NAL_ID | OVERLAP | GENEIDS | SIZE | SIZE |
| 9.37E−06 | 0.00231525 | 5.028099335 | DIRECT | PID | P53DOWN- | CASP6; | 1647; 839; | 147 | 146 |
| | | | P53 | | STREAM- | DDIT4; | 80781; 6696; | | |
| | | | EFFECTORS | | PATHWAY | COL18A1; | 54541; 1263; | | |
| | | | | | | GADD45A; | 1026 | | |
| | | | | | | PLK3; | | | |
| | | | | | | SPP1; | | | |
| | | | | | | CDKN1A | | | |
| 3.57E−05 | 0.00293572 | 4.447861411 | P53 | BIOCARTA | P53 | GADD45A; | 1647; 1019; | 13 | 13 |
| | | | SIGNALING | | PATHWAY | CDKN1A; | 1026 | | |
| | | | PATHWAY | | | CDK4 | | | |
| 3.57E−05 | 0.00293572 | 4.447861411 | TP53 | WIKI- | WP3804 | GADD45A; | 1647; 1026; | 13 | 13 |
| | | | REGULATES | PATHWAYS | | CDKN1A; | 1263 | 13 | 13 |
| | | | TRANSCRIPTION | | | PLK3 | | | |
| | | | OF CELL CYCLE | | | | | | |
| | | | GENES | | | | | | |
| 7.37E−05 | 0.00455357 | 4.132285259 | RETINOID | REACTOME | R-HSA- | RBP1; | 5947; 348; | 44 | 44 |
| | | | METABOLISM AND | | 975634 | CLPS; | 1208; 2719 | | |
| | | | TRANSPORT | | | APOE; | | | |
| | | | | | | GPC3 | | | |
| 0.0001426 | 0.00704462 | 3.845869661 | METABOLISM OF | REACTOME | R-HSA- | RPB1; | 5947; 348; | 52 | 52 |
| | | | FAT-SOLUBLE | | 6806667 | CLPS; | 1208; 2719 | | |
| | | | VITAMINS | | | APOE; | | | |
| | | | | | | GPC3 | | | |
| 0.0002572 | 0.01058812 | 3.589727046 | MIR-517 RELA- | WIKI- | WP3596 | CDKN1A; | 1026; 3397 | 5 | 5 |
| | | | TIONSHIP WITH | PATHWAYS | | ID1 | | | |
| | | | ARCN1 AND USP1 | | | | | | |
| 0.00045069 | 0.01590303 | 3.346119072 | COLLAGEN | REACTOME | R-HSA- | COL18A1; | 80781; 5479; | 70 | 70 |
| | | | BIOSYNTHESIS | | 1650814 | SERPINH1; | 871; 1299 | | |
| | | | AND MODIFYING | | | PPIB; | | | |
| | | | ENSYMES | | | COL9A3 | | | |
| 0.00053655 | 0.01656589 | 3.270392095 | ETHANOL | SMPDB | SMP00449 | ALDH1B1; | 847; 219 | 7 | 7 |
| | | | DEGRADATION | | | CAT | | | |
| 0.00074755 | 0.0202084 | 3.126357882 | EXTRACELLULAR | REACTOME | R-HSA- | COL18A1; | 5479; 871; | 295 | 294 |
| | | | MATRIX | | 1474244 | COL9A3; | 1299; 6678; | | |
| | | | ORGANIZATION | | | ADAM10; | 6696; 102; | | |
| | | | | | | SPARC; | 80781 | | |
| | | | | | | SPP1; | | | |
| | | | | | | SERPINH1; | | | |
| | | | | | | PPIB | | | |
| 0.00081815 | 0.0202084 | 3.087164937 | COLLAGEN | REACTOME | R-HSA- | COL18A1; | 80781; 102; | 36 | 36 |
| | | | DEGRADATION | | 1442490 | ADAM10; | 1299 | | |
| | | | | | | COL9A3 | | | |
| 0.00128691 | 0.02634768 | 2.890451927 | BINDING AND | REACTOME | R-HSA- | SPARC; | 348; 259; | 42 | 42 |
| | | | UPTAKE OF | | 2173782 | APOE; | 6678 | | |
| | | | LIGANDS BY | | | AMBP | | | |
| | | | SCAVENGER | | | | | | |
| | | | RECEPTORS | | | | | | |
| 0.00136719 | 0.02634768 | 2.864172626 | COLLAGEN | REACTOME | R-HSA- | COL18A1; | 871; 5479; | 94 | 94 |
| | | | FORMATION | | 1474290 | SERPINH1; | 1299; 80781 | | |
| | | | | | | PPIB; | | | |
| | | | | | | COL9A3 | | | |
| 0.00138672 | 0.02634768 | 2.858011158 | REACTIVE | HUMANCY | DETOX1- | GPX2; | 847; 2877 | 12 | 11 |
| | | | OXYGEN | | PWY-1 | CAT | | | |
| | | | SPECIES | | | | | | |
| | | | DEGRADATION | | | | | | |
| 0.00184777 | 0.02936699 | 2.733352899 | VISUAL PHOTO- | REACTOME | R-HSA- | RBP1; | 5947; 348; | 102 | 102 |
| | | | TRANSDUCTION | | 2187338 | CLPS; | 2719; 1208 | | |
| | | | | | | APOE; | | | |
| | | | | | | GPC3 | | | |
| 0.00191508 | 0.02936699 | 2.717812136 | CELL CYCLE | WIKI- | WP179 | GADD45A; | 1026; 1019; | 103 | 103 |
| | | | | PATHWAYS | | CDKN1A; | 1647; 8555 | | |
| | | | | | | CDK4; | | | |
| | | | | | | CDC14B | | | |
| 0.00195363 | 0.02936699 | 2.70915735 | RB TUMOR | BIOCARTA | RBPATH- | CDKN1A; | 1019; 1026 | 13 | 13 |
| | | | SUPPRESSOR/ | | WAY | CDK4 | | | |
| | | | CHECKPOINT | | | | | | |
| | | | SIGNALING IN | | | | | | |
| | | | RESPONSE TO | | | | | | |
| | | | DNA DAMAGE | | | | | | |
| 0.0022014 | 0.02936699 | 2.657301775 | DEGRADATION | REACTOME | R-HSA- | COL18A1; | 1299; 80781; | 107 | 107 |
| | | | OF THE EXTRA- | | 1474228 | ADAM10; | 6696; 102 | | |
| | | | CELLULAR MATRIX | | | SPP1; | | | |
| | | | | | | COL9A3 | | | |
| 0.002259 | 0.02936699 | 2.646083889 | TP53 REGULATES | REACTOME | R-HSA- | GADD45A; | 1263; 1647; | 51 | 51 |
| | | | TRANSCRIPTION | | 6791312 | PLK3; | 1026 | | |
| | | | OF CELL CYCLE | | | CDKN1A | | | |
| | | | GENES | | | | | | |
| 0.002259 | 0.02936699 | 2.646083889 | NOTCH-MEDIATED | PID | HES_HEY- | NEUROG3; | 3397; 55502; | 51 | 51 |
| | | | HES/HEY NETWORK | | PATHWAY | HES6; | 50674 | | |
| | | | | | | ID1 | | | |
| 0.00252267 | 0.03115497 | 2.598139565 | BILE ACID | EHMN | BILE ACID | DBI; | 641371; | 53 | 53 |
| | | | BIOSYNTHESIS | | BIOSYNTHE- | ALDH1B1; | 1622; 219 | | |
| | | | | | SIS | ACOT1 | | | |
| 0.00266134 | 0.03130241 | 2.574899912 | FATTY ACYLCOA | REACTOME | R-HSA- | DBI; TECR; | 641371; | 55 | 54 |
| | | | BIOSYNTHESIS | | 75105 | ACOT1 | 1622; 9524 | | |
| 0.00310529 | 0.03441547 | 2.507897216 | VALIDATED | PID | TAP63- | GADD45A; | 1647; 1026; | 57 | 57 |
| | | | TRANSCRIOTIONAL | | PATHWAY | CDKN1A; | 2877 | | |
| | | | TARGETS OF | | | GPX2 | | | |
| | | | TAP63 ISOFORMS | | | | | | |
| 0.00375024 | 0.03441547 | 2.425940769 | CELL CYCLE - | KEGG | PATH: | GADD45A; | 1026; 1647; | 124 | 124 |
| | | | HOMO SAPIENS | | HSA04110 | CDKN1A; | 8555; 1019 | | |
| | | | (HUMAN) | | | CDK4; | | | |
| | | | | | | CDC14B | | | |
| 0.00376924 | 0.03441547 | 2.423746639 | PROPANOATE | EHMN | PROPANO- | ALDH1B1; | 219; 1622 | 18 | 18 |
| | | | METABOLISM | | ATE | DBI | | | |
| | | | | | METABO- | | | | |
| | | | | | LISM | | | | |
| 0.00376924 | 0.03441547 | 2.423746639 | SCF(SKP2)- | REACTOME | R-HSA- | CDKN1A; | 1019; 1026 | 18 | 18 |
| | | | MEDIATED | | 187577 | CDK4 | | | |
| | | | DEGRADATION | | | | | | |
| | | | OF P27/P21 | | | | | | |
| 0.00376924 | 0.03441547 | 2.423746639 | REGULATION OF | BIOCARTA | PLK3- | CASP6; | 1263; 839 | 18 | 18 |
| | | | CELL CYCLE PRO- | | PATHWAY | PLK3 | | | |
| | | | GRESSION BY PLK3 | | | | | | |
| 0.00419877 | 0.03441547 | 2.37687761 | TP53 | WIKI- | WP1742 | GADD45A; | 1647; 1026 | 19 | 19 |
| | | | NETWORK | PATHWAYS | | CDKN1A | | | |
| 0.00419877 | 0.03441547 | 2.37687761 | ATM SIGNALING | WIKI- | WP2516 | GADD45A; | 1647; 1026 | 19 | 19 |
| | | | PATHWAY | PATHWAYS | | CDKN1A | | | |
| 0.00469983 | 0.03441547 | 2.327918116 | INTEGRATED | WIKI- | WP1984 | RASGRP3; | 1263; 25780; | 66 | 66 |
| | | | BREAST CANCER | PATHWAYS | | PLK3; | 1647 | | |
| | | | PATHWAY | | | | | | |
| 0.00481196 | 0.03441547 | 2.31767816 | FOXOSIGNALING | KEGG | PATH: | GADD45A; | 1026; 1647; | 134 | 133 |
| | | | PATHWAY - | | HSA04068 | CDKN1A; | 847; 1263 | | |
| | | | HOMO SAPIENS | | | PLK3; CAT | | | |
| | | | (HUMAN) | | | | | | |
| 0.00481196 | 0.03441547 | 2.31767816 | PLATELET | REACTOME | R-HSA- | TMSB4X; | 7114; 5768; | 133 | 133 |
| | | | DEGRANULATION | | 114608 | CLU; | 6678; 1191 | | |
| | | | | | | SPARC; | | | |
| | | | | | | QSOX1 | | | |
| 0.00490227 | 0.03441547 | 2.309603092 | INTEGRIN CELL | REACTOME | R-HSA- | COL18A1; | 80781; 6696; | 68 | 67 |
| | | | SURFACE | | 216083 | SPP1; | 1299 | | |
| | | | INTERACTIONS | | | COL9A3 | | | |
| 0.00510991 | 0.03441547 | 2.29158702 | G1 TO S CELL | WIKI- | WP45 | GADD45A; | 1019; 1026; | 68 | 68 |
| | | | CYCLE CONTROL | PATHWAYS | | CDKN1A; | 1647 | | |
| | | | | | | CDK4 | | | |
| 0.00510991 | 0.03441547 | 2.29158702 | DNA DAMAGE | WIKI- | WP707 | GADD45A; | 1019; 1647; | 68 | 68 |
| | | | RESPONSE | PATHWAYS | | CDKN1A; | 1026 | | |
| | | | | | | CDK4 | | | |
| 0.00512243 | 0.03441547 | 2.29052415 | NOTCH2 | REACTOME | R-HSA- | ADAM10; | 4192; 102 | 21 | 21 |
| | | | ACTIVATION AND | | 2979096 | MDK | | | |
| | | | TRANSMISSION OF | | | | | | |
| | | | SIGNAL TO THE | | | | | | |
| | | | NUCLEUS | | | | | | |
| 0.00532279 | 0.03441547 | 2.273861029 | P53 SIGNALING | KEGG | PATH: | GADD45A; | 1026; 1019; | 69 | 69 |
| | | | PATHWAY - HOMO | | HSA04115 | CDKN1A; | 1647 | | |
| | | | SAPIENS (HUMAN) | | | CDK4 | | | |
| 0.00548126 | 0.03441547 | 2.26111991 | RESPONSE TO | REACTOME | R-HSA- | TMSB4X; | 7114; 5768; | 138 | 138 |
| | | | ELEVATED PLATELET | | 76005 | CLU; | 6678; 1191 | | |
| | | | CYTOSOLIC CA2+ | | | SPARC; | | | |
| | | | | | | QSOX1 | | | |
| 0.00554094 | 0.03441547 | 2.25641663 | BETA1 INTEGRIN | PID | INTEGRIN1_{—} | COL18A1; | 6696; 80781; | 70 | 70 |
| | | | CELL SURFACE | | PATHWAY | MDK; | 4192 | | |
| | | | INTERACTIONS | | | SPP1 | | | |
| 0.0056161 | 0.03441547 | 2.250565538 | CELL CYCLE: | BIOCARTA | G2PATH- | GADD45A; | 1647; 1026 | 22 | 22 |
| | | | G2/M CHECKPOINT | | WAY | CDKN1A | | | |
| 0.0056161 | 0.03441547 | 2.250565538 | TRYPTOPHAN | EHMN | TRYPTO- | ALDH1B1; | 4257; 219 | 22 | 22 |
| | | | METABOLISM | | PHAN META- | MGST1 | | | |
| | | | | | BOLISM | | | | |
| 0.00593092 | 0.03441547 | 2.226877976 | SIGNAL | REACTOME | R-HSA- | RASGRP3; | 84894; 9350; | 2538 | 2524 |
| | | | TRANSDUCTION | | 162582 | SPHK1; | 4192; 25780; | | |
| | | | | | | AMOTL2; | 1026; 6696; | | |
| | | | | | | MFNG; | 25805; 885; | | |
| | | | | | | CDKN1A; | 102; 8877; | | |
| | | | | | | ADAM10; | 5947; 8555; | | |
| | | | | | | CCK; | 1208; 1019; | | |
| | | | | | | CER1; | 1299; 51421; | | |
| | | | | | | SOX4; | 4242; 92474; | | |
| | | | | | | MDK; | 2676; 6659; | | |
| | | | | | | APOE; | 348; 2719 | | |
| | | | | | | LINGO1; | | | |
| | | | | | | CDK4; | | | |
| | | | | | | CLPS; | | | |
| | | | | | | PPP1R14A; | | | |
| | | | | | | COL9A3; | | | |
| | | | | | | RBP1; | | | |
| | | | | | | BAMBI; | | | |
| | | | | | | GPC3; | | | |
| | | | | | | CDC14B; | | | |
| | | | | | | SPP1; | | | |
| | | | | | | GFRA3 | | | |
| 0.00613069 | 0.03441547 | 2.212490542 | CYCLINS AND CELL | BIOCARTA | CELL- | CDKN1A; | 1019; 1026 | 23 | 23 |
| | | | CYCLE REGULATION | | CYCLE- | CDK4 | | | |
| | | | | | PATHWAY | | | | |
| 0.00613069 | 0.03441547 | 2.212490542 | HYPOXIA AND P53 | BIOCARTA | P53- | GADD45A; | 1647; 1026 | 23 | 23 |
| | | | IN THE CARDIO- | | HYPOXIA- | CDKN1A | | | |
| | | | VASCULAR SYSTEM | | PATHWAY | | | | |
| 0.00613069 | 0.03441547 | 2.212490542 | BIOSYNTHESIS OF | KEGG | PATH: | TECR; | 9524; 641371 | 23 | 23 |
| | | | UNSATURATED FATTY | | HSA01040 | ACOT1 | | | |
| | | | ACIDS - HOMO | | | | | | |
| | | | SAPIENS (HUMAN) | | | | | | |
| 0.006666 | 0.0349761 | 2.176134999 | BUTANOATE | EHMN | BUTANOATE | ALDH1B1; | 219; 1622 | 24 | 24 |
| | | | METABOLISM | | METABO- | DBI | | | |
| | | | | | LISM | | | | |
| 0.00671203 | 0.03497611 | 2.173146207 | VALIDATED | PID | MYC_RE- | GADD45A; | 1647; 1026; | 75 | 75 |
| | | | TARGETS OF C-MYC | | PRESS- | CDKN1A; | 1191 | | |
| | | | TRANSCRIPTIONAL | | PATHWAY | CLU | | | |
| | | | REPRESSION | | | | | | |
| 0.00722179 | 0.03497611 | 2.141355316 | DISULFIRAM | SMPDB | SMP00429 | ALDH1B1; | 219; 847 | 25 | 25 |
| | | | ACTION PATHWAY | | | CAT | | | |
| 0.00722179 | 0.03497611 | 2.141355316 | STATIN PATHWAY, | PHARMGKB | PA2031 | APOE; | 341; 348 | 25 | 25 |
| | | | PHARMACO- | | | APOC1 | | | |
| | | | DYNAMICS | | | | | | |
| 0.00722179 | 0.03497611 | 2.141355316 | CELL CYCLE: | BIOCARTA | G1PATH- | CDKN1A; | 1026; 1019 | 25 | 25 |
| | | | G1/S CHECKPOINT | | WAY | CDK4 | | | |
| 0.00722179 | 0.03497611 | 2.141355316 | PROPANOATE | INOH | NONE | ALDH1B1; | 3939; 219 | 25 | 25 |
| | | | METABOLISM | | | LDHA | | | |
| 0.00722179 | 0.03497611 | 2.141355316 | FATTY ACID | KEGG | PATH: | TECR; | 9524; 641371 | 25 | 25 |
| | | | ELONGATION - HOMO | | HSA00062 | ACOT1 | | | |
| | | | SAPIENS (HUMAN) | | | | | | |
| 0.00779785 | 0.03566794 | 2.108025171 | PPAR ALPHA | WIKI- | WP2878 | DBI; CDK4 | 1019; 1622 | 26 | 26 |
| | | | PATHWAY | PATHWAYS | | | | | |
| 0.00779785 | 0.03566794 | 2.108025171 | CYCLIN A: CDK2- | REACTOME | R-HSA- | CDKN1A; | 1026; 1019 | 26 | 26 |
| | | | ASSOCIATED EVENTS | | 69656 | CDK4 | | | |
| | | | AT S PHASE ENTRY | | | | | | |
| 0.00779785 | 0.03566794 | 2.108025171 | MATURITY ONSET | KEGG | PATH: | NEUROG3; | 50674; 3171 | 26 | 26 |
| | | | DIABETES OF THE | | HSA04950 | FOXA3 | | | |
| | | | YOUNG - HOMO | | | | | | |
| | | | SAPIENS (HUMAN) | | | | | | |
| 0.0085821 | 0.03854159 | 2.066404689 | TRIGLYCERIDE | REACTOME | R-HSA- | DBI; | 641371; | 83 | 82 |
| | | | BIOSYNTHESIS | | 75109 | TECR; | 1622; 9524 | | |
| | | | | | | ACOT1 | | | |
| 0.00900992 | 0.03904299 | 2.045279066 | INFLUENCE OF | BIOCARTA | RACCYCD | CDKN1A; | 1026; 1019 | 28 | 28 |
| | | | RAS AND RHO | | PATHWAY | CDK4 | | | |
| | | | PROTEINS ON G1 TO S | | | | | | |
| | | | TRANSITION | | | | | | |
| 0.00900992 | 0.03904299 | 2.045279066 | NOTCH-NCORE | SIGNALINK | NONE | ADAM10; | 4242; 102 | 28 | 28 |
| | | | | | | MFNG | | | |
| 0.00936298 | 0.03987338 | 2.028585863 | METABOLISM OF | REACTOME | R-HSA- | RBP1; | 2719; 5947; | 164 | 161 |
| | | | VITAMINS AND | | 196854 | CLPS; | 348; 1208 | | |
| | | | COFACTORS | | | GPC3; | | | |
| | | | | | | APOE | | | |
| 0.0096455 | 0.04038032 | 2.015675204 | CYCLIN E ASSO- | REACTOME | R-HSA- | CDKN1A; | 1026; 1019 | 29 | 29 |
| | | | CIATED EVENTS | | 69202 | CDK4 | | | |
| | | | DURINT G1/S | | | | | | |
| | | | TRANSITION | | | | | | |

TABLE 4

| | SEURAT | BIMODAL | | | |
|---|---|---|---|---|---|
| | FOLD | LIKELIHOOD | | | |
| | CHANGE | RATIO | WILCOXON | MAST | |
| | CLUSTER | ADJUSTED | ADJUSTED | ADJUSTED | |
| GENE NAME | X VS. ALL | P-VALUE | P-VALUE | P-VALUE | CLUSTER ID |
| SPP1 | 4.5742486 | 0 | 0 | 4.54E−118 | DUCTAL 1 |
| SPARC | 3.67597871 | 0 | 0 | 1.47E−131 | DUCTAL 1 |
| TMEM45A | 3.23772906 | 2.45E−267 | 1.84E−301 | 6.31E−68 | DUCTAL 1 |
| 1700011H14RIK | 2.66511761 | 9.27E−265 | 4.18E−250 | 4.48E−53 | DUCTAL 1 |
| ANXA2 | 2.61031438 | 2.48E−233 | 8.87E−231 | 6.17E−27 | DUCTAL 1 |
| S100A11 | 2.57697996 | 0 | 5.41E−269 | 1.18E−92 | DUCTAL 1 |
| MALAT1 | 2.46702601 | 0 | 4.60E−298 | 1.35E−191 | DUCTAL 1 |
| PDZK1IP1 | 2.36499088 | 2.36E−209 | 5.59E−191 | 3.86E−46 | DUCTAL 1 |
| CDKN1A | 2.34816666 | 1.12E−183 | 4.41E−20 | 2.62E−54 | DUCTAL 1 |
| ENPP2 | 2.3481392 | 1.24E−198 | 2.06E−125 | 5.30E−85 | DUCTAL 1 |
| MEG3 | 2.27991956 | 6.34E−234 | 1.00E−204 | 7.27E−132 | DUCTAL 1 |
| FXYD2 | 2.27252826 | 1.45E−144 | 5.94E−165 | 2.42E−15 | DUCTAL 1 |
| CLU | 2.23691983 | 5.63E−218 | 2.91E−172 | 1.35E−82 | DUCTAL 1 |
| GAS6 | 2.23045634 | 2.39E−172 | 9.01E−106 | 2.06E−61 | DUCTAL 1 |
| RBP1 | 2.22717951 | 1.74E−235 | 5.69E−182 | 2.96E−56 | DUCTAL 1 |
| S100A10 | 2.2043717 | 5.29E−165 | 1.43E−157 | 8.76E−26 | DUCTAL 1 |
| KRT7 | 2.19983985 | 4.34E−194 | 5.25E−159 | 4.94E−37 | DUCTAL 1 |
| CYM | 2.17903903 | 8.32E−39 | 1 | 1.77E−05 | DUCTAL 1 |
| CYR61 | 2.17363728 | 2.19E−129 | 1.87E−118 | 2.76E−16 | DUCTAL 1 |
| TINAGL1 | 2.17096222 | 6.78E−156 | 1.63E−159 | 1.57E−25 | DUCTAL 1 |
| GSTA3 | 2.15182377 | 1.16E−188 | 4.66E−152 | 2.09E−38 | DUCTAL 1 |
| ATP1B1 | 2.1249283 | 2.83E−222 | 6.70E−189 | 1.73E−59 | DUCTAL 1 |
| LURAP1L | 2.10760262 | 1.30E−123 | 4.00E−92 | 5.74E−15 | DUCTAL 1 |
| CYSTM1 | 2.07965495 | 1.61E−250 | 1.82E−189 | 2.39E−81 | DUCTAL 1 |
| CXCL12 | 2.07309274 | 4.83E−134 | 4.06E−145 | 1.45E−25 | DUCTAL 1 |
| KRT18 | 2.01687548 | 1.28E−220 | 7.90E−198 | 6.13E−53 | DUCTAL 1 |
| TM4SF4 | 2.00919748 | 6.11E−146 | 2.41E−88 | 4.28E−38 | DUCTAL 1 |
| CYR61 | 3.64235201 | 3.53E−197 | 1.49E−165 | 5.41E−105 | DUCTAL 2 |
| ATF3 | 3.11981601 | 7.74E−132 | 6.97E−153 | 2.45E−77 | DUCTAL 2 |
| FOS | 2.91107534 | 1.50E−129 | 4.58E−115 | 7.74E−83 | DUCTAL 2 |
| NR4A1 | 2.8138114 | 2.25E−153 | 5.08E−171 | 3.49E−92 | DUCTAL 2 |
| JUNB | 2.40679226 | 2.86E−108 | 2.85E−96 | 1.35E−56 | DUCTAL 2 |
| 8430408G22RIK | 2.37749198 | 4.85E−34 | 1.14E−38 | 7.14E−10 | DUCTAL 2 |
| JUN | 2.25145709 | 5.82E−119 | 9.90E−105 | 2.70E−55 | DUCTAL 2 |
| BTG2 | 2.24346408 | 2.39E−79 | 2.90E−58 | 6.53E−43 | DUCTAL 2 |
| HES1 | 2.13479968 | 6.82E−69 | 3.16E−51 | 3.06E−26 | DUCTAL 2 |
| PPP1R15A | 2.13475793 | 3.19E−97 | 1.24E−86 | 1.67E−53 | DUCTAL 2 |
| EGR1 | 2.12490869 | 2.29E−94 | 6.80E−92 | 6.98E−61 | DUCTAL 2 |
| DYNLL1 | 2.11366075 | 1.45E−154 | 3.23E−115 | 4.06E−113 | DUCTAL 2 |
| SPP1 | 2.0999975 | 7.01E−209 | 4.55E−85 | 3.61E−45 | DUCTAL 2 |
| KLF6 | 2.06789809 | 1.27E−56 | 1.32E−46 | 1.95E−29 | DUCTAL 2 |
| KRT8 | 2.05871288 | 2.60E−151 | 4.20E−122 | 2.48E−85 | DUCTAL 2 |
| 2810417H13RIK | 2.74928489 | 4.32E−174 | 3.96E−132 | 1.50E−227 | PROLIF. |
| | | | | | DUCTAL |
| SPC25 | 2.49675899 | 4.26E−126 | 8.09E−119 | 4.77E−180 | PROLIF. |
| | | | | | DUCTAL |
| CDK1 | 2.34683179 | 9.38E−127 | 9.50E−113 | 3.30E−200 | PROLIF. |
| | | | | | DUCTAL |
| TOP2A | 2.26884946 | 1.54E−104 | 2.27E−106 | 7.24E−191 | PROLIF. |
| | | | | | DUCTAL |
| RRM2 | 2.267689 | 2.92E−105 | 6.25E−95 | 1.37E−149 | PROLIF. |
| | | | | | DUCTAL |
| NUSAP1 | 2.18384483 | 6.65E−74 | 6.04E−61 | 2.89E−120 | PROLIF. |
| | | | | | DUCTAL |
| LIG1 | 2.13873889 | 2.34E−157 | 1.86E−108 | 2.34E−139 | PROLIF. |
| | | | | | DUCTAL |
| TK1 | 2.08401561 | 8.64E−139 | 1.94E−113 | 6.38E−134 | PROLIF. |
| | | | | | DUCTAL |
| PRC1 | 2.06306819 | 1.86E−100 | 6.48E−94 | 5.89E−143 | PROLIF. |
| | | | | | DUCTAL |
| GMNN | 2.0565317 | 1.36E−139 | 1.16E−101 | 3.68E−131 | PROLIF. |
| | | | | | DUCTAL |
| UBE2C | 2.03068945 | 9.47E−32 | 1.05E−30 | 8.08E−78 | PROLIF. |
| | | | | | DUCTAL |
| SPP1 | 2.02645622 | 3.97E−192 | 1.83E−72 | 3.52E−142 | PROLIF. |
| | | | | | DUCTAL |
| UBE2C | 6.03181715 | 0 | 5.42E−242 | 1.64E−227 | PROLIF |
| | | | | | ACINAR |
| CCNB1 | 3.26754701 | 1.83E−220 | 2.37E−186 | 3.95E−160 | PROLIF |
| | | | | | ACINAR |
| NUSAP1 | 2.83235587 | 1.11E−245 | 0 | 1.30E−118 | PROLIF |
| | | | | | ACINAR |
| CKS2 | 2.77358962 | 1.00E−225 | 2.38E−157 | 6.00E−144 | PROLIF |
| | | | | | ACINAR |
| TOP2A | 2.71752364 | 6.13E−189 | 7.99E−197 | 1.58E−78 | PROLIF |
| | | | | | ACINAR |
| CDK1 | 2.68799936 | 2.08E−194 | 2.37E−166 | 9.37E−97 | PROLIF |
| | | | | | ACINAR |
| ARL6IP1 | 2.65718331 | 9.56E−188 | 5.30E−132 | 2.69E−251 | PROLIF |
| | | | | | ACINAR |
| SPC25 | 2.63538288 | 1.11E−185 | 1.41E−199 | 9.33E−100 | PROLIF |
| | | | | | ACINAR |
| AURKA | 2.53276828 | 7.74E−210 | 4.27E−285 | 8.79E−95 | PROLIF |
| | | | | | ACINAR |
| BIRC5 | 2.52279362 | 1.76E−187 | 2.62E−148 | 4.28E−116 | PROLIF |
| | | | | | ACINAR |
| H2AFX | 2.47566376 | 2.88E−177 | 1.53E−138 | 1.65E−130 | PROLIF |
| | | | | | ACINAR |
| PLK1 | 2.47141197 | 1.31E−207 | 5.53E−269 | 7.24E−99 | PROLIF |
| | | | | | ACINAR |
| CDC20 | 2.34020305 | 2.00E−124 | 5.61E−129 | 1.37E−80 | PROLIF |
| | | | | | ACINAR |
| CDCA8 | 2.33746543 | 2.01E−156 | 2.54E−147 | 4.73E−107 | PROLIF |
| | | | | | ACINAR |
| TUBA1C | 2.21424053 | 9.33E−142 | 7.67E−138 | 2.75E−119 | PROLIF |
| | | | | | ACINAR |
| PBK | 2.20226659 | 2.87E−127 | 6.69E−130 | 4.55E−43 | PROLIF |
| | | | | | ACINAR |
| CENPF | 2.17584059 | 2.82E−160 | 3.96E−196 | 2.16E−96 | PROLIF |
| | | | | | ACINAR |
| HMGB2 | 2.15815702 | 3.79E−210 | 4.89E−131 | 6.83E−39 | PROLIF |
| | | | | | ACINAR |
| HMMR | 2.11885246 | 1.30E−156 | 2.81E−190 | 3.61E−69 | PROLIF |
| | | | | | ACINAR |
| KIF22 | 2.116951 | 8.59E−168 | 1.27E−200 | 1.53E−93 | PROLIF |
| | | | | | ACINAR |
| BUB3 | 2.10519912 | 1.91E−143 | 9.04E−122 | 3.18E−142 | PROLIF |
| | | | | | ACINAR |
| PRC1 | 2.09521226 | 3.45E−180 | 6.32E−240 | 4.76E−72 | PROLIF |
| | | | | | ACINAR |
| SMC4 | 2.05604272 | 2.73E−130 | 1.67E−130 | 2.78E−75 | PROLIF |
| | | | | | ACINAR |
| AURKB | 2.03819235 | 9.01E−172 | 3.94E−222 | 5.57E−50 | PROLIF |
| | | | | | ACINAR |
| CTRB1 | 5.74993288 | 0 | 0 | 0 | ACINAR |
| PNLIPRP1 | 5.63326234 | 0 | 0 | 0 | ACINAR |
| CLPS | 5.53917954 | 0 | 0 | 0 | ACINAR |
| CPA2 | 5.37010671 | 0 | 0 | 0 | ACINAR |
| SERPINA6 | 5.08110441 | 0 | 0 | 0 | ACINAR |
| CPA1 | 5.05263211 | 0 | 0 | 0 | ACINAR |
| NUPR1 | 4.89032011 | 0 | 0 | 0 | ACINAR |
| REEP5 | 3.44111343 | 0 | 0 | 0 | ACINAR |
| CELA1 | 3.37399106 | 0 | 0 | 4.96E−249 | ACINAR |
| SERPINI2 | 3.23017738 | 0 | 0 | 9.63E−284 | ACINAR |
| SPINK1 | 3.21102208 | 0 | 0 | 1.09E−160 | ACINAR |
| CEL | 2.851163 | 0 | 0 | 8.66E−235 | ACINAR |
| GSTM1 | 2.81010809 | 0 | 0 | 6.21E−294 | ACINAR |
| PTF1A | 2.73621938 | 0 | 0 | 3.17E−219 | ACINAR |
| IFITM3 | 2.70942349 | 0 | 0 | 4.95E−299 | ACINAR |
| SEPP1 | 2.57552155 | 0 | 0 | 8.37E−208 | ACINAR |
| GCAT | 2.41434554 | 0 | 0 | 7.97E−176 | ACINAR |
| TMEM97 | 2.41052235 | 0 | 0 | 9.94E−186 | ACINAR |
| GGH | 2.34801558 | 0 | 0 | 1.20E−149 | ACINAR |
| GAMT | 2.32774802 | 0 | 0 | 4.37E−190 | ACINAR |
| FKBP11 | 2.27632269 | 0 | 0 | 2.15E−158 | ACINAR |
| XBP1 | 2.23150371 | 0 | 0 | 7.40E−164 | ACINAR |
| SERPINB1A | 2.18661438 | 0 | 0 | 7.18E−165 | ACINAR |
| ASNS | 2.17171072 | 0 | 0 | 2.53E−125 | ACINAR |
| DAP | 2.16225051 | 0 | 0 | 4.24E−167 | ACINAR |
| SERP1 | 2.13621614 | 0 | 0 | 3.23E−257 | ACINAR |
| VTN | 2.11827963 | 0 | 0 | 9.04E−177 | ACINAR |
| TMED6 | 2.10968474 | 0 | 3.72E−287 | 6.22E−125 | ACINAR |
| GC | 2.03635005 | 4.71E−306 | 6.56E−254 | 2.71E−111 | ACINAR |
| MEST | 2.00985467 | 9.94E−194 | 3.65E−171 | 6.62E−76 | ACINAR |

TABLE 5

| | | | | | | MEMBERS_{—} | | |
|---|---|---|---|---|---|---|---|---|
| | | - LOG | | | MEMBERS_{—} | INPUT_{—} | | EFFEC- |
| | | (P- | | | INPUT_{—} | OVERLAP_{—} | | TIVE_{—} |
| P-VALUE | Q-VALUE | VALUE) | PATHWAY | SOURCE | OVERLAP | GENEIDS | SIZE | SIZE |
| 4.91E−05 | 0.01218005 | 4.30880246 | MATURITY ONSET | KEGG | FOXA2; | 15242; 15376; | 26 | 26 |
| | | | DIABETES OF THE | | GCK; HHEX; | 103988; 15285 | | |
| | | | YOUNG - MUS | | MNX1 | 103988; 15285 | | |
| | | | MUSCULUS (MOUSE) | | MNX1 | | | |
| 0.00016694 | 0.01541533 | 3.77743739 | BIOGENIC AMINE | WIKI- | DDC; TPH1; | 21990; 14415; | 15 | 14 |
| | | | SYNTHESIS | PATHWAYS | GAD1 | 13195 | | |
| 0.00018686 | 0.01541533 | 3.7284933 | AMPHETAMINE | KEGG | DDC; ARC; | 12313; 14281; | 68 | 67 |
| | | | ADDICTION - MUS | | FOS; JUN; | 16476; 13195; | | |
| | | | MUSCULUS (MOUSE) | | CALM1 | 11838 | | |
| 0.00037295 | 0.01541533 | 3.42834762 | SEROTONIN AND | REACTOME | DDC; TPH1 | 13195; 21990 | 4 | 4 |
| | | | MELATONIN | | | | | |
| | | | BIOSYNTHESIS | | | | | |
| 0.00037295 | 0.01541533 | 3.42834762 | SEROTONIN AND | MOUSECYC | DDC; TPH1 | 13195; 21990 | 5 | 4 |
| | | | MELATONIN | | | | | |
| | | | BIOSYNTHESIS | | | | | |
| 0.00037295 | 0.01541533 | 3.42834762 | BIOSYNTHESIS OF | MOUSECYC | DDC; TPH1 | 13195; 21990 | 5 | 4 |
| | | | SEROTONIN AND | | | | | |
| | | | MELATONIN | | | | | |
| 0.00108385 | 0.03560609 | 2.96503052 | ESTROGEN SIGNALING | KEGG | HSP90AB1; | 12313; 16440; | 99 | 98 |
| | | | PATHWAY - | | ITPR3; FOS; | 16476; 15516; | | |
| | | | MUS MUSCULUS | | CALM1 | 14281 | | |
| | | | (MOUSE) | | | | | |
| 0.00114858 | 0.03560609 | 2.93983738 | PROTEASOME | WIKI- | PSMD4; | 57296; 19186; | 58 | 58 |
| | | | DEGRADATION | PATHWAYS | UBE2B; | 19185; 22210 | | |
| | | | | | PSMD8; | | | |
| | | | | | PSME1 | | | |
| 0.00153742 | 0.03843282 | 2.81320804 | MAP TARGETS/ | REACTOME | DUSP6; JUN; | 16476; 14281; | 29 | 29 |
| | | | NUCLEAR EVENTS | | FOS | 67603 | | |
| | | | MEDIATED BY MAP | | | | | |
| | | | KINASES | | | | | |
| 0.00160063 | 0.03843282 | 2.79570813 | MAPK SIGNALING | WIKI- | FOS; GCK; | 16476; 103988; | 158 | 158 |
| | | | PATHWAY | PATHWAYS | NR4A1; JUN; | 67603; 14281; | | |
| | | | | | DUSP5; | 15370; 240672 | | |
| | | | | | DUSP6 | | | |
| 0.00170468 | 0.03843282 | 2.76835674 | FOLDING OF ACTIN | REACTOME | CCT8; CCT5 | 12469; 12465 | 8 | 8 |
| | | | BY CCT/TRIC | | | | | |
| 0.00271143 | 0.05260961 | 2.56680228 | ACTIVATION OF | REACTOME | JUN; FOS | 16476; 14281 | 10 | 10 |
| | | | THE AP-1 FAMILY | | | | | |
| | | | OF TRANSCRIPTION | | | | | |
| | | | FACTORS | | | | | |
| 0.00275776 | 0.05260961 | 2.55944322 | EGFR1 SIGNALING | WIKI- | FOS; GRB10; | 16476; 16668; | 176 | 176 |
| | | | PATHWAY | PATHWAYS | JUN; SHOC2; | 14281; 14786; | | |
| | | | | | GRB7; KRT18 | 14783; 56392 | | |
| 0.00393581 | 0.06325448 | 2.40496559 | SEROTONIN AND | WIKI- | ARC; FOS | 11838; 14281 | 12 | 12 |
| | | | ANXIETY-RELATED | PATHWAYS | | | | |
| | | | EVENTS | | | | | |
| 0.00393581 | 0.06325448 | 2.40496559 | TETRAHYDRO- | REACTOME | GCH1; | 14528; 12313 | 12 | 12 |
| | | | BIOPTERIN (BH4) | | CALM1 | | | |
| | | | SYNTHESIS, RECYCLING, | | | | | |
| | | | SALVAGE AND | | | | | |
| | | | REGULATION | | | | | |
| 0.00462743 | 0.06325448 | 2.33466029 | FORMATION OF ATP | REACTOME | ATP5B; | 11947; 11946 | 15 | 13 |
| | | | BY CHEMIOSMOTIC | | ATP5A1 | | | |
| | | | COUPLING | | | | | |
| 0.00511433 | 0.06325448 | 2.29121128 | PROTEASOME - MUS | KEGG | PSMD4; | 57296; 19185; | 44 | 44 |
| | | | MUSCULUS (MOUSE) | | PSMD8; | 19186 | | |
| | | | | | PSME1 | | | |
| 0.00537084 | 0.06325448 | 2.26995755 | AMINE-DERIVED | REACTOME | DDC; TPH1 | 21990; 13195 | 14 | 14 |
| | | | HORMONES | | | | | |
| 0.00548654 | 0.06325448 | 2.26070114 | TRIF-MEDIATED | REACTOME | DUSP6; JUN; | 16476; 14281; | 89 | 89 |
| | | | TLR3/TLR4 SIGNALING | | UBB; FOS | 67603; 22187 | | |
| 0.00548654 | 0.06325448 | 2.26070114 | MYD88- | REACTOME | DUSP6; JUN; | 16476; 14281; | 89 | 89 |
| | | | INDEPENDENT | | UBB; FOS | 67603; 22187 | | |
| | | | CASCADE | | | | | |
| 0.00548654 | 0.06325448 | 2.26070114 | TOLL LIKE | REACTOME | DUSP6; JUN; | 16476; 14281; | 89 | 89 |
| | | | RECEPTOR 3 (TLR3) | | UBB; FOS | 67603; 22187 | | |
| | | | CASCADE | | | | | |
| 0.00561128 | 0.06325448 | 2.2509377 | SIGNALING BY | REACTOME | ITPR3; | 22187; 15370; | 144 | 143 |
| | | | ERBB2 | | NR4A1; | 14786; 12313; | | |
| | | | | | GRB7; UBB; | 16440 | | |
| | | | | | CALM1 | | | |
| 0.00615543 | 0.06637157 | 2.21074172 | SELENIUM | WIKI- | SARS; JUN; | 16476; 20226; | 47 | 47 |
| | | | METABOLISM- | PATHWAYS | FOS | 14281 | | |
| | | | SELENOPROTEINS | | | | | |
| 0.00652849 | 0.06746104 | 2.18518738 | MAP KINASE | REACTOME | DUSP6; JUN; | 16476; 14281; | 48 | 48 |
| | | | ACTIVATION IN | | FOS | 67603 | | |
| | | | TLR CASCADE | | | | | |
| 0.00690134 | 0.06846131 | 2.16106648 | ACTIVATED TLR4 | REACTOME | DUSP6; JUN; | 16476; 14281; | 95 | 95 |
| | | | SIGNALLING | | UBB; FOS | 67603; 22187 | | |
| 0.00742183 | 0.07012614 | 2.12948882 | CIRCADIAN | KEGG | CACNA1H; | 12313; 16440; | 99 | 97 |
| | | | ENTRAINMENT - MUS | | ITPR3; FOS; | 58226, 14281 | | |
| | | | MUSCULUS (MOUSE) | | CALM1 | | | |
| 0.0076347 | 0.07012614 | 2.117208 | FC EPSILON | REACTOME | ITPR3; | 16476; 14281; | 155 | 154 |
| | | | RECEPTOR (FCERI) | | NR4A1; JUN; | 15370; 12313; | | |
| | | | SIGNALING | | CALM1; FOS | 16440 | | |
| 0.00884565 | 0.07834717 | 2.05327032 | SEROTONIN AND | WIKI- | ARC; FOS | 11838; 14281 | 19 | 18 |
| | | | ANXIETY | PATHWAYS | | | | |
| 0.00983551 | 0.08036762 | 2.00720299 | ENOS ACTIVATION | REACTOME | GCH1; | 14528; 12313 | 19 | 19 |
| | | | AND REGULATION | | CALM1 | | | |
| 0.00983551 | 0.08036762 | 2.00720299 | METABOLISM OF | REACTOME | GCH1; | 14528; 12313 | 19 | 19 |
| | | | NITRICOXIDE | | CALM1 | | | |

What is claimed is:

1. A method of enriching the pancreatic endocrine progenitor cell population in a cell sample comprising
(a) detecting cells in the sample expressing a pancreatic endocrine progenitor cell marker; and
(b) separating a pancreatic endocrine progenitor cell from at least one cell that does not express the pancreatic endocrine progenitor cell marker, thereby enriching the pancreatic endocrine progenitor cell population of the cell sample.

2. The method of claim 1, wherein the pancreatic endocrine progenitor cell is a human cell.

3. The method of claim 1, wherein the pancreatic endocrine progenitor cell is an alpha cell progenitor, a beta cell progenitor, a delta cell progenitor, a PP cell progenitor, or an epsilon cell progenitor.

4. The method of claim 3, wherein the pancreatic endocrine progenitor cell marker is the E26 transformation-specific transcription factor Fev.

5. The method of claim 4, wherein the pancreatic endocrine progenitor cell is a beta cell progenitor.

6. The method of claim 5, wherein the Fev⁺ beta cell progenitor further comprises Gng12⁺, Tssc4⁺, Ece1⁺, Tmcm108⁺, Wipi1⁺, or Papss2⁺.

7. The method of claim 6, wherein the beta cell progenitor is Fev⁺, Gng12⁺.

8. The method of claim 5, wherein the Fev⁺ beta cell progenitor further comprises Pax4⁺, Chga⁺, Chgb⁺, Neurod1⁺, Runx1t1⁺, or Vim⁺.

9. The method of claim 5 wherein the Fev⁺ beta cell progenitor does not express detectable Ngn3, Ins1 or Gcg.

10. The method of claim 9, wherein the beta cell progenitor is Fev⁺, Ngn⁻.

11. The method of claim 10, wherein the Fev⁺, Ngn⁻ beta cell progenitor expresses a gene in the serotonin pathway, the insulin signaling pathway, sphingosine-1-phosphate signaling pathway, or Activating Transcription Factor-2.

12. The method of claim 5, wherein the Fev⁺ beta cell progenitor further comprises Pdx1⁺ or Mafb⁺.

13. The method of claim 1, wherein the at least one cell that does not express the pancreatic endocrine progenitor cell marker is a CD140⁺ mesenchyme cell.

14. The method of claim 5, wherein the beta cell progenitor cell is a human cell.

15. A method of producing a pancreatic endocrine progenitor cell comprising culturing a stem cell under conditions that induce differentiation of the stem cell into a pancreatic endocrine progenitor cell comprising the E26 transformation-specific transcription factor Fev.

16. The method of claim 15, wherein the stem cell is an embryonic stem cell (ESC) or an inducible pluripotent stem cell (iPSC).

17. The method of claim 15, wherein the pancreatic endocrine progenitor cell is an alpha cell progenitor, a beta cell progenitor, a delta cell progenitor, a PP cell progenitor, or an epsilon cell progenitor.

18. The method of claim 17, wherein the pancreatic endocrine progenitor cell is a beta cell progenitor.

19. The method of claim 18, wherein the Fev⁺ beta cell progenitor further comprises Gng12⁺, Tssc4⁺, Ece1⁺, Tmcm108⁺, Wipi1⁺, or Papss2⁺.

20. The method of claim 19, wherein the beta cell progenitor is Fev⁺, Gng12⁺.

21. The method of claim 18, wherein the Fev⁺ beta cell progenitor further comprises Pax4⁺, Chga⁺, Chgb⁺, Neurod1⁺, Runx1t1⁺, or Vim⁺.

22. The method of claim 18 wherein the Fev⁺ beta cell progenitor does not express detectable Ngn3, Ins1 or Gcg.

23. The method of claim 22, wherein the beta cell progenitor is Fev⁺, Ngn⁻.

24. The method of claim 23, wherein the Fev⁺, Ngn⁻ beta cell progenitor expresses a gene in the serotonin pathway, the insulin signaling pathway, sphingosine-1-phosphate signaling pathway, or Activating Transcription Factor-2.

25. The method of claim 18, wherein the Fev⁺ beta cell progenitor further comprises Pdx1⁺ or Mafb⁺.

26. The method of claim 18, wherein the Fev⁺ beta cell progenitor is a human cell.

27. An isolated Fev⁺ pancreatic endocrine progenitor cell produced according to claim 17 or claim 18.

28. A method of inducing formation of a hormone-producing cell comprising contacting a progenitor of a hormone-producing cell with an effective amount of Fev to produce a hormone-producing cell.

29. The method of claim 28, wherein the hormone-producing cell is a INS+ cell.

30. The method of claim 29, wherein the hormone-producing progenitor cell is an ES4 cell.

31. The method of claim 28, wherein the hormone-producing cell is a beta cell.

32. The method of claim 31, wherein the hormone-producing progenitor cell is a beta-like cell.

33. The method of claim 28, wherein the method is performed in vitro.

34. The method of claim 28 further comprising removing a cell expressing at least one of PHOX2A, TLX2 or TBX2.

35. A method of screening for a signaling compound that induces FEV+ progenitor cell replication comprising:
(a) contacting a FEV+ progenitor cell with a candidate compound;
(b) culturing the FEV+ progenitor cell under conditions suitable for cell proliferation;
(c) measuring the cell proliferation of the FEV+ progenitor cell in the presence or absence of the candidate compound; and
(d) identifying the compound as a signaling compound for FEV+ progenitor cell proliferation if the cell proliferation in the presence of the compound is greater than the cell proliferation in the absence of the compound.

36. The method of claim 35 wherein the FEV+ progenitor cell is a FEV-MYC progenitor cell, a FEV-GFP progenitor cell, a FEV-KO progenitor cell, or a FEV-tNFGR progenitor cell.

37. A method of screening for a signaling compound that enhances FEV+ progenitor cell differentiation into beta cells comprising:
(a) contacting FEV+ progenitor cells with a candidate compound;
(b) incubating the FEV+ progenitor cells under conditions suitable for cell differentiation;
(c) measuring the level of differentiation of the FEV+ progenitor cells to beta cells in the presence or absence of the candidate compound; and
(d) identifying the compound as a signaling compound for FEV+ progenitor cell differentiation into beta cells if the cell differentiation in the presence of the compound is greater than the cell differentiation in the absence of the compound.

38. The method of claim 37 wherein the FEV+ progenitor cell is a FEV-MYC progenitor cell, a FEV-GFP progenitor cell, a FEV-KO progenitor cell, or a FEV-tNFGR progenitor cell.
